(19) United States
(12) Patent Application Publication
(10) Pub. No.: US 2024/0025891 A1
(43) Pub. Date: Jan. 25, 2024

(54) HETEROCYCLIC COMPOUNDS AND THEIR USE FOR TREATMENT OF HELMINTHIC INFECTIONS AND DISEASES

(71) Applicants: Celgene Corporation, Summit, NJ (US); Zoetis LLC, Kalamazoo, MI (US)

(72) Inventors: Natalie Hawryluk, San Diego, CA (US); Stacie Canan, La Jolla, CA (US); Kevin R. Condroski, Lafayette, CO (US); Matthew Bedore, Portage, MI (US); Graham Kyne, Kalamazoo, MI (US); Sanjay Menon, Kalamazoo, MI (US)

(21) Appl. No.: 18/032,998

(22) PCT Filed: Oct. 22, 2021
(86) PCT No.: PCT/US2021/056127

§ 371 Date: Apr. 20, 2023

Related U.S. Application Data
(60) Provisional application No. 63/105,013, filed on Oct. 23, 2020.

(51) Int. Cl. C07D 417/14; C07D 471/04; C07D 491/048; C07D 401/14; C07D 413/14; A61P 33/10; C07D 487/04; C07D 491/056; A61K 45/06; A61K 31/444; A61K 31/437; A61K 31/496; A61K 31/4545; A61K 31/497; A61K 31/5377; A61K 31/506; A61K 31/4355; A61K 31/501; A61K 31/4985

(52) U.S. Cl. CPC C07D 417/14; C07D 471/04; C07D 491/048; C07D 401/14; C07D 413/14; A61P 33/10; C07D 487/04; C07D 491/056; A61K 45/06; A61K 31/444; A61K 31/437; A61K 31/496; A61K 31/4545; A61K 31/497; A61K 31/5377; A61K 31/506; A61K 31/4355; A61K 31/501; A61K 31/4985

(57) ABSTRACT

Provided herein are Heterocyclic Compounds of formula (I), formula (II), formula (III), formula (IlIa), formula (Mb), formula (IIIc), formula (Hid), formula (IV), formula (IVa), formula (IVb), and formula (IVc), and pharmaceutically acceptable salts, tautomers, isotopologues, and stereoisomers thereof, compositions comprising an effective amount of a Heterocyclic Compound of formula (I), formula (II), formula (III), formula (IlIa), formula (Mb), formula (IIIc), formula (Hid), formula (IV), formula (IVa), formula (IVb), and formula (IVc), and methods for treating or preventing animal and human filarial worm infections and diseases.

### CROSS REFERENCE TO RELATED APPLICATIONS

This application claims the benefit of U.S. Provisional Application No. 63/105,013, filed Oct. 23, 2020, the disclosure of which is incorporated herein by reference in its entirety.

### FIELD

Disclosed herein are compounds and methods for the prevention and/or treatment of helminthic infections and diseases caused by helminthic infection. Also provided herein are such compounds for use in such methods. Also disclosed herein are pharmaceutical compositions comprising such compounds for use in such methods of preventing or treating helminthic infection and/or diseases associated with helminthic infection.

### BACKGROUND

There are several types of parasitic worms (helminths), with the most common worldwide the intestinal nematodes or soil-transmitted helminths (STH), schistosomes (parasites of schistosomiasis) and filarial worms, which cause lymphatic filariasis (LF) and onchocerciasis. Filariasis is a parasitic disease that is caused by thread-like filarial nematodes or roundworms. Filariasis is a vector-borne disease that is transmitted via insect bites. Infective larvae of the nematodes can be introduced into the human body via bites of blood sucking insects like mosquitoes or flies. Filariasis can also affect domestic animals like dogs. In dogs, dirofilariasis which is also called heartworm disease, is caused by nematodes called Dirofilaria immitis and Dirofilaria repens. Dirofilariasis is considered endemic in 49 states of the United States. The vectors as well are blood sucking insects like mosquitoes.

The major causes of human filariasis are the filarial nematodes Wuchereria bancrofti, Brugia malayi, Brugia timori, Onchocerca volvulus and Mansonella species that have human hosts. The nematodes Wuchereria bancrofti, Brugia malayi and Onchocerca volvulus are responsible for most of the debilitating filarial infections in more than 80 developing countries of the tropics and sub-tropics where 1.1 billion are at risk of infection and about 150 million are infected. All three species are a source of severe pathologies that result in high morbidity and increased mortality. The infection can cause severe morbidity in up to 50% of those infected with the nematodes.

W. bancrofti and B. malayi infections can develop into lymphatic filariasis, often seen as hydrocoele in men and/or lymphoedema and in extreme cases elephantiasis. O. volvulus infections can develop into severe dermatitis and/or onchocerciasis, the visual impairment giving the latter disease its common name River Blindness. Community directed mass drug administration programs are designed to control these infections and eliminate them as a public health problem.

Current efforts aim to eliminate these parasitic nematodes through the use of drugs like diethylcarbamazine, ivermectin, and albendazole that kill the larvae, but not the adult worms. The antihelmintic drug diethylcarbamazine is used to combat lymphatic filariasis in countries without co-endemic O. volvulus infections, i.e. outside of Africa. Ivermectin is used to combat onchocerciasis. The greatest efficacy of both drugs is against the first stage larvae found in the blood stream or in the dermis. Since the worms can live up to 14 years and are fecund for most of their lifespan, populations in endemic regions must be treated with high coverage (at least 65%) for many years to break transmission of the disease to uninfected persons.

Two of the major constraints of treatment of filarial diseases are (i) the absence of a macrofilaricidal drug (or for onchocerciasis, one which permanently sterilizes the worm) and (ii) the risk of worms developing drug-resistance. For example, currently available treatments for onchocerciasis include ivermectin, which kills worm larvae, but has little or no activity against adult Onchocerca volvulus parasites. Thus, infected patients must be retreated with ivermectin for several years until the adult worms die naturally. In addition, there are also potential signs of resistance to ivermectin within the parasite in a few areas. Osei-Atweneboana M Y, et al., Phenotypic Evidence of Emerging Ivermectin Resistance in Onchocerca volvulus, PLoS Negl Trop Dis 5(3): e998 (2011). In addition, there is a danger in treating patients co-infected with both (i) Wuchereria bancrofti, Brugia malayi, Brugia timori, and/or Onchocerca volvulus; and (ii) Loa loa with ivermectin. In such co-infected patients, ivermectin treatment can cause severe reactions, including encephalopathy, leading to coma or even death.

Heartworm infection, caused by the endoparasite Dirofilaria immitis (D. immitis), can be a severe and life-threatening disease in animals such as dogs and cats. Heartworm has a complicated life cycle involving several life stages before they mature into adults that will eventually infect the pulmonary artery of the host animal. Heartworm transmission also requires the mosquito to act as an intermediate host to complete this life cycle. For example, the beginning of the heartworm life cycle and transmission process involves a mosquito biting a previously infected dog and ingesting blood containing heartworm microfilariae (larva stage 1). Within the mosquito, the microfilariae will molt into infective larva stage 3 (L3) worms over a two week period. Once the mosquito bites another dog, infective L3 worms will move through the bite wound to enter the host and migrate into the tissues where they will begin molting into larva stage 4 (L4) worms, usually within 1 to 3 days post infection. Subsequently, L4 worms will continue their migration through tissues and molt into sexually immature or “adolescent” adults (larva stage 5, immature adult), approximately 50-70 days post infection. Sexually mature worms will eventually migrate to the heart and lungs of the dog, as early as 70 days post infection. Approximately 6-7 months post infection D. immitis adults reach maturity and sexually reproduce in the pulmonary artery leading to microfilaria (MF) production and circulation in the blood of the dog, thus completing the heartworm life cycle.

The most commonly used heartworm preventatives are the macrocyclic lactones (MLs) such as ivermectin, moxidectin and selamectin. These agents are administered on a monthly basis whereby they kill D. immitis L3 and L4 worms acquired by the host within the previous 30 days. Their primary action is to disrupt the heartworm life cycle by killing L3 and L4 worms thus preventing adult formation and subsequent disease. While very effective at preventing heartworm disease, owners are advised to test dogs for existing heartworm infections (i.e. heartworm positive dogs) prior to starting treatment with MLs due to their potential to kill circulating microfilariae. A rapid decrease in the numbers of microfilariae in the blood can lead to hypersensitivity-type reactions and circulatory shock (e.g. anaphylaxis), presumably due to dead or dying microfilariae. These potential adverse effects can be life-threatening to the dog and as such are presented as caution statements on many ML product labels. Therefore, the discovery of a novel heartworm preventative that would selectively target L3 and L4 stage worms versus microfilariae would offer a potential safety advantage. By not killing circulating microfilariae in heartworm positive dogs, a targeted treatment would prevent the adverse effects known to occur with other heartworm preventatives that lack D. immitis stage selectivity.

Thus, alternative, and more effective, treatments for filarial worm diseases are needed.

Citation or identification of any reference in this application is not to be construed as an admission that the reference is prior art to the present application.

### SUMMARY

Provided herein are compounds of formula (I):

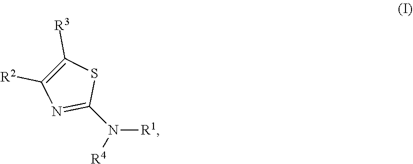

and pharmaceutically acceptable salts, tautomers, isotopologues, and stereoisomers thereof, wherein R¹, R², R³ and R⁴ are as defined herein.

Also provided herein are compounds of formula (II):

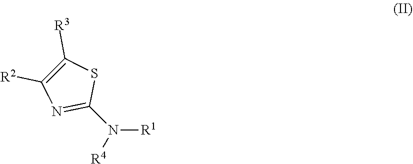

and pharmaceutically acceptable salts, tautomers, isotopologues, and stereoisomers thereof, wherein R¹, R², R³ and R⁴ are as defined herein.

Also provided herein are compounds of formula (III):

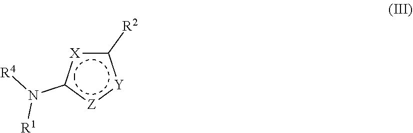

and pharmaceutically acceptable salts, tautomers, isotopologues, and stereoisomers thereof, wherein X, Y, Z, R¹, R², R³ and R⁴ are as defined herein.

Also provided herein are compounds of formula (IV):

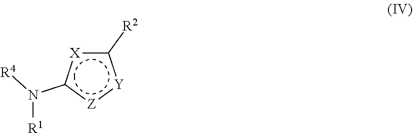

and pharmaceutically acceptable salts, tautomers, isotopologues, and stereoisomers thereof, wherein X, Y, Z, R¹, R², R³ and R⁴ are as defined herein.

In one aspect, provided herein are Heterocyclic Compounds as described in the instant disclosure, such as, for example, a Heterocyclic Compound of formula (I), formula (II), formula (III), formula (IIIa), formula (IIIb), formula (IIIc), formula (IIId), formula (IV), formula (IVa), formula (IVb), or formula (IVc), or a compound from Table 1, Table 2, Table 3, Table 4, or Table 5.

In one aspect, provided herein are pharmaceutical compositions comprising an effective amount of a Heterocyclic Compound, as described herein, and a pharmaceutically acceptable carrier, excipient or vehicle. In some embodiments the pharmaceutical composition is suitable for oral, parenteral, mucosal, transdermal or topical administration.

In one aspect, provided herein are methods of treating a subject infected with a helminth. In another aspect, provided herein are uses of Heterocyclic Compounds for treating or preventing helminthic infections, comprising administering to a subject affected by helminthic infections an effective amount of a Heterocyclic Compound as described herein. In one aspect the helminthic infection is a filarial infection.

In one aspect, provided herein are methods of treating a subject infected with a filarial worm. In another aspect, provided herein are uses of Heterocyclic Compounds for treating or preventing filarial infections, comprising administering to a subject affected by filarial infections an effective amount of a Heterocyclic Compound as described herein.

In certain embodiments, the methods described herein include administering a therapeutically effective amount of a compound of formula (I), (II), (III), (IIIa), (IIIb), (IIIc), (IIId), (IV), (IVa), (IVb), (IVc), or a compound from Table 1, Table 2, Table 3, Table 4, or Table 5, or a pharmaceutically acceptable salt, tautomer, isotopologue, or stereoisomer thereof, to the subject.

The compounds of the present invention are useful for the treatment of helminthic diseases where the helminths are categorized as cestodes (tapeworms), nematodes (roundworms) and trematodes (flatworms or flukes). Non-limiting examples of filarial nematodes within the Onchocercidae family include the genus Brugia spp. (i.e., B. malayi, B. pahangi, B. timori, and the like), Wuchereria spp. (i.e., W. bancrofti, and the like), Dirofilaria spp. (D. immitis, D. repens, D. ursi, D. tenuis, D. spectans, D. lutrae, and the like), Dipetalonema spp. (i.e., D reconditum, D. repens, and the like), Onchocerca spp. (i.e., O. gibsoni, O. gutturosa, O. volvulus, and the like), Elaeophora spp. (E. bohmi, E. elaphi, E. poeli, E. sagitta, E. schneideri, and the like), Mansonella spp. (i.e., M. ozzardi, M. perstans, and the like), and Loa spp. (i.e., L. loa). In certain embodiments, the filarial worm is Onchocerca volvulus. In certain embodiments, the filarial worm is Wuchereria bancrofti. In certain embodiments, the filarial worm is Brugia malayi. In certain embodiments, the filarial worm is Brugia timori. In certain embodiments, the filarial worm is Mansonella. In certain embodiments, the filarial worm is Dirofilaria immitis.

In one aspect, provided herein are uses of Heterocyclic Compounds for treating or preventing helminthic infections, comprising administering to a subject affected by helminthic infection an effective amount of a Heterocyclic Compound as described herein. In another aspect, provided herein are uses of Heterocyclic Compounds for treating or preventing filarial worm infections, wherein the methods comprise administering to a subject affected by filarial worm infections an effective amount of a Heterocyclic Compound as described herein.

In one aspect, provided herein is a Heterocyclic Compound for use as a medicament. In a particular embodiment, provided herein is the Heterocyclic Compound for use in a method for the treatment or prevention of a helminthic infection, the method comprising administering to a subject an effective amount of the Heterocyclic Compound. In a particular embodiment, provided herein is the Heterocyclic Compound for use in a method for the treatment or prevention of a filarial worm infection, the method comprising administering to a subject an effective amount of the Heterocyclic Compound.

In another aspect provided herein are methods for preparing Heterocyclic Compounds as described herein.

The present embodiments can be understood more fully by reference to the detailed description and examples, which are intended to exemplify non-limiting embodiments.

### BRIEF DESCRIPTION OF THE FIGURES

FIG. 1 shows the L. sigmodontis (a rodent filarial nematode) life cycle from microfilariae (L1) to adult stage.

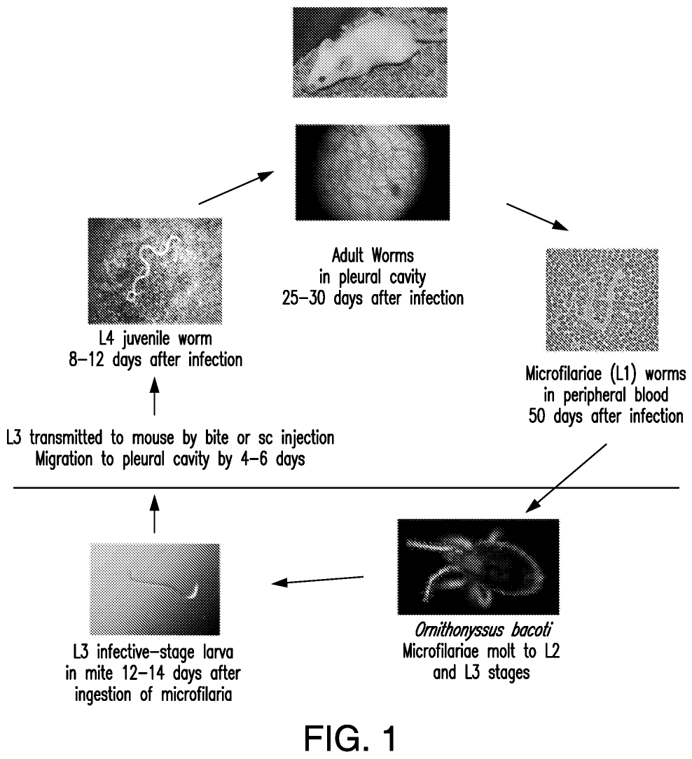

### DETAILED DESCRIPTION

### Definitions

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

As used herein and unless otherwise specified, an “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 or carbon atoms. Representative alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. An “alkenyl” group is an alkyl group that contains one or more carbon-carbon double bonds. An “alkynyl” group is an alkyl group that contains one or more carbon-carbon triple bonds. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₂CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen; hydroxy; alkoxy; cycloalkyloxy, aryloxy, heterocyclyloxy, heteroaryloxy, heterocycloalkyoxy, cycloalkylalkyloxy, aralkyloxy, heterocyclylalkyloxy, heteroarylalkyloxy, heterocycloalkyalkyloxy; oxo (═O); amino, alkylamino, cycloalkylamino, arylamino, heterocyclylamino, heteroarylamino, heterocycloalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂.

As used herein and unless otherwise specified, a “cycloalkyl” group is a saturated, or partially saturated cyclic alkyl group of from 3 to 10 carbon atoms having a single cyclic ring or multiple condensed or bridged rings which can be optionally substituted. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

As used herein and unless otherwise specified, an “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryl groups include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

As used herein and unless otherwise specified, a “heteroaryl” group is an aromatic ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indol-2-onyl), isoindolin-1-onyl, azaindolyl, pyrrolopyridyl (e.g., 1H-pyrrolo[2,3-b]pyridyl), indazolyl, benzimidazolyl (e.g., 1H-benzo[d]imidazolyl), azabenzimidazolyl, imidazopyridyl (e.g., 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl, 3,4-dihydroisoquinolin-1(2H)-onyl, tetrahydroquinolinyl, quinoxalinyl, and quinazolinyl groups. A heteroaryl group can be substituted or unsubstituted.

As used herein and unless otherwise specified, a “heterocyclyl” is an aromatic ring system (also referred to as heteroaryl) or non-aromatic cycloalkyl (also referred to as heterocycloalkyl) in which one to four of the ring carbon atoms are independently replaced with a heteroatom. Suitable heteroatoms include oxygen, sulfur and nitrogen. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. Heterocyclyl groups encompass unsaturated, partially saturated and saturated ring systems, such as, for example, imidazolyl, imidazolinyl and imidazolidinyl (e.g., imidazolidin-4-onyl or imidazolidin-2,4-dionyl) groups. The phrase heterocyclyl includes fused ring species, including those comprising fused aromatic and non-aromatic groups, such as, for example, 1- and 2-aminotetraline, benzotriazolyl (e.g., 1H-benzo[d][1,2,3]triazolyl), benzimidazolyl (e.g., 1H-benzo[d]imidazolyl), 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, azepanyl, oxetanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, benzisoxazolyl (e.g., benzo[d]isoxazolyl), thiazolyl, thiazolinyl, isothiazolyl, thiadiazolyl, oxadiazolyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dioxyl, dithianyl, pyranyl, pyridyl, pyrimidyl, pyridazinyl, pyrazinyl, triazinyl, dihydropyridyl, dihydrodithiinyl, dihydrodithionyl, 1,4-dioxaspiro[4.5]decanyl, homopiperazinyl, quinuclidyl, indolyl (e.g., indol-2-onyl), isoindolin-1-onyl, indolinyl, isoindolyl, isoindolinyl, azaindolyl, pyrrolopyridyl (e.g, 1H-pyrrolo[2,3-b]pyridyl), indazolyl, indolizinyl, benzotriazolyl (e.g. 1H-benzo[d][1,2,3]triazolyl), benzimidazolyl (e.g., 1H-benzo[d]imidazolyl or 1H-benzo[d]imidazol-2(3H)-onyl), benzofuranyl, benzothiophenyl, benzothiazolyl, benzoxadiazolyl, benzoxazinyl, benzodithiinyl, benzoxathiinyl, benzothiazinyl, benzoxazolyl (e.g., benzo[d]oxazolyl), benzothiazolyl, benzothiadiazolyl, benzo[1,3]dioxolyl, pyrazolopyridyl (e.g., 1H-pyrazolo[3,4-b]pyridyl, 1H-pyrazolo[4,3-b]pyridyl), azabenzimidazolyl, imidazopyridyl (e.g., 1H-imidazo[4,5-b]pyridyl), triazolopyridyl, isoxazolopyridyl, purinyl, xanthinyl, adeninyl, guaninyl, quinolinyl, isoquinolinyl, 3,4-dihydroisoquinolin-1(2H)-onyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, tetrahydropyrimidin-2(1H)-one and tetrahydroquinolinyl groups. Representative non-aromatic heterocyclyl groups do not include fused ring species that comprise a fused aromatic group. Examples of non-aromatic heterocyclyl groups include aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, or tetrahydropyrimidin-2(1H)-one. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

As used herein and unless otherwise specified, a “cycloalkylalkyl” group is a radical of the formula: -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopropylmethyl, cyclobutylmethyl, cyclopentylmethyl, cyclohexylmethyl, cyclopropylethyl, cyclobutylethyl, cyclopentylethyl, cyclohexylethyl, cyclopentylpropyl, cyclohexylpropyl and the like.

As used herein and unless otherwise specified, an “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and aralkyl groups wherein the aryl group is fused to a cycloalkyl group such as indan-4-yl ethyl.

As used herein and unless otherwise specified, a “heterocyclylalkyl” group is a radical of the formula: -alkyl-heterocyclyl, wherein alkyl and heterocyclyl are defined above. A “heteroarylalkyl” group is a radical of the formula: -alkyl-heteroaryl, wherein alkyl and heteroaryl are defined above. A “heterocycloalkylalkyl” group is a radical of the formula: -alkyl-heterocycloalkyl, wherein alkyl and heterocycloalkyl are defined above. Substituted heterocyclylalkyl groups may be substituted at the alkyl, the heterocyclyl, or both the alkyl and the heterocyclyl portions of the group. Representative heterocylylalkyl groups include but are not limited to morpholin-4-yl ethyl, morpholin-4-yl propyl, furan-2-yl methyl, furan-3-yl methyl, pyridin-3-yl methyl, tetrahydrofuran-2-yl ethyl, and indol-2-yl propyl.

As used herein and unless otherwise specified, a “halogen” is fluorine, chlorine, bromine or iodine.

As used herein and unless otherwise specified, a “hydroxyalkyl” group is an alkyl group as described above substituted with one or more hydroxy groups.

As used herein and unless otherwise specified, an “alkoxy” group is —O-(alkyl), wherein alkyl is defined above. An “alkylthio” group is —S-(alkyl), wherein alkyl is defined above.

As used herein and unless otherwise specified, an “alkoxyalkyl” group is -(alkyl)-O-(alkyl), wherein alkyl is defined above.

As used herein and unless otherwise specified, a “cycloalkyloxy” group is —O-(cycloalkyl), wherein cycloalkyl is defined above.

As used herein and unless otherwise specified, an “aryloxy” group is —O-(aryl), wherein aryl is defined above.

As used herein and unless otherwise specified, a “heterocyclyloxy” group is —O-(heterocyclyl), wherein heterocyclyl is defined above. A “heteroaryloxy” group is —O-(heteroaryl), wherein heteroaryl is defined above. A “heterocycloalkyloxy” group is —O-(heterocycloalkyl), wherein heterocycloalkyl is defined above.

As used herein and unless otherwise specified, an “amino” group is a radical of the formula: —NH₂, —NH(R^{#}), or —N(R^{#})₂, wherein each R^{#} is independently an alkyl, cycloalkyl, cycloalkylalkyl, aryl, aralkyl, heterocyclyl (e.g., heteroaryl or heterocycloalkyl), or heterocyclylalkyl (e.g., heteroarylalkyl or heterocycloalkylalkyl) group defined above, each of which is independently substituted or unsubstituted.

In one embodiment, an “amino” group is an “alkylamino” group, which is a radical of the formula: —NH-alkyl or —N(alkyl)₂, wherein each alkyl is independently defined above. The term “cycloalkylamino”, “arylamino”, “heterocyclylamino”, “heteroarylamino”, “heterocycloalkylamino”, or the like, mirrors the above description for “alkylamino” where the term “alkyl” is replaced with “cycloalkyl”, “aryl”, “heterocyclyl”, “heteroaryl”, “heterocycloalkyl”, or the like, respectively.

As used herein and unless otherwise specified, a “carboxy” group is a radical of the formula: —C(O)OH.

As used herein and unless otherwise specified, an “acyl” group is a radical of the formula: —C(O)(R^{#}) or —C(O)H, wherein R^{#} is defined above. A “formyl” group is a radical of the formula: —C(O)H.

As used herein and unless otherwise specified, an “amido” group is a radical of the formula: —C(O)—NH₂, —C(O)—NH(R^{#}), —C(O)—N(R^{#})₂, —NH—C(O)H, —NH—C(O)—(R^{#}), —N(R^{#})—C(O)H, or —N(R^{#})—C(O)—(R^{#}), wherein each R^{#} is independently defined above.

In one embodiment, an “amido” group is an “aminocarbonyl” group, which is a radical of the formula: —C(O)—NH₂, —C(O)—NH(R^{#}), —C(O)—N(R^{#})₂, wherein each R^{#} is independently defined above.

In one embodiment, an “amido” group is an “acylamino” group, which is a radical of the formula: —NH—C(O)H, —NH—C(O)—(R^{#}), —N(R^{#})—C(O)H, or —N(R^{#})—C(O)—(R^{#}), wherein each R^{#} is independently defined above.

As used herein and unless otherwise specified, a “sulfonylamino” group is a radical of the formula: —NHSO₂(R^{#}) or —N(R^{#})SO₂(R^{#}), wherein each R^{#} is defined above.

As used herein and unless otherwise specified, an “ester” group is a radical of the formula: —C(O)—O—(R^{#}) or —O—C(O)—(R^{#}), wherein R^{#} is defined above.

In one embodiment, an “ester” group is an “alkoxycarbonyl” group, which is a radical of the formula: —C(O)—O-(alkyl), wherein alkyl is defined above. The term “cycloalkyloxycarbonyl”, “aryloxycarbonyl”, “heterocyclyloxycarbonyl”, “heteroaryloxycarbonyl”, “heterocycloalkyloxycarbonyl”, or the like, mirrors the above description for “alkoxycarbonyl” where the term “alkoxy” is replaced with “cycloalkyloxy”, “aryloxy”, “heterocyclyloxy”, “heteroaryloxy”, “heterocycloalkyloxy”, or the like, respectively.

As used herein and unless otherwise specified, a “carbamate” group is a radical of the formula: —O—C(O)—NH₂, —O—C(O)—NH(R^{#}), —O—C(O)—N(R^{#})₂, —NH—C(O)—O—(R^{#}), or —N(R^{#})—C(O)—O—(R^{#}), wherein each R^{#} is independently defined above.

As used herein and unless otherwise specified, a “urea” group is a radical of the formula: —NH(CO)NH₂, —NHC(O)NH(R^{#}), —NHC(O)N(R^{#})₂, —N(R^{#})C(O)NH₂, —N(R^{#})C(O)NH(R^{#}), or —N(R^{#})C(O)N(R^{#})₂, wherein each R^{#} is independently defined above.

As used herein and unless otherwise specified, a “sulfinyl” group is a radical of the formula: —S(O)R^{#}, wherein R^{#} is defined above.

As used herein and unless otherwise specified, a “sulfonyl” group is a radical of the formula: —S(O)₂R^{#}, wherein R^{#} is defined above.

As used herein and unless otherwise specified, an “aminosulfonyl” group is a radical of the formula: —SO₂NH₂, —SO₂NH(R^{#}), or —SO₂N(R^{#})₂, wherein each R^{#} is independently defined above.

When the groups described herein, with the exception of alkyl groups, are said to be “substituted,” they may be substituted with any appropriate substituent or substituents. Illustrative examples of substituents are those found in the exemplary compounds and embodiments disclosed herein, as well as halogen; alkyl, alkenyl, alkynyl, cycloalkyl, aryl, heterocyclyl, heteroaryl, heterocycloalkyl, cycloalkylalkyl, aralkyl, heterocyclylalkyl, heteroarylalkyl, heterocycloalkyalkyl, optionally further substituted; hydroxy; alkoxy; cycloalkyloxy, aryloxy, heterocyclyloxy, heteroaryloxy, heterocycloalkyoxy, cycloalkylalkyloxy, aralkyloxy, heterocyclylalkyloxy, heteroarylalkyloxy, heterocycloalkyalkyloxy; oxo (═O); oxide (e.g., a nitrogen atom substituted with an oxide is called N-oxide); amino, alkylamino, cycloalkylamino, arylamino, heterocyclylamino, heteroarylamino, heterocycloalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂.

As used herein, the term “Heterocyclic Compound” includes compounds of formula (I) formula (II), formula (III), formula (IIIa), formula (IIIb), formula (IIIc), formula (IIId), formula (IV), formula (IVa), formula (IVb), and formula (IVc), as well as to further embodiments of compounds of formula (I) formula (II), formula (III), formula (IIIa), formula (IIIb), formula (IIIc), formula (IIId), formula (IV), formula (IVa), formula (IVb), and formula (IVc), provided herein. For example, the term “Heterocyclic Compound” includes deuterated compounds of formula (I), formula (II), formula (III), formula (IIIa), formula (IIIb), formula (IIIc), formula (IIId), formula (IV), formula (IVa), formula (IVb), and formula (IVc), Table 1, Table 2, Table 3, Table 4, and Table 5. In one embodiment, an “Heterocyclic Compound” is a compound set forth in Table 1, Table 2, Table 3, Table 4, or Table 5. In certain embodiments, the term “Heterocyclic Compound” includes pharmaceutically acceptable salts, tautomers, isotopologues, and/or stereoisomers of the Heterocyclic Compounds provided herein.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds of formula (I), formula (II), formula (III), formula (IIIa), formula (IIIb), formula (IIIc), formula (IIId), formula (IV), formula (IVa), formula (IVb), formula (IVc), Table 1, Table 2, Table 3, Table 4, or Table 5 include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, maleic, phosphoric, sulfuric, and methanesulfonic acids. Examples of specific salts thus include hydrochloride and mesylate salts. Others are well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereomerically pure” means one stereoisomer of a Heterocyclic Compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The Heterocyclic Compounds can have chiral centers and can occur as racemates, individual enantiomers or diastereomers, and mixtures thereof. All such isomeric forms are included within the embodiments disclosed herein, including mixtures thereof.

The use of stereomerically pure forms of such Heterocyclic Compounds, as well as the use of mixtures of those forms, are encompassed by the embodiments disclosed herein. For example, mixtures comprising equal or unequal amounts of the enantiomers of a particular Heterocyclic Compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, IN, 1972).

It should also be noted the Heterocyclic Compounds can include E and Z isomers, or a mixture thereof, and cis and trans isomers or a mixture thereof. In certain embodiments, the Heterocyclic Compounds are isolated as either the E or Z isomer. In other embodiments, the Heterocyclic Compounds are a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

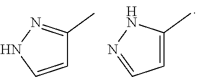

As readily understood by one skilled in the art, a wide variety of functional groups and other structures may exhibit tautomerism and all tautomers of compounds of formula (I), formula (II) formula (III), formula (IIIa), formula (IIIb), formula (IIIc), formula (IIId), formula (IV), formula (IVa), formula (IVb), and formula (IVc), are within the scope of the present invention.

It should also be noted the Heterocyclic Compounds can contain unnatural proportions of atomic isotopes at least one of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the Heterocyclic Compounds as described herein, whether radioactive or not, are intended to be encompassed within the scope of the embodiments provided herein. In some embodiments, there are provided isotopologues of the Heterocyclic Compounds, for example, the isotopologues are carbon-13, or nitrogen-15 enriched Heterocyclic Compounds. As used herein, “deuterated”, means a compound wherein at least one hydrogen (H) has been replaced by deuterium (indicated by D or ²H), that is, the compound is enriched in deuterium in at least one position It should be noted that if there is a discrepancy between a depicted structure and a name for that structure, the depicted structure is to be accorded more weight.

As used herein, “inhibit” and “inhibition” mean that a specified response of a designated activity (e.g., worm motility) is comparatively decreased in the presence of a Heterocyclic Compound. Inhibition of worm motility, for example motility of Onchocerca volvulus, Brugia malayi and/or Brugia timori, can be determined by the assays described herein.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In one embodiment, the disorder, disorder or condition is a helminthic infection.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In one embodiment, the disorder, disorder or condition is a helminthic infection.

The term “effective amount” in connection with a Heterocyclic Compound means an amount capable of treating or preventing a disorder, disease or condition, or symptoms thereof, disclosed herein. In one embodiment, the disorder, disorder or condition is a helminthic infection.

The term “subject” or “patient” includes humans and other primates as well as domesticated and semi-domesticated animals including, but not limited to, poultry, honeybees, cows, sheep, cattle, goats, pigs, horses, dogs, cats, rabbits, rats, mice and the like. The term “poultry” encompasses all types of domestic fowl, including, but not limited to chickens, turkey, ducks, geese, the ratite group of birds and game birds. In certain embodiments, the subject is a human. In certain embodiments, the subject is a dog. In certain embodiments, the subject is a cat. In certain embodiments, the subject is a livestock. In certain embodiments, the subject is a cow. In certain embodiments, the subject is a sheep. In another embodiment, the subject is a goat.

The term “combination” or administration “in combination” includes administration as a mixture, simultaneous administration using separate formulations, and consecutive administration in any order.

The term “helminthic infections” or “helminth infection” as used herein refers to infections that are caused by parasitic worms. An infection caused by a helminth, known as “helminthiasis” (plural “helminthiases”), is any macroparasitic disease of humans and other animals in which a part of the body is infected with parasitic worms, known as helminths. There are numerous species of these parasites, which are broadly classified into tapeworms, flukes, and roundworms.

The term “filariasis” as used herein refers to helminth infections that are caused by filarial nematodes. Non-limiting examples of filarial nematodes within the Onchocercidae family include the genus Brugia spp. (i.e., B. malayi, B. pahangi, B. timori, and the like), Wuchereria spp. (i.e., W. bancrofti, and the like), Dirofilaria spp. (D. immitis, D. repens, D. ursi, D. tenuis, D. spectans, D. lutrae, and the like), Dipetalonema spp. (i.e., D. reconditum, D. repens, and the like), Onchocerca spp. (i.e., O. gibsoni, O. gutturosa, O. volvulus, and the like), Elaeophora spp. (E. bohmi, E. elaphi, E. poeli, E. sagitta, E. schneideri, and the like), Mansonella spp. (i.e., M. ozzardi, M. perstans, and the like), and Loa spp. (i.e., L. loa). An infection is the colonization of a host organism by parasite species. Infections with human filarial nematodes can cause lymphatic filariasis or onchocerciasis. The term “lymphatic filariasis” refers to an infection with the nematodes Wuchereria bancrofti, Brugia malayi or Brugia timori. The term “onchocerciasis” refers to an infection with the nematode Onchocerca volvulus. Lymphatic filariasis may cause hydrocoele, lymphoedema, and elephantiasis. Onchocerciasis may cause skin inflammation and blindness, so called River Blindness. In dogs, an infection with nematode species called Dirofilaria immitis or Dirofilaria repens causes dirofilariasis. In sheep and goats and infection with a nematoide species called Haemonchus contortus causes haemonchosis.

The term “worm” or “nematode” as used interchangeably herein refers to all life stages of the organism, such as an egg, an unfertilized egg, a fertilized egg, a larva or juvenile worm, a larva in any one of four larval stages (L1, L2, L3, L4), a worm in sexually immature stage (stage L5), a worm in mature stage, a worm in fully mature stage, an adult worm, a worm in pre-parasitic stage, or a worm in parasitic stage.

The term “microfilaria” or “mf” as used herein refers to an early stage in the life cycle of certain parasitic nematodes. Microfilaria is considered to be the first larval stage also referred to as L1. The terms “microfilaria,” “mf,” or “L1” are used alternatively and/or interchangeably.

The term “macrofilaria” as used herein refers to the adult stage in the life cycle of certain parasitic nematodes.

Unless otherwise defined, the technical and scientific terms used herein have the same meaning as commonly understood by one of ordinary skill in the art to which this application belongs.

Surprisingly, it was found that the compounds disclosed herein are effective in the treatment of helminthic infections, for example, filarial infections. In vitro and in vivo results demonstrated that the compounds disclosed herein are effective against filarial nematodes. In some embodiments, the compounds disclosed herein surprisingly presented distinct activity between parasitic nematodes in adult and juvenile stage. In some such embodiments, the compounds disclosed herein are selectively effective against adult filarial nematodes (also referred to as macrofilaricidal activity). In other embodiments, the compounds disclosed herein are selectively effective against the juvenile stage filarial nematodes (also referred to as microfilaricidal activity). Therefore, the compounds disclosed herein have the potential to be potent anti-filarial drugs.

### Compounds

Provided herein are compounds having the following formula (I):

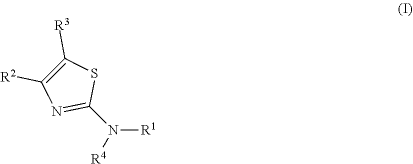

and pharmaceutically acceptable salts, tautomers, isotopologues, and stereoisomers thereof, wherein:
R¹ is isoquinolyl; pyrrolopyridyl; 2-pyrimidyl, or 2-pyridyl, wherein the 2-pyridyl is substituted with one or more substituents independently selected from halogen, CN, substituted or unsubstituted C₁₋₄ alkyl, substituted or unsubstituted C₃₋₇ cycloalkyl, substituted or unsubstituted C₆₋₁₀ aryl, substituted or unsubstituted monocyclic heteroaryl, substituted or unsubstituted 3-6 membered heterocyclyl, —OR⁵, —SR, —CONR⁶₂, —CON(C₁₋₃ alkyl)(substituted or unsubstituted C₃₋₇ cycloalkyl), —NRCO(C₁₋₃ alkyl), —CO(substituted or unsubstituted 3-6 membered heterocyclyl), —SO₂NR₂ and SO₂R⁵;
R² is 5,6-dihydro-4H-pyrrolo[1,2-b]pyrazolyl, 2-pyrimidyl, or 2-pyridyl, substituted with one or more substituents independently selected from halogen, substituted or unsubstituted C₁₋₄ alkyl, substituted or unsubstituted C₆₋₁₀ aryl, —OR⁵, —SR, —CONR₂, and —SO₂R⁵, or two atoms together with the carbons to which they are attached form a substituted or unsubstituted 5-6 membered heterocyclyl;
R³ is H, —CN, substituted or unsubstituted C₁₋₄ alkyl, (C₁₋₃ alkyl)O(C₁₋₃ alkyl)(substituted or unsubstituted C₃₋₇ cycloalkyl), —(C₁₋₃ alkyl)OR, (C₁₋₃ alkyl)(substituted or unsubstituted 3-6 membered heterocyclyl), —C(O)(substituted or unsubstituted 3-10 membered heterocyclic), —C(O)OR, substituted or unsubstituted C₆₋₁₀ aryl, (C₁₋₃ alkyl)NR⁶₂, —(C₁₋₃ alkyl)N(C₁₋₃ alkyl)(substituted or unsubstituted C₃₋₇ cycloalkyl), CONR⁶₂, or —C(O)N(C₁₋₃ alkyl)NR₂;
R⁴ is H or substituted or unsubstituted C₁₋₃ alkyl, or substituted or unsubstituted —(C₁₋₃ alkyl) C₆₋₁₀ aryl;
R⁵ is H, substituted or unsubstituted C₁₋₅ alkyl, substituted or unsubstituted C₃₋₇ cycloalkyl, or substituted or unsubstituted 3-6 membered heterocyclyl;
each R⁶ is independently selected from H, substituted or unsubstituted C₁₋₅ alkyl, substituted or unsubstituted C₃₋₆ cycloalkyl, substituted or unsubstituted 3-6 membered heterocyclyl, and (C₁₋₃ alkyl)(substituted or unsubstituted 3-6 membered heterocyclyl); and
each R is independently selected from H and substituted or unsubstituted C₁₋₄ alkyl;
provided the compound is not 4-methyl-N-[4-(4-methyl-2-pyridinyl)-2-thiazolyl]-2-pyridinamine or N-(5-chloropyridin-2-yl)-4-(pyrimidin-2-yl)thiazol-2-amine, which have structures

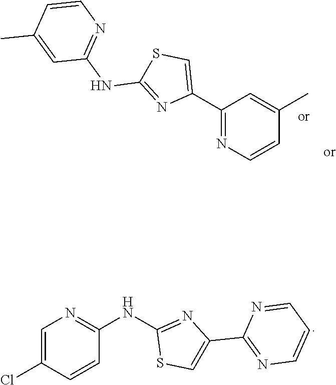

In one embodiment of compounds of formula (I), R is isoquinolyl; 1H-pyrrolo[3,2-c]pyridyl; or 1H-pyrrolo[2,3-c]pyridyl.

In one embodiment of compounds of formula (I), R¹ is 2-pyrimidyl, wherein the 2-pyrimidyl is unsubstituted or substituted with one or more substituents independently selected from substituted or unsubstituted C₁₋₄ alkyl, substituted or unsubstituted C₃₋₇ cycloalkyl, substituted or unsubstituted C₆₋₁₀ aryl, substituted or unsubstituted monocyclic heteroaryl, substituted or unsubstituted 3-6 membered heterocyclyl, —OR⁵, —CONR⁶₂, —NRCO(C₁₋₃ alkyl), and —CO(substituted or unsubstituted 3-6 membered heterocyclyl).

In one embodiment of compounds of formula (I), R¹ is 2-pyrimidyl, wherein the 2-pyrimidyl is unsubstituted or substituted with one or more substituents independently selected from substituted or unsubstituted —CH₃, —CH₂CH₃, —CH₂CH₂CH₃, —CH(CH₃)₂, CF₃, cyclopropyl, cylobutyl, cyclopentyl, —OR⁵, substituted or unsubstituted phenyl, substituted or unsubstituted pyrazolyl, substituted or unsubstituted dihydropyranyl, substituted or unsubstituted piperidyl, substituted or unsubstituted piperazinyl, —CONR⁶₂, —CON(C₁₋₃ alkyl)(substituted or unsubstituted C₃₋₇ cycloalkyl), —NRCO(C₁₋₃ alkyl), —CO(substituted or unsubstituted 3-6 membered heterocyclyl).

In one embodiment of compounds of formula (I), R¹ is 2-pyrimidyl, wherein the 2-pyrimidyl is unsubstituted or substituted with one or more substituents independently selected from substituted or unsubstituted —CH₃, —CH₂CH₃, —CH₂CH₂CH₃, —CH(CH₃)₂, CF₃, cyclopropyl, —OR⁵, substituted or unsubstituted phenyl, substituted or unsubstituted pyrazolyl, substituted or unsubstituted dihydropyranyl, substituted or unsubstituted piperidyl, substituted or unsubstituted piperazinyl, —CONR⁶₂, and —NCH₃COCH₃.

In one embodiment of compounds of formula (I), R¹ is 2-pyridyl, wherein the 2-pyridyl is substituted with one or more substituents independently selected from halogen, —CN, substituted or unsubstituted C₁₋₄ alkyl, substituted or unsubstituted C₃₋₇ cycloalkyl, substituted or unsubstituted C₆₋₁₀ aryl, substituted or unsubstituted monocyclic heteroaryl, substituted or unsubstituted 3-6 membered heterocyclyl, —OR⁵, —SR, —CONR⁶₂, —CON(C₁₋₃ alkyl)(substituted or unsubstituted C₃₋₇ cycloalkyl), —NRCO(C₁₋₃ alkyl), —CO(substituted or unsubstituted 3-6 membered heterocyclyl), —SO₂NR₂, and SO₂R⁵.

In some embodiments of compounds of formula (I), R¹ is 2-pyridyl, substituted with one or more substituents independently selected from F, Br, Cl, —CN, —CH₃, —CH₂CH₃, —CH₂CH₂CH₃, —CH₂CH₂CH₂CH₃, —CH(CH₃)₂, CF₃, cyclopropyl, cylobutyl, cyclopentyl, —OR⁵, —SR, substituted or unsubstituted phenyl, substituted or unsubstituted pyrazolyl, substituted or unsubstituted dihydropyranyl, substituted or unsubstituted piperidyl, substituted or unsubstituted piperazinyl, substituted or unsubstituted pyrrolidinonyl, —CONR⁶₂, —CON(C₁₋₃ alkyl)(substituted or unsubstituted C₃₋₇ cycloalkyl), —NRCO(C₁₋₃ alkyl), —CO(substituted or unsubstituted 3-6 membered heterocyclyl)-SO₂NR₂, and —SO₂R⁵.

In some embodiments, R¹ is 2-pyridyl substituted with one or more OR⁵. In some such embodiments, R⁵ is H, —CH₃, —CH₂CH₃, —CH₂CH₂CH₃, —CH(CH₃)₂, tetrahydrofuranyl, tetrahydropyranyl, or 1-methylpiperidyl. In some such embodiments, R⁵ is H, —CH₃, —CH(CH₃)₂, or tetrahydropyranyl. In some such embodiments, R⁵ is H, —CH₃, —CH₂CH₃, —CH₂CH₂CH₃, —CH(CH₃)₂, piperidyl, 1-methylpiperidyl, tetrahydrofuranyl, or tetrahydropyranyl. In some such embodiments, R⁵ is H or —CH₃. In some such embodiments, R⁵ is —CH₃. In some embodiments, R⁵ is —CH(CH₃)₂. In some such embodiments, R⁵ is tetrahydropyranyl. In some such embodiments, R⁵ is 1-methylpiperidyl.

In some embodiments of compounds of formula (I), R¹ is 2-pyridyl substituted with one or more —CONR⁶₂. In some such embodiments, each R⁶ is independently H, substituted or unsubstituted C₁₋₅ alkyl selected from —CH₃, —CH₂CH₃, —CH₂CH₂CH₃, and —CH(CH₃)₂; substituted or unsubstituted C₃₋₆ cycloalkyl selected from cyclopropyl, cyclobutyl, cyclopentyl, and cyclohexyl; wherein the alkyl and cycloalkyl are optionally substituted with one or more substituents independently selected from OH, OCH₃, and F. In some embodiments, each R⁶ is independently H, substituted or unsubstituted C₁₋₅ alkyl selected from —CH(CH₃)₂; substituted or unsubstituted C₃₋₆ cycloalkyl selected from cyclopentyl and cyclohexyl; wherein the alkyl and cycloalkyl are optionally substituted with one or more substituents independently selected from OH, OCH₃, and F.

In some embodiments, R¹ is 2-pyridyl, substituted with one or more substituents independently selected from F, Br, Cl, —CN, —CH₃, —CH₂CH₃, —CH₂CH₂CH₃, —CH₂CH₂CH₂CH₃, —CH(CH₃)₂, CF₃, cyclopropyl, cylobutyl, cyclopentyl, —OH, —OCH₃, —OCH₂CH₃, —OCH₂CH₂CH₃, —OCH(CH₃)₂, —O-tetrahydrofuranyl, —O-tetrahydropyranyl, —SCH₃, substituted or unsubstituted phenyl, substituted or unsubstituted pyrazolyl, substituted or unsubstituted dihydropyranyl, substituted or unsubstituted piperidyl, substituted or unsubstituted piperazinyl, substituted or unsubstituted pyrrolidinonyl, —CONH₂, —CONHCH₃, —CON(CH₃)₂, —CONHCH₂CH₃, —CON(CH₂CH₃)₂, —CONHCH₂CH₂CH₃—CONHCH(CH₃)₂, —CONHCH₂CH₂OH, —CONHCH₂CH₂OCH₃, —CONHCH(CH₃)CH₂OH, —CONHCH₂CF₃, —CONHCH₂CH₂-pyrrolidyl, —CONH(cyclopropyl), —CONH(cyclobutyl), —CONH(cyclopentyl), —CONH(cyclohexyl), —CO(azetidyl), —CO(piperidyl), —CO(piperazinyl), —CO(morpholinyl), —CONH(CH₂)-cyclopropyl, —N(CH₃)COCH₃, —SO₂N(CH₃)₂, and —SO₂(aziridinyl); wherein the cyclopropyl, cyclobutyl, cyclopentyl, cyclohexyl, azetidyl, piperidyl and piperazinyl are optionally fluorinated.

In some embodiments, R¹ is 2-pyridyl, substituted with one or more substituents independently selected from F, Br, Cl, —CN, —CH₃, —CH₂CH₃, —CH(CH₃)₂, CF₃, cyclopropyl, cyclobutyl, cyclopentyl, —OH, —OCH₃, —OCH(CH₃)₂, —O-tetrahydropyranyl, —SCH₃, substituted or unsubstituted phenyl, substituted or unsubstituted pyrazolyl, substituted or unsubstituted dihydropyranyl, substituted or unsubstituted piperidyl, substituted or unsubstituted piperazinyl, substituted or unsubstituted pyrrolidinonyl, —CONH₂, —CONHCH(CH₃)₂, —CONHCH₂CH₂OH, —CONHCH₂CH₂OCH₃, —CONHCH(CH₃)CH₂OH, —CONHCH₂CF₃, —CONHCH₂CH₂-pyrrolidyl, —CONH(cyclopentyl), —CONH(cyclohexyl), —CO(azetidyl), CO(piperidyl), —CO(piperazinyl), —CO(morpholinyl), —N(CH₃)COCH₃, —SO₂N(CH₃)₂, and —SO₂(aziridinyl); wherein the cyclopropyl, cyclobutyl, cyclopentyl, cyclohexyl, azetidyl, piperidyl and piperazinyl are optionally fluorinated.

In some embodiments, R¹ is 2-pyridyl, substituted with one or more substituents independently selected from F, Br, Cl, —CN, —CH₃, —CH₂CH₃, —CH(CH₃)₂, CF₃, cyclopropyl, cyclopentyl, —OH, —OCH₃, —OCH(CH₃)₂, —O-tetrahydropyranyl, —SCH₃, phenyl; phenyl(COOH); 1-methylpyrazolyl; dihydropyranyl; 1-methyl-piperidyl; piperidyl substituted with COOH, —CONHCH₃, or CONHCH₂CF₃; 1-methyl-piperazinyl; piperazinyl substituted with —COC(CH₃)₂OH and CO-cyclopropyl-CF₃; —CONH₂, —CON(CH₃)₂, —CONHCH(CH₃)₂, —CONHCH₂CH₂OH, —CONHCH₂CH₂OCH₃, —CONHCH(CH₃)CH₂OH, —CONHCH₂CF₃, —CONHCH₂CH₂-pyrrolidyl, —CONH(cyclopentyl), —CONH(difluorocyclohexyl), —CO(difluoroazetidyl), —CO(difluoropiperidyl), —CO(piperazinyl), —CO(morpholinyl), —N(CH₃)COCH₃, —SO₂N(CH₃)₂, and —SO₂(aziridinyl).

In some embodiments of compounds of formula (I), R² is 2-pyridyl substituted with one or more substituents independently selected from F, Cl, —CN, CH₃, —CH₂CH₃, —CF₃, —CHF₂, substituted or unsubstituted phenyl, —OR⁵, —SR, —SO₂R⁵, and —CONR₂. In some embodiments, R² is 2-pyridyl substituted with one or more substituents independently selected from F, Cl, —CN, —CH₃, —CH₂CH₃, —CF₃, —CHF₂, —OCH₃, —OCH₂CH₃, —OCH₂CH₂CH₃, —OCH(CH₃)₂, —OCH₂CF₃, —O-cyclopropyl, —O-oxetanyl, —O-(1-methyl-azetidinyl), —O-(1-methyl-piperidyl), —O-tetrahydrofuranyl, —O-tetrahydropyranyl, —SCH₃, —CONH₂, —CONHCH₃, —CON(CH₃)₂, —CONHCH₂CH₃, —CON(CH₂CH₃)₂, —SO₂CH₃, and substituted or unsubstituted phenyl. In some such embodiments, R² is 2-pyridyl substituted with one or more substituents independently selected from F, Cl, —CN, CH₃, —CH₂CH₃, —CF₃, —CHF₂, —OCH₃, —OCH₂CH₃, —OCH(CH₃)₂, —OCH₂CF₃, —O-cyclopropyl, —O-oxetanyl, —O-(1-methyl-azetidinyl), —O-(1-methyl-piperidyl), —O-tetrahydropyranyl, —SCH₃, —CONH₂, —CON(CH₃)₂, —SO₂CH₃, and substituted or unsubstituted phenyl. In some such embodiments, R² is substituted with one or more substituents independently selected from F, Cl, —CN, CH₃, —CH₂CH₃, —CF₃, —CHF₂, —OCH₃, —OCH₂CH₃, —OCH(CH₃)₂, —OCH₂CF₃, —O-cyclopropyl, —O-oxetanyl, —O-(1-methyl-azetidinyl), —O-(1-methyl-piperidyl), —O-tetrahydropyranyl, —SCH₃, —CONH₂, —CON(CH₃)₂, —SO₂CH₃, phenyl; and phenyl, substituted with cyclopropyl(COOH).

In some embodiments of compounds of formula (I), R² is 2-pyridyl, wherein two atoms together with the carbons to which they are attached form a substituted or unsubstituted 5-6 membered heterocyclyl. In some such embodiments, R² is a substituted or unsubstituted 2,3-dihydrofuro[2,3-c]pyridyl, 2,3-dihydro-1H-pyrrolo[2,3-c]pyridyl, or 1,3-dihydro-2H-pyrrolo[2,3-c]pyridyl-2-one.

In some embodiments of compounds of formula (I), R² is 5,6-dihydro-4H-pyrrolo[1,2-b]pyrazolyl, or 2-pyrimidyl.

In some embodiments of compounds of formula (I), R³ is H, —CN, —CH₃, —CH₂CH₃, —CH(CH₃)₃, —CH₂OH, —CH₂CH₂OH, —CH₂OCH₂-cyclopropyl, —CH₂OCH₂-cyclobutyl, —CH₂CH₂O-cyclobutyl, —CH₂CH₂OCH₂-cyclopropyl, —CH₂OCH₂CH₂-cyclopropyl, —CH₂N(CH₃)₂, —CH₂-azetidyl, —CH₂-piperidyl, —CH₂(dimethylmorpholinyl), —CH₂(dimethylpiperazyl), —CH₂-pirrolidyl, —CH₂(morpholinyl), —COOH, —CO(dimethylmorpholinyl), —CO(morpholinyl), —CO(1,3-dioxolane-piperidyl), —CO(piperidyl), —CO(pirrolidyl), —CO(1-methyl-piperazyl), —CO(octahydropyrrolo[1,2-a]pyrazyl), —CONHCH₂-cyclohexyl, —CONHCH₂-tetrahydropiranyl, —CONHCH₂-cyclopentyl, —CONH(CH₂)₂N(CH₃)₂, —CON(CH₃)₂, or phenyl. In some embodiments of compounds of formula (I), R³ is H, —CH₃, —CH₂OH, —CH₂OCH₂-cyclopropyl, —CH₂-azetidyl, —CH₂-piperidyl; or phenyl.

In some embodiments of compounds of formula (I), R⁴ is H, —CH₃, or —CH₂-phenyl.

In some embodiments of compounds of formula (I), wherein when R¹ is 2-pyridyl, substituted with one or more substituents independently selected from F, Br, Cl, —CN, —CH₃, —CH₂CH₃, —CH₂CH₂CH₃, —CH₂CH₂CH₂CH₃, —CH(CH₃)₂, CF₃, cyclopropyl, cylobutyl, cyclopentyl, —OR⁵, —SR, substituted or unsubstituted phenyl, substituted or unsubstituted pyrazolyl, substituted or unsubstituted dihydropyranyl, substituted or unsubstituted piperidyl, substituted or unsubstituted piperazinyl, substituted or unsubstituted pyrrolidinonyl, —CONR⁶₂, —CON(C₁₋₃ alkyl)(substituted or unsubstituted C₃₋₇ cycloalkyl), —NRCO(C₁₋₃ alkyl), —CO(substituted or unsubstituted 3-6 membered heterocyclyl), —SO₂NR₂, and —SO₂R⁵; R² is 2-pyridyl substituted with one or more substituents independently selected from F, Cl, —CN, —CH₃, —CH₂CH₃, —CF₃, —CHF₂, substituted or unsubstituted phenyl, —OR⁵, —SR, —CONR₂, and —SO₂R⁵. In some other such embodiments, R³ is H, —CN, —CH₃, —CH₂CH₃, —CH(CH₃)₃, —CH₂OH, —CH₂CH₂OH, —CH₂OCH₂-cyclopropyl, —CH₂OCH₂-cyclobutyl, —CH₂CH₂O-cyclobutyl, —CH₂CH₂OCH₂-cyclopropyl, —CH₂OCH₂CH₂-cyclopropyl, —CH₂N(CH₃)₂, —CH₂-azetidyl, —CH₂-piperidyl, —CH₂(dimethylmorpholinyl), —CH₂(dimethylpiperazyl), —CH₂-pirrolidyl, —CH₂(morpholinyl), —COOH, —CO(dimethylmorpholinyl), —CO(morpholinyl), —CO(1,3-dioxolane-piperidyl), —CO(piperidyl), —CO(pirrolidyl), —CO(1-methyl-piperazyl), —CO(octahydropyrrolo[1,2-a]pyrazyl), —CONHCH₂-cyclohexyl, —CONHCH₂-tetrahydropiranyl, —CONHCH₂-cyclopentyl, —CONH(CH₂)₂N(CH₃)₂, —CON(CH₃)₂, or phenyl. In some other such embodiments, R⁴ is H, —CH₃, or CH₂-phenyl. In some other such embodiments, R⁴ is H. In some other such embodiments, R⁴ is —CH₃. In some other such embodiments, R⁴ is —CH₂-phenyl.

In some embodiments of compounds of formula (I), wherein when R¹ is 2-pyridyl, substituted with one or more substituents independently selected from F, Br, Cl, —CN, —CH₃, —CH₂CH₃, —CH₂CH₂CH₃, —CH₂CH₂CH₂CH₃, —CH(CH₃)₂, CF₃, cyclopropyl, cylobutyl, cyclopentyl, —OH, —OCH₃, —OCH₂CH₃, —OCH₂CH₂CH₃, —OCH(CH₃)₂, —O-tetrahydrofuranyl, —O-tetrahydropyranyl, —SCH₃, substituted or unsubstituted phenyl, substituted or unsubstituted pyrazolyl, substituted or unsubstituted dihydropyranyl, substituted or unsubstituted piperidyl, substituted or unsubstituted piperazinyl, substituted or unsubstituted pyrrolidinonyl, —CONH₂, —CONHCH₃, —CON(CH₃)₂, —CONHCH₂CH₃, —CON(CH₂CH₃)₂, —CONHCH₂CH₂CH₃, —CONHCH(CH₃)₂, —CONHCH₂CH₂OH, —CONHCH₂CH₂OCH₃, —CONHCH(CH₃)CH₂OH, —CONHCH₂CF₃, —CONHCH₂CH₂-pyrrolidyl, —CONH(cyclopropyl), —CONH(cyclobutyl), —CONH(cyclopentyl), —CONH(cyclohexyl), —CO(azetidyl), —CO(piperidyl), —CO(piperazinyl), —CO(morpholinyl), —CONH(CH₂)-cyclopropyl, —N(CH₃)COCH₃, —SO₂N(CH₃)₂, and —SO₂(aziridinyl); wherein the cyclopropyl, cyclobutyl, cyclopentyl, cyclohexyl, azetidyl, piperidyl and piperazinyl are optionally fluorinated; R² is 2-pyridyl substituted with one or more substituents independently selected from F, Cl, —CN, —CH₃, —CH₂CH₃, —CF₃, —CHF₂, —OCH₃, —OCH₂CH₃, —OCH₂CH₂CH₃, —OCH(CH₃)₂, —OCH₂CF₃, —O-cyclopropyl, —O-oxetanyl, —O-(1-methyl-azetidinyl), —O-(1-methyl-piperidyl), —O-tetrahydrofuranyl, —O-tetrahydropyranyl, —SCH₃, —CONH₂, —CONHCH₃, —CON(CH₃)₂, —CONHCH₂CH₃, —CON(CH₂CH₃)₂, —SO₂CH₃, and substituted or unsubstituted phenyl. In some such embodiments, R³ is H, —CH₃, —CH₂CH₃, —CH₂OH, —CH₂CH₂OH, —CH₂OCH₂-cyclopropyl, —CH₂CH₂OCH₂-cyclopropyl, —CH₂OCH₂CH₂-cyclopropyl, —CH₂-azetidyl, —CH₂-piperidyl; or phenyl. In some other such embodiments, R⁴ is H, —CH₃, or CH₂-phenyl. In some other such embodiments, R⁴ is H. In some other such embodiments, R⁴ is —CH₃. In some other such embodiments, R⁴ is —CH₂-phenyl.

In some embodiments of compounds of formula (I), wherein when R¹ is 2-pyridyl, substituted with one or more substituents independently selected from F, Br, Cl, —CN, —CH₃, —CH₂CH₃, —CH₂CH₂CH₃, —CH(CH₃)₂, CF₃, cyclopropyl, cyclopentyl, —OH, —OCH₃, —OCH₂CH₃, —OCH(CH₃)₂, —O-tetrahydropyranyl, —SCH₃, phenyl, phenyl(COOH); pyrrolidinonyl, 1-methylpyrazolyl; dihydropyranyl; 1-methyl-piperidyl; piperidyl substituted with COOH, —CONHCH₃, or CONHCH₂CF₃; 1-methyl-piperazinyl; piperazinyl substituted with COC(CH₃)₂OH or CO-cyclopropyl-CF₃; —CONH₂, —CONHCH(CH₃)₂, —CONHCH₂CH₂OH, —CONHCH₂CH₂OCH₃, —CONHCH(CH₃)CH₂OH, —CONHCH₂CF₃, —CONHCH₂CH₂-pyrrolidyl, —CONH(cyclopentyl), —CONH(difluorocyclohexyl), —NCH₃COCH₃, —SO₂N(CH₃)₂, —SO₂(aziridinyl), —CO(difluoroazetidyl), CO(difluoropiperidyl), —CO(piperazinyl), and —CO(morpholinyl); R² is 2-pyridyl substituted with one or more substituents independently selected from F, Cl, —CN, —CH₃, —CH₂CH₃, —CF₃, —CHF₂, —OCH₃, —OCH₂CH₃, —OCH(CH₃)₂, —OCH₂CF₃, —O-cyclopropyl, —O-oxetanyl, —O-(1-methyl-azetidinyl), —O-(1-methyl-piperidyl), —O— tetrahydropyranyl, —SCH₃, —CONH₂, —CON(CH₃)₂, —SO₂CH₃, phenyl; and phenyl, substituted with cyclopropyl(COOH); R³ is H, —CH₃, —CH₂OH, —CH₂OCH₂-cyclopropyl, —CH₂-azetidyl, —CH₂-piperidyl; or phenyl; R⁴ is H, —CH₃, or CH₂-phenyl. In some other such embodiments, R⁴ is H. In some other such embodiments, R⁴ is —CH₃. In some other such embodiments, R⁴ is —CH₂-phenyl.

In some embodiments of compounds of formula (I), wherein when R¹ is 2-pyridyl, substituted with one or more substituents independently selected from F, Br, Cl, —CN, —CH₃, —CH₂CH₃, —CH₂CH₂CH₃, —CH(CH₃)₂, CF₃, cyclopropyl, cyclopentyl, —OH, —OCH₃, —OCH₂CH₃, —OCH(CH₃)₂, —O-tetrahydropyranyl, —SCH₃, phenyl, phenyl(COOH); pyrrolidinonyl, 1-methylpyrazolyl; dihydropyranyl; 1-methyl-piperidyl; piperidyl substituted with COOH, —CONHCH₃, or CONHCH₂CF₃; 1-methyl-piperazinyl; piperazinyl substituted with COC(CH₃)₂₀H or CO-cyclopropyl-CF₃; —CONH₂, —CONHCH(CH₃)₂, —CONHCH₂CH₂OH, —CONHCH₂CH₂OCH₃, —CONHCH(CH₃)CH₂OH, —CONHCH₂CF₃, —CONHCH₂CH₂-pyrrolidyl, —CONH(cyclopentyl), —CONH(difluorocyclohexyl), —NCH₃COCH₃, —SO₂N(CH₃)₂, —SO₂(aziridinyl), —CO(difluoroazetidyl), CO(difluoropiperidyl), —CO(piperazinyl), and —CO(morpholinyl); R² is 2-pyridyl, wherein two atoms together with the carbons to which they are attached form a substituted or unsubstituted 5-6 membered heterocyclyl. In some such embodiments, R² is a substituted or unsubstituted 2,3-dihydrofuro[2,3-c]pyridyl, 2,3-dihydro-1H-pyrrolo[2,3-c]pyridyl, or 1,3-dihydro-2H-pyrrolo[2,3-c]pyridyl-2-one. In some such embodiments, R² is 2,2-dimethyl-2,3-dihydrofuro[2,3-c]pyridyl, 1-methyl-2,2-dimethyl-2,3-dihydro-1H-pyrrolo[2,3-c]pyridyl, 1-methyl-3,3-dimethyl-1,3-dihydro-2H-pyrrolo[2,3-c]pyridyl-2-one, or 1-methyl-2,3-dihydro-1H-pyrrolo[2,3-c]pyridyl; R³ is H, —CH₃, —CH₂OH, —CH₂OCH₂-cyclopropyl, —CH₂-azetidyl, —CH₂-piperidyl; or phenyl; R⁴ is H, —CH₃, or CH₂-phenyl. In some other such embodiments, R⁴ is H. In some other such embodiments, R⁴ is —CH₃. In some other such embodiments, R⁴ is —CH₂-phenyl.

In some embodiments of compounds of formula (I), wherein when R¹ is 2-pyridyl, substituted with one or more substituents independently selected from —CH₃, —CF₃, and —NCH₃COCH₃; R² is 2,2-dimethyl-2,3-dihydrofuro[2,3-c]pyridyl, 1-methyl-2,2-dimethyl-2,3-dihydro-1H-pyrrolo[2,3-c]pyridyl, 1-methyl-3,3-dimethyl-1,3-dihydro-2H-pyrrolo[2,3-c]pyridyl-2-one, or 1-methyl-2,3-dihydro-1H-pyrrolo[2,3-c]pyridyl; R³ is H; R⁴ is H.

In some embodiments of compounds of formula (I), wherein when R¹ is 2-pyridyl, substituted with one or more substituents independently selected from F, Br, Cl, —CN, —CH₃, —CH₂CH₃, —CH₂CH₂CH₃, —CH(CH₃)₂, CF₃, cyclopropyl, cyclopentyl, —OH, —OCH₃, —OCH₂CH₃, —OCH(CH₃)₂, —O-tetrahydropyranyl, —SCH₃, phenyl, phenyl(COOH); pyrrolidinonyl, 1-methylpyrazolyl; dihydropyranyl; 1-methyl-piperidyl; piperidyl substituted with COOH, —CONHCH₃, or CONHCH₂CF₃; 1-methyl-piperazinyl; piperazinyl substituted with COC(CH₃)₂OH or CO-cyclopropyl-CF₃; —CONH₂, —CONHCH(CH₃)₂, —CONHCH₂CH₂OH, —CONHCH₂CH₂OCH₃, —CONHCH(CH₃)CH₂OH, —CONHCH₂CF₃, —CONHCH₂CH₂-pyrrolidyl, —CONH(cyclopentyl), —CONH(difluorocyclohexyl), —NCH₃COCH₃, —SO₂N(CH₃)₂, —SO₂(aziridinyl), —CO(difluoroazetidyl), —CO(difluoropiperidyl), —CO(piperazinyl), and —CO(morpholinyl); R² is 5,6-dihydro-4H-pyrrolo[1,2-b]pyrazolyl. In some such embodiments, R³ is H, —CH₃, —CH₂OH, —CH₂OCH₂-cyclopropyl, —CH₂-azetidyl, —CH₂-piperidyl; or phenyl; R⁴ is H, —CH₃, or CH₂-phenyl. In some other such embodiments, R⁴ is H. In some other such embodiments, R⁴ is —CH₃. In some other such embodiments, R⁴ is —CH₂-phenyl.

In some embodiments of compounds of formula (I), wherein when R¹ is 2-pyridyl, substituted with one or more substituents independently selected from —CF₃ and —NCH₃COCH₃; R² is 5,6-dihydro-4H-pyrrolo[1,2-b]pyrazolyl; R³ is H; R⁴ is H.

In some embodiments of compounds of formula (I), wherein when R¹ is 2-pyridyl, substituted with one or more substituents independently selected from F, Br, Cl, —CN, —CH₃, —CH₂CH₃, —CH₂CH₂CH₃, —CH(CH₃)₂, CF₃, cyclopropyl, cyclopentyl, —OH, —OCH₃, —OCH₂CH₃, —OCH(CH₃)₂, —O-tetrahydropyranyl, —SCH₃, phenyl, phenyl(COOH); pyrrolidinonyl, 1-methylpyrazolyl; dihydropyranyl; 1-methyl-piperidyl; piperidyl substituted with COOH, —CONHCH₃, or CONHCH₂CF₃; 1-methyl-piperazinyl; piperazinyl substituted with COC(CH₃)₂OH or CO-cyclopropyl-CF₃; —CONH₂, —CONHCH(CH₃)₂, —CONHCH₂CH₂OH, —CONHCH₂CH₂OCH₃, —CONHCH(CH₃)CH₂OH, —CONHCH₂CF₃, —CONHCH₂CH₂-pyrrolidyl, —CONH(cyclopentyl), —CONH(difluorocyclohexyl), —NCH₃COCH₃, —SO₂N(CH₃)₂, —SO₂(aziridinyl), —CO(difluoroazetidyl), CO(difluoropiperidyl), —CO(piperazinyl), and —CO(morpholinyl); R² is 2-pyrimidyl. In some such embodiments, R³ is H, —CH₃, —CH₂OH, —CH₂OCH₂-cyclopropyl, —CH₂-azetidyl, —CH₂-piperidyl; or phenyl; R⁴ is H, —CH₃, or CH₂-phenyl. In some other such embodiments, R⁴ is H. In some other such embodiments, R⁴ is —CH₃. In some other such embodiments, R⁴ is —CH₂-phenyl. In some embodiments of compounds of formula (I), wherein when R¹ is 2-pyridyl, substituted with one or more —CH₃; R² is 2-pyrimidyl; R³ is H; R⁴ is H.

Further embodiments provided herein include combinations of at least one of the particular embodiments set forth above.

Representative compounds of formula (I) are set forth in Table 1.

Provided herein are compounds having the following formula (II):

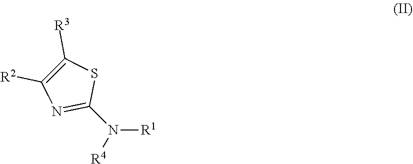

and pharmaceutically acceptable salts, tautomers, isotopologues, and stereoisomers thereof, wherein:
R¹ is isoquinolyl; pyrrolopyridyl; 2-pyrimidyl, or 2-pyridyl, wherein the 2-pyridyl is substituted with one or more substituents independently selected from H, halogen, substituted or unsubstituted C₁₋₄ alkyl, substituted or unsubstituted C₃₋₇ cycloalkyl, substituted or unsubstituted C₆₋₁₀ aryl, substituted or unsubstituted monocyclic heteroaryl, substituted or unsubstituted 3-6 membered heterocyclyl, —OR⁵, —CONR⁶₂, —CON(C₁₋₃ alkyl)(substituted or unsubstituted C₃₋₇ cycloalkyl), —NRCO(C₁₋₃ alkyl), and —CO(substituted or unsubstituted 3-6 membered heterocyclyl);
R² is 2-pyridyl, substituted with one or more substituents independently selected from H, halogen, substituted or unsubstituted C₁₋₄ alkyl, substituted or unsubstituted C₆₋₁₀ aryl, —OR⁵, and —CONR₂;
R³ is H, —CN, substituted or unsubstituted C₁₋₄ alkyl, (C₁₋₃ alkyl)O(C₁₋₃ alkyl)(substituted or unsubstituted C₃₋₇ cycloalkyl), —(C₁₋₃ alkyl)OR, (C₁₋₃ alkyl)(substituted or unsubstituted 3-6 membered heterocyclyl)-C(O)(substituted or unsubstituted 3-10 membered heterocyclic), —C(O)OR, substituted or unsubstituted C₆₋₁₀ aryl, (C₁₋₃ alkyl)NR⁶₂, —(C₁₋₃ alkyl)N(C₁₋₃ alkyl)(substituted or unsubstituted C₃₋₇ cycloalkyl), CONR⁶₂, or —C(O)N(C₁₋₃ alkyl)NR₂;
R⁴ is H or substituted or unsubstituted C₁₋₃ alkyl;
R⁵ is H, substituted or unsubstituted C₁₋₅ alkyl, or substituted or unsubstituted 3-6 membered heterocyclyl;
each R⁶ is independently selected from H, substituted or unsubstituted C₁₋₅ alkyl; substituted or unsubstituted C₃₋₆ cycloalkyl, substituted or unsubstituted 3-6 membered heterocyclyl, and (C₁₋₃ alkyl)(substituted or unsubstituted 3-6 membered heterocyclyl); and
each R is independently selected from H and substituted or unsubstituted C₁₋₄ alkyl;
provided that when R¹ and R² are both 2-pyridyl, either R¹ or R² is not substituted with H.

In one embodiment of compounds of formula (II), R¹ is 2-pyrimidyl.

In one embodiment of compounds of formula (II), R¹ is isoquinolyl; 1H-pyrrolo[3,2-c]pyridyl; or 1H-pyrrolo[2,3-c]pyridyl.

In one embodiment of compounds of formula (II), R¹ is 2-pyridyl, wherein the 2-pyridyl is substituted with one or more substituents independently selected from H, halogen, substituted or unsubstituted C₁₋₄ alkyl, substituted or unsubstituted C₃₋₇ cycloalkyl, substituted or unsubstituted C₆₋₁₀ aryl, substituted or unsubstituted monocyclic heteroaryl, substituted or unsubstituted 3-6 membered heterocyclyl, —OR⁵, —CONR⁶₂, —CON(C₁₋₃ alkyl)(substituted or unsubstituted C₃₋₇ cycloalkyl), —NRCO(C₁₋₃ alkyl), and —CO(substituted or unsubstituted 3-6 membered heterocyclyl).

In some embodiments of compounds of formula (II), R¹ is 2-pyridyl, substituted with one or more substituents independently selected from H, Br, F, —CH₃, —CH₂CH₃, —CH₂CH₂CH₃, —CH(CH₃)₂, CF₃, cyclopropyl, cylobutyl, cyclopentyl, —OR⁵, substituted or unsubstituted phenyl, substituted or unsubstituted naphthyl; substituted or unsubstituted pyridazinyl; substituted or unsubstituted pyrazinyl; substituted or unsubstituted pyrimidyl, substituted or unsubstituted pyrazolyl, substituted or unsubstituted dihydropyranyl, substituted or unsubstituted piperidyl, substituted or unsubstituted piperazinyl, —CONR⁶₂, —CON(C₁₋₃ alkyl)(substituted or unsubstituted C₃₋₇ cycloalkyl), —NRCO(C₁₋₃ alkyl), and —CO(substituted or unsubstituted 3-6 membered heterocyclyl).

In some embodiments, R¹ is 2-pyridyl substituted with one or more OR⁵. In some such embodiments, R⁵ is H, —CH₃, —CH₂CH₃, —CH₂CH₂CH₃, —CH(CH₃)₂, tetrahydrofuranyl, or tetrahydropyranyl. In some such embodiments, R⁵ is H, —CH₃, —CH(CH₃)₂, or tetrahydropyranyl. In some such embodiments, R⁵ is H, —CH₃, —CH₂CH₃, —CH₂CH₂CH₃, —CH(CH₃)₂, piperidyl, 1-methyl-piperidyl, tetrahydrofuranyl, or tetrahydropyranyl. In some such embodiments, R⁵ is H or —CH₃. In some such embodiments, R⁵ is —CH₃. In some embodiments, R⁵ is —CH(CH₃)₂. In some such embodiments, R⁵ is tetrahydropyranyl. In some such embodiments, R⁵ is 1-methyl-piperidyl.

In some embodiments of compounds of formula (II), R¹ is 2-pyridyl substituted with one or more —CONR⁶₂. In some such embodiments, each R⁶ is independently H, substituted or unsubstituted C₁₋₅ alkyl selected from —CH₃, —CH₂CH₃, —CH₂CH₂CH₃, or —CH(CH₃)₂; substituted or unsubstituted C₃₋₆ cycloalkyl selected from cyclopropyl, cyclobutyl, cyclopentyl, and cyclohexyl; wherein the alkyl and cycloalkyl are optionally substituted with one or more substituents independently selected from OH, OCH₃, and F. In some embodiments, each R⁶ is independently H, substituted or unsubstituted C₁₋₅ alkyl selected from CH(CH₃)₂; substituted or unsubstituted C₃₋₆ cycloalkyl selected from cyclopentyl, or cyclohexyl; wherein the alkyl and cycloalkyl are optionally substituted with one or more substituents independently selected from OH, OCH₃, and F.

In some embodiments, R¹ is 2-pyridyl, substituted with one or more substituents independently selected from H, Br, F, —CH₃, —CH₂CH₃, —CH₂CH₂CH₃, —CH₂CH₂CH₂CH₃—CH(CH₃)₂, CF₃, cyclopropyl, cylobutyl, cyclopentyl, —OH, —OCH₃, —OCH₂CH₃, —OCH₂CH₂CH₃, —OCH(CH₃)₂, —O-tetrahydrofuranyl, —O-tetrahydropyranyl, substituted or unsubstituted phenyl, substituted or unsubstituted naphthyl; substituted or unsubstituted pyridazinyl; substituted or unsubstituted pyrazinyl; substituted or unsubstituted pyrimidyl, substituted or unsubstituted pyrazolyl, substituted or unsubstituted dihydropyranyl, substituted or unsubstituted piperidyl, substituted or unsubstituted piperazinyl, —CONH₂, —CONHCH₃, —CON(CH₃)₂, —CONHCH₂CH₃, —CON(CH₂CH₃)₂, —CONHCH₂CH₂CH₃—CONHCH(CH₃)₂, —CONHCH₂CH₂OH, —CONHCH₂CH₂OCH₃, —CONHCH(CH₃)CH₂OH, —CONHCH₂CF₃, —CONHCH₂CH₂-pyrrolidyl, —CONH(cyclopropyl), —CONH(cyclobutyl), —CONH(cyclopentyl), —CONH(cyclohexyl), —CONH(CH₂)-cyclopropyl-CO(azetidyl), —CO(piperidyl), —CO(piperazinyl), —CO(morpholinyl), and —NCH₃COCH₃; wherein the cyclopropyl, cyclobutyl, cyclopentyl, cyclohexyl, azetidyl, piperidyl and piperazinyl are optionally fluorinated.

In some embodiments, R¹ is 2-pyridyl, substituted with one or more substituents independently selected from H, Br, F, —CH₃, —CH₂CH₃, —CH₂CH₂CH₃, —CH₂CH₂CH₂CH₃, —CH(CH₃)₂, CF₃, cyclobutyl, cyclopentyl, —OH, —OCH₃, —OCH(CH₃)₂, —O-tetrahydropyranyl, substituted or unsubstituted phenyl, substituted or unsubstituted naphthyl; substituted or unsubstituted pyridazinyl; substituted or unsubstituted pyrazinyl; substituted or unsubstituted pyrimidyl, substituted or unsubstituted pyrazolyl, substituted or unsubstituted dihydropyranyl, substituted or unsubstituted piperidyl, substituted or unsubstituted piperazinyl, —CONH₂, —CONHCH₂CH₃, —CONHCH(CH₃)₂, —CONHCH₂CH₂OH, —CONHCH₂CH₂OCH₃, —CONHCH(CH₃)CH₂OH, —CONHCH₂CF₃, —CONHCH₂CH₂-pyrrolidyl, —CONH(cyclopropyl), —CONH(cyclopentyl), —CONH(cyclohexyl), —CONH(CH₂)-cyclopropyl, —CO(azetidyl), CO(piperidyl), —CO(piperazinyl), —CO(morpholinyl), and —NCH₃COCH₃; wherein the cyclopropyl, cyclobutyl, cyclopentyl, cyclohexyl, azetidyl, piperidyl and piperazinyl are optionally fluorinated.

In some embodiments, R¹ is 2-pyridyl, substituted with one or more substituents independently selected from H, Br, F, —CH₃, —CH₂CH₃, —CH₂CH₂CH₃, —CH₂CH₂CH₂CH₃, —CH(CH₃)₂, CF₃, cyclobutyl, cyclopentyl, —OH, —OCH₃, —OCH(CH₃)₂, —O-tetrahydropyranyl, phenyl, phenyl(COOH), phenyl(phenyl), phenyl(CONHCH₃), naphthyl; pyridazinyl; pyrazinyl; pyrimidyl; 1-methylpyrazolyl; dihydropyranyl; 1-methyl-piperidyl; piperidyl substituted with COOH, CONHMe, or CONHCH₂CF₃; 1-methyl-piperazinyl; piperazinyl substituted with COC(CH₃)₂OH or CO-cyclopropyl-CF₃; —CONH₂, —CONMe₂, —CONHCH₂CH₃, —CONHCH(CH₃)₂, —CONHCH₂CH₂OH, —CONHCH₂CH₂OCH₃, —CONHCH(CH₃)CH₂OH, —CONHCH₂CF₃, —CONHCH₂CH₂-pyrrolidyl, —CONH(cyclopropyl), —CONH(cyclopentyl), —CONH(CH₂)-cyclopropyl, —CONH(difluorocyclohexyl), —CO(difluoroazetidyl), CO(difluoropiperidyl), —CO(piperazinyl), —CO(morpholinyl), and —NCH₃COCH₃.

In some embodiments of compounds of formula (II), R² is substituted with one or more substituents independently selected from H, F, methyl, ethyl, substituted or unsubstituted phenyl, —OR⁵, and —CONR₂. In some embodiments, R² is substituted with one or more substituents independently selected from H, F, —CH₃, —CH₂CH₃, —OCH₃, —OCH₂CH₃, —OCH₂CH₂CH₃, —OCH(CH₃)₂, —O-(1-methyl-piperidyl), —O-tetrahydrofuranyl, —O— tetrahydropyranyl, —CONH₂, —CONHCH₃, —CON(CH₃)₂, —CONHCH₂CH₃, —CON(CH₂CH₃)₂, and substituted or unsubstituted phenyl. In some such embodiments, R² is substituted with one or more substituents independently selected from H, F, CH₃, —CH₂CH₃, —OCH₃, —OCH₂CH₃, —OCH(CH₃)₂, —O-(1-methyl-piperidyl), —O-tetrahydropyranyl, —CONH₂, —CON(CH₃)₂, and substituted or unsubstituted phenyl. In some such embodiments, R² is substituted with one or more substituents independently selected from H, F, CH₃, —CH₂CH₃, —OCH₃, —OCH₂CH₃, —OCH(CH₃)₂, —O-(1-methyl-piperidyl), —O-tetrahydropyranyl, —CONH₂, —CON(CH₃)₂, phenyl; and phenyl, substituted with cyclopropyl(COOH).

In some embodiments of compounds of formula (II), R³ is H, —CN, CH₃, —CH₂CH₃, —CH(CH₃)₃, —CH₂OH, —CH₂CH₂OH, —CH₂OCH₂-cyclopropyl, —CH₂OCH₂-cyclobutyl, —CH₂CH₂O-cyclobutyl, —CH₂CH₂OCH₂-cyclopropyl, —CH₂OCH₂CH₂-cyclopropyl, —CH₂N(CH₃)₂, —CH₂-azetidyl, —CH₂-piperidyl, —CH₂(dimethylmorpholinyl), —CH₂(dimethylpiperazyl), —CH₂-pirrolidyl, —CH₂(morpholinyl), —COOH, —CO(dimethylmorpholinyl), —CO(morpholinyl), —CO(1,3-dioxolane-piperidyl), —CO(piperidyl), —CO(pirrolidyl), —CO(1-methyl-piperazyl), —CO(octahydropyrrolo[1,2-a]pyrazyl), —CONHCH₂-cyclohexyl, —CONHCH₂-tetrahydropiranyl, —CONHCH₂-cyclopentyl, —CONH(CH₂)₂N(CH₃)₂, —CON(CH₃)₂, or phenyl. In some embodiments of compounds of formula (I), R³ is H, —CN, —CH₃, —CH(CH₃)₃, —CH₂OH, —CH₂OCH₂-cyclobutyl, —CH₂CH₂O-cyclobutyl, —CH₂OCH₂-cyclopropyl, —CH₂N(CH₃)₂, —CH₂-azetidyl, —CH₂-piperidyl, —CH₂(dimethylmorpholinyl), —CH₂(dimethylpiperazyl), —CH₂-pirrolidyl, —CH₂(morpholinyl), —COOH, —CO(dimethylmorpholinyl), —CO(morpholinyl), —CO(1,3-dioxolane-piperidyl), —CO(piperidyl), —CO(pirrolidyl), —CO(1-methyl-piperazyl), —CO(octahydropyrrolo[1,2-a]pyrazyl), —CONHCH₂-cyclohexyl, —CONHCH₂-tetrahydropiranyl, —CONHCH₂-cyclopentyl, —CONH(CH₂)₂N(CH₃)₂, —CON(CH₃)₂, or phenyl.

In some embodiments of compounds of formula (II), R⁴ is H, —CH₃, or —CH(CH₃)₂.

In some embodiments of compounds of formula (II), R⁴ is H. In some embodiments of compounds of formula (II), R⁴ is CH₃. In some embodiments of compounds of formula (II), R⁴ is —CH(CH₃)₂.

In one embodiment of compounds of formula (II), R¹ is isoquinolyl, 1H-pyrrolo[3,2-c]pyridyl, 1H-pyrrolo[2,3-c]pyridyl and R² is substituted with one or more substituents independently selected from H, F, methyl, ethyl, substituted or unsubstituted phenyl, —OR⁵, and —CONR₂. In some embodiments, R² is substituted with one or more substituents independently selected from H, F, —CH₃, —CH₂CH₃, —OCH₃, —OCH₂CH₃, —OCH₂CH₂CH₃, —OCH(CH₃)₂, —O-(1-methyl-piperidyl), —O-tetrahydrofuranyl, —O-tetrahydropyranyl, —CONH₂, —CONHCH₃, —CON(CH₃)₂, —CONHCH₂CH₃, —CON(CH₂CH₃)₂, and substituted or unsubstituted phenyl. In some embodiments of compounds of formula (II), R³ is H, CH₃, —CH₂CH₃, —CH₂OH, —CH₂CH₂OH, —CH₂OCH₂-cyclopropyl, —CH₂CH₂OCH₂-cyclopropyl, —CH₂OCH₂CH₂-cyclopropyl, —CH₂-azetidyl or —CH₂-piperidyl. In some other such embodiments, R⁴ is H or —CH₃. In some other such embodiments, R⁴ is H. In some other such embodiments, R⁴ is —CH₃. In some other such embodiments, R³ is H, —CH₃, —CH₂OH, —CH₂OCH₂-cyclopropyl, —CH₂-azetidyl or —CH₂-piperidyl. In some other such embodiments, R⁴ is H.

In one embodiment of compounds of formula (II), R¹ is isoquinolyl, 1H-pyrrolo[3,2-c]pyridyl, or 1H-pyrrolo[2,3-c]pyridyl and R² is substituted with H; R³ is H, CH₃, —CH₂CH₃, —CH₂OH, —CH₂CH₂OH, —CH₂OCH₂-cyclopropyl, —CH₂CH₂OCH₂-cyclopropyl, —CH₂OCH₂CH₂-cyclopropyl, —CH₂-azetidyl or —CH₂-piperidyl; R⁴ is H or —CH₃. In one embodiment, R³ is H. In one embodiment, R⁴ is H. In one embodiment, R⁴ is —CH₃.

In some embodiments of compounds of formula (II), wherein when R¹ is 2-pyridyl, substituted with one or more substituents independently selected from H, Br, F, —CH₃, —CH₂CH₃, —CH₂CH₂CH₃, —CH(CH₃)₂, CF₃, cyclopropyl, cylobutyl, cyclopentyl, —OR⁵, substituted or unsubstituted phenyl, substituted or unsubstituted naphthyl; substituted or unsubstituted pyridazinyl; substituted or unsubstituted pyrazinyl; substituted or unsubstituted pyrimidyl, substituted or unsubstituted pyrazolyl, substituted or unsubstituted dihydropyranyl, substituted or unsubstituted piperidyl, substituted or unsubstituted piperazinyl, —CONR⁶₂, —CON(C₁₋₃ alkyl)(substituted or unsubstituted C₃₋₇ cycloalkyl), —NRCO(C₁₋₃ alkyl), and —CO(substituted or unsubstituted 3-6 membered heterocyclyl); R² is substituted with one or more substituents independently selected from H, F, —CH₃, —CH₂CH₃, substituted or unsubstituted phenyl; —OR⁵, and —CONR₂. In some other such embodiments, R³ is H, —CN, CH₃, —CH₂CH₃, —CH(CH₃)₃, —CH₂OH, —CH₂CH₂OH, —CH₂OCH₂-cyclopropyl, —CH₂OCH₂-cyclobutyl, —CH₂CH₂O-cyclobutyl, —CH₂CH₂OCH₂-cyclopropyl, —CH₂OCH₂CH₂-cyclopropyl, —CH₂N(CH₃)₂, —CH₂-azetidyl, —CH₂-piperidyl, —CH₂(dimethylmorpholinyl), —CH₂(dimethylpiperazyl), —CH₂-pirrolidyl, —CH₂(morpholinyl), —COOH, —CO(dimethylmorpholinyl), —CO(morpholinyl), —CO(1,3-dioxolane-piperidyl), —CO(piperidyl), —CO(pirrolidyl), —CO(1-methyl-piperazyl), —CO(octahydropyrrolo[1,2-a]pyrazyl), —CONHCH₂-cyclohexyl, —CONHCH₂-tetrahydropiranyl, —CONHCH₂-cyclopentyl, —CONH(CH₂)₂N(CH₃)₂, —CON(CH₃)₂, or phenyl. In some other such embodiments, R⁴ is H, —CH₃, or —CH(CH₃)₂. In some other such embodiments, R⁴ is H. In some other such embodiments, R⁴ is —CH₃. In some other such embodiments, R⁴ is —CH(CH₃)₂.

In some embodiments of compounds of formula (II), wherein when R¹ is 2-pyridyl, substituted with one or more substituents independently selected from H, Br, F, —CH₃, —CH₂CH₃, —CH₂CH₂CH₃, —CH₂CH₂CH₂CH₃—CH(CH₃)₂, CF₃, cyclopropyl, cylobutyl, cyclopentyl, —OH, —OCH₃, —OCH₂CH₃, —OCH₂CH₂CH₃, —OCH(CH₃)₂, —O-tetrahydrofuranyl, —O-tetrahydropyranyl, substituted or unsubstituted phenyl, substituted or unsubstituted naphthyl; substituted or unsubstituted pyridazinyl; substituted or unsubstituted pyrazinyl; substituted or unsubstituted pyrimidyl, substituted or unsubstituted pyrazolyl, substituted or unsubstituted dihydropyranyl, substituted or unsubstituted piperidyl, substituted or unsubstituted piperazinyl, —CONH₂, —CONHCH₃, —CON(CH₃)₂, —CONHCH₂CH₃, —CON(CH₂CH₃)₂, —CONHCH₂CH₂CH₃, —CONHCH(CH₃)₂, —CONHCH₂CH₂OH, —CONHCH₂CH₂OCH₃, —CONHCH(CH₃)CH₂OH, —CONHCH₂CF₃, —CONHCH₂CH₂-pyrrolidyl, —CONH(cyclopropyl), —CONH(cyclobutyl), —CONH(cyclopentyl), —CONH(cyclohexyl), —CONH(CH₂)-cyclopropyl-CO(azetidyl), —CO(piperidyl), —CO(piperazinyl), —CO(morpholinyl), and —NCH₃COCH₃; wherein the cyclopropyl, cyclobutyl, cyclopentyl, cyclohexyl, azetidyl, piperidyl and piperazinyl are optionally fluorinated; R² is substituted with one or more substituents independently selected from H, F, —CH₃, —CH₂CH₃, —OCH₃, —OCH₂CH₃, —OCH₂CH₂CH₃, —OCH(CH₃)₂, —O-(1-methyl-piperidyl), —O-tetrahydrofuranyl, —O-tetrahydropyranyl, —CONH₂, —CONHCH₃, —CON(CH₃)₂, —CONHCH₂CH₃, —CON(CH₂CH₃)₂; and substituted or unsubstituted phenyl. In some such embodiments, R³ is H, —CN, CH₃, —CH₂CH₃, —CH(CH₃)₃, —CH₂OH, —CH₂CH₂OH, —CH₂OCH₂-cyclopropyl, —CH₂OCH₂-cyclobutyl, —CH₂CH₂O-cyclobutyl, —CH₂CH₂OCH₂-cyclopropyl, —CH₂OCH₂CH₂-cyclopropyl, —CH₂N(CH₃)₂, —CH₂-azetidyl, —CH₂-piperidyl, —CH₂(dimethylmorpholinyl), —CH₂(dimethylpiperazyl), —CH₂-pirrolidyl, —CH₂(morpholinyl), —COOH, —CO(dimethylmorpholinyl), —CO(morpholinyl), —CO(1,3-dioxolane-piperidyl), —CO(piperidyl), —CO(pirrolidyl), —CO(1-methyl-piperazyl), —CO(octahydropyrrolo[1,2-a]pyrazyl), —CONHCH₂-cyclohexyl, —CONHCH₂-tetrahydropiranyl, —CONHCH₂-cyclopentyl, —CONH(CH₂)₂N(CH₃)₂, —CON(CH₃)₂, or phenyl. In some other such embodiments, R⁴ is H, —CH₃, or —CH(CH₃)₂. In some other such embodiments, R⁴ is H. In some other such embodiments, R⁴ is —CH₃. In some other such embodiments, R⁴ is —CH(CH₃)₂.

In some embodiments of compounds of formula (II), wherein when R¹ is 2-pyridyl, substituted with one or more substituents independently selected from H, Br, F, —CH₃, —CH₂CH₃, —CH₂CH₂CH₃, —CH₂CH₂CH₂CH₃, —CH(CH₃)₂, CF₃, cyclobutyl, cyclopentyl, —OH, —OCH₃, —OCH(CH₃)₂, —O-tetrahydropyranyl, phenyl, phenyl(COOH), phenyl(phenyl), phenyl(CONHCH₃), naphthyl; pyridazinyl; pyrazinyl; pyrimidyl; 1-methylpyrazolyl; dihydropyranyl; 1-methyl-piperidyl; piperidyl substituted with COOH, CONHMe, or CONHCH₂CF₃; 1-methyl-piperazinyl; piperazinyl substituted with COC(CH₃)₂₀H or CO-cyclopropyl-CF₃; —CONH₂, —CONMe₂, —CONHCH₂CH₃, —CONHCH(CH₃)₂, —CONHCH₂CH₂OH, —CONHCH₂CH₂OCH₃, —CONHCH(CH₃)CH₂OH, —CONHCH₂CF₃, —CONHCH₂CH₂-pyrrolidyl, —CONH(cyclopropyl), —CONH(cyclopentyl), —CONH(CH₂)-cyclopropyl, —CONH(difluorocyclohexyl), —CO(difluoroazetidyl), —CO(difluoropiperidyl), —CO(piperazinyl), —CO(morpholinyl), and —NCH₃COCH₃; R² is substituted with one or more substituents independently selected from H, F, —CH₃, —CH₂CH₃, —OCH₃, —OCH₂CH₃, —OCH(CH₃)₂, —O-(1-methyl-piperidyl), —O-tetrahydropyranyl, —CONH₂, —CON(CH₃)₂; phenyl; and phenyl, substituted with cyclopropyl(COOH); wherein when R¹ and R² are both 2-pyridyl, either R¹ or R² is not substituted with H; and wherein R³ is H, —CN, CH₃, —CH₂CH₃, —CH(CH₃)₃, —CH₂OH, —CH₂CH₂OH, —CH₂OCH₂-cyclopropyl, —CH₂OCH₂-cyclobutyl, —CH₂CH₂O-cyclobutyl, —CH₂CH₂OCH₂-cyclopropyl, —CH₂OCH₂CH₂-cyclopropyl, —CH₂N(CH₃)₂, —CH₂-azetidyl, —CH₂-piperidyl, —CH₂(dimethylmorpholinyl), —CH₂(dimethylpiperazyl), —CH₂-pirrolidyl, —CH₂(morpholinyl), —COOH, —CO(dimethylmorpholinyl), —CO(morpholinyl), —CO(1,3-dioxolane-piperidyl), —CO(piperidyl), —CO(pirrolidyl), —CO(1-methyl-piperazyl), —CO(octahydropyrrolo[1,2-a]pyrazyl), —CONHCH₂-cyclohexyl, —CONHCH₂-tetrahydropiranyl, —CONHCH₂-cyclopentyl, —CONH(CH₂)₂N(CH₃)₂, —CON(CH₃)₂, or phenyl; R⁴ is H, —CH₃, or —CH(CH₃)₂. In some other such embodiments, R⁴ is H. In some other such embodiments, R⁴ is —CH₃. In some other such embodiments, R⁴ is —CH(CH₃)₂.

Further embodiments provided herein include combinations of at least one of the particular embodiments set forth above.

Representative compounds of formula (II) are set forth in Table 2 and Table 3.

Each of the compounds in Table 1, Table 2, and Table 3 was tested in one or more of the in vitro parasitic motility assays and was found to have activity therein.

Provided herein are compounds having the following formula (III):

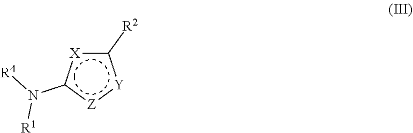

and pharmaceutically acceptable salts, tautomers, isotopologues, and stereoisomers thereof, wherein:
X is CR³, N, or S;
Y is N, or S;
Z is CR³, or S;
R¹ is 2-pyridyl, unsubstituted or substituted with one or more substituents independently selected from halogen, substituted or unsubstituted C₁₋₄ alkyl, substituted or unsubstituted C₃₋₇ cycloalkyl, substituted or unsubstituted C₆₋₁₀ aryl, substituted or unsubstituted monocyclic heteroaryl, substituted or unsubstituted 3-6 membered heterocyclyl, —OR⁵, —CONR⁶₂, and —CO(substituted or unsubstituted 3-6 membered heterocyclyl);
R² is 2-pyridyl, unsubstituted or substituted with one or more substituents independently selected from halogen, substituted or unsubstituted C₁₋₄ alkyl, substituted or unsubstituted C₆₋₁₀ aryl, —OR⁵, and —CONR₂;
R³ is H, substituted or unsubstituted C₁₋₄ alkyl, or (C₁₋₃ alkyl)O(C₁₋₃ alkyl)(substituted or unsubstituted C₃₋₇ cycloalkyl), (C₁₋₃ alkyl)OR;
R⁴ is H, substituted or unsubstituted C₁₋₃ alkyl;
R⁵ is H, substituted or unsubstituted C₁₋₅ alkyl, or unsubstituted 3-6 membered heterocyclyl;
each R⁶ is independently selected from H, substituted or unsubstituted C₁₋₅ alkyl; substituted or unsubstituted C₃₋₆ cycloalkyl, substituted or unsubstituted 3-6 membered heterocyclyl, and (C₁₋₃ alkyl)(substituted or unsubstituted 3-6 membered heterocyclyl); and
each R is independently selected from H and substituted or unsubstituted C₁₋₄ alkyl.

In one embodiment, the compound is a compound of formula (IIIa):

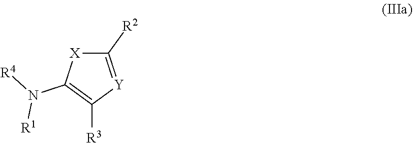

and pharmaceutically acceptable salts, tautomers, isotopologues, and stereoisomers thereof, wherein: X is N or S; Y is N or S.

In some embodiments, the compound is a compound of formula (IIIb):

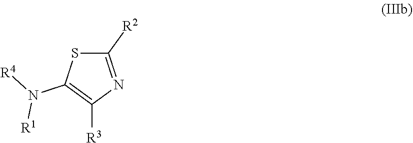

and pharmaceutically acceptable salts, tautomers, isotopologues, and stereoisomers thereof.

In some embodiments, the compound is a compound of formula (IIIc):

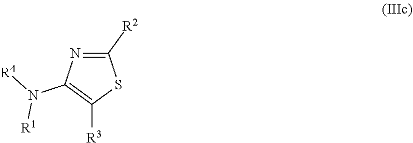

and pharmaceutically acceptable salts, tautomers, isotopologues, and stereoisomers thereof.

In some embodiments, the compound is a compound of formula (IIId):

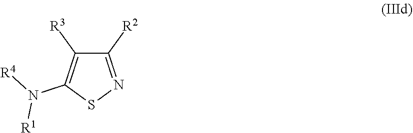

and pharmaceutically acceptable salts, tautomers, isotopologues, and stereoisomers thereof.

In some embodiments of compounds of formula (III), (IIIa), (IIIb), (IIIc), and (IIId), R¹ is 2-pyridyl unsubstituted or substituted with one or more substituents independently selected from halogen, substituted or unsubstituted C₁₋₄ alkyl, substituted or unsubstituted C₃₋₇ cycloalkyl, substituted or unsubstituted C₆₋₁₀ aryl, substituted or unsubstituted monocyclic heteroaryl, substituted or unsubstituted 3-6 membered heterocyclyl, —OR⁵, —CONR⁶₂, and —CO(substituted or unsubstituted 3-6 membered heterocyclyl);
R² is 2-pyridyl, unsubstituted or substituted with one or more substituents independently selected from halogen, substituted or unsubstituted C₁₋₄ alkyl, substituted or unsubstituted C₆₋₁₀ aryl, —OR⁵, and —CONR₂;
R³ is H, substituted or unsubstituted C₁₋₄ alkyl, or (C₁₋₃ alkyl)O(C₁₋₃ alkyl)(substituted or unsubstituted C₃₋₇ cycloalkyl), (C₁₋₃ alkyl)OR;
R⁴ is H, substituted or unsubstituted C₁₋₃ alkyl;
R⁵ is H, substituted or unsubstituted C₁₋₅ alkyl, or unsubstituted 3-6 membered heterocyclyl;
each R⁶ is independently selected from H, substituted or unsubstituted C₁₋₅ alkyl; substituted or unsubstituted C₃₋₆ cycloalkyl, substituted or unsubstituted 3-6 membered heterocyclyl, and (C₁₋₃ alkyl)(substituted or unsubstituted 3-6 membered heterocyclyl); and
each R is independently selected from H and substituted or unsubstituted C₁₋₄ alkyl.

In some embodiments of compounds of formula (III), (IIIa), (IIIb), (IIIc), and (IIId), R¹ is 2-pyridyl, substituted or unsubstituted with one or more substituents independently selected from halogen, substituted or unsubstituted C₁₋₄ alkyl, substituted or unsubstituted C₃₋₇ cycloalkyl, substituted or unsubstituted C₆₋₁₀ aryl, substituted or unsubstituted monocyclic heteroaryl, substituted or unsubstituted 3-6 membered heterocyclyl, —OR⁵, —CONR⁶₂, and —CO(substituted or unsubstituted 3-6 membered heterocyclyl).

In some embodiments of compounds of formula (III), (IIIa), (IIIb), (IIIc), and (IIId), R¹ is 2-pyridyl, unsubstituted or substituted with one or more substituents independently selected from —CH₃, —CH₂CH₃, —CH₂CH₂CH₃, —CH(CH₃)₂, cyclopropyl, cylobutyl, cyclopentyl, —OR⁵, substituted or unsubstituted phenyl, substituted or unsubstituted pyrazolyl, substituted or unsubstituted dihydropyranyl, substituted or unsubstituted piperidyl, substituted or unsubstituted piperazinyl, —CONR⁶₂, and —CO(substituted or unsubstituted 3-6 membered heterocyclyl).

In some embodiments, R¹ is 2-pyridyl, unsubstituted or substituted with one or more substituents independently selected from —CH₃, —CH₂CH₃, —CH₂CH₂CH₃, —CH(CH₃)₂, cyclopropyl, cyclobutyl, cyclopentyl, —OR⁵, substituted or unsubstituted phenyl, and —CONR⁶₂.

In some embodiments, R¹ is 2-pyridyl, unsubstituted or substituted with one or more substituents independently selected from —CH₃, —CH₂CH₃, —CH(CH₃)₂, cyclopropyl, cyclobutyl, —OR⁵, and substituted or unsubstituted phenyl.

In some embodiments, R¹ is 2-pyridyl, unsubstituted or substituted with one or more OR⁵. In some such embodiments, R⁵ is H, —CH₃, —CH₂CH₃, —CH₂CH₂CH₃, —CH(CH₃)₂, tetrahydrofuranyl, tetrahydropyranyl, or 1-methylpiperidyl. In some such embodiments, R⁵ is H, —CH₃, —CH₂CH₃, —CH(CH₃)₂, or tetrahydropyranyl.

In some such embodiments, R⁵ is H or —CH₃. In some such embodiments, R⁵ is —CH₃. In some embodiments, R⁵ is —CH(CH₃)₂.

In some embodiments of compounds of formula (III), (IIIa), (IIIb), (IIIc), and (IIId), R¹ is 2-pyridyl substituted with one or more —CONR⁶₂. In some such embodiments, each R⁶ is independently selected from H, substituted or unsubstituted C₁₋₅ alkyl; substituted or unsubstituted C₃₋₆ cycloalkyl, substituted or unsubstituted 3-6 membered heterocyclyl, and (C₁₋₃ alkyl)(substituted or unsubstituted 3-6 membered heterocyclyl). In some such embodiments, each R⁶ is independently H, substituted or unsubstituted C₁₋₅ alkyl selected from —CH₃, —CH₂CH₃, —CH₂CH₂CH₃, and —CH(CH₃)₂; substituted or unsubstituted C₃₋₆ cycloalkyl selected from cyclopropyl, cyclobutyl, cyclopentyl, and cyclohexyl; wherein the alkyl and cycloalkyl are optionally substituted with one or more substituents independently selected from OH, OCH₃, and F. In some embodiments, each R⁶ is independently H, substituted or unsubstituted C₁₋₅ alkyl selected from —CH(CH₃)₂; substituted or unsubstituted C₃₋₆ cycloalkyl selected from cyclopentyl and cyclohexyl; wherein the alkyl and cycloalkyl are optionally substituted with one or more substituents independently selected from OH, OCH₃, and F.

In some embodiments, R¹ is 2-pyridyl, unsubstituted or substituted with one or more substituents independently selected from —CH₃, —CH₂CH₃, —CH(CH₃)₂, cyclopropyl, cyclobutyl, —OH, —OCH₃, —OCH₂CH₃, —OCH₂CH₂CH₃, —OCH(CH₃)₂, —O-tetrahydrofuranyl, —O— tetrahydropyranyl, and substituted or unsubstituted phenyl.

In some embodiments, R¹ is 2-pyridyl, unsubstituted or substituted with one or more substituents independently selected from —CH₃, —CH₂CH₃, —CH(CH₃)₂, cyclopropyl, cyclobutyl, —OCH₃, —OCH(CH₃)₂, —O-tetrahydrofuranyl, —O-tetrahydropyranyl, and substituted and unsubstituted phenyl.

In some embodiments, R¹ is 2-pyridyl, unsubstituted or substituted with one or more substituents independently selected from —CH₃, —OCH(CH₃)₂, and substituted or unsubstituted phenyl.

In some embodiments of compounds of formula (III), (IIIa), (IIIb), (IIIc), and (IIId), R² is 2-pyridyl, unsubstituted or substituted with one or more substituents independently selected from —CH₃, —CH₂CH₃, —CH₂CH₂CH₃, —CH(CH₃)₂, cyclopropyl, cyclobutyl, cyclopentyl, substituted or unsubstituted phenyl, —OR⁵, and —CONR₂.

In some such embodiments R² is 2-pyridyl, unsubstituted or substituted with one or more substituents independently selected from —CH₃, —CH₂CH₃, —CH(CH₃)₂, cyclopropyl, cyclobutyl, substituted or unsubstituted phenyl, —OCH₃, —OCH₂CH₃, —OCH₂CH₂CH₃, —OCH(CH₃)₂, —O-tetrahydrofuranyl, —O-tetrahydropyranyl, substituted or unsubstituted phenyl, and —CONR₂.

In some such embodiments R² is 2-pyridyl, unsubstituted or substituted with one or more substituents independently selected from —CH₃, —OCH₃, —O-tetrahydrofuranyl, and substituted or unsubstituted phenyl.

In some embodiments of compounds of formula (III), (IIIa), (IIIb), (IIIc), and (IIId), R³ is H, —CH₃, —CH₂CH₃, —CH(CH₃)₃, —CH₂OH, —CH₂CH₂OH, —CH₂OCH₂-cyclopropyl, or —CH₂OCH₂-cyclobutyl.

In some embodiments of compounds of formula (III), (IIIa), (IIIb), (IIIc), and (IIId), R³ is H, —CH₃, —CH₂CH₃, —CH(CH₃)₃, or —CH₂OH.

In some embodiments of compounds of formula (III), (IIIa), (IIIb), (IIIc), and (IIId), R³ is H, or —CH₃. In some such embodiments, R³ is H.

In some embodiments of compounds of formula (III), (IIIa), (IIIb), (IIIc), and (IIId), R⁴ is H, or —CH₃. In some such embodiments, R⁴ is H. In some such embodiments, R⁴ is —CH₃.

In some embodiments of compounds of formula (III), (IIIa), (IIIb), (IIIc), and (IIId), wherein when R¹ is 2-pyridyl, unsubstituted or substituted with one or more substituents independently selected from —CH₃, —CH₂CH₃, —CH₂CH₂CH₃, —CH(CH₃)₂, cyclopropyl, cylobutyl, cyclopentyl, —OR⁵, substituted or unsubstituted phenyl, substituted or unsubstituted pyrazolyl, substituted or unsubstituted dihydropyranyl, substituted or unsubstituted piperidyl, substituted or unsubstituted piperazinyl, —CONR⁶₂, and —CO(substituted or unsubstituted 3-6 membered heterocyclyl); R² is 2-pyridyl, unsubstituted or substituted with one or more substituents independently selected from —CH₃, —CH₂CH₃, —CH₂CH₂CH₃, —CH(CH₃)₂, cyclopropyl, cylobutyl, cyclopentyl, substituted or unsubstituted phenyl, —OR⁵, and —CONR₂. In some other such embodiments, R³ is H, —CH₃, —CH₂CH₃, —CH(CH₃)₃, —CH₂OH, —CH₂CH₂OH, —CH₂OCH₂-cyclopropyl, or —CH₂OCH₂-cyclobutyl. In some such embodiments, R³ is H, or —CH₃. In some such embodiments, R³ is H. In some such embodiments, R³ is —CH₃. In some other such embodiments, R⁴ is H, or —CH₃. In some other such embodiments, R⁴ is H. In some other such embodiments, R⁴ is —CH₃.

In some embodiments of compounds of formula (III), (IIIa), (IIIb), (IIIc), and (IIId), wherein when R¹ is 2-pyridyl, unsubstituted or substituted with one or more substituents independently selected from —CH₃, —CH₂CH₃, —CH(CH₃)₂, cyclopropyl, cylobutyl, —OH, —OCH₃, —OCH₂CH₃, —OCH₂CH₂CH₃, —OCH(CH₃)₂, —O-tetrahydrofuranyl, —O-tetrahydropyranyl, and substituted or unsubstituted phenyl; R² is 2-pyridyl, unsubstituted or substituted with one or more substituents independently selected from —CH₃, —OCH₃, —O-tetrahydrofuranyl, or substituted and unsubstituted phenyl; R³ is H, or —CH₃; R⁴ is H, or —CH₃. In some such embodiments, R³ is H. In some such embodiments, R³ is —CH₃. In some such embodiments, R⁴ is H. In some such embodiments, R⁴ is —CH₃.

In some embodiments of compounds of formula (III), (IIIa), (IIIb), (IIIc), and (IIId), wherein when R¹ is 2-pyridyl, unsubstituted or substituted with one or more substituents independently selected from —CH₃, —OCH(CH₃)₂, and substituted or unsubstituted phenyl; R² is 2-pyridyl, unsubstituted or substituted with one or more substituents independently selected from —CH₃, —OCH₃, —O-tetrahydrofuranyl, and substituted or unsubstituted phenyl; R³ is H, or —CH₃; R⁴ is H, or —CH₃. In some such embodiments, R³ is H. In some such embodiments, R³ is —CH₃. In some such embodiments, R⁴ is H. In some such embodiments, R⁴ is —CH₃.

Further embodiments provided herein include combinations of at least one of the particular embodiments set forth above.

Representative compounds of formula (III), (IIIa), (IIIb), (IIIc), and (IIId), are set forth in Table 1.

In one embodiment, the compound is a compound of formula (IV):

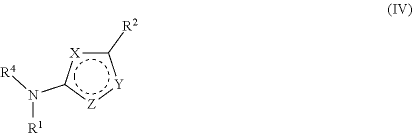

and pharmaceutically acceptable salts, tautomers, isotopologues, and stereoisomers thereof, wherein:
X is O, or CR³;
Y is NRⁿ, or CR³;
Z is N, or NRⁿ;
R¹ is 2-pyridyl, unsubstituted or substituted with one or more substituents independently selected from halogen, substituted or unsubstituted C₁₋₄ alkyl, substituted or unsubstituted C₃₋₇ cycloalkyl, substituted or unsubstituted C₆₋₁₀ aryl, substituted or unsubstituted monocyclic heteroaryl, substituted or unsubstituted 3-6 membered heterocyclyl, —OR⁵, —CONR⁶₂, —CO(substituted or unsubstituted 3-6 membered heterocyclyl), and —NR₂;
R² is 2-pyridyl, unsubstituted or substituted with one or more substituents independently selected from halogen, substituted or unsubstituted C₁₋₄ alkyl, substituted or unsubstituted C₆₋₁₀ aryl, —OR⁵, and —CONR₂;
R³ is H, substituted or unsubstituted C₁₋₄ alkyl, or (C₁₋₃ alkyl)O(C₁₋₃ alkyl)(substituted or unsubstituted C₃₋₇ cycloalkyl), (C₁₋₃ alkyl)OR;
R⁴ is H, substituted or unsubstituted C₁₋₃ alkyl;
R⁵ is H, substituted or unsubstituted C₁₋₅ alkyl, or unsubstituted 3-6 membered heterocyclyl;
each R⁶ is independently selected from H, substituted or unsubstituted C₁₋₅ alkyl; substituted or unsubstituted C₃₋₆ cycloalkyl, substituted or unsubstituted 3-6 membered heterocyclyl, and (C₁₋₃ alkyl)(substituted or unsubstituted 3-6 membered heterocyclyl); and
each R is independently selected from H and substituted or unsubstituted C₁₋₄ alkyl;
each Rⁿ is independently H, substituted or unsubstituted C₁₋₄ alkyl, or substituted or unsubstituted C₆₋₁₀ aryl.

In some embodiments, the compound is a compound of formula (IVa):

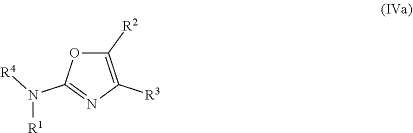

and pharmaceutically acceptable salts, tautomers, isotopologues, and stereoisomers thereof.

In some embodiments, the compound is a compound of formula (IVb):

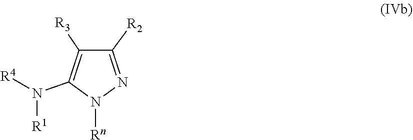

and pharmaceutically acceptable salts, tautomers, isotopologues, and stereoisomers thereof.

In some embodiments, the compound is a compound of formula (IVc):

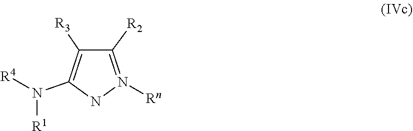

and pharmaceutically acceptable salts, tautomers, isotopologues, and stereoisomers thereof.

In some embodiments of compounds of formula (IV), (IVa), (IVb), and (IVc), R¹ is 2-pyridyl, unsubstituted or substituted with one or more substituents independently selected from halogen, substituted or unsubstituted C₁₋₄ alkyl, substituted or unsubstituted C₃₋₇ cycloalkyl, substituted or unsubstituted C₆₋₁₀ aryl, substituted or unsubstituted monocyclic heteroaryl, substituted or unsubstituted 3-6 membered heterocyclyl, —OR⁵, —CONR⁶₂, and —CO(substituted or unsubstituted 3-6 membered heterocyclyl) and —NR₂;
R² is 2-pyridyl, unsubstituted or substituted with one or more substituents independently selected from halogen, substituted or unsubstituted C₁₋₄ alkyl, substituted or unsubstituted C₆₋₁₀ aryl, —OR⁵, and —CONR₂;
R³ is H, substituted or unsubstituted C₁₋₄ alkyl, or (C₁₋₃ alkyl)O(C₁₋₃ alkyl)(substituted or unsubstituted C₃₋₇ cycloalkyl), (C₁₋₃ alkyl)OR;
R⁴ is H, substituted or unsubstituted C₁₋₃ alkyl;
R⁵ is H, substituted or unsubstituted C₁₋₅ alkyl, or unsubstituted 3-6 membered heterocyclyl;
each R⁶ is independently selected from H, substituted or unsubstituted C₁₋₅ alkyl; substituted or unsubstituted C₃₋₆ cycloalkyl, substituted or unsubstituted 3-6 membered heterocyclyl, and (C₁₋₃ alkyl) (substituted or unsubstituted 3-6 membered heterocyclyl); and
each R is independently selected from H and substituted or unsubstituted C₁₋₄ alkyl;
each Rⁿ is independently H, substituted or unsubstituted C₁₋₄ alkyl, or substituted or unsubstituted C₆₋₁₀ aryl.

In some embodiments of compounds of formula (IV), (IVa), (IVb), and (IVc), R¹ is 2-pyridyl, unsubstituted or substituted with one or more substituents independently selected from —CH₃, —CH₂CH₃, —CH₂CH₂CH₃, —CH(CH₃)₂, cyclopropyl, cylobutyl, cyclopentyl, —OR⁵, substituted or unsubstituted phenyl, substituted or unsubstituted pyrazolyl, substituted or unsubstituted dihydropyranyl, substituted or unsubstituted piperidyl, substituted or unsubstituted piperazinyl, —CONR⁶₂, —CO(substituted or unsubstituted 3-6 membered heterocyclyl) and —N(CH₃)₂.

In some embodiments, R¹ is 2-pyridyl, unsubstituted or substituted with one or more substituents independently selected from —CH₃, —CH₂CH₃, —CH₂CH₂CH₃, —CH(CH₃)₂, cyclopropyl, cyclobutyl, cyclopentyl, —OR⁵, —CONR⁶₂, substituted or unsubstituted phenyl, and —N(CH₃)₂.

In some embodiments, R¹ is 2-pyridyl, unsubstituted or substituted with one or more substituents independently selected from —CH₃, —CH₂CH₃, —CH(CH₃)₂, cyclopropyl, cyclobutyl, —OR⁵, substituted or unsubstituted phenyl, and —N(CH₃)₂.

In some embodiments, R¹ is 2-pyridyl, unsubstituted or substituted with one or more OR⁵. In some such embodiments, R⁵ is H, —CH₃, —CH₂CH₃, —CH₂CH₂CH₃, —CH(CH₃)₂, tetrahydrofuranyl, tetrahydropyranyl, or 1-methylpiperidyl. In some such embodiments, R⁵ is H, —CH₃, —CH₂CH₃, —CH(CH₃)₂, or tetrahydropyranyl. In some such embodiments, R⁵ is H or —CH₃. In some such embodiments, R⁵ is —CH₃. In some embodiments, R⁵ is —CH(CH₃)₂.

In some embodiments of compounds of formula (IV), (IVa), (IVb), and (IVc), R¹ is 2-pyridyl substituted with one or more —CONR⁶₂. In some such embodiments, each R⁶ is independently selected from H, substituted or unsubstituted C₁₋₅ alkyl; substituted or unsubstituted C₃₋₆ cycloalkyl, substituted or unsubstituted 3-6 membered heterocyclyl, and (C₁₋₃ alkyl) (substituted or unsubstituted 3-6 membered heterocyclyl). In some such embodiments, each R⁶ is independently H, substituted or unsubstituted C₁₋₅ alkyl selected from —CH₃, —CH₂CH₃, —CH₂CH₂CH₃, and —CH(CH₃)₂; substituted or unsubstituted C₃₋₆ cycloalkyl selected from cyclopropyl, cyclobutyl, cyclopentyl, and cyclohexyl; wherein the alkyl and cycloalkyl are optionally substituted with one or more substituents independently selected from OH, OCH₃, and F. In some embodiments, each R⁶ is independently H, substituted or unsubstituted C₁₋₅ alkyl selected from —CH(CH₃)₂; substituted or unsubstituted C₃₋₆ cycloalkyl selected from cyclopentyl and cyclohexyl; wherein the alkyl and cycloalkyl are optionally substituted with one or more substituents independently selected from OH, OCH₃, and F.

In some embodiments of compounds of formula (IV), (IVa), (IVb), and (IVc), R⁴ is H, or —CH₃. In some such embodiments, R⁴ is H. In some such embodiments, R⁴ is —CH₃. In some such embodiments, Rⁿ is H, —CH₃, or unsubstituted or substituted phenyl. In some such embodiments, Rⁿ is H. In some such embodiments, Rⁿ is —CH₃. In some such embodiments, Rⁿ is unsubstituted or substituted phenyl.

In some embodiments, provided herein are compounds of formula (IV), (IVa), (IVb), and (IVc), wherein R¹ is 2-pyridyl, unsubstituted or substituted with one or more substituents independently selected from —CH₃, —OCH(CH₃)₂, substituted or unsubstituted phenyl, and —N(CH₃)₂.

In some embodiments, provided herein are compounds of formula (IVa), wherein R¹ is 2-pyridyl, unsubstituted or substituted with one or more substituents independently selected from —CH₃, —OCH(CH₃)₂, substituted or unsubstituted phenyl, and —N(CH₃)₂.

In some embodiments, provided herein are compounds of formula (IVb) and (IVc), wherein R¹ is 2-pyridyl, unsubstituted or substituted with one or more substituents independently selected from —CH₃, —OCH(CH₃)₂, substituted or unsubstituted phenyl.

In some embodiments of compounds of formula (IV), (IVa), (IVb), and (IVc), R² is 2-pyridyl, unsubstituted or substituted with one or more substituents independently selected from halogen, substituted or unsubstituted C₁₋₄ alkyl, substituted or unsubstituted C₆₋₁₀ aryl, —OR⁵, and —CONR₂.

In some such embodiments R² is 2-pyridyl, unsubstituted or substituted with one or more substituents independently selected from —CH₃, —CH₂CH₃, —CH₂CH₂CH₃, —CH(CH₃)₂, cyclopropyl, cyclobutyl, cyclopentyl, substituted or unsubstituted phenyl, —OR⁵, and —CONR₂.

In some such embodiments R² is 2-pyridyl, unsubstituted or substituted with one or more substituents independently selected from —CH₃, —CH₂CH₃, —CH(CH₃)₂, cyclopropyl, cyclobutyl, —OCH₃, —OCH₂CH₃, —OCH₂CH₂CH₃, —OCH(CH₃)₂, —O-tetrahydrofuranyl, —O— tetrahydropyranyl, substituted or unsubstituted phenyl, and —CONR₂.

In some such embodiments R² is 2-pyridyl, unsubstituted or substituted with one or more substituents independently selected from —CH₃, —OCH₃, —OCH(CH₃)₂, —O— tetrahydrofuranyl, and substituted or unsubstituted phenyl.

In some embodiments of compounds of formula (IV), (IVa), (IVb), and (IVc), R³ is H, —CH₃, —CH₂CH₃, —CH(CH₃)₃, —CH₂OH, —CH₂CH₂OH, —CH₂OCH₂-cyclopropyl, or —CH₂OCH₂-cyclobutyl.

In some embodiments of compounds of formula (IV), (IVa), (IVb), and (IVc), R³ is H, —CH₃, —CH₂CH₃, —CH(CH₃)₃, or —CH₂OH.

In some embodiments of compounds of formula (IV), (IVa), (IVb), and (IVc), R³ is H, or —CH₃. In some such embodiments, R³ is H.

In some embodiments of compounds of formula (IV), (IVa), (IVb), and (IVc), R⁴ is H, or —CH₃. In some such embodiments, R⁴ is H. In some such embodiments, R⁴ is —CH₃.

In some embodiments, provided herein are compounds of formula (IVa), wherein R² is 2-pyridyl, unsubstituted or substituted with one or more substituents independently selected from —CH₃, —OCH₃, and —OCH(CH₃)₂.

In some embodiments, provided herein are compounds of formula (IVb) and (IVc), wherein R² is 2-pyridyl, unsubstituted or substituted with one or more substituents independently selected from —CH₃, —OCH₃, and —O-tetrahydrofuranyl.

In some embodiments of compounds of formula (IV), (IVa), (IVb), and (IVc), R³ is H, or —CH₃. In some such embodiments, R³ is H. In some such embodiments, R⁴ is H. In some such embodiments, R⁴ is —CH₃.

In some embodiments of compounds of formula (IV), (IVb), and (IVc), Rⁿ is H, —CH₃, or unsubstituted or substituted phenyl. In some such embodiments, Rⁿ is H. In some such embodiments, Rⁿ is —CH₃. In some such embodiments, Rⁿ is unsubstituted or substituted phenyl.

In some embodiments of compounds of formula (IV), (IVa), (IVb), and (IVc), wherein when R¹ is 2-pyridyl, unsubstituted or substituted with one or more substituents independently selected from —CH₃, —CH₂CH₃, —CH₂CH₂CH₃, —CH(CH₃)₂, cyclopropyl, cylobutyl, cyclopentyl, —OR⁵, substituted or unsubstituted phenyl, substituted or unsubstituted pyrazolyl, substituted or unsubstituted dihydropyranyl, substituted or unsubstituted piperidyl, substituted or unsubstituted piperazinyl, —CONR⁶₂, —CO(substituted or unsubstituted 3-6 membered heterocyclyl) and —N(CH₃)₂; R² is 2-pyridyl, unsubstituted or substituted with one or more substituents independently selected from —CH₃, —CH₂CH₃, —CH₂CH₂CH₃, —CH(CH₃)₂, cyclopropyl, cyclobutyl, cyclopentyl, substituted or unsubstituted phenyl, —OR⁵, and —CONR₂. In some other such embodiments, R³ is H, —CH₃, —CH₂CH₃, —CH(CH₃)₃, —CH₂OH, —CH₂CH₂OH, —CH₂OCH₂-cyclopropyl, or —CH₂OCH₂-cyclobutyl. In some such embodiments, R³ is H, or —CH₃. In some such embodiments, R³ is H. In some such embodiments, R³ is —CH₃. In some other such embodiments, R⁴ is H, or —CH₃. In some other such embodiments, R⁴ is H. In some other such embodiments, R⁴ is —CH₃. In some such embodiments, Rⁿ is H, —CH₃, or unsubstituted or substituted phenyl. In some such embodiments, Rⁿ is H. In some such embodiments, Rⁿ is —CH₃. In some such embodiments, Rⁿ is phenyl.

In some embodiments of compounds of formula (IV), (IVa), (IVb), and (IVc), wherein when R¹ is 2-pyridyl, unsubstituted or substituted with one or more substituents independently selected from —CH₃, —OCH(CH₃)₂, substituted or unsubstituted phenyl, and —N(CH₃)₂; R² is 2-pyridyl, unsubstituted or substituted with one or more substituents independently selected from —CH₃, —OCH₃, —OCH(CH₃)₂, —O-tetrahydrofuranyl, and substituted or unsubstituted phenyl; R³ is H, or —CH₃; R⁴ is H, or —CH₃. In some such embodiments, R³ is H. In some such embodiments, R³ is —CH₃. In some such embodiments, R⁴ is H. In some such embodiments, R⁴ is —CH₃. In some such embodiments, Rⁿ is H. In some such embodiments, Rⁿ is —CH₃. In some such embodiments, Rⁿ is phenyl.

In some embodiments of compounds of formula (IVa), wherein R¹ is 2-pyridyl, unsubstituted or substituted with one or more substituents independently selected from —CH₃, —OCH(CH₃)₂, substituted or unsubstituted phenyl, and —N(CH₃)₂; R² is 2-pyridyl, unsubstituted or substituted with one or more substituents independently selected from —CH₃, —OCH₃, —OCH(CH₃)₂; R³ is H, or —CH₃; R⁴ is H, or —CH₃ In some such embodiments, R³ is H. In some such embodiments, R³ is —CH₃. In some such embodiments, R⁴ is H. In some such embodiments, R⁴ is —CH₃.

In some embodiments of compounds of formula (IVb) and (IVc), wherein R¹ is 2-pyridyl, unsubstituted or substituted with one or more substituents independently selected from R¹ is 2-pyridyl, unsubstituted or substituted with one or more substituents independently selected from —CH₃, —OCH(CH₃)₂, substituted or unsubstituted phenyl; R² is 2-pyridyl, unsubstituted or substituted with one or more substituents independently selected from —CH₃, —OCH₃, and —O— tetrahydrofuranyl; R³ is H, or —CH₃; R⁴ is H, or —CH₃ In some such embodiments, R³ is H. In some such embodiments, R³ is —CH₃. In some such embodiments, R⁴ is H. In some such embodiments, R⁴ is —CH₃. In some such embodiments, Rⁿ is H. In some such embodiments, Rⁿ is —CH₃. In some such embodiments, Rⁿ is phenyl.

Further embodiments provided herein include combinations of at least one of the particular embodiments set forth above.

Representative compounds of formula (IV), (IVa), (IVb), and (IVc) are set forth in Tables 4 and 5.

### Methods for Making Compounds

The Heterocyclic Compounds of formula (I), formula (II), formula (III), formula (IIIa), formula (IIIb), formula (IIIc), formula (IIId), formula (IV), formula (IVa), formula (IVb), formula (IVc), Table 1, Table 2, Table 3, Table 4, and Table 5 can be made using conventional organic syntheses and commercially available starting materials. By way of example and not limitation, Heterocyclic Compounds of formula (I), formula (II), formula (III), formula (IIIa), formula (IIIb), formula (IIIc), formula (IIId), formula (IV), formula (IVa), formula (IVb), and formula (IVc), Table 1, Table 2, Table 3, Table 4, and Table 5 can be prepared as outlined in Schemes 1, 2, 3, 4, 5, and 6 shown below, as well as in the examples set forth herein. It should be noted that one skilled in the art would know how to modify the procedures set forth in the illustrative schemes and examples to arrive at the desired products.

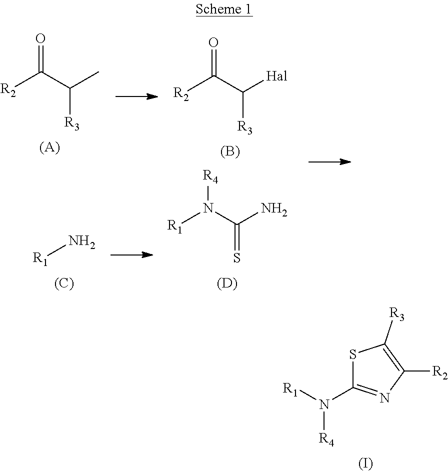

As shown in Scheme 1, compounds of formula (I), formula (II), Table 1, Table 2, and Table 3, wherein R¹, R², R³ and R⁴ are as defined herein, can be prepared starting from appropriately derivatized α-haloketones (B) and thioureas (D), wherein Hal is either Br or Cl. α-Haloketones (B) are commercially available or may be prepared according to known methods (see, e.g., K.-w. Jeong et al., European Journal of Medicinal Chemistry 102 (2015) 387-397). For example, α-haloketones (B) wherein Hal is Br can be obtained by treatment of appropriately substituted heteroaryl ethones (A) with brominating agents, such as bromine or pyridinium tribromide with HBr/acetic acid in a solvent, such as THF, 1-bromopyrrolidine-2,5-dione or NBS in a solvent such as DCM or THF, at temperatures ranging from 0° C. to 25° C. α-Haloketones wherein Hal is Cl can be accessed via Weinreb ketone synthesis, starting from R²—Br, utilizing 2-chloro-N-methoxy-N-methyl-acetamide in the presence of a base, such as nBuLi, in a solvent, such as THF, at reduced temperatures, such as −78° C. Thioureas (D) are commercially available or may be prepared according to known methods (Id.). Treatment of R¹NH₂ with benzoyl isothiocyanate in solvents, such as EtOH, MeOH, DCM, or acetone, in the presence of a base, such as NaOH or NaH, at temperatures ranging from 25° C. to 80° C., provides thioureas (D). Compounds of formula (I), formula (II), Table 1, Table 2, and Table 3 are obtained by treatment of thiourea (D) with α-haloketones (B) in a solvent, such as EtOH, THF, acetone or DMF, optionally in the presence of a base, such as DIPEA, NaH, NaOH or Na₂CO₃, at temperatures ranging from 25° C. to 80° C.

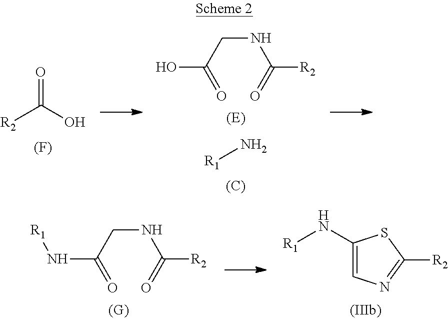

As shown in Scheme 2, compounds of formula (IIIb), Table 1, wherein R¹ and R² are as defined herein, can be prepared starting from appropriately derivatized glycine (E) and amine (C). Substituted glycines (E) can prepared according to known methods (see, e.g., Dhar, T. G. Murali et al., Bioorganic & Medicinal Chemistry Letters, 12(21), 3125-3128). For example, glycine (E) can be obtained by treatment of appropriately substituted heteroaryl carboxylic acid (F) with an aminoacetic acid ester in the presence of coupling agents, such as HOBT and EDC, in the presence of a base, such as DIPEA, and a solvent, such as DCM or THF, at temperatures ranging from 0° C. to 25° C. Amide (G) is obtained by the subsequent coupling of (E) with R¹NH₂ (C) in the presence of coupling agents such HATU and solvents, such as NMM and DMF, at temperatures ranging from 0° C. to 25° C. Compounds of formula (IIIb), Table 1, are obtained by treatment of amide (G) with Lawesson's reagent in solvent, such as toluene, at temperatures ranging from 25° C. to 110° C.

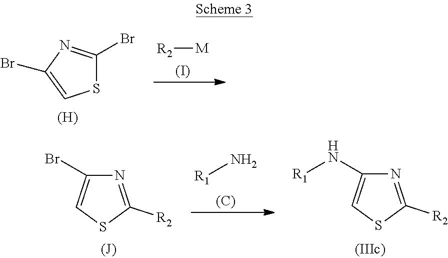

Alternatively, as shown in Scheme 3, compounds of formula (IIIc), Table 1, wherein R¹ and R² are as defined herein, can be prepared via a series of metal mediated cross coupling reactions from commercially available 2,4-dibromothiazole (H). For example, 2,4-dibromothiazole (H) can be treated with organometallic compound (I) wherein M is Sn in the presence of a metal catalyst, such as Pd(PPh₃)₂Cl₂, in a solvent, such as DMF, at temperatures ranging from 25° C. to about 90° C. to give compound (J) followed by subsequent treatment with amine (C) in the presence of metal catalyst and ligand, such as Pd₂(dba)₃ and Xantphos, and in the presence of a base, such as Cs₂CO₃, and in a solvent, such as 1,4-dioxane, at temperatures ranging from about 25° C. to about 110° C. to give compounds of formula (IIIc), Table 1.

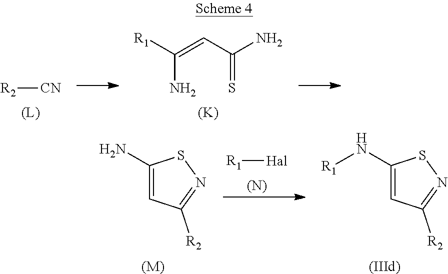

Alternatively, as shown in Scheme 4, compounds of formula (IIId), Table 1, wherein R¹ and R² are as defined herein, can be prepared from appropriately substituted acrylothioamide (K). Acrylothioamides (K) can be prepared according to known methods. (see, e.g., Kuklish, Steven L. et al., Tetrahedron Letters, 56(20), 2605-2607; 2015). For example, substituted nitrile (L) can be treated with acetonitrile in a solvent, such as benzene, in the presence of a base, such as t-BuOK, at temperatures ranging from 0° C. to 25° C. followed by subsequent reaction with phosphorous pentasulfide and Na₂S in a solvent, such as THF, at temperatures ranging from 0° C. to 25° C. to provide acrylothioamide (K). Treatment of acrylothioamide (K) with H₂O₂ in the presence of a solvent, such as MeOH, at 25° C. provides cyclized amino-isothiazole (M). Metal mediated coupling of amino-isothiazole (M) can be performed with halogenated heteroaryl (N), wherein Hal is Br in the presence of metal catalyst and ligand, such as Pd₂(dba)₃ and Xantphos or BINAP, and in the presence of a base, such as Cs₂CO₃, and in a solvent, such as 1,4-dioxane, at temperatures ranging from about 25° C. to about 110° C. to give compounds of formula (IIId), Table 1.

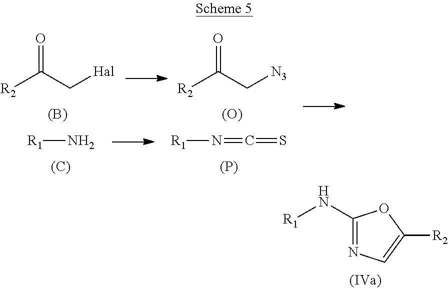

Alternatively, as shown in Scheme 5, compounds of formula (IVa), Table 4, wherein R¹ and R² are as defined herein, can be prepared from appropriately substituted azidoketones (O) and isothiocyanate (P). Azidoketones (O) may be prepared according to known methods (see, e.g., Harris, Philip A. et al., Journal of Medicinal Chemistry, 48(5), 1610-1619; 2005). For example, azidoketones (O) can be obtained by treatment of appropriately substituted α-haloketones (B) wherein Hal is Br with NaN₃ in a solvent, such as EtOH, and in the presence of a base, such as NaHCO₃, at temperatures ranging from 0° C. to 25° C. Appropriately substituted isothiocyanates (P) are commercially available or may be prepared according to known methods (see for example, J. Org. Chem. (2017), 82, 5898-5903). Reaction of appropriately substituted amines (C), with thiophosgene, optionally in the presence of a base, such as DIPEA, in a solvent, such as DCM, at temperatures ranging from about −5 to about 20° C. provides isothiocyanates (P). Condensation of isothiocyante (P) and azidoketone (0) in the presence of PPh₃, in a solvent, such as DCM, at temperatures ranging 0° C. to 25° C. provides compounds of formula (IVa), Table 4.

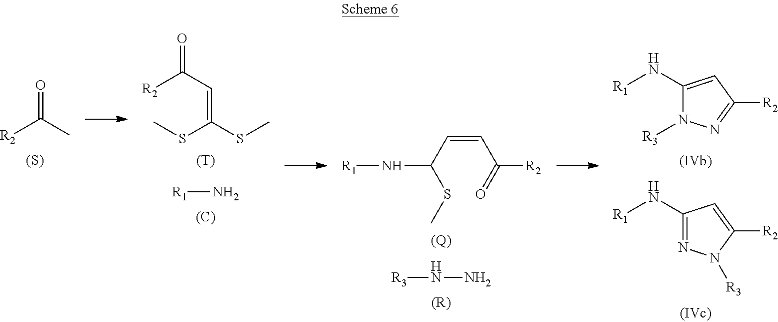

Alternatively, as shown in Scheme 6, compounds of formula (IVb and IVc), Table 5, wherein R¹, R² and R³ are as defined herein, can be prepared by condensation of appropriately substituted N,S-acetal (Q) and hydrazine (R). N,S-acetal (Q) can be prepared using know methods (see, e.g., Surmont, Riccardo et al., Journal of Organic Chemistry, 76(10), 4105-4111; 2011). For example, ketone (S) can be treated with carbon disulfide and Mel in the presence of a base, such as NaH, in a solvent, such as DMSO or THF, at temperatures ranging from about 0° C. to 25° C. to give compound (T) followed by treatment with amine (C) in a solvent, such as THF, in the presence of a base, such as nBuLi, at temperatures ranging from 0° C. to 25° C. to provide N,S acetal (Q). Condensation of N,S acetal (Q) with appropriately substituted hydrazine (R) in the presence of an acid, such as AcOH, and in a solvent, such as tBuOH, at temperatures ranging from 0° C. to 180° C. provides compounds of formula (IVb and IVc), Table 5.

In one aspect, provided herein are methods for preparing a compound of formula (I):

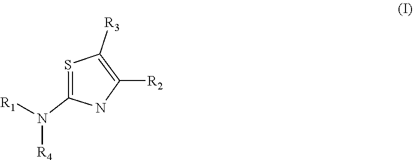

the methods comprising contacting a compound of formula (B):

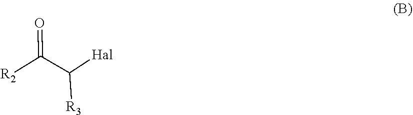

with a compound of formula (D):

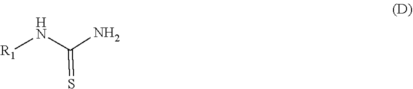

in a solvent, optionally in the presence of a base, under conditions suitable to provide a compound of formula (I), wherein:
R¹ is isoquinolyl; pyrrolopyridyl; 2-pyrimidyl, or 2-pyridyl, wherein the 2-pyridyl is substituted with one or more substituents independently selected from halogen, CN, substituted or unsubstituted C₁₋₄ alkyl, substituted or unsubstituted C₃₋₇ cycloalkyl, substituted or unsubstituted C₆₋₁₀ aryl, substituted or unsubstituted monocyclic heteroaryl, substituted or unsubstituted 3-6 membered heterocyclyl, —OR⁵, —SR, —CONR⁶₂, —CON(C₁₋₃ alkyl)(substituted or unsubstituted C₃₋₇ cycloalkyl), —NRCO(C₁₋₃ alkyl), —CO(substituted or unsubstituted 3-6 membered heterocyclyl), —SO₂NR₂ and SO₂R⁵;
R² is 5,6-dihydro-4H-pyrrolo[1,2-b]pyrazolyl, 2-pyrimidyl, or 2-pyridyl, substituted with one or more substituents independently selected from halogen, substituted or unsubstituted C₁₋₄ alkyl, substituted or unsubstituted C₆₋₁₀ aryl, —OR⁵, —SR, —CONR₂, and —SO₂R⁵, or two atoms together with the carbons to which they are attached form a substituted or unsubstituted 5-6 membered heterocyclyl;
R³ is H, —CN, substituted or unsubstituted C₁₋₄ alkyl, (C₁₋₃ alkyl)O(C₁₋₃ alkyl)(substituted or unsubstituted C₃₋₇ cycloalkyl), —(C₁₋₃ alkyl)OR, (C₁₋₃ alkyl)(substituted or unsubstituted 3-6 membered heterocyclyl), —C(O)(substituted or unsubstituted 3-10 membered heterocyclic), —C(O)OR, substituted or unsubstituted C₆₋₁₀ aryl, (C₁₋₃ alkyl)NR⁶₂, —(C₁₋₃ alkyl)N(C₁₋₃ alkyl)(substituted or unsubstituted C₃₋₇ cycloalkyl), CONR⁶₂, or —C(O)N(C₁₋₃ alkyl)NR₂;
R⁴ is H or substituted or unsubstituted C₁₋₃ alkyl, or substituted or unsubstituted —(C₁₋₃ alkyl) C₆₋₁₀ aryl;
R⁵ is H, substituted or unsubstituted C₁₋₅ alkyl, substituted or unsubstituted C₃₋₇ cycloalkyl, or substituted or unsubstituted 3-6 membered heterocyclyl;
each R⁶ is independently selected from H, substituted or unsubstituted C₁₋₅ alkyl, substituted or unsubstituted C₃₋₆ cycloalkyl, substituted or unsubstituted 3-6 membered heterocyclyl, and (C₁₋₃ alkyl)(substituted or unsubstituted 3-6 membered heterocyclyl); and
each R is independently selected from H and substituted or unsubstituted C₁₋₄ alkyl;
provided the compound is not 4-methyl-N-[4-(4-methyl-2-pyridinyl)-2-thiazolyl]-2-pyridinamine or N-(5-chloropyridin-2-yl)-4-(pyrimidin-2-yl)thiazol-2-amine.

In some embodiments, the solvent is EtOH, THF, acetone, or DMF. In some embodiments, the base is DIPEA, NaH, NaOH, or Na₂CO₃. In some embodiments, the contacting is performed at a temperature ranging from 25° C. to 80° C.

In some embodiments, the methods further comprise preparing a compound of formula (B):

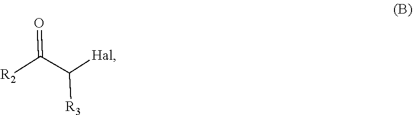

the methods comprising contacting a compound of formula (A):

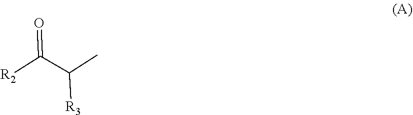

wherein Hal is Br, with brominating agents, in a solvent, under conditions suitable to provide a compound of formula (A).

In some embodiments, the brominating agent is Br₂ and the solvent is HBr/acetic acid. In another embodiment, the brominating agent is pyridinium tribromide and the solvent is HBr/acetic acid or THF. In yet another embodiment, the brominating agent is NBS and the solvent is THE or DCM. In yet another embodiment, the brominating agent is 1-bromopyrrolidine-2,5-dione and the solvent is DCM or THF.

In some embodiments, the contacting is performed at a temperature ranging from 0° C. to 25° C.

In some embodiments, wherein the methods further comprise preparing a compound of formula (B):

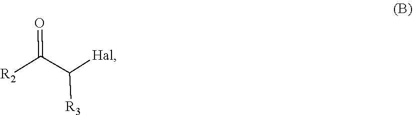

the method comprises contacting R²—Br
wherein Hal is Cl, with 2-chloro-N-methoxy-N-methyl-acetamide in the presence of a base, in a solvent, under conditions suitable to provide a compound of formula (B).

In one embodiment, the base is nBuLi. In one embodiment, the solvent is THF. In some embodiments, the contacting is performed at a reduced temperature. In one embodiment, the contacting is performed at −78° C.

In some embodiments, the methods further comprise preparing a compound of formula (D):

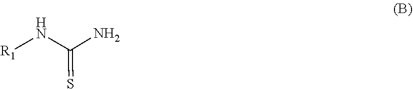

the method comprising contacting R¹NH₂ with benzoylisothiocyanate, in a solvent, under conditions suitable to provide a compound of formula (D).

In some embodiments, the method further comprises the presence of a base. In one embodiment, the base is NaOH or NaH.

In one embodiment, the solvent is THF, EtOH, MeOH, DCM, or acetone. In some embodiments, the contacting is performed at a temperature ranging from 25° C. to 80° C.

In another aspect, provided herein are methods for preparing a Heterocyclic Compound of formula (IIIb):

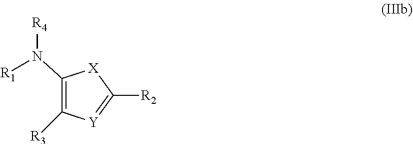

wherein
X is S;
Y is N;
R¹ is 2-pyridyl, unsubstituted or substituted with one or more substituents independently selected from halogen, substituted or unsubstituted C₁₋₄ alkyl, substituted or unsubstituted C₃₋₇ cycloalkyl, substituted or unsubstituted C₆₋₁₀ aryl, substituted or unsubstituted monocyclic heteroaryl, substituted or unsubstituted 3-6 membered heterocyclyl, —OR⁵, —CONR⁶₂, and —CO(substituted or unsubstituted 3-6 membered heterocyclyl);
R² is 2-pyridyl, unsubstituted or substituted with one or more substituents independently selected from halogen, substituted or unsubstituted C₁₋₄ alkyl, substituted or unsubstituted C₆₋₁₀ aryl, —OR⁵, and —CONR₂;
R³ is H, substituted or unsubstituted C₁₋₄ alkyl, or (C₁₋₃ alkyl)O(C₁₋₃ alkyl)(substituted or unsubstituted C₃₋₇ cycloalkyl), (C₁₋₃ alkyl)OR;
R⁴ is H, substituted or unsubstituted C₁₋₃ alkyl;
R⁵ is H, substituted or unsubstituted C₁₋₅ alkyl, or unsubstituted 3-6 membered heterocyclyl;
each R⁶ is independently selected from H, substituted or unsubstituted C₁₋₅ alkyl; substituted or unsubstituted C₃₋₆ cycloalkyl, substituted or unsubstituted 3-6 membered heterocyclyl, and (C₁₋₃ alkyl)(substituted or unsubstituted 3-6 membered heterocyclyl); and
each R is independently selected from H and substituted or unsubstituted C₁₋₄ alkyl.
the methods comprising contacting a compound of formula (G)

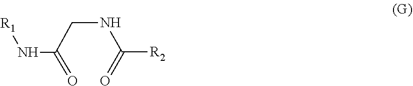

with Lawesson's reagent, in a solvent, under conditions suitable to provide a Heterocyclic Compound of formula (IIIb).

In some such embodiments, the solvent is toluene. In some embodiments, the contacting is performed at temperature ranging from about 25 to about 110° C.

In some embodiments, the methods further comprise preparing a compound of formula (G):

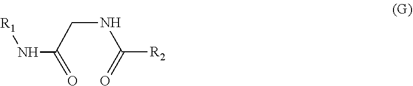

the methods comprising coupling a compound of formula (E):

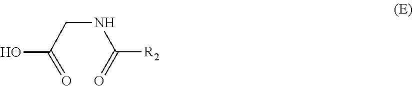

with R¹—NH₂, in a solvent, with coupling agents under conditions suitable to provide a compound of formula (G).

In some embodiments, the coupling agent is HATU and the solvent is NMM. In yet another embodiment, the solvent is DMF. In some embodiments, the contacting is performed at a temperature ranging from 0° C. to 25° C.

In some embodiments, wherein the methods further comprise preparing a compound of formula (E):

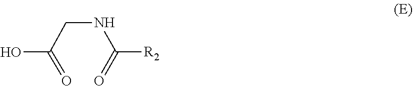

the method comprises contacting R²—COOH with an aminoacetic acid with coupling agents, in the presence of a base, in a solvent, under conditions suitable to provide a compound of formula (E).

In some embodiments, the coupling agents are HOBT and EDC and the solvent is DCM or DMF. In some embodiments, the base is DIPEA. In some embodiments, the contacting is performed at a temperature ranging from 0° C. to 25° C.

In another aspect, provided herein are methods for preparing a Heterocyclic Compound of formula (IIIc):

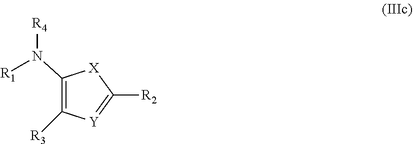

wherein
X is N;
Y is S;
R¹ is 2-pyridyl, unsubstituted or substituted with one or more substituents independently selected from halogen, substituted or unsubstituted C₁₋₄ alkyl, substituted or unsubstituted C₃₋₇ cycloalkyl, substituted or unsubstituted C₆₋₁₀ aryl, substituted or unsubstituted monocyclic heteroaryl, substituted or unsubstituted 3-6 membered heterocyclyl, —OR⁵, —CONR⁶₂, and —CO(substituted or unsubstituted 3-6 membered heterocyclyl);
R² is 2-pyridyl, unsubstituted or substituted with one or more substituents independently selected from halogen, substituted or unsubstituted C₁₋₄ alkyl, substituted or unsubstituted C₆₋₁₀ aryl, —OR⁵, and —CONR₂;
R³ is H, substituted or unsubstituted C₁₋₄ alkyl, or (C₁₋₃ alkyl)O(C₁₋₃ alkyl)(substituted or unsubstituted C₃₋₇ cycloalkyl), (C₁₋₃ alkyl)OR;
R⁴ is H, substituted or unsubstituted C₁₋₃ alkyl;
R⁵ is H, substituted or unsubstituted C₁₋₅ alkyl, or unsubstituted 3-6 membered heterocyclyl;
each R⁶ is independently selected from H, substituted or unsubstituted C₁₋₅ alkyl; substituted or unsubstituted C₃₋₆ cycloalkyl, substituted or unsubstituted 3-6 membered heterocyclyl, and (C₁₋₃ alkyl)(substituted or unsubstituted 3-6 membered heterocyclyl); and
each R is independently selected from H and substituted or unsubstituted C₁₋₄ alkyl.
the methods comprising contacting a compound of formula (J)

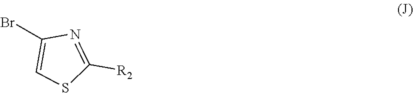

with R¹—NH₂, in a solvent, with metal catalysts and ligands under conditions suitable to provide a compound of formula (IIIc).

In some embodiments, the metal catalyst is Pd₂(dba)₃ and the ligand is Xantphos. In some embodiments, the solvent is 1,4-dioxane and the base is Cs₂CO₃. In some embodiments, the contacting is performed at a temperature ranging from 25° C. to 110° C.

In some embodiments, wherein the methods further comprise preparing a compound of formula (J):

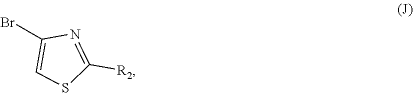

the method comprises contacting compound of formula (H):

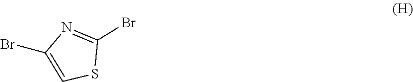

with R²-M, wherein M is Sn, with metal catalysts under conditions suitable to provide a compound of formula (J).

In some embodiments, the metal catalyst is Pd(PPh₃)₂Cl₂. In some embodiments, the solvent is DMF and the contacting is performed at a temperature ranging from 25° C. to 90° C.

In another aspect, provided herein are methods for preparing a Heterocyclic Compound of formula (IIId):

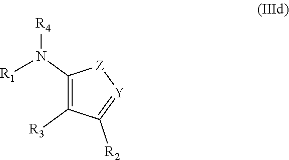

wherein
Y is N;
Z is S,
R¹ is 2-pyridyl, unsubstituted or substituted with one or more substituents independently selected from halogen, substituted or unsubstituted C₁₋₄ alkyl, substituted or unsubstituted C₃₋₇ cycloalkyl, substituted or unsubstituted C₆₋₁₀ aryl, substituted or unsubstituted monocyclic heteroaryl, substituted or unsubstituted 3-6 membered heterocyclyl, —OR⁵, —CONR⁶₂, and —CO(substituted or unsubstituted 3-6 membered heterocyclyl);
R² is 2-pyridyl, unsubstituted or substituted with one or more substituents independently selected from halogen, substituted or unsubstituted C₁₋₄ alkyl, substituted or unsubstituted C₆₋₁₀ aryl, —OR⁵, and —CONR₂;
R³ is H, substituted or unsubstituted C₁₋₄ alkyl, or (C₁₋₃ alkyl)O(C₁₋₃ alkyl)(substituted or unsubstituted C₃₋₇ cycloalkyl), (C₁₋₃ alkyl)OR;
R⁴ is H, substituted or unsubstituted C₁₋₃ alkyl;
R⁵ is H, substituted or unsubstituted C₁₋₅ alkyl, or unsubstituted 3-6 membered heterocyclyl;
each R⁶ is independently selected from H, substituted or unsubstituted C₁₋₅ alkyl; substituted or unsubstituted C₃₋₆ cycloalkyl, substituted or unsubstituted 3-6 membered heterocyclyl, and (C₁₋₃ alkyl)(substituted or unsubstituted 3-6 membered heterocyclyl); and
each R is independently selected from H and substituted or unsubstituted C₁₋₄ alkyl.
the methods comprising contacting a compound of formula (M)

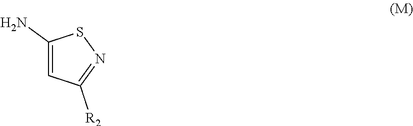

with R¹-Hal, wherein Hal is Br, in a solvent, with metal catalysts and ligands under conditions suitable to provide a compound of formula (IIId).

In some embodiments, the metal catalyst is Pd₂(dba)₃ and the ligand is Xantphos or BINAP. In some embodiments, the solvent is 1,4-dioxane and the base is Cs₂CO₃. In some embodiments, the contacting is performed at a temperature ranging from 25° C. to 110° C.

In some embodiments, wherein the methods further comprise preparing a compound of formula (M)

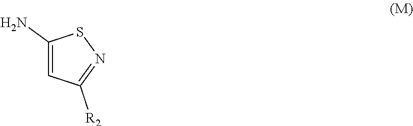

the method comprises contacting compound of formula (K):

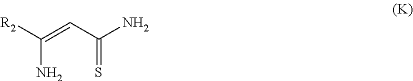

with H₂O₂ under conditions suitable to provide a compound of formula (K).

In some embodiments, the solvent is MeOH and the contacting is performed at 25° C.

In some embodiments, wherein the methods further comprise preparing a compound of formula (K)

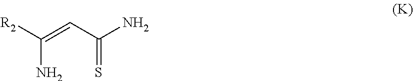

the method comprises contacting compound R²—CN
a) with a base in a first solvent and temperature; and
b) contacting the product of step a) with phosphorous pentasulfide and Na₂S in a second solvent under conditions suitable to provide a compound of formula (K).

In some embodiments, the first base is t-BuOK and the solvent is benzene or ACN. In one embodiment, the contacting in step a) is performed at a temperature ranging from about 0 to about 25° C.

In some embodiments, the second solvent is THF. In one embodiment, the contacting in step b) is performed at a temperature ranging from room temperature to about 0-25° C.

In another aspect, provided herein are methods for preparing a Heterocyclic Compound of formula (IVa):

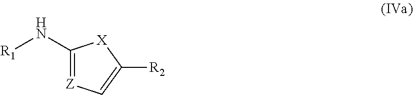

wherein
X is O;
Z is N,
R¹ and R² are as defined herein.

The methods comprising contacting a compound of formula (O)

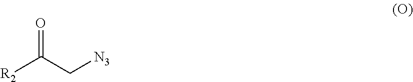

with a compound of formula (P):

R₁—N═C═S (P)
in the presence of PPh₃, in a solvent, under conditions suitable to provide a Heterocyclic Compound of formula (IVa).

In some embodiments, the solvent is DCM and the contacting is performed at a temperature ranging from 0° C. to 25° C.

In some embodiments, wherein the methods further comprise preparing a compound of formula (O)

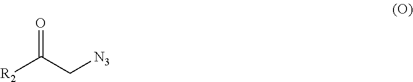

the method comprises contacting compound with a compound of formula (B):

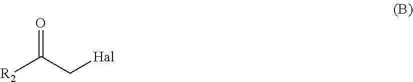

wherein Hal is Br, in the presence of NaN₃, in a solvent, under conditions suitable to provide compound of formula (O).

In some embodiments, the solvent is EtOH and the base is NaHCO₃. In some embodiments, the contacting is performed at a temperature ranging from 0° C. to 25° C.

In some embodiments, wherein the methods further comprise preparing a compound of formula (P)

R₁—N═C═S (P)
the method comprises contacting R¹—NH₂ with thiophosgene, with a base, in a solvent, under conditions suitable to provide a compound of formula (P).

In some embodiments, the solvent is DCM and the base is DIPEA. In some embodiments, the contacting is performed at a temperature ranging from −5° C. to 20° C.

In another aspect, provided herein are methods for preparing a Heterocyclic Compound of formula (IVb and IVc):

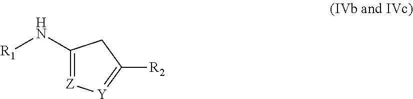

wherein
Y is N or NRⁿ;
Z is N or NRⁿ;
R¹, R² and Rⁿ are as defined herein.

The methods comprising contacting a compound of formula (Q)

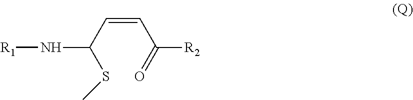

with a compound of formula (R):

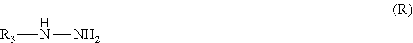

in the presence of an acid, in a solvent, under conditions suitable to provide a Heterocyclic Compound of formula (IVb and IVc).

In some embodiments, the solvent is tBuOH and the acid is AcOH. In some embodiments, the contacting is performed at a temperature ranging from 0° C. to 180° C.

In some embodiments, wherein the methods further comprise preparing a compound of formula (Q)

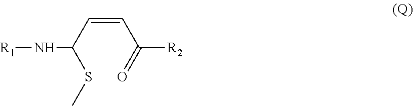

the methods comprising contacting a compound of formula (T)

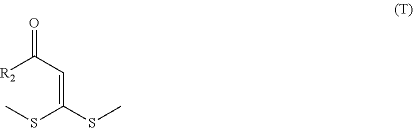

with R¹—NH₂, in a solvent and a base under conditions suitable to provide a compound of formula (Q).

In some embodiments, the solvent is THE and the base is nBuLi. In some embodiments, the contacting is performed at a temperature ranging from 0° C. to 25° C.

In some embodiments, wherein the methods further comprise preparing a compound of formula (T)

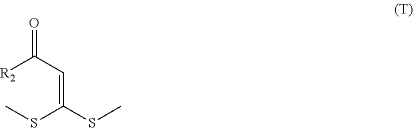

the methods comprising contacting a compound of formula (S)

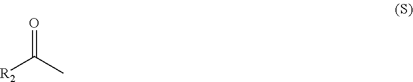

with carbon disulfide and Mel under conditions suitable to provide a compound of formula (T).

In some embodiments, the base is NaH and the solvent is THE or DMSO. In one embodiment, the contacting step is performed at a temperature ranging from about 0 to about 25° C.

### Methods of Use

The Heterocyclic Compounds, including compounds of formula (I), formula (II), formula (III), (IIIa), (IIIb), (IIIc), (IIId), (IV), (IVa), (IVb), (IVc), and Table 1, Table 2, Table 3, Table 4, and Table 5 have utility as pharmaceuticals to treat, prevent or improve conditions in animals and humans. The Heterocyclic Compounds provided herein have utility for use in the treatment or prevention of all diseases, disorders or conditions disclosed herein.

In one aspect, provided herein is a method of treating a disease caused by a helminthic infection. In certain embodiments, a compound as described herein is used in human medical therapy, particularly in the treatment of helminthic infection. In certain embodiments, a compound as provided herein is used in animal medical therapy, particularly in the treatment of helminthic infections. In certain embodiments, the method includes administering a therapeutically effective amount of a compound as described to a subject having a disease caused by a helminthic infection.

In one aspect, provided herein is a method of treating a disease caused by a filarial worm infection. In certain embodiments, a compound as described herein is used in human medical therapy, particularly in the treatment of filarial worm infection. In certain embodiments, a compound as provided herein is used in animal medical therapy, particularly in the treatment of filarial worm infections. In certain embodiments, the method includes administering a therapeutically effective amount of a compound as described to a subject having a disease caused by a filarial worm infection.

In one embodiment, provided herein is a method for the treatment or prevention of helminthic infections and diseases, the methods comprising administering to a subject an effective amount of a Heterocyclic Compound, or a pharmaceutically acceptable salt, tautomer, isotopologue, or stereoisomer thereof. In some such embodiments, the helminthic infection is a filarial worm infection.

In one aspect, provided herein is a method of treating a disease caused by helminthic infection. In certain embodiments, a Heterocyclic Compound, or a pharmaceutically acceptable salt, tautomer, isotopologue, or stereoisomer thereof, is used in human medical therapy, particularly in the treatment of helminthic infections. In certain embodiments, a Heterocyclic Compound, or a pharmaceutically acceptable salt, tautomer, isotopologue, or stereoisomer thereof, is used in animal medical therapy, particularly in the treatment of helminthic infections. In certain embodiments, the method includes administering a therapeutically effective amount of a Heterocyclic Compound, or a pharmaceutically acceptable salt, tautomer, isotopologue, or stereoisomer thereof, to a subject having a disease caused by helminthic infection.

In one aspect, provided herein is a method of treating a disease caused by a filarial worm infection. In certain embodiments, a Heterocyclic Compound, or a pharmaceutically acceptable salt, tautomer, isotopologue, or stereoisomer thereof, is used in human medical therapy, particularly in the treatment of a filarial worm infections. In certain embodiments, an a Heterocyclic Compound, or a pharmaceutically acceptable salt, tautomer, isotopologue, or stereoisomer thereof, is used in animal medical therapy, particularly in the treatment of a filarial worm infection. In certain embodiments, the method includes administering a therapeutically effective amount of a Heterocyclic Compound, or a pharmaceutically acceptable salt, tautomer, isotopologue, or stereoisomer thereof, to a subject having a disease caused by a filarial worm infection.

In another aspect, also provided is a method of preventing a disease caused by helminthic infection. In certain embodiments, a Heterocyclic Compound, or a pharmaceutically acceptable salt, tautomer, isotopologue, or stereoisomer thereof, is used in human medical therapy, particularly in the prevention of helminthic infection. In certain embodiments, a Heterocyclic Compound, or a pharmaceutically acceptable salt, tautomer, isotopologue, or stereoisomer thereof, is used in animal medical therapy, particularly in the prevention of helminthic infection. In certain embodiments, the method includes administering a therapeutically effective amount of a Heterocyclic Compound, or a pharmaceutically acceptable salt, tautomer, isotopologue, or stereoisomer thereof, to a subject to prevent a disease caused by helminthic infection.

In another aspect, also provided is a method of preventing a disease caused by a filarial worm infection. In certain embodiments, a Heterocyclic Compound, or a pharmaceutically acceptable salt, tautomer, isotopologue, or stereoisomer thereof, is used in human medical therapy, particularly in the prevention of a filarial worm infection. In certain embodiments, a Heterocyclic Compound, or a pharmaceutically acceptable salt, tautomer, isotopologue, or stereoisomer thereof, is used in animal medical therapy, particularly in the prevention of a filarial worm infection. In certain embodiments, the method includes administering a therapeutically effective amount of a Heterocyclic Compound, or a pharmaceutically acceptable salt, tautomer, isotopologue, or stereoisomer thereof, to a subject to prevent a disease caused by a filarial worm infection.

In another aspect, provided herein is a method of treating or preventing a parasitic disease. In certain embodiments, the parasitic disease is associated with a worm. In certain embodiments, the parasitic disease is caused by a worm. In certain embodiments, the parasitic worm is categorized as cestode (tapeworm), nematode (roundworm) and trematode (flatworm or fluke). In certain embodiments, the parasitic disease is associated with a helminth. In certain embodiments, the parasitic disease is associated with a nematode. In certain embodiments, the nematode is Wuchereria bancrofti. In certain embodiments, the nematode is Brugia malayi. In certain embodiments, the nematode is Brugia timori. In certain embodiments, the nematode is Onchocerca volvulus. In certain embodiments, the nematode is Dirofilaria immitis. In certain embodiments, the parasitic disease is associated with a trematode. In certain embodiments, the parasitic disease is associated with Schistosoma. In certain embodiments, the parasitic disease is associated with Schistosoma mansoni. In certain embodiments, the parasitic disease is enterobiasis, oxyuriasis, ascariasis, dracunculiasis, filariasis, onchocerciasis, schistosomiasis, or trichuriasis. In certain embodiments, the parasitic disease is schistosomiasis. In certain embodiments, the parasitic disease is urinary schistosomiasis. In certain embodiments, the parasitic disease is intestinal schistosomiasis. In certain embodiments, the parasitic disease is Asian intestinal schistosomiasis. In certain embodiments, the parasitic disease is visceral schistosomiasis. In certain embodiments, the parasitic disease is acute schistosomiasis. In certain embodiments, the parasitic disease is lymphatic filariasis. In certain embodiments, the parasitic disease is bancroftian filariasis. In certain embodiments, the parasitic disease is subcutaneous filariasis. In certain embodiments, the parasitic disease is serious cavity filariasis. In certain embodiments, the parasitic disease is elephantiasis. In certain embodiments, the parasitic disease is elephantiasis tropica. In certain embodiments, the parasitic disease is onchocerciasis. In certain embodiments, the parasitic disease is dirofilariasis. In certain embodiments, the dirofilariasis is dirofilariasis in in dogs. In some embodiments, the dirofilariasis is caused by Dirofilaria immitis or Diofilaria repens.

In certain aspects, the present methods comprise a step of administering a Heterocyclic Compound, or a pharmaceutically acceptable salt, tautomer, isotopologue, or stereoisomer thereof, to a subject. In certain embodiments, the methods comprise administering a Heterocyclic Compound, or a pharmaceutically acceptable salt, tautomer, isotopologue, or stereoisomer thereof, to a subject for no more than fourteen (14) days. In certain embodiments, the methods comprise administering a Heterocyclic Compound, or a pharmaceutically acceptable salt, tautomer, isotopologue, or stereoisomer thereof, to a subject for no more than seven (7) days. In certain embodiments, the subject is in need of treatment for an helminthic infection. In certain embodiments, the subject is in need of treatment for a filarial infection. In certain embodiments, the subject has an helminthic infection. In certain embodiments, the subject is at risk for having an helminthic infection. In certain embodiments, the subject has a filarial infection. In certain embodiments, the subject is at risk for having a filarial infection. In certain embodiments, the subject is a pediatric subject. In certain embodiments, the subject is less than nine (9) years of age. In certain embodiments, the subject is less than eight (8) years of age. In certain embodiments, the subject is a pregnant woman. In certain embodiments, the subject is a post-partum woman. In certain embodiments, the subject is a woman of childbearing potential. In certain embodiments, the subject is an individual attempting to conceive a child.

The compounds disclosed herein exhibit potency against helminths, and, therefore, have the potential to kill and/or inhibit the growth, molt, or motility of such helminths. The compounds disclosed herein exhibit potency against filarial worms, and, therefore, have the potential to kill and/or inhibit the growth, molt, or motility of such filarial worms. Thus, in one aspect provided is a method of killing a filarial worm, comprising: contacting the filarial worm with a Heterocyclic Compound, or a pharmaceutically acceptable salt, tautomer, isotopologue, or stereoisomer thereof, in an amount effective to kill the filarial worm. In another aspect, provided herein is a method of inhibiting growth or molt of a filarial worm, comprising: contacting the filarial worm with a Heterocyclic Compound, or a pharmaceutically acceptable salt, tautomer, isotopologue, or stereoisomer thereof, in an amount effective to inhibit growth or molt of the filarial worm. In another aspect, provided herein is a method of inhibiting motility of a filarial worm, comprising: contacting the filarial worm with a Heterocyclic Compound, or a pharmaceutically acceptable salt, tautomer, isotopologue, or stereoisomer thereof, in an amount effective to inhibit motility of the filarial worm. In certain embodiments, the worm is an egg. In certain embodiments, the egg is an unfertilized egg. In certain embodiments, the egg is fertilized egg. In certain embodiments, the worm is a larva. In certain embodiments, the worm is in a larval or juvenile stage. In certain embodiments, the worm is a larva in any one of four larval stages (L1, L2, L3, L4). In certain embodiments, the worm is a larva of stage L1 or microfilaria. In certain embodiments, microfilaria is a larva of stage L1. In certain embodiments, the worm is a larva of stage L2. In certain embodiments, the worm is a larva of stage L3. In certain embodiments, the worm is a larva of stage L4. In certain embodiments the worm is in sexually immature stage (stage L5). In certain embodiments, the worm is mature. In certain embodiments, the worm is fully mature. In certain embodiments, the worm is in adult stage. In certain embodiments, the worm is in pre-parasitic stage. In certain embodiments, the worm is in parasitic stage. In certain embodiments, the worm is contacted with a Heterocyclic Compound, or a pharmaceutically acceptable salt, tautomer, isotopologue, or stereoisomer thereof, inside a subject. In certain embodiments, the worm is contacted with a Heterocyclic Compound, or a pharmaceutically acceptable salt, tautomer, isotopologue, or stereoisomer thereof, outside a subject.

As discussed herein, compounds provided herein are useful for treating and preventing certain diseases and disorders in humans and animals. In certain embodiments, a Heterocyclic Compound, or a pharmaceutically acceptable salt, tautomer, isotopologue, or stereoisomer thereof, is used to treat a disease caused by helminthic infection. In certain embodiments, a Heterocyclic Compound, or a pharmaceutically acceptable salt, tautomer, isotopologue, or stereoisomer thereof, is used to treat a disease caused by filarial worm infection, including, but not limited to, heartworm disease, onchocerciasis, and lymphatic filariasis. In certain embodiments, treatment or prevention of such diseases and disorders can be effected by administering a Heterocyclic Compound, or a pharmaceutically acceptable salt, tautomer, isotopologue, or stereoisomer thereof, either alone or in combination with another active agent as part of a combination therapy. The term “combination” as in the phrase “in combination with another active agent” includes co-administration of a first agent and a second agent, which for example may be dissolved or intermixed in the same pharmaceutically acceptable carrier, or administration of a first agent, followed by the second agent, or administration of the second agent, followed by the first agent. The present methods and compositions, therefore, include methods of combination therapeutic treatment and combination pharmaceutical compositions. The term “combination therapy” refers to the administration of two or more therapeutic substances, such as a compound described herein and another drug (e.g., an antihelminthic agent such as ivermectin, albendazole, flubendazole, diethylcarbamazine, or emodepside). The other drug(s) may be administered concomitant with, prior to, or following the administration of the macrolide antibiotic.

In one embodiment, provided is a method for the treatment or prevention of helminthic infections and diseases, the methods comprising administering to a subject an effective amount of a Heterocyclic Compound, or a pharmaceutically acceptable salt, tautomer, isotopologue, or stereoisomer thereof, in combination with one or more antihelminthic agent. In some such embodiments, the helminthic infection is a filarial worm infection. In one embodiment, the treatment of helminthic infections comprises administration of an antihelminthic agent such as benzimidazoles, for example, flubendazole, albendazole, mebendazole, thiabendazole, fenbendazole, or triclabendazole. In one embodiment, the treatment of helminthic infections comprises administration of one or more antihelminthic agents, for example, ivermectin, abamectin, diethylcarbamazine (DEC), suramin, pyrantel pamoate, levamisole, niclosamide, nitazoxanide, oxyclozanide, praziquantel, emodepside, monepantel, derquantel, or pelletierine sulphate. In certain embodiments, a Heterocyclic Compound, or a pharmaceutically acceptable salt, tautomer, isotopologue, or stereoisomer thereof, is used to treat helminthic infections in combination with one or more antihelminthic agents. In some embodiments, the antihelminthic agent is a benzimidazole, for example, flubendazole, albendazole, mebendazole, thiabendazole, fenbendazole, or triclabendazole. In some embodiments, the antihelminthic agent is one or more of ivermectin, abamectin, diethylcarbamazine (DEC), suramin, pyrantel pamoate, levamisole, niclosamide, nitazoxanide, oxyclozanide, praziquantel, emodepside, monepantel, derquantel, or pelletierine sulphate. In certain embodiments, a Heterocyclic Compound, or a pharmaceutically acceptable salt, tautomer, isotopologue, or stereoisomer thereof, is used in a method of treatment or prevention of filarial worm infections and diseases, the method comprising administering to a subject an effective amount of a Heterocyclic Compound, or a pharmaceutically acceptable salt, tautomer, isotopologue, or stereoisomer thereof in combination with one or more antihelminthic agents. In some such embodiments, the antihelminthic agent is selected from flubendazole, albendazole, mebendazole, thiabendazole, fenbendazole, triclabendazole, ivermectin, abamectin, diethylcarbamazine (DEC), suramin, pyrantel pamoate, levamisole, niclosamide, nitazoxanide, oxyclozanide, praziquantel, emodepside, monepantel, derquantel, or pelletierine sulphate. In one embodiment, the antihelminthic agent is a Wolbachia targeting agent. In one embodiment, the Wolbachia targeting agent is doxycycline.

### Pharmaceutical Compositions and Routes of Administration

Provided herein are pharmaceutical compositions comprising an effective amount of a Heterocyclic Compound, as described herein, and a pharmaceutically acceptable carrier, excipient or vehicle. The Heterocyclic Compounds can be administered to a subject enterally (for example, orally, rectally), topically, or parenterally (for example, intravenously, intramuscularly, subcutaneously), in the conventional form of preparations, such as capsules, microcapsules, tablets, granules, powder, troches, pills, suppositories, injections, suspensions, syrups, patches, creams, lotions, ointments, gels, sprays, solutions and emulsions. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinyl pyrrolidone or aluminum stearate), a dispersing agent (e.g., hydroxypropylmethylcellulose), a diluent (e.g., water), a cosolvent (e.g., propylene glocyl/glycofurol), a buffer, a copolymer (e.g., poly(lactic-co-glycolic acid, i.e PLGA), and base wax (e.g., cocoa butter, white petrolatum or polyethylene glycol). The effective amount of the Heterocyclic Compound in the pharmaceutical composition may be at a level that will exercise the desired effect; for example, about 0.005 mg/kg of a subject's body weight to about 20 mg/kg of a subject's body weight in unit dosage for both oral and parenteral administration.

The dose of a Heterocyclic Compound to be administered to a subject is rather widely variable and can be subject to the judgment of a health-care practitioner. In general, the Heterocyclic Compound can be administered one to four times a day in a dose of about 0.5 mg/kg of a subject's body weight to about 20 mg/kg of a subject's body weight in a subject, but the above dosage may be properly varied depending on the age, body weight and medical condition of the subject and the type of administration. In one embodiment, the dose is about 0.1 mg/kg of a subject's body weight to about 3 mg/kg of a subject's body weight, about 0.5 mg/kg of a subject's body weight to about 2 mg/kg of a subject's body weight, about 1 mg/kg of a subject's body weight to about 2 mg/kg of a subject's body weight or about 1.5 mg/kg of a subject's body weight to about 2 mg/kg of a subject's body weight. In one embodiment, the dose is about 1 mg/kg of a subject's body weight to about 3 mg/kg of a subject's body weight. In one embodiment, the dose is about 0.5 mg/kg of a subject's body weight to about 1 mg/kg of a subject's body weight. In one embodiment, the dose is about 1 mg/kg of a subject's body weight to about 2 mg/kg of a subject's body weight. In one embodiment, the dose is about 1.5, 1.6, 1.7, 1.8, 1.9, 2.0, 2.1, 2.2, 2.3, 2.4, 2.5, 2.6, 2.7, 2.8, 2.9, 3.0 mg/kg of a subject's body weight. In one embodiment, one dose is given per day. In any given case, the amount of the Heterocyclic Compound administered will depend on such factors as the solubility of the active component, the formulation used and the route of administration.

In another embodiment, provided herein are methods for the treatment or prevention of a disease or disorder comprising the administration of about 1 mg/day to about 1200 mg/day of a Heterocyclic Compound to a subject affected by helminthic infection. In another embodiment, provided herein are methods for the treatment or prevention of a disease or disorder comprising the administration of about 0.375 mg/day to about 750 mg/day, about 0.75 mg/day to about 375 mg/day, about 3.75 mg/day to about 75 mg/day, about 7.5 mg/day to about 55 mg/day or about 18 mg/day to about 37 mg/day of a Heterocyclic Compound to a subject affected by helminthic infection. In one embodiment, the methods for the treatment of a disease or disorder comprise the administration of about 0.375 mg/day to about 750 mg/day of a Heterocyclic Compound to a subject affected by helminthic infection. In one embodiment, the methods for the treatment of a disease or disorder comprise the administration of about 0.75 mg/day to about 375 mg/day of a Heterocyclic Compound to a subject affected by helminthic infection. In one embodiment, the methods for the treatment of a disease or disorder comprise the administration of about 3.75 mg/day to about 75 mg/day of a Heterocyclic Compound to a subject affected by helminthic infection. In one embodiment, the methods for the treatment of a disease or disorder comprise the administration of about 7.5 mg/day to about 55 mg/day of a Heterocyclic Compound to a subject affected by helminthic infection. In one embodiment, the methods for the treatment of a disease or disorder comprise the administration of about 18 mg/day to about 37 mg/day of a Heterocyclic Compound to a subject affected by helminthic infection.

In another embodiment, provided herein are unit dosage formulations that comprise between about 1 mg and 500 mg, or between about 500 mg and about 1000 mg of a Heterocyclic Compound. In one embodiment, provided herein is a unit dosage formulation that comprise between about 1 mg and 500 mg of a Heterocyclic Compound. In one embodiment, provided herein is a unit dosage formulation that comprise between about 500 mg and about 1000 mg of a Heterocyclic Compound. In another embodiment, provided herein are unit dosage formulations that comprise between about 1 mg and 200 mg, about 35 mg and about 1400 mg, about 125 mg and about 1000 mg, about 250 mg and about 1000 mg, or about 500 mg and about 1000 mg of a Heterocyclic Compound. In one embodiment, the unit dosage formulations comprises between about 1 mg and 200 mg of a Heterocyclic Compound. In one embodiment, the unit dosage formulations comprises between about 35 mg and about 1400 mg of a Heterocyclic Compound. In one embodiment, the unit dosage formulations comprises between about 125 mg and about 1000 mg of a Heterocyclic Compound. In one embodiment, the unit dosage formulations comprises between about 250 mg and about 1000 mg of a Heterocyclic Compound. In one embodiment, the unit dosage formulations comprises between about 500 mg and about 1000 mg of a Heterocyclic Compound.

In a particular embodiment, provided herein are unit dosage formulations comprising about 100 mg or 400 mg of a Heterocyclic Compound.

In another embodiment, provided herein are unit dosage formulations that comprise 1 mg, 5 mg, 10 mg, 15 mg, 20 mg, 30 mg, 35 mg, 40 mg, 50 mg, 70 mg, 100 mg, 125 mg, 130 mg, 140 mg, 175 mg, 200 mg, 250 mg, 280 mg, 350 mg, 500 mg, 560 mg, 700 mg, 750 mg, 1000 mg or 1400 mg of a Heterocyclic Compound. In one embodiment, the unit dosage formulations comprise 1 mg of a Heterocyclic Compound. In one embodiment, the unit dosage formulations comprise 5 mg of a Heterocyclic Compound. In one embodiment, the unit dosage formulations comprise 10 mg of a Heterocyclic Compound. In one embodiment, the unit dosage formulations comprise 15 mg of a Heterocyclic Compound. In one embodiment, the unit dosage formulations comprise 20 mg of a Heterocyclic Compound. In one embodiment, the unit dosage formulations comprise 25 mg of a Heterocyclic Compound. In one embodiment, the unit dosage formulations comprise 30 mg of a Heterocyclic Compound. In one embodiment, the unit dosage formulations comprise 35 mg of a Heterocyclic Compound. In one embodiment, the unit dosage formulations comprise 40 mg of a Heterocyclic Compound. In one embodiment, the unit dosage formulations comprise 50 mg of a Heterocyclic Compound. In one embodiment, the unit dosage formulations comprise 70 mg of a Heterocyclic Compound. In one embodiment, the unit dosage formulations comprise 100 mg of a Heterocyclic Compound. In one embodiment, the unit dosage formulations comprise 125 mg of a Heterocyclic Compound. In one embodiment, the unit dosage formulations comprise 130 mg of a Heterocyclic Compound. In one embodiment, the unit dosage formulations comprise 140 mg of a Heterocyclic Compound. In one embodiment, the unit dosage formulations comprise 175 mg of a Heterocyclic Compound. In one embodiment, the unit dosage formulations comprise 200 mg of a Heterocyclic Compound. In one embodiment, the unit dosage formulations comprise 250 mg of a Heterocyclic Compound. In one embodiment, the unit dosage formulations comprise 280 mg of a Heterocyclic Compound. In one embodiment, the unit dosage formulations comprise 350 mg of a Heterocyclic Compound. In one embodiment, the unit dosage formulations comprise 500 mg of a Heterocyclic Compound. In one embodiment, the unit dosage formulations comprise 560 mg of a Heterocyclic Compound. In one embodiment, the unit dosage formulations comprise 700 mg of a Heterocyclic Compound. In one embodiment, the unit dosage formulations comprise 750 mg of a Heterocyclic Compound. In one embodiment, the unit dosage formulations comprise 1000 mg of a Heterocyclic Compound. In one embodiment, the unit dosage formulations comprise 1400 mg of a Heterocyclic Compound.

An Heterocyclic Compound can be administered once, twice, three, four or more times daily. In a particular embodiment, doses of 600 mg or less are administered as a once daily dose and doses of more than 600 mg are administered twice daily in an amount equal to one half of the total daily dose.

An Heterocyclic Compound can be administered orally for reasons of convenience. In one embodiment, when administered orally, a Heterocyclic Compound is administered with a meal and water. In another embodiment, the Heterocyclic Compound is dispersed in water or juice (e.g., apple juice or orange juice) and administered orally as a suspension.

The Heterocyclic Compound can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, topically to the ears, nose, eyes, or skin, or by local ocular (i.e., subconjunctival, intravitreal, retrobulbar, or intracameral). The mode of administration is left to the discretion of the health-care practitioner, and can depend in-part upon the site of the medical condition.

In one embodiment, provided herein are capsules containing a Heterocyclic Compound without an additional carrier, excipient or vehicle.

In another embodiment, provided herein are compositions comprising an effective amount of a Heterocyclic Compound and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories, suspensions, gels, intra-ruminal devices (e.g., for prolonged prophylaxis or controlled release), implants, topical pour-ons, transdermal delivery gels, spot-ons, implants (including devices, gels, liquids (e.g., PLGA), and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a Heterocyclic Compound with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the dye. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a Heterocyclic Compound as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the Heterocyclic Compound can be delayed or prolonged by proper formulation. For example, a slowly soluble pellet of the Heterocyclic Compound can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the Heterocyclic Compound in oily or emulsified vehicles, or adding amounts of PLGA, that allow it to disperse slowly in the serum.

### EXAMPLES

The following Examples are presented by way of illustration, not limitation. Compounds are named using the automatic name generating tool provided in Chemdraw Ultra 17.0 (Cambridgesoft), which generates systematic names for chemical structures, with support for the Cahn-Ingold-Prelog rules for stereochemistry. One skilled in the art can modify the procedures set forth in the illustrative examples to arrive at the desired products.

### ABBREVIATIONS USED

| | |
|---|---|
| AcOH | Acetic acid |
| Ac₂O | Acetic anhydride |
| BINAP | (2,2′-Bis(diphenylphosphino)-1,1′-binaphthyl) |
| nBuLi | n-Butyl lithium |
| CDI | Carbonyldiimidazole |
| DBU | 1,8-Diazabicyclo (5.4.0) undec-7-ene |
| DCE | 1,2-dichloroethane |
| DCM | Dichloromethane |
| DIPEA | Diisopropylethylamine |
| DMF | N,N-Dimethylformamide |
| DMSO | Dimethylsulfoxide |
| EDCI | Ethyl-(N′,N′-dimethylamino)propylcarbodiimide |
| | hydrochloride |
| ESI | Electrospray ionization |
| Et₂O | Diethyl ether |
| EtOH | Ethanol |
| EtOAc | Ethyl acetate |
| HATU | O-(7-Azabenzotriazol-1-yl)-N,N,N′,N′- |
| | tetramethyluronium hexafluorophosphate |
| HEPES | (4-(2-hydroxyethyl)-1-piperazineethanesulfonic acid) |
| HBr | Hydrobromic acid |
| HCl | Hydrochloric acid |
| H₂O | Water |
| H₂O₂ | Hydrogen peroxide |
| HOBt | 1-Hydroxybenzotriazole |
| HPLC | High performance liquid chromatography |
| K₂CO₃ | Potassium carbonate |
| KHCO₃ | Potassium bicarbonate |
| LiEt₃BH | Lithium triethylborohydride (Superhydride) |
| LCMS | Liquid chromatography mass spectrometry |
| mCPBA | Meta-chloroperoxybenzoic acid |
| MeCN or ACN | Acetonitrile |
| MeMgBr | Methyl magnesium bromide |
| MeOH | Methanol |
| MS | Mass spectrometry |
| MTBE | Methyl tertiary butyl ether |
| Na₂CO₃ | Sodium carbonate |
| Na₂S | Sodium sulfide |
| NaH | Sodium hydride |
| NaHCO₃ | Sodium bicarbonate |
| NaOH | Sodium hydroxide |
| Na₂SO₄ | Sodium sulphate |
| NBS | N-Bromosuccinimide |
| NH₄Cl | Ammonium chloride |
| NMM | N-Methylmorpholine |
| NMR | Nuclear magnetic resonance |
| P₂S₅ | Phosphorous pentasulfide |
| Pd/C | Palladium (0) on carbon |
| Pd(PPh₃)₄ | Tetrakis(triphenylphosphine)palladium (0) |
| Pd₂(dba)₃ | Tris(dibenzylideneacetone)dipalladium (0) |
| Pd(PPh₃)₂Cl₂ | Bis(triphenylphosphine)palladium(II) dichloride |
| TEA | Triethylamine |
| TFA | Trifluoracetic acid |
| THF | Tetrahydrofuran |
| THP | Tetrahydropyran |
| TLC | Thin layer chromatography |
| UPLC | Ultra Performance Liquid Chromatography |
| UHPLC | Ultra High Performance Liquid Chromatography |
| Xantphos | 4,5-Bis(diphenylphosphino)-9,9-dimethylxanthene |

### Compound Synthesis

### Example 1. N-(4-Methylpyridin-2-yl)-4-(5-methylpyridin-2-yl)thiazol-2-amine

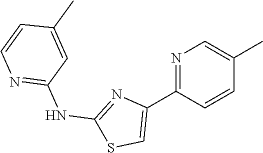 [SMILES: Cc1ccc(-c2csc(Nc3cc(C)ccn3)n2)nc1]

A solution of 2-bromo-1-(5-methylpyridin-2-yl)ethanone (0.214 g, 1 mmol) and 1-phenylthiourea (0.152 g, 1.00 mmol) in EtOH (10 ml) was stirred at 78° C. for 1 h. The reaction mixture was quenched with saturated NaHCO₃, washed with EtOAc, and then washed with saturated aqueous NaCl. The organic layer was combined, dried over magnesium sulfate, filtered, and concentrated. The crude residue was purified using reverse-phased semi-preparative chromatography. The fraction containing clean product was loaded onto a Phenomenex Strata-X-C ion exchange column. The column was washed successively with water and MeOH. The product was isolated and purified via standard methods to afford N-(4-methylpyridin-2-yl)-4-(5-methylpyridin-2-yl)thiazol-2-amine (0.180 g, 0.637 mmol, 63.7% yield); ¹H NMR (400 MHz, DMSO-d₆) δ ppm 2.29 (s, 3H) 2.32 (s, 3H) 6.76-6.82 (m, 1H) 6.87-6.92 (m, 1H) 7.55 (s, 1H) 7.65-7.72 (m, 1H) 7.86 (d, J=8.20 Hz, 1H) 8.17 (d, J=5.86 Hz, 1H) 8.41-8.45 (m, 1H) 11.32 (br s, 1H). LCMS (ESI) m/z 283.4 [M+H]⁺.

### Example 2. N-(4-Methylpyridin-2-yl)-4-(pyridin-2-yl)thiazol-2-amine

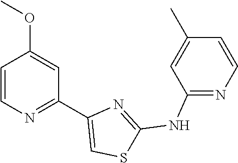 [SMILES: COc1ccnc(-c2csc(Nc3cc(C)ccn3)n2)c1]

1-(4-Methylpyridin-2-yl)thiourea. A mixture of benzoyl isothiocyanate (5 g, 1.0 mol) and 4-methylpyridin-2-amine (3.3 g, 1.0 mol) in EtOH (50 mL) under a nitrogen atmosphere was stirred at 80° C. for 12 h. After that, the reaction was poured into ice-water and stirred for an additional 30 min. The benzoyl thiourea precipitate was collected by filtration and washed with water. The crude material was dissolved in MeOH and treated with 1 N NaOH. The reaction mixture was heated to reflux for 1 h. After cooling, the reaction mixture was poured into ice-water and aqueous 1 N HCl was added to produce pH of approximately 3-4. The reaction was stirred for 30 min and pH was adjusted to 8-9 using saturated Na₂CO₃, resulting in a precipitate. The precipitate was collected by filtration, washed with water, and dried to give the title compound 1-(4-methylpyridin-2-yl)thiourea (3.2 g, 62%).

4-(4-Methoxypyridin-2-yl)-N-(4-methylpyridin-2-yl)thiazol-2-amine. The solution of 2-chloro-1-(4-methoxypyridin-2-yl)ethanone (0.186 g, 1 mmol) and 1-(4-methylpyridin-2-yl)thiourea (0.167 g, 1.000 mmol) in EtOH (10 ml) was stirred at 78° C. for 1 h. The reaction mixture was quenched with saturated aqueous NaCl and then washed with EtOAc. The product was isolated and purified via standard methods to afford 4-(4-methoxypyridin-2-yl)-N-(4-methylpyridin-2-yl)thiazol-2-amine (0.25 g, 0.838 mmol, 84% yield). ¹H NMR (400 MHz, DMSO-d₆) δ ppm 2.29 (s, 4H) 6.76-6.82 (m, 1H) 6.86-6.93 (m, 3H) 7.51 (d, J=2.73 Hz, 1H) 7.62 (s, 1H) 8.13-8.21 (m, 1H) 8.40 (d, J=6.25 Hz, 1H) 11.40 (s, 1H). LCMS (ESI) m/z 299.3 [M+H]⁺.

### Example 3. N-(4-Isopropoxypyridin-2-yl)-4-(5-methoxypyridin-2-yl)thiazol-2-amine

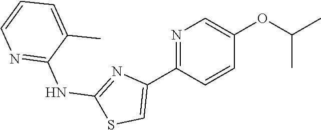 [SMILES: Cc1cccnc1Nc1nc(-c2ccc(OC(C)C)cn2)cs1]

5-Isopropoxypicolinaldehyde. To a mixture of 5-hydroxypicolinaldehyde (9 g, 73.10 mmol) and K₂CO₃ (10.1 g, 73.10 mmol) in DMF (100 mL) stirred under nitrogen atmosphere at 25° C. was added isopropyl iodide (12.4 g, 73.10 mmol) dropwise over 30 min. The reaction mixture was stirred at 100° C. for 3 h. The reaction mixture was poured into ice-water and extracted with EtOAc. The combined organic layer was washed with water then brine, dried over anhydrous Na₂SO₄, and concentrated under vacuum. The product was purified by silica gel chromatography to afford 5-isopropoxypicolinaldehyde (8.5 g, 70% yield).

1-(5-Isopropoxypyridin-2-yl)ethanol. To a solution of 5-isopropoxypicolinaldehyde (8.5 g, 51.45 mmol) in THE (150 mL), stirred under a nitrogen atmosphere at 0° C., was added methyl magnesium chloride (25.7 mL, 77.18 mmol) dropwise over 15 min. The reaction mixture was stirred at 24° C. for 16 h. The reaction was quenched with saturated NH₄Cl solution at 0° C. and then extracted with EtOAc. The combined organic layer was washed with water and brine, dried over anhydrous Na₂SO₄, and concentrated under vacuum to afford 1-(5-isopropoxypyridin-2-yl)ethanol (8.5 g, 91% yield).

1-(5-Isopropoxypyridin-2-yl)ethanone. To a solution of 2,2,6,6-tetramethylpiperidine-1-oxyl (750 mg, 4.69 mmol) and trichloroisocyanuric acid (12 g, 51.65 mmol) at 0° C. were added to a solution of 1-[5-(1-methylethoxy)pyridin-2-yl]ethanol (8.5 g, 46.96 mmol) dissolved in acetone (100 mL) and the mixture was stirred at 0° C. for 10 min. The reaction solution was concentrated under vacuum. The residue was stirred with an aqueous solution of NaHCO₃ and the resulting solution was extracted with EtOAc. The combined organic layer was washed with brine, dried over Na₂SO₄, and concentrated under reduced pressure to afford 1-(5-isopropoxypyridin-2-yl)ethanone (5 g) which was used further without purification.

2-Bromo-1-(5-isopropoxypyridin-2-yl)ethanone. To a solution of 1-(5-isopropoxypyridin-2-yl)ethanone (5 g, 27.93 mmol) in THE (50 mL) under a nitrogen atmosphere at 24° C., was added pyridinium tribromide (8.9 g, 27.93 mmol) in one portion. The reaction mixture was stirred at 24° C. for 16 h. The resulting solid mass was collected by filtration, rinsed with THF, and dried under vacuum to get 2-bromo-1-(5-isopropoxypyridin-2-yl)ethanone (4 g) that was used further without purification.

4-(5-Isopropoxypyridin-2-yl)-N-(3-methylpyridin-2-yl)thiazol-2-amine. To a solution of 1-(3-methylpyridin-2-yl)thiourea (2 g, 11.95 mmol) in EtOH (50 mL) under a nitrogen atmosphere at 25° C., was added 2-bromo-1-(5-isopropoxypyridin-2-yl)ethanone (3.7 g, 14.35 mmol) in one portion. The reaction mixture was stirred at 80° C. for 4 h. The reaction mixture was poured into ice-water and adjusted to pH of 8 with aqueous ammonia. The resulting crude material was collected by filtration, washed with water, and dried under vacuum. The product was isolated and purified via standard methods to afford 4-(5-methoxypyridin-2-yl)-N-(3-methylpyridin-2-yl)thiazol-2-amine (1.05 g, 27% yield). MS (ESI) m/z 327.29 [M+H]⁺.

### Example 4. 4-(5-Methoxypyridin-2-yl)-N-(3-methylpyridin-2-yl)thiazol-2-amine

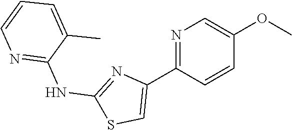 [SMILES: COc1ccc(-c2csc(Nc3ncccc3C)n2)nc1]

N-(3-Methylpyridin-2-ylcarbamothioyl)benzamide. To a solution of 3-methylpyridin-2-amine (10 g, 92.47 mmol) in acetone (100 mL), stirred under a nitrogen atmosphere at 24° C., was added benzoyl isothiocyanate (16.6 g, 101.71 mmol) dropwise. The reaction mixture was stirred at 70° C. for 3 h. The reaction mixture was diluted with water and extracted with EtOAc. The combined organic layer was washed with water and brine, dried over anhydrous Na₂SO₄, and concentrated under vacuum. The crude product was triturated with n-pentane, the solid was collected by filtration, and the product was dried under vacuum to afford N-(3-methylpyridin-2-ylcarbamothioyl)benzamide (20 g, 80% yield).

1-(3-Methylpyridin-2-yl)thiourea. To a stirred solution of N-(3-methylpyridin-2-ylcarbamothioyl)benzamide (10 g, 36.90 mmol) in MeOH (100 ml) was added 1N NaOH solution (50 mL) dropwise at 24° C. The reaction mixture was stirred at 80° C. for 2 h. The reaction mixture was poured into ice-water and the pH of the solution was adjusted to neutral with diluted HCl. The resulting solid mass was collected by filtration and dried under vacuum to afford 1-(3-methylpyridin-2-yl)thiourea (5 g, 29.94 mmol, 81% yield).

2-Bromo-1-(5-methoxypyridin-2-yl)ethanone. To a solution of 1-(5-methoxypyridin-2-yl)ethanone (3 g, 19.867 mmol) in 33% HBr in AcOH (10.8 mL) stirred at 0° C., was added a suspension of pyridinium tribromide (8.2 g, 25.827 mmol) in AcOH (210 mL) dropwise. The reaction mixture was stirred at 24° C. for 5 h. The reaction mixture was diluted with Et₂O and the solution was kept at −4° C. in a refrigerator for 16 h. The resulting crude material was collected by filtration, washed with Et₂O, and dried under vacuum to afford 2-bromo-1-(5-methoxypyridin-2-yl)ethanone (4 g) and used without purification in the next step. MS (ESI) m/z 230.36 [M+1]⁺.

4-(5-Methoxypyridin-2-yl)-N-(3-methylpyridin-2-yl)thiazol-2-amine. To a solution of 1-(3-methylpyridin-2-yl)thiourea (2 g, 11.95 mmol) in EtOH (50 mL) stirred under nitrogen atmosphere at 24° C., was added 2-bromo-1-(5-methoxypyridin-2-yl)ethanone (3.7 g, 14.35 mmol) in one portion. The reaction mixture was stirred at 80° C. for 4 h. The reaction mixture was poured into ice-water and adjusted to a pH of 10 with aqueous ammonia. The crude material was collected by filtration, washed with water (10 mL), and dried under vacuum. The product was isolated and purified via standard methods to afford 4-(5-methoxypyridin-2-yl)-N-(3-methylpyridin-2-yl)thiazol-2-amine (1.3 g, 37% yield). MS (ESI) m/z 299.22 [M+H]⁺.

### Example 5. 4-(5-Methoxypyridin-2-yl)-N-(5-(tetrahydro-2H-pyran-4-yloxy)pyridin-2-yl)thiazol-2-amine

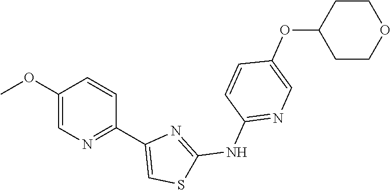 [SMILES: COc1ccc(-c2csc(Nc3ccc(OC4CCOCC4)cn3)n2)nc1]

Tetrahydro-2H-pyran-4-yl methanesulfonate. To a stirred, cooled solution of tetrahydro-2H-pyran-4-ol (7.00 g, 68.6 mmol) and TEA (20.7 g, 206 mmol) in DCM (50 mL) was added methanesulfonyl chloride (10.1 g, 89.2 mmol) dropwise. The mixture was stirred for 16 h at 24° C. The reaction was partitioned between water and DCM. The organic layer was separated, dried over anhydrous Na₂SO₄, and concentrated. The residue was purified by silica gel chromatography to give the desired product (6.4 g, 35.6 mmol, 52%).

5-(Tetrahydro-2H-pyran-4-yloxy)pyridin-2-amine. 6-aminopyridin-3-ol (2.93 g, 26.7 mmol) and K₂CO₃ (9.2 g, 66.7 mmol) were added to a solution of tetrahydro-2H-pyran-4-yl methanesulfonate (4.00 g, 22.2 mmol) in N,N-dimethylformide (20 mL). The mixture was stirred at 100° C. for 16 h. The reaction was cooled to 24° C. and filtered to remove the inorganic salt. The filtrate was partitioned between water and DCM. The organic layer was separated, dried over anhydrous Na₂SO₄, and concentrated. The residue was purified by silica gel chromatography to afford the title compound (1.25 g, 6.44 mmol, 29% yield).

N-(5-(Tetrahydro-2H-pyran-4-yloxy)pyridin-2-ylcarbamothioyl)benzamide. Benzyl thioisocyanate (1.05 g, 6.44 mmol) was added to a solution of 5-(tetrahydro-2H-pyran-4-yloxy)pyridin-2-amine (1.25 g, 6.44 mmol) in DCM (10 mL). The mixture was stirred at 24° C. for 4 h. The solvent was removed under reduced pressure and the residue was purified by silica gel column chromatography to afford the titled compound (1.60 g, 4.48 mmol, 69% yield).

1-(5-(Tetrahydro-2H-pyran-4-yloxy)pyridin-2-yl)thiourea. N-(5-(tetrahydro-2H-pyran-4-yloxy)pyridin-2-ylcarbamothioyl)benzamide (900 mg, 2.52 mmol) was dissolved in aqueous NaOH (2.5 M, 10 mL) and the mixture was stirred at 80° C. for 1 h. The reaction was cooled to 0° C., and the precipitate was collected by filtration. The filter cake was washed with water and dried to give the desired product (560 mg, 2.21 mmol, 88% yield).

4-(5-Methoxypyridin-2-yl)-N-(5-(tetrahydro-2H-pyran-4-yloxy)pyridin-2-yl)-thiazol-2-amine. To a solution of 1-(5-(tetrahydro-2H-pyran-4-yloxy)pyridin-2-yl) thiourea (80 mg, 0.316 mmol) in EtOH (5 mL) was added 2-bromo-1-(5-methoxy-pyridin-2-yl)ethanone hydrobromide (99 mg, 0.316 mmol). The mixture was stirred at 24° C. for 1 h. The product was isolated and purified via standard methods to give (56 mg, 0.146 mmol, 46% yield). MS (ESI) m/z 385.1 [M+H]⁺.

### Example 6. N-(4-Isopropoxypyridin-2-yl)-4-(5-methoxypyridin-2-yl)thiazol-2-amine

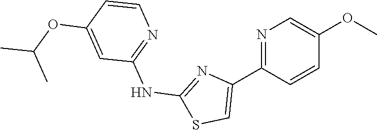 [SMILES: COc1ccc(-c2csc(Nc3cc(OC(C)C)ccn3)n2)nc1]

4-Isopropoxypyridin-2-amine. To a solution of 4-chloropyridin-2-amine (10 g, 77.7 mmol) in DMSO (500 mL) under nitrogen atmosphere at 24° C., was added sodium propan-2-olate (63.7 g, 777.0 mmol) and the reaction solution was stirred at 150° C. for 4 h. The reaction mixture was poured into ice-water and extracted with EtOAc. The combined organic layer was washed with water and brine, dried over anhydrous Na₂SO₄, and concentrated under vacuum. The crude product was purified by silica gel chromatography and obtained 4-isopropoxypyridin-2-amine (7 g, 59% yield).

N-(4-Isopropoxypyridin-2-ylcarbamothioyl)benzamide. To a solution of 4-isopropoxypyridin-2-amine (10 g, 65.78 mmol) in THE (100 mL) under a nitrogen atmosphere at 24° C., was added benzoyl isothiocyanate (10.7 g, 65.78 mmol) dropwise. The reaction mixture was stirred at 70° C. for 3 h. The reaction mixture was diluted with water. The product was collected by filtration, washed with water, and dried under vacuum to afford N-(4-isopropoxypyridin-2-ylcarbamothioyl)benzamide (12 g, 58% yield) which was used in the next step without further purification.

1-(4-Isopropoxypyridin-2-yl)thiourea. To a solution of N-(4-isopropoxypyridin-2-ylcarbamothioyl)benzamide (6 g, 19.04 mmol) in MeOH (50 mL) at 24° C., was added 1 N NaOH (20 mL) and the mixture was stirred at 80° C. for 2 h. The reaction mixture was poured into ice-water and the pH was adjusted to 7 with 1N HCl. The resulting crude material was collected by filtration, washed with water, and dried under vacuum to afford 1-(4-isopropoxypyridin-2-yl)thiourea (3.5 g, 87% yield).

N-(4-Isopropoxypyridin-2-yl)-4-(5-methoxypyridin-2-yl)thiazol-2-amine. To a solution of 1-(4-isopropoxypyridin-2-yl)thiourea (2 g, 9.478 mmol) in EtOH (50 ml) under a nitrogen atmosphere at 24° C., was added 2-bromo-1-(5-methoxypyridin-2-yl)ethanone (2.2 g, 9.478 mmol) in one portion. The reaction mixture was stirred at 80° C. for 1 h. The reaction mixture was poured into ice-water and adjusted to pH 10 with aqueous ammonia. The product was isolated and purified via standard methods to afford N-(4-isopropoxypyridin-2-yl)-4-(5-methoxypyridin-2-yl)thiazol-2-amine (2.5 g, 78% yield). MS (ESI) m/z 343.20 [M+H]⁺.

### Example 7. 6-(2-((3-Methylpyridin-2-yl)amino)thiazol-4-yl)nicotinamide

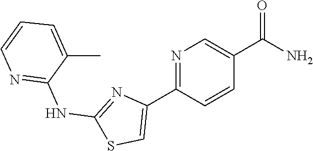 [SMILES: Cc1cccnc1Nc1nc(-c2ccc(C(N)=O)cn2)cs1]

6-(2-Bromoacetyl)nicotinamide. Bromine (0.147 ml, 2.85 mmol) was added to a suspension of 6-acetylnicotinonitrile (0.416 g, 2.85 mmol) and HBr in AcOH (33%) (5 ml). The reaction was stirred at 70° C. for 1 h. The reaction mixture was quenched with saturated NaHCO₃ and then washed with EtOAc. The organic phase was combined and washed with saturated aqueous NaCl. The organic layer was dried over magnesium sulfate, filtered, and concentrated. The residue was purified by silica gel column chromatography to afford 6-(2-bromoacetyl)nicotinamide (0.577 g, 2.374 mmol, 83% yield). ¹H NMR (400 MHz, DMSO-d₆) δ ppm 5.06 (s, 2H) 7.83 (br. s., 1H) 8.35 (br. s., 1H) 8.41 (dd, J=8.20, 1.56 Hz, 1H) 9.12 (d, J=1.95 Hz, 1H); MS (ESI) m/z 243.0 [M+H]⁺.

6-(2-((3-Methylpyridin-2-yl)amino)thiazol-4-yl)nicotinamide. A solution of 6-(2-bromoacetyl)nicotinamide (0.243 g, 1 mmol), 1-(3-methylpyridin-2-yl)thiourea (0.167 g, 1.000 mmol) in EtOH (10 ml) was stirred at 78° C. for 1 h. The reaction mixture was purified using reverse-phased semi-preparative HPLC. The fraction containing clean product was loaded onto a Phenomenex Strata-X-C ion exchange column. The column was washed successively with water and MeOH. The product was isolated and purified via standard methods to afford 6-(2-((3-methylpyridin-2-yl)amino)thiazol-4-yl)nicotinamide (0.125 g, 0.401 mmol, 40.1% yield); ¹H NMR (400 MHz, DMSO-d₆) δ ppm 2.38 (s, 3H) 6.93 (dd, J=7.42, 5.08 Hz, 1H) 7.54-7.62 (m, 2H) 7.80 (s, 1H) 8.11 (d, J=8.20 Hz, 1H) 8.16 (s, 1H) 8.20 (d, J=3.12 Hz, 1H) 8.32 (dd, J=8.20, 1.95 Hz, 1H) 9.05 (d, J=2.34 Hz, 1H) 10.58 (s, 1H). MS (ESI) m/z 312.2 [M+H]⁺.

### Example 8. N,N-Dimethyl-6-(2-((3-methylpyridin-2-yl)amino)thiazol-4-yl)nicotinamide

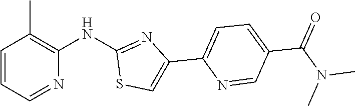 [SMILES: Cc1cccnc1Nc1nc(-c2ccc(C(=O)N(C)C)cn2)cs1]

6-Bromo-N,N-dimethylnicotinamide. 4-Methylmorpholine (7.51 g, 74.3 mmol) was added to a solution of 6-bromonicotinic acid (5.00 g, 24.8 mmol) and dimethylamine hydrochloride (3.03 g, 37.1 mmol) in THE (100 mL). Then EDCI (5.7 g, 29.7 mmol) and HOBt (4 g, 29.7 mmol) were added to the mixture. The mixture was stirred at 25° C. for 16 h under nitrogen. The reaction mixture was concentrated under reduced pressure. The residue was poured into water and the aqueous phase was extracted with EtOAc. The combined organic layers were washed with brine, dried over Na₂SO₄, filtered, and concentrated under reduced pressure to give a residue. The residue was purified by silica gel chromatography to get 6-bromo-N,N-dimethylnicotinamide (5 g, crude).

6-(1-Ethoxyvinyl)-N,N-dimethylnicotinamide. CuI (623 mg, 3.27 mmol) and Pd(PPh₃)₂Cl₂ (1.5 g, 2.18 mmol) under nitrogen were added to a mixture of 6-bromo-N,N-dimethylnicotinamide (5.00 g, 21.8 mmol) and tributyl(1-ethoxyvinyl)stannane (8.67 g, 24.0 mmol) in acetonitrile (100 mL). The mixture was stirred at 90° C. for 48 h under nitrogen. The mixture was poured into water and potassium fluoride (2 g) was added. The mixture was filtered through a pad of celite and the filter cake was washed with EtOAc. The combined organic phase was washed with brine, dried with anhydrous Na₂SO₄, filtered, and concentrated in vacuo. The residue was purified using column chromatography to provide 6-(1-ethoxyvinyl)-N,N-dimethylnicotinamide (5.00 g, 15.9 mmol, 73% yield, 70% purity).

6-(2-Bromoacetyl)-N,N-dimethylnicotinamide. NBS (1.45 g, 8.17 mmol) was added in portions at 0° C. to a mixture of 6-(1-ethoxyvinyl)-N,N-dimethylnicotinamide (2.57 g, 8.17 mmol) in THE (49 mL) and water (16 mL). The mixture was stirred at 26° C. for 0.5 h. The mixture was poured into water and the aqueous phase was extracted with EtOAc. The combined organic phase was washed with brine, dried over anhydrous Na₂SO₄, filtered, and concentrated in vacuo to get 6-(2-bromoacetyl)-N,N-dimethylnicotinamide (3.3 g, crude).

N-((3-Methylpyridin-2-yl)carbamothioyl)benzamide. To a solution of benzoyl chloride (2.00 g, 14.2 mmol) in acetone (20 mL) was added ammonia thiocyanic acid (291 mg, 17.1 mmol) under nitrogen. The mixture was stirred at 60° C. for 1 h. A solution of 3-methylpyridin-2-amine (1.54 g, 14.2 mmol) in acetone (5 mL) was added dropwise into the above mixture at 20° C. under nitrogen. The mixture was stirred at 60° C. for 2 h. The mixture was poured into water and the aqueous phase was extracted with EtOAc. The combined organic phases were washed with brine, dried over anhydrous Na₂SO₄, filtered, and concentrated in vacuo to give a residue. The residue was purified by silica gel chromatography to get N-((3-methylpyridin-2-yl)carbamothioyl)benzamide (1.8 g, crude).

1-(3-Methylpyridin-2-yl)thiourea. To a solution of N-((3-methylpyridin-2-yl)carbamothioyl)benzamide (1.30 g, 4.79 mmol) in MeOH (40 mL) was added a solution of NaOH (1 g, 26.0 mmol) in water (10 mL). The mixture was stirred at 80° C. for 3 h. The reaction mixture was concentrated under reduced pressure and 1N HCl was added to the mixture until a pH of 7 was obtained. The residue was diluted with water. The aqueous phase was extracted with EtOAc. The combined organic layers were washed with brine, dried over Na₂SO₄, filtered, and concentrated to get 1-(3-methylpyridin-2-yl)thiourea (850 mg, crude).

N,N-Dimethyl-6-(2-((3-methylpyridin-2-yl)amino)thiazol-4-yl)nicotinamide. 1-(3-methylpyridin-2-yl)thiourea (1.00 g, 5.38 mmol) was added to a mixture of 6-(2-bromoacetyl)-N,N-dimethylnicotinamide (2.50 g, 6.46 mmol) in EtOH (50 mL). The mixture was stirred at 80° C. for 1 h under nitrogen. The product was isolated and purified via standard methods to afford N,N-dimethyl-6-[2-[(3-methyl-2-pyridyl)amino]thiazol-4-yl]pyridine-3-carboxamide (1.53 g, 4.40 mmol, 82% yield, 97.5% purity). MS (ESI): m/z 340.2 [M+1]⁺.

### Example 9. N-(3-Methylpyridin-2-yl)-4-(5-((tetrahydro-2H-pyran-4-yl)oxy)pyridin-2-yl)thiazol-2-amine

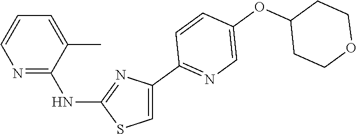 [SMILES: Cc1cccnc1Nc1nc(-c2ccc(OC3CCOCC3)cn2)cs1]

Tetrahydro-2H-pyran-4-yl methanesulfonate. Methanesulfonyl chloride (10.1 g, 89.2 mmol) was added dropwise to a stirred, cooled solution of tetrahydro-2H-pyran-4-ol (7.00 g, 68.6 mmol) and TEA (20.7 g, 206 mmol) in DCM (50 mL). The mixture was stirred at 24° C. for 16 h. The reaction was partitioned between water and DCM. The organic layer was separated, dried over anhydrous Na₂SO₄ and concentrated. The residue was purified by silica gel chromatography to give the desired product (6.4 g, 35.6 mmol, 52%).

2-Bromo-5-((tetrahydro-2H-pyran-4-yl)oxy)pyridine. K₂CO₃ (6.35 g, 46 mmol) and tetrahydro-2H-pyran-4-yl methanesulfonate (6.2 g, 34.5 mmol) were added to a solution of 6-bromopyridin-3-ol (4.0 g, 23 mmol) in DMF (45 mL). The mixture was stirred at 100° C. for 16 h. Water was added and the reaction mixture was extracted with DCM. The combined organic phase was washed with water and brine, dried over magnesium sulfate, filtered, and concentrated under reduced pressure. The crude was purified by column chromatography to afford the desired product (3.6 g, 14 mmol, 61%).

2-Chloro-1-(5-((tetrahydro-2H-pyran-4-yl)oxy)pyridin-2-yl)ethanone. To a solution of 2-bromo-5-((tetrahydro-2H-pyran-4-yl)oxy)pyridine (0.84 g, 3.2 mmol) in THE (20 mL), was added a solution of nBuLi in THE (1.3 mL, 2.5M, 3.2 mmol) at −78° C. and stirred for 2 h. To the mixture was added 2-chloro-N-methoxy-N-methyl-acetamide (0.88 g, 6.4 mmol) in THE (5 mL) and the solution was stirred at −78° C. for another 4 h. Saturated aqueous NH₄Cl was added to quench the reaction, followed by addition of EtOAc. The organic layer was washed by water and brine, dried over magnesium sulfate, filtered, and concentrated. The crude was purified by column chromatography on silica gel to afford the desired product (0.30 g, 1.18 mmol, 36%).

N-(3-Methylpyridin-2-yl)-4-(5-((tetrahydro-2H-pyran-4-yl)oxy)pyridin-2-yl)thiazol-2-amine. 2-Chloro-1-(5-((tetrahydro-2H-pyran-4-yl)oxy) pyridin-2-yl)ethanone (0.302 g, 1.18 mmol) was added to a solution of 1-(3-methylpyridin-2-yl)thiourea (0.197 g, 1.2 mmol) in EtOH (8 mL). The reaction was refluxed for 1 h. The solvent was removed under reduced pressure. The product was isolated and purified via standard methods to afford the title compound (0.065 g, 0.17 mmol, 14% yield). MS (ESI) m/z 369.1 [M+H]⁺.

### Example 10. 4-(5-Fluoropyridin-2-yl)-N-(4-(trifluoromethyl)pyridin-2-yl)-thiazol-2-amine

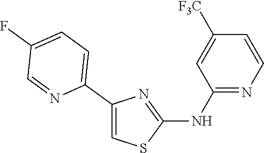 [SMILES: Fc1ccc(-c2csc(Nc3cc(C(F)(F)F)ccn3)n2)nc1]

4-(5-Fluoropyridin-2-yl)-N-(4-(trifluoromethyl)pyridin-2-yl)thiazol-2-amine. 2-Bromo-1-(5-fluoropyridin-2-yl)ethan-1-one hydro bromide (203 mg, 0.678 mmol) and DIPEA (0.118 ml, 0.678 mmol) were added to a solution of 1-(4-(trifluoromethyl)pyridin-2-yl)thiourea (100 mg, 0.452 mmol) in EtOH (5 ml) and the mixture was stirred at 85° C. for 4 h. A precipitate formed and was filtered after cooling of the reaction mixture. The product was isolated and purified via standard methods to afford 4-(5-fluoropyridin-2-yl)-N-(4-(trifluoromethyl)pyridin-2-yl)thiazol-2-amine (95 mg, 0.276 mmol, 61.1% yield). ¹H NMR (300 MHz, DMSO-d6) δ 11.82 (s, 1H), 8.65-8.54 (m, 2H), 8.02 (dd, J=8.8, 4.6 Hz, 1H), 7.83 (td, J=8.8, 3.0 Hz, 1H), 7.69 (s, 1H), 7.43 (s, 1H), 7.26 (dd, J=5.4, 1.5 Hz, 1H). LCMS (APCI): m/z 341.1 [M+H]⁺.

### Example 11. N-(5-Isopropyl-4-(trifluoromethyl)pyridin-2-yl-4-(pyridin-2-yl)thiazol-2-amine

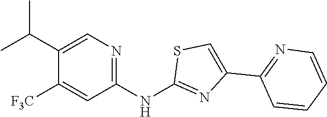 [SMILES: CC(C)c1cnc(Nc2nc(-c3ccccn3)cs2)cc1C(F)(F)F]

5-Bromo-4-(trifluoromethyl)pyridin-2-amine. To a stirred solution of 4-(trifluoromethyl)pyridin-2-amine (10 g, 61.72 mmol) in DCM (100 mL), was added NBS (10.98 g, 61.72 mmol) in one portion at 24° C. under a nitrogen atmosphere. The resulting mixture was stirred for 2 h. The reaction mixture was diluted with water and extracted with DCM. The combined organic layer was washed with water and brine, dried over anhydrous Na₂SO₄, and concentrated under vacuum. The product was purified by silica gel column chromatography to afford 5-bromo-4-(trifluoromethyl)pyridin-2-amine (10 g, 67% yield).

5-(Prop-1-en-2-yl)-4-(trifluoromethyl)pyridin-2-amine. [1,1′-Bis(diphenylphosphino)ferrocene]-dichloropalladium(II) complex with DCM (1.52 g, 2.08 mmol) was added in one portion to a degassed solution of 5-bromo-4-(trifluoromethyl)pyridin-2-amine (5 g, 20.83 mmol), 4,4,5,5-tetramethyl-2-(prop-1-en-2-yl)-1,3,2-dioxaborolane (5.25 g, 31.25 mmol), and K₂CO₃ (8.62 g, 62.49 mmol) in 1,4-dioxane:water (80 mL:20 mL). The reaction mixture was stirred at 100° C. for 16 h. The reaction mixture was filtered through a pad of celite and rinsed with EtOAc. The filtrate was concentrated under reduced pressure and purified by column chromatography to afford 5-(prop-1-en-2-yl)-4-(trifluoromethyl)pyridin-2-amine (3.5 g, 83% yield).

5-Isopropyl-4-(trifluoromethyl)pyridin-2-amine. Pd/C (0.3 eq) under a nitrogen atmosphere was added to a solution of 5-(prop-1-en-2-yl)-4-(trifluoromethyl)pyridin-2-amine (10 g, 92.47 mmol) in EtOH (100 mL) and the solution was hydrogenated using a Parr shaker apparatus at 40 psi for 16 h. The reaction mixture was filtered through a celite pad and the filtrate was concentrated under vacuum to get 5-isopropyl-4-(trifluoromethyl)pyridin-2-amine (6 g, 60% yield).

N-(5-Isopropyl-4-(trifluoromethyl)pyridin-2-ylcarbamothioyl)benzamide. To a solution of 5-isopropyl-4-(trifluoromethyl)pyridin-2-amine (5.4 g, 26.47 mmol) in THF (60 mL) under a nitrogen atmosphere at 24° C. was added benzoyl isothiocyanate (4.8 g, 29.11 mmol) dropwise. The reaction mixture was stirred at 70° C. for 3 h. The reaction mixture was diluted with water and extracted with EtOAc. The combined organic layer was washed with water and brine, dried over anhydrous Na₂SO₄, and concentrated under vacuum to afford N-(5-isopropyl-4-(trifluoromethyl)pyridin-2-ylcarbamothioyl)benzamide (4.9 g, 50% yield).

1-(5-Isopropyl-4-(trifluoromethyl)pyridin-2-yl)thiourea. To a solution of N-(5-isopropyl-4-(trifluoromethyl)pyridin-2-ylcarbamothioyl)benzamide (4.8 g, 13.07 mmol) in MeOH stirred at 24° C., was added 1N NaOH solution (20 mL) and the mixture was stirred at 80° C. for 2 h. The reaction mixture was poured into ice-water and neutralized with diluted HCl. The resulting crude material was collected by filtration, washed with water, and dried under vacuum to afford 1-(5-isopropyl-4-(trifluoromethyl)pyridin-2-yl)thiourea (3.1 g, 90% yield).

N-(5-Isopropyl-4-(trifluoromethyl)pyridin-2-yl)-4-(pyridin-2-yl)thiazol-2-amine. To a solution of 1-(5-isopropyl-4-(trifluoromethyl)pyridin-2-yl)thiourea (2 g, 76.04 mmol) in EtOH (20 mL) stirred under nitrogen atmosphere at 24° C., was added 2-bromo-1-(5-methoxypyridin-2-yl)ethanone (1.5 g, 76.04 mmol) in one portion and the solution was stirred at 80° C. for 4 h. The reaction mixture was poured into ice-water and adjusted to a pH of 10 with aqueous ammonia. The product was collected by filtration, washed with water and dried under vacuum. The product was isolated and purified via standard methods to obtain N-(5-isopropyl-4-(trifluoromethyl)pyridin-2-yl)-4-(pyridin-2-yl)thiazol-2-amine (1.6 g, 57% yield). MS (ESI) m/z 365.23[M+H]⁺.

### Example 12. 6-(4-(Pyridin-2-yl)thiazol-2-ylamino)pyridin-3-ol

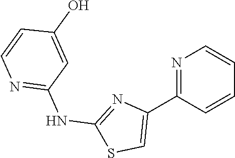 [SMILES: Oc1ccnc(Nc2nc(-c3ccccn3)cs2)c1]

6-(4-(Pyridin-2-yl)thiazol-2-ylamino)pyridin-3-ol. BBr₃ (1M in DCM) (3.17 ml, 3.17 mmol) was added to the solution of N-(5-methoxypyridin-2-yl)-4-(pyridin-2-yl)thiazol-2-amine (0.150 g, 0.528 mmol) in DCM (20 ml). The reaction was stirred at 40° C. for 15 h. The reaction mixture was quenched with saturated NaHCO₃ and was diluted with EtOAc. The layers were separated, and the organic phase was washed with saturated aqueous NaCl. The organic layer was dried over magnesium sulfate, filtered, and concentrated. The crude residue was purified by using reverse-phased semi-preparative HPLC. The fraction containing clean product was loaded onto a Phenomenex Strata-X-C ion exchange column. The column was washed successively with water and MeOH, the product was eluted with 5% ammonium hydroxide in MeOH, and the product containing eluent was concentrated under reduced pressure to afford 6-(4-(pyridin-2-yl)thiazol-2-ylamino)pyridin-3-ol (0.145 g, 0.536 mmol, 102% yield). The free base was dissolved in THE (20 mL) and hydrochloride (1M in diethylether 1.2 eq.) was added. The product was isolated and purified via standard methods to give 6-(4-(pyridin-2-yl)thiazol-2-ylamino)pyridin-3-ol HCl salt. MS (ESI) m/z 271.4 [M+H]⁺.

### Example 13. N-(3,5-Dimethylpyridin-2-yl)-4-(pyridin-2-yl)thiazol-2-amine

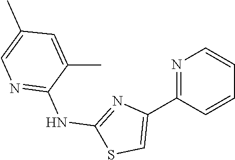 [SMILES: Cc1cnc(Nc2nc(-c3ccccn3)cs2)c(C)c1]

N-(3,5-Dimethylpyridin-2-yl)-4-(pyridin-2-yl)thiazol-2-amine. A solution of 2-bromo-1-(pyridin-2-yl)ethanone (0.200 g, 1 mmol), 1-(3,5-dimethylpyridin-2-yl)thiourea (0.181 g, 1.000 mmol) in EtOH (10 ml) was stirred at 78° C. for 1 h. The reaction mixture was quenched with saturated NaCl and then washed with EtOAc and washed with additional saturated aqueous NaCl. The organic layer was combined, dried over magnesium sulfate, filtered, and concentrated. The crude residue was purified using reverse-phased semi-preparative HPLC. The fraction containing clean product was loaded onto a Phenomenex Strata-X-C ion exchange column. The column was washed successively with water and MeOH. The product was isolated and purified via standard methods to afford N-(3,5-dimethylpyridin-2-yl)-4-(pyridin-2-yl)thiazol-2-amine (0.083 g, 0.294 mmol, 29.4% yield). MS (ESI) m/z 282.36 [M+H]⁺.

### Example 14. 5-Methyl-N-(4-methylpyridin-2-yl)-4-(pyridin-2-yl)thiazol-2-amine

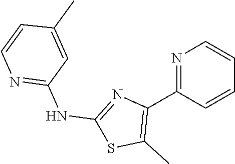 [SMILES: Cc1ccnc(Nc2nc(-c3ccccn3)c(C)s2)c1]

5-Methyl-N-(4-methylpyridin-2-yl)-4-(pyridin-2-yl)thiazol-2-amine. The solution of 2-chloro-1-(pyridin-2-yl)propan-1-one (0.170 g, 1 mmol), 1-(4-methylpyridin-2-yl)thiourea (0.167 g, 1.000 mmol) in EtOH (10 ml) was stirred at 75° C. for 15 h. The reaction mixture was quenched with saturated aqueous NaCl and then washed with EtOAc. The organic phase was combined and washed with saturated aqueous NaCl. The organic layer was dried over magnesium sulfate, filtered, and concentrated. The product was isolated and purified via standard methods to afford 5-methyl-N-(4-methylpyridin-2-yl)-4-(pyridin-2-yl)thiazol-2-amine (0.120 g, 0.425 mmol, 42.5% yield); MS (ESI) m/z 283.2 [M+H]⁺.

### Example 15. 4-(Pyridin-2-yl)-N-(1H-pyrrolo[3,2-b]pyridin-5-yl)thiazol-2-amine

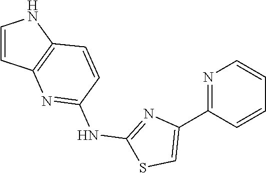 [SMILES: c1ccc(-c2csc(Nc3ccc4[nH]ccc4n3)n2)nc1]

4-(Pyridin-2-yl)-N-(1H-pyrrolo[3,2-b]pyridin-5-yl)thiazol-2-amine. A solution of 1-(1H-pyrrolo[3,2-b]pyridin-5-yl)thiourea (0.192 g, 1 mmol), 2-bromo-1-(pyridin-2-yl)ethanone (0.200 g, 1.000 mmol) in EtOH (10 ml) was stirred at 78° C. for 24 h. The reaction mixture was concentrated and the product was isolated and purified via standard methods to afford 4-(pyridin-2-yl)-N-(1H-pyrrolo[3,2-b]pyridin-5-yl)thiazol-2-amine (0.120 g, 0.409 mmol, 40.9% yield). LCMS (ESI): m/z 294.2 [M+H]⁺.

### Example 16. 2-((4-(Pyridin-2-yl)thiazol-2-yl)amino)isonicotinamide

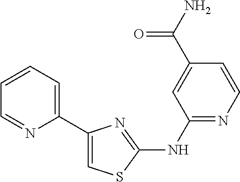 [SMILES: NC(=O)c1ccnc(Nc2nc(-c3ccccn3)cs2)c1]

2-((4-(Pyridin-2-yl)thiazol-2-yl)amino)isonicotinamide. 2-Bromo-1-(pyridin-2-yl)ethan-1-one hydrobromide (286 mg, 1.019 mmol) was added to a stirred solution of 2-thioureidoisonicotinamide (200 mg, 1.019 mmol) in EtOH (2 ml) under a nitrogen atmosphere at 24° C. and the reaction mixture was stirred at 80° C. for 4 h. The reaction mixture was poured into ice-water and adjusted to pH 10 with aqueous ammonia. The product was isolated and purified via standard methods to obtain 2-((4-(pyridin-2-yl)thiazol-2-yl)amino)isonicotinamide (79 mg, 0.266 mmol, 26.1% yield). ¹H NMR (300 MHz, DMSO-d6) δ 11.64 (s, 1H), 8.60 (d, J=4.4 Hz, 1H), 8.43 (d, J=5.3 Hz, 1H), 8.17 (s, 1H), 7.99 (d, J=7.8 Hz, 1H), 7.88 (td, J=7.7, 1.8 Hz, 1H), 7.67 (d, J=4.2 Hz, 2H), 7.50 (s, 1H), 7.32 (ddd, J=7.0, 5.1, 1.3 Hz, 2H). LCMS (ESI): m/z 298.1 [M+H]⁺.

### Example 17. 2-((4-(Pyridin-2-yl)thiazol-2-yl)amino)-N-(2,2,2-trifluoroethyl)isonicotinamide

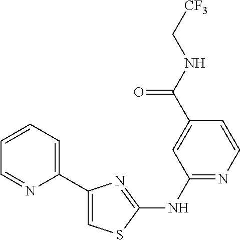 [SMILES: O=C(NCC(F)(F)F)c1ccnc(Nc2nc(-c3ccccn3)cs2)c1]

2-((4-(Pyridin-2-yl)thiazol-2-yl)amino)-N-(2,2,2-trifluoroethyl)isonicotinamide. TEA (0.047 ml, 0.335 mmol), 2,4,6-tripropyl-1,3,5,2,4,6-trioxatriphosphinane 2,4,6-trioxide (0.200 ml, 0.335 mmol) and 2,2,2-trifluoroethan-1-amine, hydrochloride (45.4 mg, 0.335 mmol) were added to a stirred solution of 2-((4-(pyridin-2-yl)thiazol-2-yl)amino)isonicotinic acid (100 mg, 0.335 mmol) in DMF (2 ml). The resulting reaction mixture was stirred at 25° C. for 14 h. The product was isolated and purified via standard methods to obtain 2-((4-(pyridin-2-yl)thiazol-2-yl)amino)-N-(2,2,2-trifluoroethyl)isonicotinamide (14 mg, 0.037 mmol, 11.01% yield). ¹H NMR (300 MHz, DMSO-d6) δ 11.68 (s, 1H), 9.35 (t, J=6.2 Hz, 1H), 8.61 (d, J=4.6 Hz, 1H), 8.48 (d, J=5.3 Hz, 1H), 8.05-7.82 (m, 2H), 7.70 (s, 1H), 7.53 (s, 1H), 7.39-7.23 (m, 2H), 4.24-4.01 (m, 2H). LCMS (ESI): m/z 380.0 [M+H]⁺.

### Example 18. N-Isopropyl-2-((4-(pyridin-2-yl)thiazol-2-yl)amino)isonicotinamide

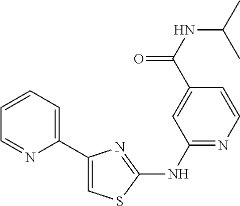 [SMILES: CC(C)NC(=O)c1ccnc(Nc2nc(-c3ccccn3)cs2)c1]

N-Isopropyl-2-((4-(pyridin-2-yl)thiazol-2-yl)amino)isonicotinamide. 2-((4-(pyridin-2-yl)thiazol-2-yl)amino)isonicotinic acid, hydrobromide (50 mg, 0.132 mmol) and HATU (75 mg, 0.198 mmol) were suspended in DMF (659 μl) after which DIPEA (69.1 μl, 0.396 mmol) was added and the reaction mixture was stirred for 10 mins. Isopropylamine (13.55 μl, 0.158 mmol) was added and the reaction was stirred for 16 h. The product was isolated and purified via standard methods to give N-isopropyl-2-((4-(pyridin-2-yl)thiazol-2-yl)amino)isonicotinamide (30 mg, 0.088 mmol, 67.0% yield). ¹H NMR (300 MHz, DMSO-d6) δ 11.60 (s, 1H), 8.59 (ddd, J=4.8, 1.8, 1.0 Hz, 1H), 8.46 (d, J=7.8 Hz, 1H), 8.41 (d, J=5.3 Hz, 1H), 7.98 (dt, J=7.9, 1.1 Hz, 1H), 7.88 (td, J=7.7, 1.8 Hz, 1H), 7.67 (s, 1H), 7.51-7.43 (m, 1H), 7.37-7.24 (m, 2H), 4.19-3.97 (m, 1H), 1.18 (d, J=6.6 Hz, 6H). MS (ESI): m/z 340.2 [M+H]⁺.

### Example 19. (4-(6-((4-(Pyridin-2-yl)thiazol-2-yl)amino)pyridin-3-yl)piperazin-1-yl)(1-(trifluoromethyl)cyclopropyl)methanone

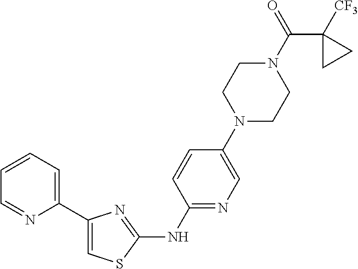 [SMILES: O=C(N1CCN(c2ccc(Nc3nc(-c4ccccn4)cs3)nc2)CC1)C1(C(F)(F)F)CC1]

(4-(6-((4-(Pyridin-2-yl)thiazol-2-yl)amino)pyridin-3-yl)piperazin-1-yl)(1-(trifluoromethyl)cyclopropyl)methanone. N-ethyl-N-isopropylpropan-2-amine (340 mg, 2.63 mmol) was added to a solution of 1-(trifluoromethyl)cyclopropane-1-carboxylic acid (135 mg, 0.876 mmol) and 2-(3H-[1,2,3]triazolo[4,5-b]pyridin-3-yl)-1,1,3,3-tetramethylisouronium hexafluorophosphate(V) (500 mg, 1.314 mmol) in DMF (1.00 ml) and the mixture stirred at 25° C. for 10 min. N-(5-(piperazin-1-yl)pyridin-2-yl)-4-(pyridin-2-yl)thiazol-2-amine (326 mg, 0.964 mmol) dissolved in DMF (1 ml) was then added to the stirred solution. The solution was then stirred at 25° C. for 5 h. The reaction mixture was then diluted with DCM and washed with a 10% LiCl solution. The organic layer was passed through a hydrophobic frit and the organics removed under vacuum. The product was isolated and purified via standard methods to afford (4-(6-((4-(pyridin-2-yl)thiazol-2-yl)amino)pyridin-3-yl)piperazin-1-yl)(1-(trifluoromethyl)cyclopropyl)methanone (112 mg, 0.234 mmol, 26.7% yield). ¹H NMR (300 MHz, DMSO-d6) δ 11.19 (s, 1H), 8.59 (ddd, J=4.8, 1.8, 0.9 Hz, 1H), 8.07-7.92 (m, 2H), 7.87 (td, J=7.6, 1.8 Hz, 1H), 7.63-7.48 (m, 2H), 7.30 (ddd, J=7.4, 4.8, 1.3 Hz, 1H), 7.08 (d, J=9.0 Hz, 1H), 3.12 (t, J=5.1 Hz, 4H), 1.40-1.28 (m, 2H), 1.26 (d, J=9.5 Hz, 2H). LCMS (ESI): m/z 475.1 [M+H]⁺.

### Example 20. 1-(6-((4-(Pyridin-2-yl)thiazol-2-yl)amino)pyridin-3-yl)piperidine-3-carboxylic acid

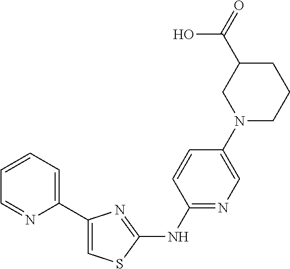 [SMILES: O=C(O)C1CCCN(c2ccc(Nc3nc(-c4ccccn4)cs3)nc2)C1]

1-(6-((4-(Pyridin-2-yl)thiazol-2-yl)amino)pyridin-3-yl)piperidine-3-carboxylic acid. A suspension of 2-bromo-1-(pyridin-2-yl)ethan-1-one hydrobromide (546 mg, 1.944 mmol) and 1-(6-thioureidopyridin-3-yl)piperidine-3-carboxylic acid (545 mg, 1.944 mmol) was heated in EtOH (20 ml) at 85° C. for 5 h. A precipitate formed and a saturated solution of NaHCO₃ was added until the pH was neutral, dissolving the precipitate. The product was isolated and purified via standard methods to afford 1-(6-((4-(pyridin-2-yl)thiazol-2-yl)amino)pyridin-3-yl)piperidine-3-carboxylic acid (350 mg, 0.881 mmol, 45.3% yield). ¹H NMR (300 MHz, DMSO-d6) δ 12.31 (s, 1H), 11.16 (s, 1H), 8.68-8.50 (m, 1H), 8.04-7.92 (m, 2H), 7.87 (td, J=7.7, 1.8 Hz, 1H), 7.57 (s, 1H), 7.49 (dd, J=9.0, 2.9 Hz, 1H), 7.30 (ddd, J=7.4, 4.8, 1.3 Hz, 1H), 7.06 (d, J=9.0 Hz, 1H), 3.69-3.38 (m, 2H), 2.93 (dd, J=12.0, 9.4 Hz, 1H), 2.78 (ddd, J=12.2, 9.7, 3.3 Hz, 1H), 2.59 (tq, J=6.8, 3.7 Hz, 1H), 1.90 (dt, J=12.5, 4.0 Hz, 1H), 1.82-1.70 (m, 1H), 1.58 (tt, J=13.6, 6.9 Hz, 2H). LCMS (ESI): m/z 382.1 [M+H]⁺.

### Example 21. 2-Hydroxy-2-methyl-1-(4-(6-((4-(pyridin-2-yl)thiazol-2-yl)amino)pyridin-3-yl)piperazin-1-yl)propan-1-one

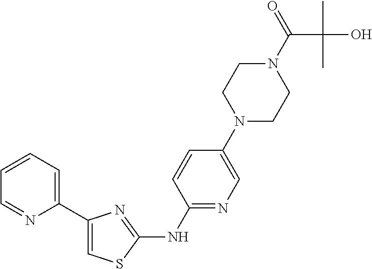 [SMILES: CC(C)(O)C(=O)N1CCN(c2ccc(Nc3nc(-c4ccccn4)cs3)nc2)CC1]

2-Hydroxy-2-methyl-1-(4-(6-((4-(pyridin-2-yl)thiazol-2-yl)amino)pyridin-3-yl)piperazin-1-yl)propan-1-one. N-ethyl-N-isopropylpropan-2-amine (354 mg, 2.74 mmol) was added to a solution of 2-hydroxy-2-methylpropanoic acid (95 mg, 0.913 mmol) and 2-(3H-[1,2,3]triazolo[4,5-b]pyridin-3-yl)-1,1,3,3-tetramethylisouronium hexafluorophosphate(V) (520 mg, 1.369 mmol) in DMF (1.0 ml) and the mixture was stirred at 24° C. for 10 min. N-(5-(piperazin-1-yl)pyridin-2-yl)-4-(pyridin-2-yl)thiazol-2-amine (340 mg, 1.0 mmol) dissolved in DMF (1 ml) was added to the stirred solution. The solution was then stirred at 24° C. for 5 h. The reaction mixture was then diluted with DCM and washed with a 10% LiCl solution. The organic layer was then passed through a hydrophobic frit and the organics removed under vacuum. The product was isolated and purified via standard methods to afford 2-hydroxy-2-methyl-1-(4-(6-((4-(pyridin-2-yl)thiazol-2-yl)amino)pyridin-3-yl)piperazin-1-yl)propan-1-one (98 mg, 0.224 mmol, 24.54% yield). ¹H NMR (300 MHz, DMSO-d6) δ 11.17 (s, 1H), 8.59 (ddd, J=4.8, 1.8, 1.0 Hz, 1H), 8.07-7.91 (m, 2H), 7.87 (td, J=7.6, 1.8 Hz, 1H), 7.57 (s, 1H), 7.53 (dd, J=9.0, 2.9 Hz, 1H), 7.30 (ddd, J=7.4, 4.7, 1.3 Hz, 1H), 7.07 (d, J=9.0 Hz, 1H), 5.45 (s, 1H), 3.31 (s, 18H), 3.10 (t, J=5.1 Hz, 4H), 1.35 (s, 5H). LCMS (ESI): m/z 425.1 [M+H]⁺.

### Example 22. 1-(6-((4-(Pyridin-2-yl)thiazol-2-yl)amino)pyridin-3-yl)-N-(2,2,2-trifluoroethyl)piperidine-3-carboxamide

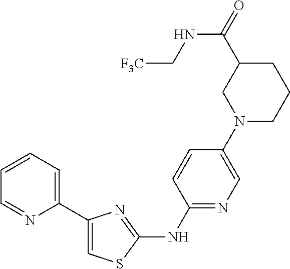 [SMILES: O=C(NCC(F)(F)F)C1CCCN(c2ccc(Nc3nc(-c4ccccn4)cs3)nc2)C1]

1-(6-((4-(Pyridin-2-yl)thiazol-2-yl)amino)pyridin-3-yl)-N-(2,2,2-trifluoroethyl)piperidine-3-carboxamide. DIPEA (0.340 ml, 1.947 mmol) was added to a solution of 1-(6-((4-(pyridin-2-yl)thiazol-2-yl)amino)pyridin-3-yl)piperidine-3-carboxylic acid hydrobromide (300 mg, 0.649 mmol) and HATU (370 mg, 0.973 mmol) in DMF (2 ml) and the mixture was stirred at 24° C. for 10 min. To the stirred solution was added 2,2,2-trifluoroethan-1-amine (77 mg, 0.779 mmol) and the solution was stirred at 24° C. for 5 h. The reaction mixture was diluted with DCM and washed with a 10% LiCl solution. The organic layer was passed through a hydrophobic frit and the organics were removed under vacuum. The product was isolated and purified via standard methods to afford 1-(6-((4-(pyridin-2-yl)thiazol-2-yl)amino)pyridin-3-yl)-N-(2,2,2-trifluoroethyl)piperidine-3-carboxamide (173 mg, 0.374 mmol, 57.7% yield). ¹H NMR (300 MHz, DMSO-d6) δ 11.15 (s, 1H), 8.69-8.48 (m, 2H), 8.03-7.92 (m, 2H), 7.87 (td, J=7.7, 1.8 Hz, 1H), 7.57 (d, J=0.7 Hz, 1H), 7.50 (dd, J=9.0, 2.9 Hz, 1H), 7.30 (ddd, J=7.4, 4.8, 1.3 Hz, 1H), 7.06 (d, J=9.0 Hz, 1H), 4.19-3.71 (m, 2H), 3.56 (dd, J=23.5, 11.3 Hz, 2H), 2.96-2.53 (m, 3H), 1.96-1.70 (m, 2H), 1.57 (q, J=11.7 Hz, 2H). LCMS (ESI): m/z 463.2 [M+H]⁺.

### Example 23. 5-(Piperidin-1-ylmethyl)-4-(pyridin-2-yl)-N-(4-(trifluoromethyl)pyridin-2-yl)thiazol-2-amine

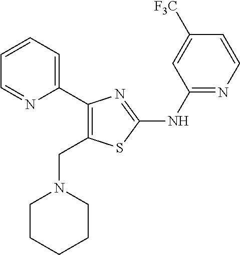 [SMILES: FC(F)(F)c1ccnc(Nc2nc(-c3ccccn3)c(CN3CCCCC3)s2)c1]

5-(Piperidin-1-ylmethyl)-4-(pyridin-2-yl)-N-(4-(trifluoromethyl)pyridin-2-yl)thiazol-2-amine. Piperidine (0.021 ml, 0.214 mmol) and AcOH (4.09 μl, 0.071 mmol) were added to a solution of 4-(pyridin-2-yl)-2-((4-(trifluoromethyl)pyridin-2-yl)amino)thiazole-5-carbaldehyde (25 mg, 0.071 mmol) and powdered molecular sieves in DCE (5 ml). The mixture was stirred for 3 h at reflux. After cooling to 24° C., sodium triacetoxyborohydride (30.2 mg, 0.143 mmol) was added and the reaction was stirred at 24° C. for 4 h. The product was isolated and purified via standard methods to give 5-(piperidin-1-ylmethyl)-4-(pyridin-2-yl)-N-(4-(trifluoromethyl)pyridin-2-yl)thiazol-2-amine (20 mg, 0.046 mmol, 64.8% yield). ¹H NMR (300 MHz, Methanol-d4) δ 8.67 (d, J=4.6 Hz, 1H), 8.55 (d, J=5.4 Hz, 1H), 8.16 (d, J=8.0 Hz, 1H), 7.98 (t, J=7.9 Hz, 1H), 7.50-7.37 (m, 1H), 7.31 (s, 1H), 7.19 (d, J=5.4 Hz, 1H), 4.45 (s, 2H), 3.19-2.83 (m, 4H), 1.92-1.74 (m, 4H), 1.71-1.52 (m, 2H). LCMS (ESI): m/z 420.2 [M−H].

### Example 24. (4-(Pyridin-2-yl)-2-((4-(trifluoromethyl)pyridin-2-yl)amino)thiazol-5-yl)methanol

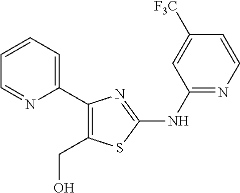 [SMILES: OCc1sc(Nc2cc(C(F)(F)F)ccn2)nc1-c1ccccn1]

(4-(Pyridin-2-yl)-2-((4-(trifluoromethyl)pyridin-2-yl)amino)thiazol-5-yl)methanol. Lithium borohydride (0.032 g, 1.471 mmol) was added to a solution of ethyl 4-(pyridin-2-yl)-2-((4-(trifluoromethyl)pyridin-2-yl)amino)thiazole-5-carboxylate and hydrobromide (0.233 g, 0.490 mmol) in dioxane (9.80 ml) at 24° C. The reaction was stirred for 18 h at 70° C. The product was isolated and purified via standard methods to give (4-(pyridin-2-yl)-2-((4-(trifluoromethyl)pyridin-2-yl)amino)thiazol-5-yl)methanol (48 mg, 0.136 mmol, 27.8% yield). MS (ESI): m/z 353.1 [M−H]. ¹H NMR (300 MHz, DMSO-d6) δ 11.60 (br s, 1H), 8.63 (ddd, J=4.9, 1.9, 1.0 Hz, 1H), 8.58 (d, J=5.4 Hz, 1H), 8.00 (d, J=8.0 Hz, 1H), 7.91 (td, J=7.7, 1.8 Hz, 1H), 7.40 (s, 1H), 7.31 (ddd, J=7.4, 4.8, 1.3 Hz, 1H), 7.23 (d, J=4.9 Hz, 1H), 5.82 (t, J=5.7 Hz, 1H), 5.06 (d, J=5.3 Hz, 2H).

### Example 25. 5-((Cyclopropylmethoxy)methyl)-4-(pyridin-2-yl)-N-(4-(trifluoromethyl)pyridin-2-yl)thiazol-2-amine

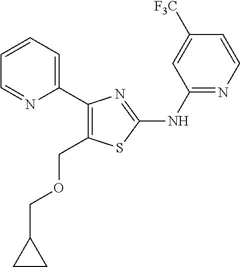 [SMILES: FC(F)(F)c1ccnc(Nc2nc(-c3ccccn3)c(COCC3CC3)s2)c1]

5-((Cyclopropylmethoxy)methyl)-4-(pyridin-2-yl)-N-(4-(trifluoromethyl)pyridin-2-yl)thiazol-2-amine. (Bromomethyl)cyclopropane (65.8 mg, 0.487 mmol), cesium carbonate (159 mg, 0.487 mmol) and potassium iodide (135 mg, 0.812 mmol) were placed in a 7 mL sealed tube containing acetone (3 ml) and heated at 75° C. for 5 mins. (4-(Pyridin-2-yl)-2-((4-(trifluoromethyl)pyridin-2-yl)amino)thiazol-5-yl)methanol (143 mg, 0.406 mmol) was then added and the mixture was heated for 16 h. The product was isolated and purified via standard methods to afford 5-((cyclopropylmethoxy)methyl)-4-(pyridin-2-yl)-N-(4-(trifluoromethyl)pyridin-2-yl)thiazol-2-amine (51 mg, 0.119 mmol, 29.4% yield). UPLC-MS (ESI): m/z 407.1 [M+H]⁺. ¹H NMR (300 MHz, DMSO-d6) δ 8.72 (d, J=5.2 Hz, 1H), 8.69-8.62 (m, 1H), 8.08 (d, J=8.0 Hz, 1H), 7.95 (td, J=7.7, 1.8 Hz, 1H), 7.73 (s, 1H), 7.39 (d, J=5.0 Hz, 1H), 7.39-7.28 (m, 2H), 5.87 (t, J=5.7 Hz, 1H), 5.11 (d, J=5.7 Hz, 2H), 4.57 (d, J=6.8 Hz, 2H), 1.50-1.24 (m, 1H), 0.68-0.42 (m, 4H).

### Example 26. N-{5-[(Cyclobutylmethoxy)methyl]-4-(pyridin-2-yl)-1,3-thiazol-2-yl}-4-methylpyridin-2-amine

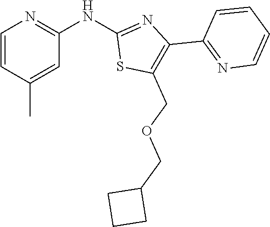 [SMILES: Cc1ccnc(Nc2nc(-c3ccccn3)c(COCC3CCC3)s2)c1]

Ethyl 2-bromo-3-oxo-3-(pyridin-2-yl)propanoate. To a stirred solution of ethyl 3-oxo-3-(pyridin-2-yl)propanoate (1 g, 5.18 mmol) in CHCl₃ (30 ml) at 0° C., bromine (0.27 mL, 5.18 mmol) dissolved in CHCl₃ (5 mL) was added dropwise and stirred at 0-5° C. for 15 min. After completion, the reaction mixture was diluted with DCM and washed with saturated aqueous NaHCO₃ solution. The organic layer was then washed with water and brine, dried over Na₂SO₄, filtered, and concentrated under reduced pressure to afford ethyl 2-bromo-3-oxo-3-(pyridin-2-yl)propanoate (1.3 g, crude). MS (ESI): m/z 272.0 [M+1]⁺.

Ethyl 2-[(4-methylpyridin-2-yl)amino]-4-(pyridin-2-yl)-1,3-thiazole-5-carboxylate. To a stirred solution of (4-methylpyridin-2-yl)thiourea (500 mg, 2.99 mmol) in EtOH (50 mL), was added ethyl 2-bromo-3-oxo-3-(pyridin-2-yl)propanoate (1.3 g, 2.99 mmol) and the resulting mixture was heated to reflux for 16 h. After completion, it was cooled to 25° C. and poured into crushed ice. It was then stirred for another 30 min and the pH was adjusted to 8 with a solution of Na₂CO₃. The crude formed was filtered and washed with cold water. The crude material was dissolved in 10% MeOH:DCM, dried over Na₂SO₄, filtered, and concentrated under reduced pressure. The crude product was triturated with Et₂O to afford ethyl 2-[(4-methylpyridin-2-yl)amino]-4-(pyridin-2-yl)-1,3-thiazole-5-carboxylate (850 mg, 83%). MS (ESI): m/z 341.0 [M+1]⁺.

{2-[(4-Methylpyridin-2-yl)amino]-4-(pyridin-2-yl)-1,3-thiazol-5-yl}methanol. To a stirred solution of ethyl 2-[(4-methylpyridin-2-yl)amino]-4-(pyridin-2-yl)-1,3-thiazole-5-carboxylate (300 mg, 0.88 mmol) in THE (20 ml) at −78° C., was added LiEt₃BH (Superhydride, 1M in THF, 3.53 mL, 3.53 mmol) dropwise and the mixture was stirred at −78° C. for 30 min. It was then warmed to 25° C. and stirred for 16 h. The reaction mixture was cooled to −78° C. and additional LiEt₃BH (Superhydride, 1M in THF, 3.53 ml, 3.53 mmol) was added and the mixture was stirred at 25° C. for 4 h. After completion, the reaction mixture was cooled to −10° C., quenched with saturated aqueous NH₄Cl solution and extracted with EtOAc. The combined organic layers were washed with brine, dried over Na₂SO₄, filtered, and concentrated under reduced pressure. The crude compound was purified by column chromatography followed by trituration with Et₂O to afford {2-[(4-methylpyridin-2-yl)amino]-4-(pyridin-2-yl)-1,3-thiazol-5-yl}methanol (190 mg, 72%). MS (ESI): m/z 299.4 [M+1]⁺.

N-{5-[(Cyclobutylmethoxy)methyl]-4-(pyridin-2-yl)-1,3-thiazol-2-yl}-4-methylpyridin-2-amine. To a stirred solution of {2-[(4-methylpyridin-2-yl)amino]-4-(pyridin-2-yl)-1,3-thiazol-5-yl}methanol (100 mg, 0.34 mmol) in DCM (10 ml) at 0° C. was added PBr₃ (0.02 ml, 0.17 mmol) dropwise and stirred at 25° C. for 20 min. Cyclobutylmethanol (3.53 ml, 36.91 mmol) was added to it and the mixture was stirred at 25° C. for 30 min. NaOH (1M, 2.7 mL, 2.7 mmol) was added and stirred at 25° C. for 2 h. After completion, the reaction mixture was evaporated under reduced pressure and diluted with EtOAc. The organic layer was washed with NaHCO₃ solution, dried over Na₂SO₄, filtered, and concentrated under reduced pressure. The crude compound was purified by standard methods to afford N-{5-[(cyclobutylmethoxy)methyl]-4-(pyridin-2-yl)-1,3-thiazol-2-yl}-4-methylpyridin-2-amine (25 mg, 20%). MS (ESI): m/z 367.2 [M+1]⁺.

### Example 27. N-[5-tert-Butyl-4-(pyridin-2-yl)-1,3-thiazol-2-yl]-4-methylpyridin-2-amine

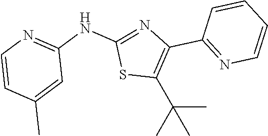 [SMILES: Cc1ccnc(Nc2nc(-c3ccccn3)c(C(C)(C)C)s2)c1]

2-Bromo-3,3-dimethyl-1-(pyridin-2-yl)butan-1-one. To a stirred solution of 3,3-dimethyl-1-(pyridin-2-yl)butan-1-one (200 mg, 1.29 mmol) in AcOH (2 ml), was added 33% HBr in AcOH (1 ml) at 0° C., followed by the dropwise addition of bromine in AcOH (1.54 ml, 1.54 mmol). The resulting mixture was warmed to 25° C. and stirred for 2 h. The reaction mixture was diluted with water and extracted with EtOAc. The combined organic layer was washed with brine, dried over anhydrous Na₂SO₄, filtered, and concentrated under reduced pressure to get the desired compound 2-bromo-3,3-dimethyl-1-(pyridin-2-yl)butan-1-one (300 mg, 91%). MS (ESI): m/z 256.0 [M+1]⁺.

N-[5-tert-Butyl-4-(pyridin-2-yl)-1,3-thiazol-2-yl]-4-methylpyridin-2-amine. (4-methylpyridin-2-yl)thiourea (198 mg, 1.181 mmol) was added to a stirred solution of 2-bromo-3,3-dimethyl-1-(pyridin-2-yl)butan-1-one (300 mg, 1.18 mmol) in DMF (5 ml). The resultant mixture was heated to 90° C. for 16 h in a sealed tube. The reaction mixture was cooled to 24° C. and poured into crushed ice. The resultant solution was adjusted to pH 8 with Na₂CO₃ solution and extracted with EtOAc. The combined organics were washed with brine, dried over Na₂SO₄, and concentrated to get the crude compound. The crude mass was purified by prep-HPLC to afford N-[5-tert-butyl-4-(pyridin-2-yl)-1,3-thiazol-2-yl]-4-methylpyridin-2-amine (16 mg, 4%). MS (ESI): m/z 325.3 [M+1]⁺.

### Example 28. 4-Butyl-N-[4-(pyridin-2-yl)-1,3-thiazol-2-yl]pyridin-2-amine

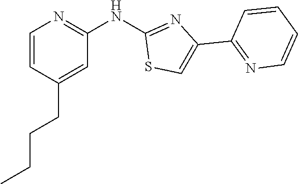 [SMILES: CCCCc1ccnc(Nc2nc(-c3ccccn3)cs2)c1]

4-Butylpyridine. To a stirred solution of LDA (2M in THF, 40.3 mL, 80.6 mmol) in THE (50 mL) was added a solution of 4-methylpyridine (5 g, 53.76 mmol) in THE (15 mL) dropwise at −78° C. under an argon atmosphere and the mixture was stirred at −78° C. for 1 h. A solution of N-iodopropane (5.74 mL, 59.14 mmol) in THE (15 ml) was added over 45 min at −78° C. The reaction mixture was slowly warmed to 25° C. and stirred for 2 h. The reaction mixture was quenched with saturated aqueous NH₄Cl solution and extracted with EtOAc. The combined organic layer was washed with brine, dried over anhydrous Na₂SO₄, filtered, and concentrated under reduced pressure. The crude mass was purified by column chromatography to afford 4-butylpyridine (6 g, 82%). MS (ESI): m/z 135.9 [M+1]⁺.

4-Butylpyridin-1-ium-1-olate. 4-Butylpyridine (3 g, 22.22 mmol) was dissolved in DCM (100 ml) at 0° C. and mCPBA (77%, 9.9 g, 22.22 mmol) was added portion wise at 0° C. The resulting mixture was warmed to 25° C. and stirred for 5 h. After completion, the reaction mixture was diluted with DCM and washed sequentially with saturated aqueous NaHCO₃, water and brine, and then dried over anhydrous Na₂SO₄, filtered, and concentrated under reduced pressure to get 4-butylpyridin-1-ium-1-olate (1.9 g, 56%). MS (ESI): m/z 151.8 [M+1]⁺.

4-Butyl-N-tert-butylpyridin-2-amine. tBuNH₂ (6.2 ml, 59.60 mmol) was added drop wise to a stirred solution of 4-butylpyridin-1-ium-1-olate (1.8 g, 11.92 mmol) in trifluorotoluene (75 ml) at 0° C. under an argon atmosphere. Tosic anhydride (7.8 g, 23.84 mmol) was added to it in one portion and stirred at 0° C. for 2 h. The reaction mixture was diluted with EtOAc and washed with water and brine, dried over anhydrous Na₂SO₄, filtered, and concentrated under reduced pressure. The crude mass was purified by column chromatography to get 4-butyl-N-tert-butylpyridin-2-amine (1.3 g, 53%). MS (ESI): m/z 207.4 [M+1]⁺.

4-Butylpyridin-2-amine. 4-Butyl-N-tert-butylpyridin-2-amine (1.2 g, 5.82 mmol) was dissolved in TFA (25 ml) and heated at 65° C. for 3 h. The reaction mixture was evaporated under reduced pressure and diluted with ice-water. The aqueous portion was adjusted to pH 8 with saturated NaHCO₃ solution and extracted with EtOAc. The combined organic layer was washed with brine, dried over Na₂SO₄, filtered, and concentrated under reduced pressure. The crude mass was purified by column chromatography to get 4-butylpyridin-2-amine (500 mg, 66%). MS (ESI): m/z 150.7 [M+1]⁺.

1-Benzoyl-3-(4-butylpyridin-2-yl)thiourea. Benzoyl isothiocyanate (597 mg, 3.66 mmol) was added to a stirred solution of 4-butylpyridin-2-amine (500 mg, 3.33 mmol) in acetone (10 mL) under an argon atmosphere. The reaction mixture was stirred at 25° C. for 3 h and concentrated under reduced pressure. The crude mass was purified by column chromatography to afford 1-benzoyl-3-(4-butylpyridin-2-yl)thiourea (500 mg, 48%). MS (ESI): m/z 314.1 [M+1]⁺.

(4-Butylpyridin-2-yl)thiourea. 1-Benzoyl-3-(4-butylpyridin-2-yl)thiourea (500 mg, 1.59 mmol) was taken up in 10% aqueous NaOH (5 mL) and stirred at 25° C. for 15 min. The reaction mixture was heated to reflux for 15 min then cooled to 0° C. The pH was adjusted to 4 with aqueous HCl solution and then adjusted to pH 8-9 with saturated aqueous KHCO₃ solution. The resulting mixture was stirred at 0° C. for 15 min. The crude was filtered, washed with cold water, and dried under vacuum to afford (4-butylpyridin-2-yl)thiourea (220 mg, 64%). MS (ESI): m/z 210.2 [M+1]⁺.

4-Butyl-N-[4-(pyridin-2-yl)-1,3-thiazol-2-yl]pyridin-2-amine. 2-Bromo-1-(pyridin-2-yl)ethan-1-one hydrobromide salt (160 mg, 0.57 mmol) was added to a stirred solution of (4-butylpyridin-2-yl)thiourea (120 mg, 0.57 mmol) in EtOH (3 ml). The resulting mixture was heated to reflux for 16 h. The reaction mixture was cooled to 25° C., poured into crushed ice, and adjusted to pH 8 with a Na₂CO₃ solution. The crude was filtered, washed with cold water, and dried under vacuum. The crude mass was purified by column chromatography to get 4-butyl-N-[4-(pyridin-2-yl)-1,3-thiazol-2-yl]pyridin-2-amine (85 mg, 43%). MS (ESI): m/z 310.7 [M+1]⁺.

### Example 29. N-[4-(3-Methylpyridin-2-yl)-1,3-thiazol-2-yl]-4-phenylpyridin-2-amine

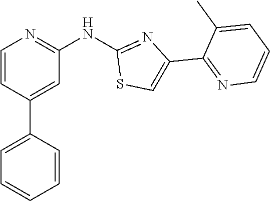 [SMILES: Cc1cccnc1-c1csc(Nc2cc(-c3ccccc3)ccn2)n1]

4-Phenylpyridin-2-amine. K₂CO₃ (1.8 g, 13.0 mmol) and phenyl boronic acid (1.27 g, 10.40 mmol) were added to a stirred solution of 4-bromopyridin-2-amine (1.5 g, 8.67 mmol) in dioxane (40 ml) and water (8 ml). The resulting mixture was purged with argon for 15 min and Pd(dppf)Cl₂ (317 mg, 0.43 mmol) was added under an inert atmosphere. The resulting mixture was heated at 100° C. for 16 h. The reaction mixture was cooled to 25° C. and diluted with EtOAc. The organics were washed with water and brine, dried over anhydrous Na₂SO₄, filtered, and concentrated under reduced pressure. The crude mass was purified by column chromatography to afford 4-phenylpyridin-2-amine (1.2 g, 81%). MS (ESI): m/z 171.1 [M+1]⁺.

(4-Phenylpyridin-2-yl)thiourea. Benzoyl isothiocyanate (1.44 g, 8.82 mmol) was added to a stirred solution of 4-phenylpyridin-2-amine (1.5 g, 8.82 mmol) in acetone (20 mL) and the resulting mixture was stirred at 25° C. for 1 h. The suspension was cooled in an ice-water bath, diluted with ice-water, and stirred for 10 min. A precipitate formed and was filtered, washed with cold water, and dried under vacuum. The crude compound was suspended in aqueous NaOH, stirred at 25° C. for 10 min, and then heated to reflux for additional 10 min. The reaction mixture was cooled to 0° C., adjusted to pH 4 with aqueous HCl solution, and then adjusted to pH 8-9 with saturated aqueous KHCO₃ solution. The mixture was stirred at 0° C. for 15 min. The crude was filtered, washed with cold water and dried under vacuum to afford (4-phenylpyridin-2-yl)thiourea (2 g, 99%). MS (ESI): m/z 230.1 [M+1]⁺.

N-[4-(3-Methylpyridin-2-yl)-1,3-thiazol-2-yl]-4-phenylpyridin-2-amine. (4-phenylpyridin-2-yl)thiourea (194 mg, 0.85 mmol) was added to a stirred solution of 2-bromo-1-(3-methylpyridin-2-yl)ethan-1-one (250 mg, 0.85 mmol) in EtOH (5 mL). The resulting mixture was heated to reflux for 4 h. Then the reaction mixture was cooled to 25° C., poured into ice-water, and adjusted to pH 8 with Na₂CO₃. The material thus formed was filtered and dried. The crude compound was purified by column chromatography to afford N-[4-(3-Methylpyridin-2-yl)-1,3-thiazol-2-yl]-4-phenylpyridin-2-amine (100 mg, 34%). MS (ESI): m/z 345.1 [M+1]⁺.

### Example 30. N-[4-(4-Methylpyridin-2-yl)-1,3-thiazol-2-yl]-3-phenylpyridin-2-amine

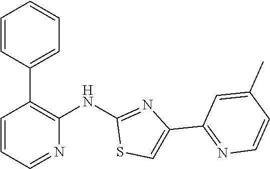 [SMILES: Cc1ccnc(-c2csc(Nc3ncccc3-c3ccccc3)n2)c1]

2-Bromo-1-(4-methylpyridin-2-yl)ethan-1-one. HBr in AcOH (33%, 5 mL) was added to a stirred solution of 1-(4-methylpyridin-2-yl)ethan-1-one (1 g, 7.41 mmol) in glacial AcOH (10 mL). The resulting mixture was cooled to 0° C. and bromine in AcOH (1 M, 8.9 mL, 8.9 mmol) was added. The resulting mixture was slowly warmed to 25° C. and stirred for 2 h. The crude was filtered, washed with Et₂O and pentane, and dried under vacuum to afford 2-bromo-1-(4-methylpyridin-2-yl)ethan-1-one hydrogen bromide (1.6 g, 73%). MS (ESI): m/z 213.9 [M+1]⁺.

3-Phenylpyridin-2-amine. K₂CO₃ (2.4 g, 17.34 mmol) and phenyl boronic acid (1.69 g, 13.87 mmol) were added to a stirred solution of 3-bromo-pyridin-2-ylamine (2 g, 11.56 mmol) in dioxane (40 ml) and water (8 ml). The resulting mixture was purged with argon for 15 min and Pd(dppf)Cl₂ (423 mg, 0.58 mmol) was added under an inert atmosphere. The resulting mixture was heated at 100° C. for 16 h, cooled to 24° C., and diluted with EtOAc. The organics were washed with water and brine, dried over anhydrous Na₂SO₄, filtered, and concentrated under reduced pressure. The crude mass was purified by column chromatography to afford 3-phenylpyridin-2-amine (1.8 g, 91%). MS (ESI): m/z 171.1 [M+1]⁺.

(3-Phenylpyridin-2-yl)thiourea. Benzoyl isothiocyanate (1.73 g, 10.59 mmol) was added to a stirred solution of 3-phenylpyridin-2-amine (1.8 g, 10.59 mmol) in acetone (20 mL) and the resulting mixture was stirred at 25° C. for 1 h. The suspension was cooled in an ice-water bath, diluted with ice-water, and stirred for 15 min. The precipitate was filtered, washed with cold water, and dried under vacuum. The crude compound (3.5 g) was suspended in 10% aqueous NaOH (20 mL), stirred at 25° C. for 15 min, and then heated to reflux for an additional 15 min. The reaction mixture was cooled to 0° C., adjusted to pH 4 with aqueous HCl solution, and then adjusted to pH 8-9 with saturated aqueous KHCO₃ solution. The mixture was stirred at 0° C. for 15 min. The solid was filtered, washed with cold water, and dried under vacuum to afford (3-phenylpyridin-2-yl)thiourea (2.2 g, 90%). MS (ESI): m/z 230.1 [M+1]⁺.

N-[4-(4-Methylpyridin-2-yl)-1,3-thiazol-2-yl]-3-phenylpyridin-2-amine. (3-phenylpyridin-2-yl)thiourea (194 mg, 0.85 mmol) was added to a stirred solution of 2-bromo-1-(4-methylpyridin-2-yl)ethan-1-one (250 mg, 0.85 mmol) in EtOH (5 mL). The resulting mixture was heated to reflux for 4 h, then cooled to 25° C., poured in ice-water, and Na₂CO₃ solution was added to adjust the pH to 8. The material thus formed was filtered and dried. The crude compound was purified by column chromatography to afford N-[4-(4-methylpyridin-2-yl)-1,3-thiazol-2-yl]-3-phenylpyridin-2-amine (150 mg, 51%). MS (ESI): m/z 345.1 [M+1]⁺.

### Example 31. 4-Methyl-N-[4-(3-phenylpyridin-2-yl)-1,3-thiazol-2-yl]pyridin-2-amine

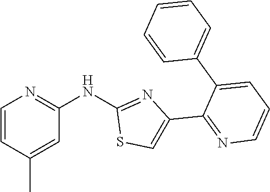 [SMILES: Cc1ccnc(Nc2nc(-c3ncccc3-c3ccccc3)cs2)c1]

N-Methoxy-N-methyl-3-phenylpyridine-2-carboxamide. To a stirred solution of 3-phenylpyridine-2-carboxylic acid (1 g, 5.02 mmol) in DMF (20 mL), was added EDC·HCl (1.4 g, 9.04 mmol) and HOBt (814 mg, 6.02 mmol) followed by N,O-dimethylhydroxylamine hydrochloride (539 mg, 5.52 mmol) and TEA (1.3 ml, 10.04 mmol). The resulting mixture was stirred at 25° C. for 3 h. The reaction mixture was quenched and extracted with EtOAc. The combined organic layer was washed with water and brine, dried over anhydrous Na₂SO₄, filtered, and concentrated under reduced pressure. The crude product was purified by column chromatography to afford N-methoxy-N-methyl-3-phenylpyridine-2-carboxamide (650 mg, 53%). MS (ESI): m/z 242.7 [M+1]⁺.

1-(3-Phenylpyridin-2-yl)ethan-1-one. A solution of N-methoxy-N-methyl-3-phenylpyridine-2-carboxamide (630 mg, 2.807 mmol) in dry THE (8 mL) was cooled to −10° C. and MeMgBr (3M in Et₂O, 1.7 mL, 5.1 mmol) was added. The mixture was stirred for an additional 1 h under at −10° C. The reaction mixture was quenched with saturated NH₄Cl solution and extracted with EtOAc. The combined organic layer was washed with water and brine, dried over anhydrous Na₂SO₄, filtered, and concentrated under reduced pressure. The crude mass was purified by column chromatography to afford 1-(3-phenylpyridin-2-yl)ethan-1-one (220 mg, 43%). MS (ESI): m/z 197.9 [M+1]⁺.

2-Bromo-1-(3-phenylpyridin-2-yl)ethan-1-one. HBr in AcOH (33%, 1.1 ml) was added to a stirred solution of 1-(3-phenylpyridin-2-yl)ethan-1-one (210 mg, 1.06 mmol) in glacial AcOH (2.1 ml). The resulting mixture was cooled to 0° C. and bromine in AcOH (1 M, 1.28 ml, 1.28 mmol) was added. The resulting mixture was slowly warmed to 25° C. and stirred for 2 h. The product was filtered, washed with Et₂O and pentane, and dried under vacuum to afford 2-bromo-1-(3-phenylpyridin-2-yl)ethan-1-one hydrobromide salt (250 mg, 66%). MS (ESI): m/z 276.1 [M+1]⁺.

4-Methyl-N-[4-(3-phenylpyridin-2-yl)-1,3-thiazol-2-yl]pyridin-2-amine. (4-methylpyridin-2-yl)thiourea (117 mg, 0.7 mmol) was added to a stirred solution of 2-bromo-1-(3-phenylpyridin-2-yl)ethan-1-one hydrobromide (250 mg, 0.7 mmol) in EtOH (8 mL). The resulting mixture was heated to reflux for 4 h. Then the reaction mixture was cooled to 25° C., poured into ice-water, and adjusted to pH 8 with a Na₂CO₃ solution. The crude material was filtered, dried and purified by column chromatography to afford 4-methyl-N-[4-(3-phenylpyridin-2-yl)-1,3-thiazol-2-yl]pyridin-2-amine (150 mg, 62%). MS (ESI): m/z 345.1 [M+1]⁺.

### Example 32. 4-(Pyrazin-2-yl)-N-[4-(pyridin-2-yl)-1,3-thiazol-2-yl]pyridin-2-amine

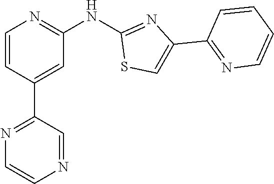 [SMILES: c1ccc(-c2csc(Nc3cc(-c4cnccn4)ccn3)n2)nc1]

Tert-butyl N-[4-(pyrazin-2-yl)pyridin-2-yl]carbamate. K₂CO₃ (651 mg, 4.72 mmol) followed by tert-butyl N-[4-(tetramethyl-1,3,2-dioxaborolan-2-yl)pyridin-2-yl]carbamate (1.2 g, 3.77 mmol) and [(t-Bu)₃P]BF₄ (18.24 mg, 0.06 mmol) were added to a stirred solution of 2-bromopyrazine (500 mg, 3.15 mmol) in dioxane (10 mL) and water (1 mL) under argon purging at 25° C. After 10 min, Pd(OAc)₂ (35 mg, 0.16 mmol) was added and the resultant mixture was heated to 100° C. for 16 h under an argon atmosphere. The reaction mixture was cooled to 25° C. and diluted with EtOAc. The organics were washed with water and brine, dried over anhydrous Na₂SO₄, and concentrated. The crude product was purified by column chromatography to afford tert-butyl N-[4-(pyrazin-2-yl)pyridin-2-yl]carbamate (500 mg, 58%). MS (ESI): m/z 273.3 [M+1]⁺.

4-(Pyrazin-2-yl)pyridin-2-amine. 4 N HCl in dioxane (4.6 mL) was added to a stirred solution of tert-butyl N-[4-(pyrazin-2-yl)pyridin-2-yl]carbamate (500 mg, 1.84 mmol) in 1,4-dioxane (10 mL) at 0° C. under an argon atmosphere. The reaction mixture was stirred at 25° C. for 4 h. After complete consumption of the starting material, volatiles were removed under reduced pressure. The obtained residue was dissolved in EtOAc and washed with saturated NaHCO₃ solution, water, and then brine solution. The organic layer was evaporated to afford 4-(pyrazin-2-yl)pyridin-2-amine (280 mg, 88%). MS (ESI): m/z 173.2 [M+1]⁺.

[4-(Pyrazin-2-yl)pyridin-2-yl]thiourea. Benzoyl isothiocyanate (255 mg, 1.57 mmol) was added to a stirred solution of 4-(pyrazin-2-yl)pyridin-2-amine (270 mg, 0.87 mmol) in acetone (10 mL) at 25° C. under an argon atmosphere. The reaction mixture was stirred at 25° C. for 45 min, cooled to 0° C., then water (5 mL) was added and the mixture was stirred for 10 min. The solution was extracted with EtOAc, washed with brine, dried over anhydrous Na₂SO₄, and concentrated under reduced pressure. The crude obtained was taken in 10% NaOH solution (5 mL) and stirred at 25° C. for 10 min followed by reflux for another 30 min. The reaction mixture was cooled to 0° C. and 1N HCl solution was added drop wise to adjust the pH to 4 and then saturated NaHCO₃ solution was added to adjust the pH to 8. The solid was filtered, washed with cold water, and dried under vacuum to afford [4-(pyrazin-2-yl)pyridin-2-yl]thiourea [250 mg, 69%]. MS (ESI): m/z 231.9 [M+1]⁺.

4-(Pyrazin-2-yl)-N-[4-(pyridin-2-yl)-1,3-thiazol-2-yl]pyridin-2-amine. A solution of [4-(pyrazin-2-yl)pyridin-2-yl]thiourea (250 mg, 1.08 mmol) and 2-bromo-1-(pyridin-2-yl)ethan-1-one (304 mg, 1.08 mmol) in EtOH (5 mL) was heated at reflux for 18 h. The reaction mixture was poured into ice-water and stirred for 30 min. The reaction mixture was adjusted to pH 8 with a Na₂CO₃ solution and the solid that appeared was filtered and washed with water. The solid was dried under vacuum and purified by column chromatography to afford 4-(pyrazin-2-yl)-N-[4-(pyridin-2-yl)-1,3-thiazol-2-yl]pyridin-2-amine (120 mg, 33%). MS (ESI): m/z 333.1 [M+1]⁺.

### Example 33. 4-Methyl-N-[4-(pyridin-2-yl)-5-(pyrrolidin-1-ylmethyl)-1,3-thiazol-2-yl]pyridin-2-amine

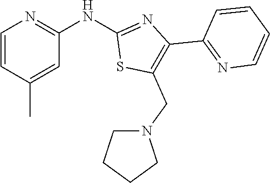 [SMILES: Cc1ccnc(Nc2nc(-c3ccccn3)c(CN3CCCC3)s2)c1]

4-Methyl-N-[4-(pyridin-2-yl)-5-(pyrrolidin-1-ylmethyl)-1,3-thiazol-2-yl]pyridin-2-amine. To a stirred solution of 4-Methyl-N-[4-(pyridin-2-yl)-1,3-thiazol-2-yl]pyridin-2-amine (100 mg, 0.37 mmol) in EtOH (10 ml), was added pyrrolidine (0.3 ml, 3.73 mmol) and aqueous HCHO (37%) (0.3 ml, 3.73 mmol) and the mixture was heated at 80° C. for 2 h. After completion, the reaction mixture was cooled to 25° C. and diluted with EtOAc. The organic layer was washed with brine, dried over Na₂SO₄, filtered, and concentrated under reduced pressure. The crude compound was purified by prep-HPLC to afford 4-methyl-N-[4-(pyridin-2-yl)-5-(pyrrolidin-1-ylmethyl)-1,3-thiazol-2-yl]pyridin-2-amine (65 mg, 49%). MS (ESI): m/z 352.1 [M+1]⁺.

### Example 34. 4-Methyl-N-[4-(3-methylpyridin-2-yl)-5-phenyl-1,3-thiazol-2-yl]pyridin-2-amine

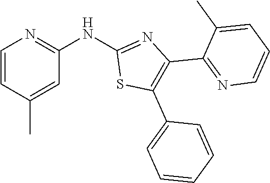 [SMILES: Cc1ccnc(Nc2nc(-c3ncccc3C)c(-c3ccccc3)s2)c1]

1-(3-Methylpyridin-2-yl)-2-phenylethan-1-one. To a stirred solution of 3-methylpyridine-2-carbonitrile (1 g, 8.46 mmol) in dry THE (30 ml), was added benzyl magnesium bromide (1M in THF, 16.9 ml, 16.9 mmol) dropwise at 0° C. under a nitrogen atmosphere. The mixture was stirred for 2 h at 0° C. The reaction mixture was quenched with saturated NH₄Cl solution and extracted with EtOAc. The combined organics were washed with water and brine, dried over anhydrous Na₂SO₄, filtered, and concentrated under reduced pressure. The crude mass was purified by column chromatography to afford 1-(3-methylpyridin-2-yl)-2-phenylethan-1-one (1.2 g, 67%). MS (ESI): m/z 212.2 [M+1]⁺.

2-Bromo-1-(3-methylpyridin-2-yl)-2-phenylethan-1-one. To a stirred solution of 1-(3-methylpyridin-2-yl)-2-phenylethan-1-one (300 mg, 1.42 mmol) in glacial AcOH (3 ml), HBr in AcOH (33%, 1.5 ml) was added and the mixture was cooled to 0° C. Bromine in AcOH (1 M, 2.84 ml, 2.84 mmol) was added and the resulting mixture was warmed to 25° C. and stirred for 2 h. The crude was filtered, washed with Et₂O, and dried to afford 2-bromo-1-(3-methylpyridin-2-yl)-2-phenylethan-1-one hydrobromide salt (350 mg, 71%). MS (ESI): m/z 292.1 [M+2]⁺.

4-Methyl-N-[4-(3-methylpyridin-2-yl)-5-phenyl-1,3-thiazol-2-yl]pyridin-2-amine. To a stirred solution of 1-(3-methylpyridin-2-yl)-2-phenylethan-1-one hydrobromide salt (250 mg, 0.68 mmol) in EtOH (10 ml), (4-methylpyridin-2-yl)thiourea (112 mg, 0.676 mmol) was added at 25° C. The resulting mixture was heated to reflux for 16 h. The reaction mixture was cooled to 25° C. and poured into crushed ice. It was adjusted to pH 8 with a Na₂CO₃ solution and extracted with EtOAc. The combined organics were washed with brine, dried over Na₂SO₄, filtered, and concentrated under reduced pressure. The crude mass was purified by column chromatography to afford 4-methyl-N-[4-(3-methylpyridin-2-yl)-5-phenyl-1,3-thiazol-2-yl]pyridin-2-amine (110 mg, 45%). MS (ESI): m/z 359.3 [M+1]⁺.

### Example 35. N-[5-Ethyl-4-(pyridin-2-yl)-1,3-thiazol-2-yl]-4-methylpyridin-2-amine

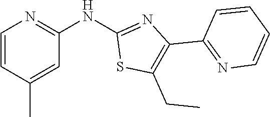 [SMILES: CCc1sc(Nc2cc(C)ccn2)nc1-c1ccccn1]

N-Methoxy-N-methylpyridine-2-carboxamide. EDC·HCl (2.27 gm, 14.62 mmol) and HOBt (1.3 gm, 9.75 mmol) were added to a stirred solution of pyridine-2-carboxylic acid (1 gm, 8.12 mmol) in DMF (20 mL). Then N,O-dimethyl hydroxylamine hydrochloride (875 mg, 8.93 mmol) followed by TEA (2.2 mL, 16.25 mmol) were added at 25° C. under an argon atmosphere. The reaction mixture was stirred for 3 h at 25° C. and quenched with water. The aqueous part was extracted with EtOAc. The combined organic layer was washed with brine, dried over anhydrous Na₂SO₄, and concentrated under reduced pressure. The residue was purified by column chromatography to afford N-methoxy-N-methylpyridine-2-carboxamide (650 mg, 48%). MS (ESI): m/z 166.8 [M+1]⁺.

1-(Pyridin-2-yl)butan-1-one. To a stirred solution of N-methoxy-N-methylpyridine-2-carboxamide (600 mg, 3.61 mmol) in dry THE (8 mL), was added n-propyl MgBr (2.4 mL, 7.22 mmol, 3M in THF) at −10° C. under argon atmosphere. The reaction was stirred at 0° C. for 1 h and quenched with saturated NH₄Cl solution. The solution was extracted with EtOAc and the combined organic layer was washed with brine, dried over anhydrous Na₂SO₄, and concentrated under reduced pressure. The residue was purified by column chromatography to afford 1-(pyridin-2-yl)butan-1-one (160 mg, 33%). MS (ESI): m/z 150.1 [M+1]⁺.

2-Bromo-1-(pyridin-2-yl)butan-1-one. To a solution of 1-(pyridin-2-yl)butan-1-one (180 mg, 1.21 mmol) in glacial AcOH (1.8 mL), was added 33% HBr in AcOH (0.9 mL) at 0° C. Then bromine in AcOH (1.4 ml, 1.45 mmol, 1 M) was added dropwise and the reaction mixture was stirred at 25° C. for 4 h. The reaction was cooled to 0° C. and quenched with saturated NaHCO₃ solution. The solution was extracted with EtOAc, washed with brine, dried over anhydrous Na₂SO₄, and concentrated under reduced pressure to get 2-bromo-1-(pyridin-2-yl)butan-1-one (120 mg, 43%). MS (ESI): m/z 228.0, 230.0 [M+1]⁺.

(4-Methylpyridin-2-yl)thiourea. Benzoyl isothiocyanate (15.09 mL, 92.59 mmol) was added to a stirred solution of 4-methylpyridin-2-amine (10 g, 92.59 mmol) in acetone (150 mL) and the resulting mixture was stirred at 25° C. for 1 h. The suspension was cooled in an ice-water bath, diluted with ice-water (20 mL), and stirred for 15 mins. The precipitate was filtered, washed with cold water, and dried under vacuum. The crude compound (23 g) was suspended in 10% aqueous NaOH (115 mL) and stirred at 25° C. for 15 min and then heated to reflux for additional 15 min. The reaction mixture was cooled to 0° C., adjusted to pH 4 with an aqueous HCl solution, and then adjusted to pH 8-9 with saturated aqueous KHCO₃ solution. The mixture was stirred at 0° C. for 15 min. The solid was filtered, washed with cold water, and dried under vacuum to afford (4-methylpyridin-2-yl)thiourea (11 g, 71%). MS (ESI): m/z 168.1 [M+1]⁺.

N-[5-Ethyl-4-(pyridin-2-yl)-1,3-thiazol-2-yl]-4-methylpyridin-2-amine. (4-Methylpyridin-2-yl)thiourea (88 mg, 0.53 mmol) was added to a solution of EtOH (5 mL) and 2-bromo-1-(pyridin-2-yl)butan-1-one (120 mg, 0.53 mmol) and the mixture was heated to reflux for 18 h. Then the reaction mixture was poured into ice-water and stirred for 30 min. The reaction mixture was adjusted to pH 8 with a Na₂CO₃ solution and the resulting material was filtered and washed with water. The crude was dried under vacuum and purified by column chromatography to afford N-[5-ethyl-4-(pyridin-2-yl)-1,3-thiazol-2-yl]-4-methylpyridin-2-amine (100 mg, 64%). MS (ESI): m/z 297.1 [M+1]⁺.

### Example 36. 5-((Dimethylamino)methyl)-4-(pyridin-2-yl)-N-(4-(trifluoromethyl)pyridin-2-yl)thiazol-2-amine

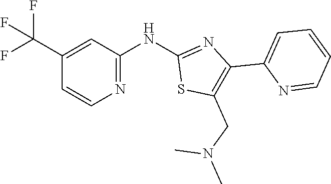 [SMILES: CN(C)Cc1sc(Nc2cc(C(F)(F)F)ccn2)nc1-c1ccccn1]

5-((Dimethylamino)methyl)-4-(pyridin-2-yl)-N-(4-(trifluoromethyl)pyridin-2-yl)thiazol-2-amine. A mixture of 5-(chloromethyl)-4-(pyridin-2-yl)-N-(4-(trifluoromethyl)pyridin-2-yl)thiazol-2-amine (0.05 g, 0.135 mmol) and dimethylamine was added to MeOH (0.081 ml, 0.162 mmol) in THE and stirred for 16 h at 25° C. The mixture was concentrated in vacuo and purified by prep HPLC to yield the acetate salt, filtered through a SCX column, and the filtrate was concentrated to yield 5-((dimethylamino)methyl)-4-(pyridin-2-yl)-N-(4-(trifluoromethyl)pyridin-2-yl)thiazol-2-amine (9 mg, 0.024 mmol, 17.59%). MS (ESI): m/z 379.9 [M+1]⁺. ¹H NMR (300 MHz, Methanol-d4) δ 8.63 (ddd, J=4.9, 1.8, 0.9 Hz, 1H), 8.54 (d, J=5.3 Hz, 1H), 8.02 (dt, J=8.0, 1.1 Hz, 1H), 7.89 (td, J=7.7, 1.8 Hz, 1H), 7.40-7.28 (m, 2H), 7.21-7.13 (m, 1H), 4.23 (s, 2H), 2.39 (s, 6H).

### Example 37. 5-(((3S,5R)-3,5-Dimethylpiperazin-1-yl)methyl)-4-(pyridin-2-yl)-N-(4-(trifluoromethyl)pyridin-2-yl)thiazol-2-amine

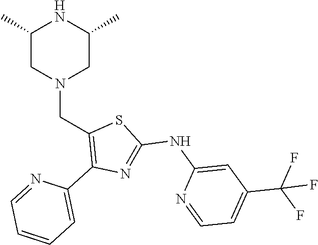 [SMILES: C[C@@H]1CN(Cc2sc(Nc3cc(C(F)(F)F)ccn3)nc2-c2ccccn2)C[C@H](C)N1]

5-(((3S,5R)-3,5-Dimethylpiperazin-1-yl)methyl)-4-(pyridin-2-yl)-N-(4-(trifluoromethyl)pyridin-2-yl)thiazol-2-amine. Tert-butyl (2R,6S)-2,6-dimethylpiperazine-1-carboxylate (184 mg, 0.856 mmol) and AcOH (0.016 ml, 0.285 mmol) were added to a solution of 4-(pyridin-2-yl)-2-((4-(trifluoromethyl)pyridin-2-yl)amino)thiazole-5-carbaldehyde (100 mg, 0.285 mmol) and powdered molecular sieves in DCE (5 ml). The mixture was stirred for 3 h at reflux. After cooling to 25° C., sodium triacetoxyborohydride (121 mg, 0.571 mmol) was added and the reaction was stirred at 25° C. for 1 h. The reaction mixture was filtered through celite and the filtrate was washed with water. The organic layer was separated, dried over Na₂SO₄, filtered, and the solvent removed in vacuo. The BOC-protected product was dissolved in DCM (2 ml) and HCl (1 ml, 4.00 mmol, 4 M in dioxane) was added. After 2 h, the reaction mixture was concentrated under reduced pressure and the solids were dissolved in MeOH (5 ml) and filtered through a SCX-2 column. The product was washed with 20 ml MeOH and 2M Ammonia/MeOH to give the free base. The filtrate was concentrated under reduced pressure and purified by prep HPLC to yield the expected product as the acetate salt. The solids were dissolved in MeOH (5 ml) and filtered through a SCX-2 column and washed with 20 ml MeOH and 2 M Ammonia/MeOH to give 5-(((3S,5R)-3,5-dimethylpiperazin-1-yl)methyl)-4-(pyridin-2-yl)-N-(4-(trifluoromethyl)pyridin-2-yl)thiazol-2-amine (40 mg, 0.089 mmol, 31.2%). MS (ESI): m/z 449.1 [M+1]⁺. ¹H NMR (300 MHz, Methanol-d4) δ 8.58 (dd, J=19.1, 5.1 Hz, 2H), 8.02-7.81 (m, 2H), 7.38-7.27 (m, 2H), 7.16 (d, J=5.3 Hz, 1H), 4.19 (s, 2H), 2.93 (d, J=9.4 Hz, 5H), 1.75 (t, J=11.3 Hz, 2H), 1.05 (d, J=6.3 Hz, 6H).

### Example 38. 4-(Pyridin-2-yl)-2-((4-(trifluoromethyl)pyridin-2-yl)amino)thiazole-5-carboxylic acid

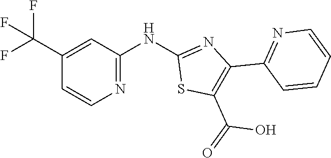 [SMILES: O=C(O)c1sc(Nc2cc(C(F)(F)F)ccn2)nc1-c1ccccn1]

4-(Pyridin-2-yl)-2-((4-(trifluoromethyl)pyridin-2-yl)amino)thiazole-5-carboxylic acid. An aqueous solution of NaOH (189 mg, 4.73 mmol, 5 ml) was added to a stirring solution of ethyl 4-(pyridin-2-yl)-2-((4-(trifluoromethyl)pyridin-2-yl)amino)thiazole-5-carboxylate and HBr (900 mg, 1.894 mmol) in EtOH (8 ml) and then the reaction mixture was allowed to stir at 60° C. for 16 h. After completion, the organic solvent was removed under reduced pressure, and the pH was adjusted to 4 with dilute HCl. The crude was filtered off and dried, then triturated with EtOH to furnish the final compound 4-(pyridin-2-yl)-2-((4-(trifluoromethyl)pyridin-2-yl)amino)thiazole-5-carboxylic acid (550 mg, 1.486 mmol, 78%). MS (ESI): m/z 366.7 [M+1]⁺. ¹H NMR (300 MHz, DMSO-d6) δ 12.21 (s, 1H), 8.79 (d, J=5.1 Hz, 1H), 8.68 (d, J=5.3 Hz, 1H), 8.42 (d, J=8.1 Hz, 1H), 8.31 (td, J=7.8, 1.7 Hz, 1H), 7.78-7.69 (m, 1H), 7.45 (s, 1H), 7.37 (d, J=5.4 Hz, 1H).

### Example 39. (4-(Pyridin-2-yl)-2-((4-(trifluoromethyl)pyridin-2-yl)amino)thiazol-5-yl)(pyrrolidin-1-yl)methanone

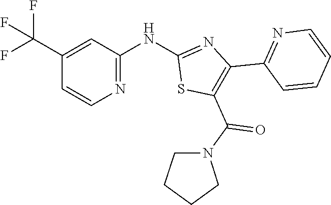 [SMILES: O=C(c1sc(Nc2cc(C(F)(F)F)ccn2)nc1-c1ccccn1)N1CCCC1]

(4-(Pyridin-2-yl)-2-((4-(trifluoromethyl)pyridin-2-yl)amino)thiazol-5-yl)(pyrrolidin-1-yl)methanone. 2,4,6-tripropyl-1,3,5,2,4,6-trioxatriphosphinane 2,4,6-trioxide (0.081 ml, 0.273 mmol) and then pyrrolidine (0.023 ml, 0.273 mmol) were added to a stirring solution of 4-(pyridin-2-yl)-2-((4-(trifluoromethyl)pyridin-2-yl)amino)thiazole-5-carboxylic acid (50 mg, 0.136 mmol) and TEA (0.058 ml, 0.409 mmol) in DMF (0.5 ml). The reaction was allowed to stir at 25° C. for 2 h. After completion it was poured onto ice, filtered off, washed with water, and triturated with EtOH to furnish the final compound (4-(pyridin-2-yl)-2-((4-(trifluoromethyl)pyridin-2-yl)amino)thiazol-5-yl)(pyrrolidin-1-yl)methanone (40 mg, 0.095 mmol, 69.3%). MS (ESI): m/z 419.7 [M+1]⁺. ¹H NMR (300 MHz, DMSO-d6) δ 12.00 (s, 1H), 8.57 (dd, J=11.6, 5.1 Hz, 2H), 8.03-7.85 (m, 2H), 7.39 (s, 1H), 7.37-7.27 (m, 2H), 3.49 (t, J=6.9 Hz, 2H), 3.06 (t, J=6.6 Hz, 2H), 1.83 (q, J=6.7 Hz, 2H), 1.78-1.61 (m, 2H).

### Example 40. N-(5-Isopropyl-4-(trifluoromethyl)pyridin-2-yl)-5-methyl-4-(pyridin-2-yl)thiazol-2-amine

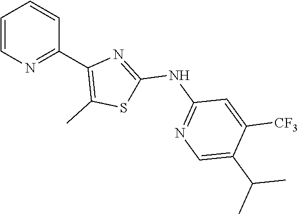 [SMILES: Cc1sc(Nc2cc(C(F)(F)F)c(C(C)C)cn2)nc1-c1ccccn1]

N-(5-Isopropyl-4-(trifluoromethyl)pyridin-2-yl)-5-methyl-4-(pyridin-2-yl)thiazol-2-amine. 2-Bromo-1-(pyridin-2-yl)propan-1-one (138 mg, 0.646 mmol) was added to a stirred solution of 1-(5-isopropyl-4-(trifluoromethyl)pyridin-2-yl)thiourea (170 mg, 0.646 mmol) in EtOH (2 ml) and the mixture was stirred at 85° C. for 3 h. The reaction mixture was cooled to 25° C. and filtered. The HBr salt was neutralized by dissolving 150 mg in 10 ml MeOH, followed by the addition of 0.200 ml NEt₃. Water was added and the precipitate filtered to give N-(5-isopropyl-4-(trifluoromethyl)pyridin-2-yl)-5-methyl-4-(pyridin-2-yl)thiazol-2-amine (85 mg, 0.220 mmol, 34.1%). MS (ESI): m/z 379.1 [M+1]⁺. ¹H NMR (300 MHz, DMSO-d6) δ 11.46 (s, 1H), 8.69-8.55 (m, 2H), 8.01-7.93 (m, 1H), 7.87 (td, J=7.7, 1.8 Hz, 1H), 7.37 (s, 1H), 7.28 (ddd, J=7.5, 4.8, 1.3 Hz, 1H), 3.12 (p, J=7.0 Hz, 1H), 2.75 (s, 3H), 1.31 (d, J=6.8 Hz, 6H).

### Example 41. 2-((5-(((2S,6R)-2,6-Dimethylmorpholino)methyl)-4-(pyridin-2-yl)thiazol-2-yl)amino)-N-(2,2,2-trifluoroethyl)isonicotinamide

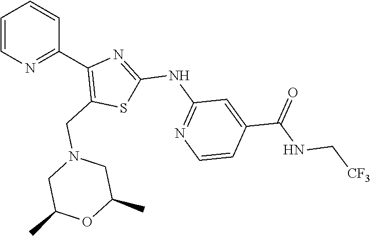 [SMILES: C[C@@H]1CN(Cc2sc(Nc3cc(C(=O)NCC(F)(F)F)ccn3)nc2-c2ccccn2)C[C@H](C)O1]

2-((5-(((2S,6R)-2,6-Dimethylmorpholino)methyl)-4-(pyridin-2-yl)thiazol-2-yl)amino)-N-(2,2,2-trifluoroethyl)isonicotinamide. TEA (0.091 ml, 0.654 mmol) and (2S,6R)-2,6-dimethylmorpholine (0.081 ml, 0.654 mmol) were added to a stirring solution of 2-((5-(chloromethyl)-4-(pyridin-2-yl)thiazol-2-yl)amino)-N-(2,2,2-trifluoroethyl)isonicotinamide (140 mg, 0.327 mmol) in a mixture of acetonitrile (1 ml) and MeOH (1.00 ml). The reaction mixture was allowed to stir at 50° C. for 2 h. After completion, the solvent was removed under reduced pressure and the crude mass was purified by column chromatography to afford the final compound 2-((5-(((2S,6R)-2,6-dimethylmorpholino)methyl)-4-(pyridin-2-yl)thiazol-2-yl)amino)-N-(2,2,2-trifluoroethyl)isonicotinamide (90 mg, 0.178 mmol, 54.3%). MS (ESI): m/z 506.8 [M+1]⁺. ¹H NMR (300 MHz, DMSO-d6) δ 11.48 (s, 1H), 9.36 (t, J=6.3 Hz, 1H), 8.63 (dt, J=4.6, 1.7 Hz, 1H), 8.46 (d, J=5.3 Hz, 1H), 7.98 (dt, J=8.0, 1.1 Hz, 1H), 7.88 (td, J=7.7, 1.9 Hz, 1H), 7.52 (s, 1H), 7.36-7.25 (m, 2H), 4.22 (s, 2H), 4.17-4.02 (m, 2H), 3.82 (ddd, J=11.0, 5.5, 3.2 Hz, 2H), 3.58 (d, J=8.1 Hz, 2H), 2.87 (d, J=11.0 Hz, 2H), 1.11 (d, J=6.3 Hz, 6H).

### Example 42. 4-(Pyridin-2-yl)-2-((4-(trifluoromethyl)pyridin-2-yl)amino)thiazole-5-carbonitrile

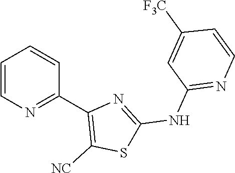 [SMILES: N#Cc1sc(Nc2cc(C(F)(F)F)ccn2)nc1-c1ccccn1]

4-(Pyridin-2-yl)-2-((4-(trifluoromethyl)pyridin-2-yl)amino)thiazole-5-carbonitrile. 1-(4-(trifluoromethyl)pyridin-2-yl)thiourea (72.3 mg, 0.327 mmol) was added to a stirring solution of 2-bromo-3-oxo-3-(pyridin-2-yl)propanenitrile and HBr (100 mg, 0.327 mmol) in EtOH (1 ml) and then the reaction mixture was allowed to stir at 85° C. for 2 h. After completion, it was filtered off and the solid was washed with EtOH. The crude was dissolved in 10 ml of 10% EtOH:DCM and washed with saturated NaHCO₃ solution. The organic layer was concentrated and dried to furnish the final compound 4-(pyridin-2-yl)-2-((4-(trifluoromethyl)pyridin-2-yl)amino)thiazole-5-carbonitrile (39 mg, 0.108 mmol, 33.1%). MS (ESI): m/z 347.8 [M+1]⁺. ¹H NMR (300 MHz, DMSO-d6) δ 12.70 (s, 1H), 8.78-8.66 (m, 2H), 8.11-7.98 (m, 2H), 7.57-7.47 (m, 1H), 7.44 (d, J=5.2 Hz, 2H).

### Example 43. 2-((5-Methyl-4-(pyridin-2-yl)thiazol-2-yl)amino)-N-(2,2,2-trifluoroethyl)isonicotinamide

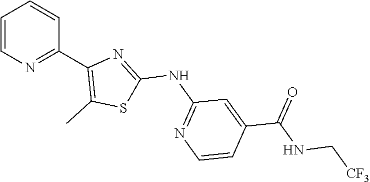 [SMILES: Cc1sc(Nc2cc(C(=O)NCC(F)(F)F)ccn2)nc1-c1ccccn1]

2-((5-methyl-4-(pyridin-2-yl)thiazol-2-yl)amino)-N-(2,2,2-trifluoroethyl)isonicotinamide. N-ethyl-N-isopropylpropan-2-amine (0.266 ml, 1.526 mmol) and 2-(3H-[1,2,3]triazolo[4,5-b]pyridin-3-yl)-1,1,3,3-tetramethylisouronium hexafluorophosphate(V) (290 mg, 0.763 mmol) were added to a stirring solution of 2-((5-methyl-4-(pyridin-2-yl)thiazol-2-yl)amino)isonicotinic acid hydrobromide (200 mg, 0.509 mmol) in DMF (2 ml) and then the reaction was allowed to stir at 25° C. for 10 min. After that, 2,2,2-trifluoroethan-1-amine (0.080 ml, 1.017 mmol) was added and the reaction was allowed to stir at 25° C. for 16 h. After completion, the reaction mixture was poured on ice, filtered off, washed with water, and dried. The solid product was triturated with MeOH to afford the desired final compound 2-((5-methyl-4-(pyridin-2-yl)thiazol-2-yl)amino)-N-(2,2,2-trifluoroethyl)isonicotinamide (55 mg, 0.140 mmol, 27.5%). MS (ESI): m/z 394 [M+1]⁺. ¹H NMR (300 MHz, DMSO-d6) δ 11.46 (s, 1H), 9.33 (t, J=6.2 Hz, 1H), 8.68-8.56 (m, 1H), 8.43 (dd, J=5.3, 0.8 Hz, 1H), 7.99 (dt, J=8.0, 1.1 Hz, 1H), 7.87 (td, J=7.7, 1.8 Hz, 1H), 7.48 (t, J=1.1 Hz, 1H), 7.36-7.24 (m, 2H), 4.22-4.02 (m, 2H), 2.75 (s, 3H).

### Example 44. 5-Methyl-4-(pyridin-2-yl)-N-(4-((tetrahydro-2H-pyran-4-yl)oxy)pyridin-2-yl)thiazol-2-amine

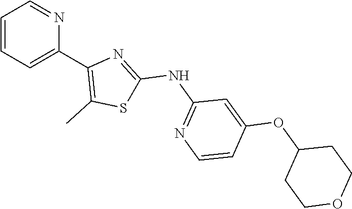 [SMILES: Cc1sc(Nc2cc(OC3CCOCC3)ccn2)nc1-c1ccccn1]

5-Methyl-4-(pyridin-2-yl)-N-(4-((tetrahydro-2H-pyran-4-yl)oxy)pyridin-2-yl)thiazol-2-amine. Pd₂(dba)₃ (151 mg, 0.165 mmol) was added to a degassed solution of Xantphos (191 mg, 0.330 mmol), Na₂CO₃ (175 mg, 1.650 mmol), 5-methyl-4-(pyridin-2-yl)thiazol-2-amine hydrobromide (494 mg, 1.815 mmol), and 2-bromo-4-((tetrahydro-2H-pyran-4-yl)oxy)pyridine (426 mg, 1.650 mmol) in dioxane (10 ml). The reaction vial was sealed and heated to 130° C. for 16 h. The crude reaction mixture was adsorbed onto silica and purified by column chromatography to afford the product. Pure fractions were collected, and the solvent evaporated to afford 5-methyl-4-(pyridin-2-yl)-N-(4-((tetrahydro-2H-pyran-4-yl)oxy)pyridin-2-yl)thiazol-2-amine (132 mg, 0.358 mmol, 21.71% yield). MS (ESI): m/z 369.2 [M+1]⁺. ¹H NMR (300 MHz, DMSO-d6) δ 10.96 (s, 1H), 8.73-8.50 (m, 1H), 8.10 (d, J=5.8 Hz, 1H), 7.97 (d, J=7.9 Hz, 1H), 7.86 (td, J=7.7, 1.9 Hz, 1H), 7.27 (s, 1H), 6.74-6.50 (m, 2H), 4.64 (dt, J=8.8, 4.5 Hz, 1H), 3.86 (dd, J=10.5, 5.8 Hz, 2H), 3.51 (ddd, J=11.8, 9.4, 2.8 Hz, 2H), 2.73 (s, 3H), 2.02 (d, J=12.5 Hz, 2H), 1.63 (dtd, J=13.0, 9.0, 3.9 Hz, 2H).

### Example 45. 5-Ethyl-4-(pyridin-2-yl)-N-(6-(trifluoromethyl)pyridin-2-yl)thiazol-2-amine

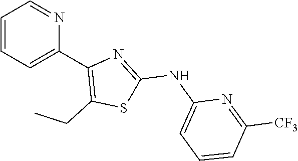 [SMILES: CCc1sc(Nc2cccc(C(F)(F)F)n2)nc1-c1ccccn1]

5-Ethyl-4-(pyridin-2-yl)-N-(6-(trifluoromethyl)pyridin-2-yl)thiazol-2-amine. A solution of 1-(6-(trifluoromethyl)pyridin-2-yl)thiourea (150 mg, 0.678 mmol), 2-bromo-1-(pyridin-2-yl)butan-1-one (170 mg, 0.746 mmol) and DIPEA (0.130 ml, 0.746 mmol) in EtOH (4 mL) were heated at 80° C. in a 7 ml sealed tube for 2 h. The resulting solution was adsorbed directly onto a minimum amount of silica and purified using column chromatography. Pure fractions were then collected and the solvent removed to afford 5-ethyl-4-(pyridin-2-yl)-N-(6-(trifluoromethyl)pyridin-2-yl)thiazol-2-amine (152 mg, 0.434 mmol, 64.0%). MS (ESI): m/z 350.9 [M+1]⁺. ¹H NMR (300 MHz, DMSO-d6) δ 11.64 (s, 1H), 8.63 (ddd, J=4.8, 1.9, 1.0 Hz, 1H), 8.03-7.91 (m, 2H), 7.87 (td, J=7.7, 1.9 Hz, 1H), 7.43-7.33 (m, 2H), 7.29 (ddd, J=7.4, 4.8, 1.3 Hz, 1H), 3.33 (q, J=7.4 Hz, 2H), 1.28 (t, J=7.4 Hz, 3H).

### Example 46. N-(3-Methyl-2-pyridyl)-4-[5-(2,2,2-trifluoroethoxy)-2-pyridyl]thiazol-2-amine

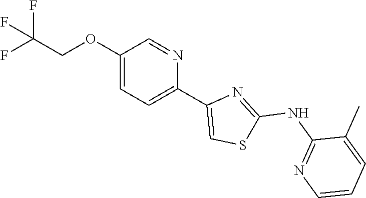 [SMILES: Cc1cccnc1Nc1nc(-c2ccc(OCC(F)(F)F)cn2)cs1]

5-(2,2,2-Trifluoroethoxy)pyridine-2-carbonitrile. To a stirred, ice-cold solution of 5-fluoropyridine-2-carbonitrile (650 mg, 5.32 mmol) in NMP (10 mL) was added NaH (60% in mineral oil, 332 mg, 7.98 mmol) at 0° C. under argon atmosphere. The resulting mixture was stirred at 24° C. for 5 min and cooled to 0° C. Trifluoroethanol (0.8 mL, 10.64 mmol) was added and the resulting mixture was allowed to warm to 24° C. The reaction mixture was heated to 90° C. for 4 h. After completion, the reaction mixture was quenched with aqueous Na₂CO₃ solution and extracted with EtOAc. The combined organic layer was washed with water and brine, dried over anhydrous Na₂SO₄, filtered, and concentrated under reduced pressure. The crude product was purified by column chromatography to afford 5-(2,2,2-trifluoroethoxy)pyridine-2-carbonitrile (750 mg, 69%). MS (ESI): m/z 203.1 [M+1]⁺.

1-[5-(2,2,2-Trifluoroethoxy)-2-pyridyl]ethanone. To a stirred solution of 5-(2,2,2-trifluoroethoxy)pyridine-2-carbonitrile (750 mg, 3.713 mmol) in THE (15 mL) was added MeMgBr (3 M in THF, 2.5 ml, 7.50 mmol) dropwise at 0° C. under an argon atmosphere and the mixture was stirred at 0° C. for 2 h. The reaction mixture was quenched with aqueous NH₄Cl solution and extracted with EtOAc. The combined organic layer was washed with water and brine, dried over anhydrous Na₂SO₄, filtered, and concentrated under reduced pressure. The crude product was purified by column chromatography to afford 1-[5-(2,2,2-trifluoroethoxy)-2-pyridyl]ethanone (250 mg, 31%). MS (ESI): m/z 220.2 [M+1]⁺.

2-Bromo-1-[5-(2,2,2-trifluoroethoxy)-2-pyridyl]ethanone. To a stirred solution of 1-[5-(2,2,2-trifluoroethoxy)-2-pyridyl]ethanone (250 mg, 1.14 mmol) in glacial AcOH (3 mL) was added HBr in glacial AcOH (1.5 mL) dropwise at 0° C. under an argon atmosphere. A solution of bromine in AcOH (1 M in AcOH, 1.4 mL, 1.4 mmol) was added to the reaction mixture dropwise at 0° C. and the resulting mixture was stirred at 24° C. for 2 h. The crude was filtered, washed with Et₂O, and dried to afford 2-bromo-1-[5-(2,2,2-trifluoroethoxy)-2-pyridyl]ethanone (550 mg, 62%). MS (ESI): m/z 298.2 [M+1]⁺.

(3-Methyl-2-pyridyl)thiourea. Benzoyl isothiocyanate (25.1 mL, 185.01 mmol) was added to a stirred solution of 3-methylpyridin-2-amine (20 g, 185.01 mmol) in acetone (200 ml) and the resulting mixture was stirred at 24° C. for 2 h. The suspension was cooled in an ice-water bath, diluted with ice-water (50 ml), and stirred for 15 min. The precipitate was filtered, washed with cold water, and dried under vacuum. The crude compound (45 g) was suspended in 10% aqueous NaOH (150 mL) and stirred at 24° C. for 15 min and then heated to reflux for an additional 15 min. The reaction mixture was cooled to 0° C., adjusted to pH 4 with an aqueous HCl solution, and then adjusted to a pH of 8-9 with saturated aqueous KHCO₃ solution. The mixture was stirred at 0° C. for 15 min. The crude was filtered, washed with cold water, and dried under vacuum to afford (3-methyl-2-pyridyl)thiourea (26 g, 84%). MS (ESI): m/z 168.0 [M+1]⁺.

N-(3-Methyl-2-pyridyl)-4-[5-(2,2,2-trifluoroethoxy)-2-pyridyl]thiazol-2-amine. 2-bromo-1-[5-(2,2,2-trifluoroethoxy)-2-pyridyl]ethenone hydrobromide (160 mg, 0.417 mmol) was added to a stirred solution of (3-methyl-2-pyridyl)thiourea (70 mg, 0.417 mmol) in EtOH (5 ml). The resulting mixture was heated to reflux for 4 h. The reaction mixture was cooled to 24° C. and poured into crushed ice. It was adjusted to a pH of 8 with aqueous Na₂CO₃ solution and extracted with EtOAc. The combined organic part was washed with brine, dried over anhydrous Na₂SO₄, filtered, and concentrated under reduced pressure. The crude mass was purified by column chromatography to afford N-(3-methyl-2-pyridyl)-4-[5-(2,2,2-trifluoroethoxy)-2-pyridyl]thiazol-2-amine (74 mg, 48%). MS (ESI): m/z 367.1 [M+1]⁺.

### Example 47. 2-[[4-(5-Ethoxy-2-pyridyl)thiazol-2-yl]amino]pyridine-3-carbonitrile

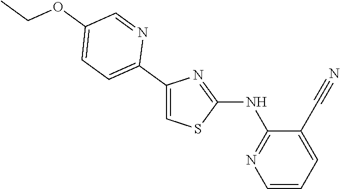 [SMILES: CCOc1ccc(-c2csc(Nc3ncccc3C#N)n2)nc1]

4-(5-Ethoxy-2-pyridyl)thiazol-2-amine. Thiourea (47 mg, 0.615 mmol) was added to a stirred solution of 2-bromo-1-(5-ethoxy-2-pyridyl)ethenone hydrobromide (200 mg, 0.615 mmol) in EtOH (5 ml). The resulting mixture was heated to reflux for 4 h. The reaction mixture was cooled to 24° C. and poured into crushed ice. It was adjusted to a pH of 8 with Na₂CO₃ solution and extracted with EtOAc. The combined organic part was washed with brine, dried over anhydrous Na₂SO₄, filtered, and concentrated under reduced pressure. The crude mass was triturated with Et₂O to afford 4-(5-ethoxy-2-pyridyl)thiazol-2-amine (110 mg, 81%). MS (ESI): m/z 222.3 [M+1]⁺.

2-[[4-(5-Ethoxy-2-pyridyl)thiazol-2-yl]amino]pyridine-3-carbonitrile. 4-(5-ethoxy-2-pyridyl)thiazol-2-amine (81 mg, 0.365 mmol) and Cs₂CO₃ (198 mg, 0.608 mmol) were added to a stirred solution of 2-bromopyridine-3-carbonitrile (55 mg, 3.04 mmol) in 1,4-dioxane (5 mL), and the mixture was degassed with argon for 10 min in a sealed tube. Xantphos (35 mg, 0.061 mmol) was added followed by Pd₂(dba)₃ (28 mg, 0.03 mmol) under an inert atmosphere. The resulting mixture was heated at 90° C. for 16 h. After completion, the reaction mixture was cooled to 24° C., diluted with water, and extracted with EtOAc. The combined organic layer was washed with brine, dried over Na₂SO₄, filtered, and concentrated under reduced pressure. The crude compound was purified by column chromatography to afford 2-[[4-(5-ethoxy-2-pyridyl)thiazol-2-yl]amino]pyridine-3-carbonitrile (40 mg, 41%). MS (ESI): m/z 324.3 [M+1]⁺.

### Example 48. N-[5-(Methoxymethyl)-4-(3-methylpyridin-2-yl)-1,3-thiazol-2-yl]-4-methylpyridin-2-amine

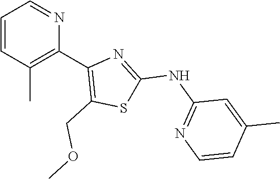 [SMILES: COCc1sc(Nc2cc(C)ccn2)nc1-c1ncccc1C]

Ethyl 3-(3-methylpyridin-2-yl)-3-oxopropanoate. To a stirred solution of 1-(3-methylpyridin-2-yl)ethan-1-one (2 g, 14.79 mmol) in diethyl carbonate (120 mL), was added NaH (60% in mineral oil, 3.55 g, 88.76 mmol) portion wise at 24° C. and the resulting mixture was heated at 90° C. for 4 h. After completion, the reaction mixture was cooled to 24° C. and quenched with a mixture of AcOH (8.4 mL) and Et₂O (84 mL), filtered through a short pad of celite and washed with EtOAc. The filtrate was concentrated under reduced pressure and the crude compound was purified by column chromatography to afford ethyl 3-(3-methylpyridin-2-yl)-3-oxopropanoate (2.8 g, 91%). MS (ESI): m/z 208.2 [M+1]⁺.

Ethyl 2-bromo-3-(3-methylpyridin-2-yl)-3-oxopropanoate. To a stirred solution of ethyl 3-(3-methylpyridin-2-yl)-3-oxopropanoate (1 g, 4.83 mmol) in CHCl₃ (30 mL) was added bromine (0.23 mL, 4.35 mmol) in CHCl₃ (10 mL) dropwise at 0° C. over a 15 min period. After completion, the reaction mixture was diluted with DCM. The organic layer was washed carefully with saturated aqueous NaHCO₃ solution, water and brine, dried over anhydrous Na₂SO₄, filtered, and concentrated under reduced pressure to afford ethyl 2-bromo-3-(3-methylpyridin-2-yl)-3-oxopropanoate (1.3 g, crude). MS (ESI): m/z 286.0 [M+1]⁺.

Ethyl 4-(3-methylpyridin-2-yl)-2-[(4-methylpyridin-2-yl)amino]-1,3-thiazole-5-carboxylate. To a stirred solution of (4-methylpyridin-2-yl)thiourea (762 mg, 4.56 mmol) in EtOH (30 mL), was added ethyl 2-bromo-3-(3-methylpyridin-2-yl)-3-oxopropanoate (1.3 g, 4.56 mmol) and the resulting mixture was heated at reflux for 3 h. After completion, the reaction mixture was cooled to 24° C., poured into ice-water, and stirred for another 30 min. The resulting solution was adjusted to pH 8 with an aqueous Na₂CO₃ solution and extracted with EtOAc. The combined organic layer was washed with brine, dried over anhydrous Na₂SO₄, filtered, and concentrated under reduced pressure. The crude compound was purified by column chromatography to afford ethyl 4-(3-methylpyridin-2-yl)-2-[(4-methylpyridin-2-yl)amino]-1,3-thiazole-5-carboxylate (1.05 g, 61% over 2 steps). MS (ESI): m/z 354.9 [M+1]⁺.

[4-(3-Methylpyridin-2-yl)-2-[(4-methylpyridin-2-yl)amino]-1,3-thiazol-5-yl]methanol. LiEt₃BH (Superhydride, 1M in THF, 5.65 mL, 5.65 mmol) was added dropwise to a stirred solution of ethyl 4-(3-methylpyridin-2-yl)-2-[(4-methylpyridin-2-yl)amino]-1,3-thiazole-5-carboxylate (500 mg, 1.41 mmol) in THE (15 mL) at −78° C. under an argon atmosphere. The resulting mixture was stirred at −78° C. for 30 min and at 24° C. for another 1 h. As starting material remained, it was again cooled to −78° C. and additional LiEt₃BH (Super hydride, 1M in THF, 5.65 mL, 5.65 mmol) was added dropwise. The reaction mixture was warmed to 24° C. and stirred for another 2 h. After completion, it was cooled to −10° C., quenched with saturated aqueous NH₄Cl solution, and extracted with EtOAc. The combined organic layer was washed with brine, dried over anhydrous Na₂SO₄, filtered, and concentrated under reduced pressure. The crude compound was purified by column chromatography to afford [4-(3-methylpyridin-2-yl)-2-[(4-methylpyridin-2-yl)amino]-1,3-thiazol-5-yl]methanol (350 mg, 79%). MS (ESI): m/z 313.3 [M+1]⁺.

N-[5-(Methoxymethyl)-4-(3-methylpyridin-2-yl)-1,3-thiazol-2-yl]-4-methylpyridin-2-amine. PBr₃ (0.015 mL, 0.16 mmol) was added dropwise to a stirred solution of [4-(3-methylpyridin-2-yl)-2-[(4-methylpyridin-2-yl)amino]-1,3-thiazol-5-yl]methanol (100 mg, 0.32 mmol) in DCM (10 mL) at 0° C. and the mixture was stirred at 24° C. for 30 min. Aqueous NaOH (103 mg in 2.8 ml water) was added to it and the mixture was stirred at 24° C. for 2 h. After completion, the reaction mixture was evaporated under reduced pressure and the residue was diluted with EtOAc and water. The resulting solution was neutralized with aqueous NaHCO₃ solution and extracted with EtOAc. The combined organic layer was washed with brine, dried over anhydrous Na₂SO₄, filtered, and concentrated under reduced pressure. The crude compound was purified by prep-HPLC to afford N-[5-(methoxymethyl)-4-(3-methylpyridin-2-yl)-1,3-thiazol-2-yl]-4-methylpyridin-2-amine (15 mg, 14%). MS (ESI): m/z 327.4 [M+1]⁺.

### Example 49. 4-(Pyridin-2-yl)-N-(pyrimidin-2-yl)thiazol-2-amine

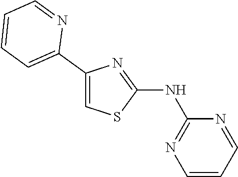 [SMILES: c1ccc(-c2csc(Nc3ncccn3)n2)nc1]

1-(Pyrimidin-2-yl)thiourea. Benzoyl isothiocyanate (1.632 g, 10.00 mmol) was added to a solution of pyrimidin-2-amine (0.951 g, 10 mmol) in THE (5 ml). The reaction was stirred at 60° C. for 1 h and the resulting material removed by filtration. The crude was then stirred with NaOH (20.00 ml, 50.0 mmol) at 60° C. for 1 h and the pH was adjusted to 8 by HCl. The crude was collected by filtration to afford 1-(pyrimidin-2-yl)thiourea (0.85 g, 5.51 mmol, 55.1% yield). MS (ESI) m/z 154.9 [M+H]⁺.

4-(Pyridin-2-yl)-N-(pyrimidin-2-yl)thiazol-2-amine. The solution of 2-bromo-1-(pyridin-2-yl)ethanone (0.200 g, 1 mmol) and 1-(pyrimidin-2-yl)thiourea (0.154 g, 1.000 mmol) in EtOH (10 mL) was stirred at 78° C. for 1 h. The reaction mixture was purified using reverse-phased semi-preparative HPLC. The fraction containing clean product was loaded onto an ion exchange column. The column was washed successively with water and MeOH. The product eluted with the 5% ammonium hydroxide in MeOH eluent and product containing eluent was concentrated under reduced pressure to afford 4-(pyridin-2-yl)-N-(pyrimidin-2-yl)thiazol-2-amine (0.034 g, 0.133 mmol, 13.32% yield). MS (ESI) m/z 256.2 [M+H]⁺. ¹H NMR (400 MHz, DMSO-d6) δ ppm 7.06 (t, J=5.08 Hz, 1H) 7.32 (dd, J=7.42, 5.08 Hz, 1H) 7.75 (s, 1H) 7.84-7.93 (m, 1H) 7.99 (d, J=8.20 Hz, 1H) 8.60 (dd, J=4.69, 0.78 Hz, 1H) 8.67 (d, J=5.47 Hz, 2H) 11.89 (s, 1H).

### Example 50. N-(4-Methylpyridin-2-yl)-4-(pyrimidin-2-yl)thiazol-2-amine

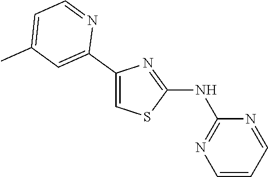 [SMILES: Cc1ccnc(-c2csc(Nc3ncccn3)n2)c1]

2-Bromo-1-(pyrimidin-2-yl)ethanone. Dibromine (0.720 g, 4.50 mmol) was added to a 0° C. solution of 1-(pyrimidin-2-yl)ethanone (0.5 g, 4.09 mmol) in HBr (33%) in AcOH (2 ml, 4.09 mmol). The reaction mixture was then stirred at 75° C. for 2 h. The reaction mixture was cooled down to 24° C. and Et₂O was added. The product was collected by filtration to afford 2-bromo-1-(pyrimidin-2-yl)ethanone hydrobromide (0.7 g, 3.48 mmol, 85% yield). MS (ESI) m/z 201.2 [M+H]⁺.

N-(4-Methylpyridin-2-yl)-4-(pyrimidin-2-yl)thiazol-2-amine. The solution of 2-bromo-1-(pyrimidin-2-yl)ethanone hydrobromide (0.201 g, 1 mmol) and 1-(4-methylpyridin-2-yl)thiourea (0.167 g, 1.000 mmol) in EtOH (10 ml) was stirred at 78° C. for 2 h. The reaction mixture was purified using reverse-phase semi-preparative HPLC. The fraction containing clean product was loaded onto an ion exchange column. The column was washed successively with water and MeOH. The product eluted with the 5% ammonium hydroxide in MeOH eluent and product containing eluent was concentrated under reduced pressure to afford N-(4-methylpyridin-2-yl)-4-(pyrimidin-2-yl)thiazol-2-amine (0.068 g, 0.252 mmol, 25.2% yield). MS (ESI) m/z 270.4 [M+H]+¹H NMR (400 MHz, DMSO-d6) δ ppm 2.29 (s, 3H) 6.79 (d, J=5.47 Hz, 1H) 6.83 (s, 1H) 7.39 (t, J=4.88 Hz, 1H) 7.87 (s, 1H) 8.18 (d, J=5.08 Hz, 1H) 8.84 (d, J=4.69 Hz, 2H) 11.61 (s, 1H).

### Example 51. 4-(5-Methoxypyridin-2-yl)-N-(4-methylpyridin-2-yl)thiazol-2-amine

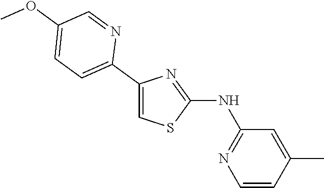

2-Chloro-1-(5-methoxypyridin-2-yl)ethanone. 1.6N nBuLi in hexane (3.66 ml, 5.85 mmol) was added dropwise to a −78° C. solution of 2-bromo-5-methoxypyridine (1 g, 5.32 mmol) in hexane (60 ml), Et₂O (30 ml), and THE (30 ml). After 10 min, 2-chloro-N-methoxy-N-methylacetamide (0.951 g, 6.91 mmol) in THE (2 mL) was added. The reaction was stirred at −78° C. for 15 min and allowed to warm up to 24° C. The reaction mixture was quenched with saturated aqueous NaCl and then washed with EtOAc. The organic phase was combined and washed with saturated aqueous NaCl. The organic layer was dried over magnesium sulfate, filtered, and concentrated. The crude was purified by column chromatography. Concentration of the desired fractions afforded 2-chloro-1-(5-methoxypyridin-2-yl)ethanone (0.8 g, 4.31 mmol, 81% yield). MS (ESI) m/z 186.0 [M+H]⁺.

4-(5-Methoxypyridin-2-yl)-N-(4-methylpyridin-2-yl)thiazol-2-amine. The solution of 2-chloro-1-(5-methoxypyridin-2-yl)ethanone (0.186 g, 1 mmol) and 1-(4-methylpyridin-2-yl)thiourea (0.167 g, 1.000 mmol) in EtOH (10 ml) was stirred at 78° C. for 1 h. The reaction mixture was concentrated. The crude was purified using reverse-phased semi-preparative HPLC. The fraction containing clean product was loaded onto an ion exchange column. The column was washed successively with water and MeOH. The product eluted with the 5% ammonium hydroxide in MeOH eluent and product containing eluent was concentrated under reduced pressure to afford 4-(5-methoxypyridin-2-yl)-N-(4-methylpyridin-2-yl)thiazol-2-amine (0.015 g, 0.050 mmol, 5.03% yield); m/z 299.3 [M+H]⁺. ¹H NMR (400 MHz, DMSO-d6) δ ppm 2.29 (s, 3H) 6.78 (dd, J=5.47, 0.78 Hz, 1H) 6.89 (s, 1H) 7.43 (s, 1H) 7.45-7.51 (m, 1H) 7.91 (d, J=8.59 Hz, 1H) 8.17 (d, J=5.47 Hz, 1H) 8.31 (d, J=3.12 Hz, 1H) 11.34 (s, 1H).

### Example 52. N-(3-(Azetidin-1-ylsulfonyl)pyridin-2-yl)-4-(5-isopropoxypyridin-2-yl)thiazol-2-amine

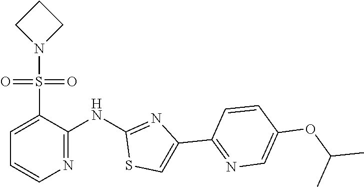 [SMILES: CC(C)Oc1ccc(-c2csc(Nc3ncccc3S(=O)(=O)N3CCC3)n2)nc1]

2-Bromo-5-isopropoxypyridine. K₂CO₃ (21.41 mL, 114.94 mmol) was added to a mixture of 6-bromopyridin-3-ol (10. g, 57.47 mmol) and 2-iodopropane (19.54 g, 114.94 mmol) in acetonitrile (120 mL). The mixture was stirred at 80° C. for 16 h. The reaction mixture was filtered and the filtrate was concentrated under vacuum to give a residue. The residue was purified by column chromatography to give 2-bromo-5-isopropoxypyridine (12 g, 55.535 mmol, 96.631% yield). LCMS (ESI): m/z 218.0 [M+1]⁺.

2-(1-Ethoxyvinyl)-5-isopropoxypyridine. A mixture of tributyl(1-ethoxyvinyl)stannane (20.06 g, 55.53 mmol), 2-bromo-5-isopropoxypyridine (12. g, 55.53 mmol), trans-dichlorobis(triphenylphosphine) palladium(II) (1.95 g, 2.78 mmol), and cuprous iodide (528.83 mg, 2.78 mmol) in 1,4-dioxane (200 mL) was stirred at 110° C. for 16 h under a nitrogen atmosphere. The residue was purified by column chromatography to give 2-(1-ethoxyvinyl)-5-isopropoxypyridine (5.5 g, 20.67 mmol, 37.22% yield). LCMS (ESI): m/z 208.2 [M+1]⁺.

2-Bromo-1-(5-isopropoxypyridin-2-yl)ethanone. To a solution of 2-(1-ethoxyvinyl)-5-isopropoxypyridine (5.5 g, 26.54 mmol) in THE (75 mL) and water (25 mL) was added NBS (4.72 g, 26.54 mmol) at 0° C. The mixture was stirred at 0° C. for 20 min. The residue was poured into water. The aqueous phase was extracted with EtOAc and the combined organic phase was washed with brine, dried over anhydrous Na₂SO₄, filtered, and concentrated under vacuum. The residue was purified by column chromatography to give 2-bromo-1-(5-isopropoxypyridin-2-yl)ethanone (5.5 g, 21.30 mmol, 80.30% yield). LCMS (ESI): m/z 258.0 [M+1]⁺.

N-(3-(Azetidin-1-ylsulfonyl)pyridin-2-yl)-4-(5-isopropoxypyridin-2-yl)thiazol-2-amine. 1-(3-(azetidin-1-ylsulfonyl)pyridin-2-yl)thiourea (464.27 mg, 1.7 mmol) was added to a mixture of 2-bromo-1-(5-isopropoxypyridin-2-yl)ethanone (440. mg, 1.70 mmol) in EtOH (5 mL). The mixture was stirred at 80° C. for 1 h. The residue was purified by column chromatography to give a crude product. The crude product was triturated with MeOH and DMF to give N-(3-(azetidin-1-ylsulfonyl)pyridin-2-yl)-4-(5-isopropoxypyridin-2-yl)thiazol-2-amine (396.52 mg, 0.92 mmol, 53.84% yield). LCMS (ESI): m/z 432.1 [M+1]⁺. ¹H NMR (400 MHz, DMSO-d₆) 10.13 (s, 1H), 8.71 (dd, J₁=1.4, J₂=4.8 Hz, 1H), 8.28-8.20 (m, 2H), 7.92 (d, J=8.8 Hz, 1H), 7.62 (s, 1H), 7.43 (dd, J₁=2.9, J₂=8.7 Hz, 1H), 7.29 (dd, J₁=4.8, J₂=7.8 Hz, 1H), 4.75-4.69 (m, 1H), 3.83 (t, J=7.6 Hz, 4H), 2.16-2.01 (m, 2H), 1.30 (d, J=6.0 Hz, 6H).

### Example 53. N-Methyl-N-(5-(trifluoromethyl)-2-((4-(1,2,2-trimethyl-2,3-dihydro-1H-pyrrolo[2,3-c]pyridin-5-yl)thiazol-2-yl)amino)pyridin-3-yl)acetamide

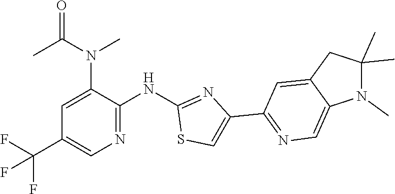 [SMILES: CC(=O)N(C)c1cc(C(F)(F)F)cnc1Nc1nc(-c2cc3c(cn2)N(C)C(C)(C)C3)cs1]

5-(1-Ethoxyvinyl)-1,2,2-trimethyl-2,3-dihydro-1H-pyrrolo[2,3-c]pyridine. A mixture of 5-chloro-1,2,2-trimethyl-2,3-dihydro-1H-pyrrolo[2,3-c]pyridine (2.6 g, 13.22 mmol), tributyl(1-ethoxyvinyl)stannane (5.73 g, 15.86 mmol) and [1,1′-bis(diphenylphosphino)ferrocene]dichloropalladium(II) (241.82 mg, 0.3300 mmol) in 1,4-dioxane (2 mL) was stirred at 100° C. for 16 h under a nitrogen atmosphere. The mixture was filtered and concentrated under reduced pressure to give a residue. The residue was purified by column chromatography to give 1-(1,2,2-trimethyl-2,3-dihydro-1H-pyrrolo[2,3-c]pyridin-5-yl)ethanone (1 g, 4.43 mmol, 33.55% yield). MS (ESI): 205.1 m/z [M+1]⁺. ¹H NMR (400 MHz, CDCl₃) δ=7.79 (d, J=0.63 Hz, 1H), 7.63 (s, 1H), 2.90 (s, 2H), 2.81 (s, 3H), 2.63 (s, 3H), 1.28 (s, 6H).

2-Bromo-1-(1,2,2-trimethyl-2,3-dihydro-1H-pyrrolo[2,3-c]pyridin-5-yl)ethanone. Phenyltrimethylammonium tribromide (1380.27 mg, 3.67 mmol) was added to a solution of 1-(1,2,2-trimethyl-2,3-dihydro-1H-pyrrolo[2,3-c]pyridin-5-yl)ethanone (750 mg, 3.67 mmol) in THE (10 mL). The mixture was stirred at 60° C. for 21 h under nitrogen. The mixture was filtered and the filtrate was concentrated under reduced pressure to give a residue which was purified by column chromatography to give 2-bromo-1-(1,2,2-trimethyl-2,3-dihydro-1H-pyrrolo[2,3-c]pyridin-5-yl)ethanone (260 mg, 0.85 mmol, 23.25% yield). MS (ESI): 283.8, 285.8 m/z [M+1]⁺. ¹H NMR (400 MHz, CDCl₃) δ=7.81 (s, 1H), 7.63 (s, 1H), 4.78 (s, 2H), 2.92 (s, 2H), 2.84 (s, 3H), 1.30 (s, 6H).

Tert-butyl methyl(5-(trifluoromethyl)-2-((4-(1,2,2-trimethyl-2,3-dihydro-1H-pyrrolo[2,3-c]pyridin-5-yl)thiazol-2-yl)amino)pyridin-3-yl)carbamate. Tert-butyl methyl(2-thioureido-5-(trifluoromethyl)pyridin-3-yl)carbamate (321.7 mg, 0.92 mmol) was added to a solution of 2-bromo-1-(1,2,2-trimethyl-2,3-dihydro-1H-pyrrolo[2,3-c]pyridin-5-yl)ethanone (260. mg, 0.9200 mmol) in EtOH (3 mL). The mixture was stirred at 80° C. for 1 h. The reaction mixture was concentrated under reduced pressure to remove the solvent. The residue was purified by column chromatography to give tert-butyl methyl(5-(trifluoromethyl)-2-((4-(1,2,2-trimethyl-2,3-dihydro-1H-pyrrolo[2,3-c]pyridin-5-yl)thiazol-2-yl)amino)pyridin-3-yl)carbamate (400 mg, 0.70 mmol, 77.0% yield). LCMS (ESI): 535.3 m/z [M+1]⁺.

N³-Methyl-5-(trifluoromethyl)-N²-(4-(1,2,2-trimethyl-2,3-dihydro-1H-pyrrolo[2,3-c]pyridin-5-yl)thiazol-2-yl)pyridine-2,3-diamine. HCl in EtOAc (1.87 mL, 7.48 mmol) was added to a solution of tert-butyl methyl(5-(trifluoromethyl)-2-((4-(1,2,2-trimethyl-2,3-dihydro-1H-pyrrolo[2,3-c]pyridin-5-yl)thiazol-2-yl)amino)pyridin-3-yl)carbamate (400. mg, 0.7500 mmol) in EtOAc (2 mL). The mixture was stirred at 20° C. for 1 h. The reaction mixture was concentrated under reduced pressure to remove the solvent to give N³-methyl-5-(trifluoromethyl)-N²-(4-(1,2,2-trimethyl-2,3-dihydro-1H-pyrrolo[2,3-c]pyridin-5-yl)thiazol-2-yl)pyridine-2,3-diamine hydrochloride (280 mg, 0.58 mmol, 77.95% yield). MS(ESI) 435.3 m/z [M+1]⁺.

N-Methyl-N-(5-(trifluoromethyl)-2-((4-(1,2,2-trimethyl-2,3-dihydro-1H-pyrrolo[2,3-c]pyridin-5-yl)thiazol-2-yl)amino)pyridin-3-yl)acetamide. TEA (0.41 mL, 2.97 mmol) followed by acetyl acetate (121.4 mg, 1.19 mmol) were added to a solution of N³-methyl-5-(trifluoromethyl)-N²-(4-(1,2,2-trimethyl-2,3-dihydro-1H-pyrrolo[2,3-c]pyridin-5-yl)thiazol-2-yl)pyridine-2,3-diamine hydrochloride (280. mg, 0.5900 mmol) in DMF (3 mL). The mixture was stirred at 40° C. for 16 h. Acetyl acetate (60.7 mg, 0.5900 mmol) was added and the mixture was stirred at 40° C. for another 24 h. The reaction mixture was concentrated under reduced pressure to remove the solvent and purified by prep-HPLC followed by lyophilization to give N-methyl-N-(5-(trifluoromethyl)-2-((4-(1,2,2-trimethyl-2,3-dihydro-1H-pyrrolo[2,3-c]pyridin-5-yl)thiazol-2-yl)amino)pyridin-3-yl)acetamide (74.47 mg, 0.15 mmol, 26.28% yield, 95.5% purity). LCMS (ESI): 477.1 m/z [M+1]⁺. ¹H NMR (400 MHz, DMSO-d₆) δ=8.75-8.63 (m, 1H), 8.23 (s, 0.5H), 8.01 (s, 0.4H), 7.76 (d, J=6.4 Hz, 1H), 7.67 (s, 1H), 7.44-7.40 (m, 1H), 3.28 (s, 1.5H), 3.09 (s, 1.6H), 2.87 (s, 2H), 2.70 (s, 3H), 2.22 (s, 1.4H), 1.74 (s, 1.6H), 1.21 (s, 6H)

### Example 54. N-Methyl-N-(5-(trifluoromethyl)-2-((4-(1,3,3-trimethyl-2-oxo-2,3-dihydro-1H-pyrrolo[2,3-c]pyridin-5-yl)thiazol-2-yl)amino)pyridin-3-yl)acetamide

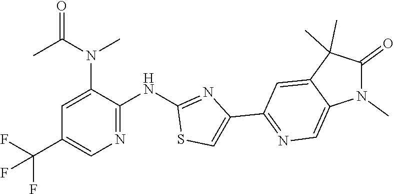 [SMILES: CC(=O)N(C)c1cc(C(F)(F)F)cnc1Nc1nc(-c2cc3c(cn2)N(C)C(=O)C3(C)C)cs1]

5-Chloro-1,3,3-trimethyl-1H-pyrrolo[2,3-c]pyridin-2(3H)-one. NaH (1.42 g, 35.59 mmol) was added to a mixture of 5-chloro-1H-pyrrolo[2,3-c]pyridin-2(3H)-one (1.2 g, 7.12 mmol) in DMF (40 mL) at 0° C. The mixture was stirred at 25° C. for 1 h. The mixture was cooled to 0° C. and iodomethane (5.05 g, 35.59 mmol) was added. The mixture was stirred at 25° C. for 2 h. The mixture was cooled and poured into ice-water slowly. The aqueous phase was extracted with EtOAc and the combined organic phase was washed with brine, dried over anhydrous Na₂SO₄, filtered and concentrated under vacuum. The residue was purified by column chromatography to give 5-chloro-1,3,3-trimethyl-1H-pyrrolo[2,3-c]pyridin-2(3H)-one (1.3 g, 6.17 mmol, 86.69% yield). LCMS (ESI): m/z 211.2 [M+1]⁺.

5-(1-Ethoxyvinyl)-1,3,3-trimethyl-1H-pyrrolo[2,3-c]pyridin-2(3H)-one. A mixture of 5-chloro-1,3,3-trimethyl-1H-pyrrolo[2,3-c]pyridin-2(3H)-one (1.3 g, 6.17 mmol), tributyl(1-ethoxyvinyl)stannane (2.67 g, 7.41 mmol) and [1,1′-bis(diphenylphosphino)ferrocene]dichloropalladium(II) (0.45 g, 0.62 mmol) in 1,4-dioxane (20 mL) was stirred at 100° C. for 16 h under nitrogen atmosphere. The reaction mixture was filtered and the filtrate was concentrated under reduced pressure. The residue was purified by column chromatography to give 5-(1-ethoxyvinyl)-1,3,3-trimethyl-1H-pyrrolo[2,3-c]pyridin-2(3H)-one (1.2 g, 4.87 mmol, 78.95% yield). LCMS (ESI): m/z 247.2 [M+1]⁺.

5-(2-Bromoacetyl)-1,3,3-trimethyl-1H-pyrrolo[2,3-c]pyridin-2(3H)-one. NBS (0.69 g, 3.9 mmol) was added to a solution of 5-(1-ethoxyvinyl)-1,3,3-trimethyl-1H-pyrrolo[2,3-c]pyridin-2(3H)-one (1.2 g, 3.9 mmol) in THE (15 mL) and water (5 mL) at 0° C. The mixture was stirred at 0° C. for 10 min. The residue was poured into water. The aqueous phase was extracted with EtOAc and the combined organic phase was washed with brine, dried over anhydrous Na₂SO₄, filtered, and concentrated under vacuum. The residue was purified by column chromatography to give 5-(2-bromoacetyl)-1,3,3-trimethyl-1H-pyrrolo[2,3-c]pyridin-2(3H)-one (1.1 g, 3.70 mmol, 94.97% yield). LCMS (ESI): m/z 297.0 [M+1]⁺.

Tert-butylmethyl(5-(trifluoromethyl)-2-((4-(1,3,3-trimethyl-2-oxo-2,3-dihydro-1H-pyrrolo[2,3-c]pyridin-5-yl)thiazol-2-yl)amino)pyridin-3-yl)carbamate. Tert-butyl methyl(2-thioureido-5-(trifluoromethyl)pyridin-3-yl)carbamate (636.7 mg, 1.82 mmol) was added to a solution of 5-(2-bromoacetyl)-1,3,3-trimethyl-1H-pyrrolo[2,3-c]pyridin-2(3H)-one (900. mg, 3.03 mmol) in EtOH (15 mL). The mixture was stirred at 80° C. for 1 h. The reaction mixture was concentrated under reduced pressure to give crude tert-butyl methyl(5-(trifluoromethyl)-2-((4-(1,3,3-trimethyl-2-oxo-2,3-dihydro-1H-pyrrolo[2,3-c]pyridin-5-yl)thiazol-2-yl)amino)pyridin-3-yl)carbamate (1.6 g, 2.91 mmol, 96.29% yield). MS (ESI): m/z 549.3 [M+1]⁺.

1,3,3-Trimethyl-5-(2-((3-(methylamino)-5-(trifluoromethyl)pyridin-2-yl)amino)thiazol-4-yl)-1H-pyrrolo[2,3-c]pyridin-2(3H)-one. A solution of tert-butyl methyl(5-(trifluoromethyl)-2-((4-(1,3,3-trimethyl-2-oxo-2,3-dihydro-1H-pyrrolo[2,3-c]pyridin-5-yl)thiazol-2-yl)amino)pyridin-3-yl)carbamate (1.4 g, 2.55 mmol) in HCl in EtOAc (20. mL, 80 mmol) was stirred at 20° C. for 2 h. The reaction mixture was concentrated under reduced pressure. The residue was poured into NaHCO₃ solution. The aqueous phase was extracted with EtOAc and the combined organic phase was washed with brine, dried over anhydrous Na₂SO₄, filtered and concentrated under vacuum. The residue was purified by column chromatography to give 1,3,3-trimethyl-5-(2-((3-(methylamino)-5-(trifluoromethyl)pyridin-2-yl)amino)thiazol-4-yl)-1H-pyrrolo[2,3-c]pyridin-2(3H)-one (620 mg, 1.37 mmol, 53.68% yield). MS (ESI): m/z 449.2 [M+1]⁺.

N-Methyl-N-(5-(trifluoromethyl)-2-((4-(1,3,3-trimethyl-2-oxo-2,3-dihydro-1H-pyrrolo[2,3-c]pyridin-5-yl)thiazol-2-yl)amino)pyridin-3-yl)acetamide. Acetyl acetate (177.56 mg, 1.74 mmol) was added to a mixture of 1,3,3-trimethyl-5-(2-((3-(methylamino)-5-(trifluoromethyl)pyridin-2-yl)amino)thiazol-4-yl)-1H-pyrrolo[2,3-c]pyridin-2(3H)-one (600. mg, 1.34 mmol) and TEA (406.15 mg, 4.01 mmol) in DMF (3 mL). The mixture was stirred at 40° C. for 36 h. The mixture was concentrated under vacuum and the residue was triturated with MeOH and dried by lyophilization to give N-methyl-N-(5-(trifluoromethyl)-2-((4-(1,3,3-trimethyl-2-oxo-2,3-dihydro-1H-pyrrolo[2,3-c]pyridin-5-yl)thiazol-2-yl)amino)pyridin-3-yl)acetamide (256.38 mg, 0.51 mmol, 38.32% yield). LCMS (ESI): m/z 491.1 [M+1]+¹H NMR (400 MHz, DMSO-d₆) 11.78-11.36 (m, 1H), 8.77-8.65 (m, 1H), 8.35 (s, 1H), 8.29-8.00 (m, 2H), 7.71-7.64 (m, 1H), 3.29 (s, 1.4H), 3.22 (s, 3H), 3.10 (s, 1.5H), 2.23 (s, 1.2H), 1.74 (s, 1.5H), 1.33 (s, 6H).

### Example 55. N-(6-((4-(4-Isopropoxypyridin-2-yl)thiazol-2-yl)amino)-5-(trifluoromethyl)pyridin-3-yl)-N-methylacetamide

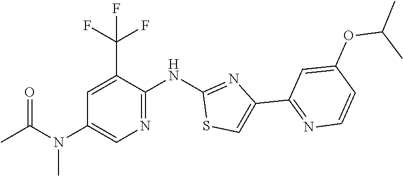 [SMILES: CC(=O)N(C)c1cnc(Nc2nc(-c3cc(OC(C)C)ccn3)cs2)c(C(F)(F)F)c1]

2-Bromo-4-isopropoxypyridine. K₂CO₃ (15.89 g, 114.94 mmol) was added to a mixture of 2-bromopyridin-4-ol (10.0 g, 57.47 mmol) and 2-iodopropane (10. g, 57.47 mmol) in acetonitrile (120 mL). The mixture was stirred at 80° C. for 16 h. The reaction mixture was filtered, and the filtrate was concentrated under reduced pressure to give a residue. The residue was purified by column chromatography to give 2-bromo-4-isopropoxypyridine (10 g, 46.27 mmol, 80.52% yield). ¹H NMR (400 MHz, CDCl₃) δ=8.07 (d, J=5.9 Hz, 1H), 6.89 (d, J=2.2 Hz, 1H), 6.66 (dd, J₁=2.3, J₂=5.8 Hz, 1H), 4.56-4.50 (m, 1H), 1.29 (d, J=6.1 Hz, 6H).

2-(1-Ethoxyvinyl)-4-isopropoxypyridine. A mixture of 2-bromo-4-isopropoxypyridine (6. g, 27.77 mmol), tributyl(1-ethoxyvinyl)stannane (10.03 g, 27.77 mmol), bis(triphenylphosphine)palladium(II)dichloride (0.97 g, 1.39 mmol) and copper(I)iodide (264.42 mg, 1.39 mmol) in 1,4-dioxane (150 mL) was stirred at 110° C. for 16 h under a nitrogen atmosphere. The reaction mixture was concentrated under reduced pressure. The residue was purified by column chromatography to give 2-(1-ethoxyvinyl)-4-isopropoxypyridine (6 g, 28.94 mmol, 104.25% yield). ¹H NMR (400 MHz, CDCl₃) δ=8.36 (d, J=5.7 Hz, 1H), 7.20 (d, J=2.4 Hz, 1H), 6.69 (dd, J₁=2.6, J₂=5.6 Hz, 1H), 4.70-4.64 (m, 1H), 4.35 (d, J=2.0 Hz, 1H), 3.97 (q, J=7.0 Hz, 2H), 1.44 (t, J=7.0 Hz, 3H), 1.37 (d, J=6.0 Hz, 7H).

2-Bromo-1-(4-isopropoxypyridin-2-yl)ethanone. To a solution of 2-(1-ethoxyvinyl)-4-isopropoxypyridine (6. g, 28.95 mmol) in THE (90 mL) and water (30 mL) was added NBS (5.15 g, 28.95 mmol) at 0° C. The mixture was stirred at 0° C. for 20 min. The residue was poured into water. The aqueous phase was extracted with EtOAc and the combined organic phase was washed with brine, dried over anhydrous Na₂SO₄, filtered, and concentrated under vacuum. The residue was purified by column chromatography to give 2-bromo-1-(4-isopropoxypyridin-2-yl)ethanone (3 g, 11.62 mmol, 40.15% yield). ¹H NMR (400 MHz, CDCl₃) δ=8.45 (d, J=5.6 Hz, 1H), 7.57 (d, J=2.6 Hz, 1H), 6.96 (dd, J₁=2.6, J₂=5.6 Hz, 1H), 4.85 (s, 2H), 4.75-4.69 (m, 1H), 1.41-1.36 (m, 6H).

Tert-butyl (6-((4-(4-isopropoxypyridin-2-yl)thiazol-2-yl)amino)-5-(trifluoromethyl)pyridin-3-yl)(methyl)carbamate. Tert-butylmethyl(6-thioureido-5-(trifluoromethyl)pyridin-3-yl)carbamate (678.7 mg, 1.94 mmol) was added to a mixture of 2-bromo-1-(4-isopropoxypyridin-2-yl)ethanone (500 mg, 1.94 mmol) in EtOH (5 mL). The mixture was stirred at 80° C. for 1 h. The reaction mixture was concentrated under reduced pressure. The residue was purified by column chromatography to give tert-butyl (6-((4-(4-isopropoxypyridin-2-yl)thiazol-2-yl)amino)-5-(trifluoromethyl)pyridin-3-yl)(methyl)carbamate (850 mg, 1.67 mmol, 86.11% yield). LCMS (ESI): m/z 510.2 [M+1]⁺.

N2-(4-(4-Isopropoxypyridin-2-yl)thiazol-2-yl)-N5-methyl-3-(trifluoromethyl)pyridine-2,5-diamine. A mixture of tert-butyl(6-((4-(4-isopropoxypyridin-2-yl)thiazol-2-yl)amino)-5-(trifluoromethyl)pyridin-3-yl)(methyl)carbamate (720. mg, 1.41 mmol) in HCl in EtOAc (20.0 mL, 80 mmol) at 0° C. was stirred at 25° C. for 2 h. The reaction mixture was concentrated under reduced pressure to give N2-(4-(4-isopropoxypyridin-2-yl)thiazol-2-yl)-N5-methyl-3-(trifluoromethyl)pyridine-2,5-diamine (350 mg, 0.78 mmol, 55.55% yield, HCl).

N-(6-((4-(4-Isopropoxypyridin-2-yl)thiazol-2-yl)amino)-5-(trifluoromethyl)pyridin-3-yl)-N-methylacetamide. Acetyl acetate (732.67 mg, 7.18 mmol) and TEA (1. mL, 7.18 mmol) were added to a mixture of N2-(4-(4-isopropoxypyridin-2-yl)thiazol-2-yl)-N5-methyl-3-(trifluoromethyl)pyridine-2,5-diamine (800. mg, 1.79 mmol) in DMF (10 mL). The mixture was stirred at 30° C. for 16 h. The reaction mixture was concentrated under reduced pressure. The residue was purified by prep-HPLC followed by lyophilization. A solution of NaOH (5 mL, 20 mmol) in water (5 mL) was added to the solid in THE (5 mL) and the mixture was stirred at 20° C. for 16 h. The mixture was poured into water and the aqueous phase was extracted with EtOAc and the combined organic phase was washed with brine, dried over anhydrous Na₂SO₄, filtered, and concentrated under vacuum. The residue was triturated with MTBE followed by lyophilization to give N-(6-((4-(4-isopropoxypyridin-2-yl)thiazol-2-yl)amino)-5-(trifluoromethyl)pyridin-3-yl)-N-methylacetamide (186.7 mg, 0.41 mmol, 22.93% yield). LCMS (ESI): 452.3 m/z [M+1]⁺. ¹H NMR (400 MHz, DMSO-d₆) δ=8.29 (d, J=5.5 Hz, 1H), 8.22 (s, 1H), 7.70-7.56 (m, 2H), 7.33 (s, 1H), 6.74 (d, J=2.2 Hz, 1H), 4.90-4.80 (m, 1H), 3.11 (s, 3H), 2.18 (s, 0.2H), 1.80 (s, 2.8H), 1.34 (d, J=6.0 Hz, 6H).

### Example 56. N-(6-((4-(5-Isopropoxypyridin-2-yl)thiazol-2-yl)amino)-5-(trifluoromethyl) pyridin-3-yl)-N-methylacetamide

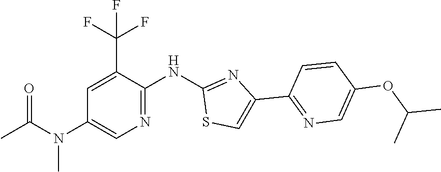 [SMILES: CC(=O)N(C)c1cnc(Nc2nc(-c3ccc(OC(C)C)cn3)cs2)c(C(F)(F)F)c1]

Tert-butyl methyl(6-thioureido-5-(trifluoromethyl)pyridin-3-yl)carbamate. NH₄OH (1.16 mL, 7.5 mmol) was added to a solution of tert-butyl (6-isothiocyanato-5-(trifluoromethyl)pyridin-3-yl)(methyl)carbamate (2.5 g, 7.5 mmol) in DCM (20 mL). The mixture was stirred at 20° C. for 1 h, concentrated under vacuum, and dried by lyophilization to give tert-butyl methyl(6-thioureido-5-(trifluoromethyl)pyridin-3-yl)carbamate (1.9 g, 5.423 mmol, 72.306% yield). ¹H NMR (DMSO-d₆, 400 MHz) δ 9.43-9.12 (m, 2H), 8.58 (d, J=2.3 Hz, 1H), 8.20 (d, J=2.4 Hz, 1H), 3.24 (s, 3H), 1.41 (s, 9H); LCMS (ESI): m/z 351.1 [M+1]⁺.

Tert-butyl (6-((4-(5-isopropoxypyridin-2-yl)thiazol-2-yl)amino)-5-(trifluoromethyl)pyridin-3-yl)(methyl)carbamate. Tert-butyl methyl(6-thioureido-5-(trifluoromethyl)pyridin-3-yl)carbamate (407.22 mg, 1.16 mmol) was added to a solution of 2-bromo-1-(5-isopropoxypyridin-2-yl)ethanone (300 mg, 1.16 mmol) in EtOH (10 mL). The mixture was stirred at 80° C. for 1 h. The mixture was concentrated under vacuum to give tert-butyl (6-((4-(5-isopropoxypyridin-2-yl)thiazol-2-yl)amino)-5-(trifluoromethyl)pyridin-3-yl)(methyl)carbamate (600 mg, 1.1775 mmol, 101.31% yield). LCMS (ESI): m/z 509.9 [M+1]⁺.

N²-(4-(5-Isopropoxypyridin-2-yl)thiazol-2-yl)-N⁵-methyl-3-(trifluoromethyl) pyridine-2,5-diamine. HCl in EtOAc (10. mL, 40 mmol, 4 M) was added to a solution of crude tert-butyl (6-((4-(5-isopropoxypyridin-2-yl)thiazol-2-yl)amino)-5-(trifluoromethyl)pyridin-3-yl)(methyl)carbamate (600. mg, 1.18 mmol) in EtOAc (10 mL). The mixture was stirred at 35° C. for 2 h. The mixture was cooled and concentrated under reduced pressure. The residue was poured into saturated aqueous NaHCO₃. The aqueous phase was extracted with EtOAc and the combined organic phase was washed with brine, dried over anhydrous Na₂SO₄, filtered and concentrated under vacuum. The residue was purified by column chromatography to give N²-(4-(5-isopropoxypyridin-2-yl)thiazol-2-yl)-N⁵-methyl-3-(trifluoromethyl)pyridine-2,5-diamine (320 mg, 0.7816 mmol, 66.374% yield). ¹H NMR (DMSO-d₆, 400 MHz) δ 9.92-9.62 (m, 1H), 8.25 (d, J=2.8 Hz, 1H), 7.98 (d, J=2.5 Hz, 1H), 7.94 (d, J=8.6 Hz, 1H), 7.46 (dd, J₁=8.8, J₂=2.9 Hz, 1H), 7.35 (s, 1H), 7.31 (d, J=2.6 Hz, 1H), 4.76-4.70 (m, 1H), 2.76 (s, 3H), 1.30 (d, J=6.0 Hz, 6H); LCMS (ESI): m/z 410.0 [M+1]⁺.

N-(6-((4-(5-Isopropoxypyridin-2-yl)thiazol-2-yl)amino)-5-(trifluoromethyl) pyridin-3-yl)-N-methylacetamide. Acetyl acetate (299.22 mg, 2.93 mmol) was added to a solution of N²-(4-(5-isopropoxypyridin-2-yl)thiazol-2-yl)-N⁵-methyl-3-(trifluoromethyl)pyridine-2,5-diamine (300. mg, 0.7300 mmol) and TEA (0.25 mL, 3.66 mmol) in DMF (1 mL). The mixture was stirred at 30° C. for 6 h. The mixture was concentrated and the residue was triturated with DMF (5 mL), then filtered. Then the filter cake was triturated by DMF (2 mL) again, then filtered. The filter cake was dried by lyophilization to give N-[6-[[4-(5-isopropoxy-2-pyridyl)thiazol-2-yl]amino]-5-(trifluoromethyl)-3-pyridyl]-N-methyl-acetamide (181.23 mg, 0.3982 mmol, 54.346% yield). ¹H NMR (DMSO-d₆, 400 MHz) δ 8.62-8.52 (m, 1H), 8.28 (d, J=2.7 Hz, 1H), 8.16 (s, 1H), 7.99 (d, J=8.8 Hz, 1H), 7.50-7.43 (m, 2H), 4.78-4.72 (m, 1H), 3.38 (s, 0.6H), 3.17 (s, 2.3H), 2.21 (s, 0.6H), 1.82 (s, 2.3H), 1.31 (d, J=6.0 Hz, 6H); LCMS (ESI): m/z 452.2 [M+1]⁺.

### Example 57. N-(2-((4-(2,2-Dimethyl-2,3-dihydrofuro[2,3-c]pyridin-5-yl)thiazol-2-yl)amino)-5-(trifluoromethyl)pyridin-3-yl)-N-methylacetamide

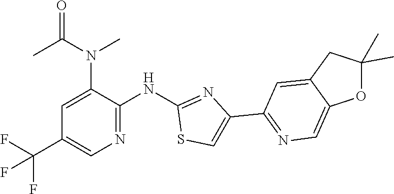 [SMILES: CC(=O)N(C)c1cc(C(F)(F)F)cnc1Nc1nc(-c2cc3c(cn2)OC(C)(C)C3)cs1]

1-(5-Bromo-2-chloropyridin-4-yl)-2-methylpropan-2-ol. To a solution of 5-bromo-2-chloro-4-methylpyridine (24 g, 116.24 mmol) in THE (200 mL) was added LDA (69.74 mL, 139.49 mmol, 2 M in THF) at 0° C. The mixture was stirred at 0° C. for 1 h. Then acetone (13.5 g, 232.48 mmol) was added to the mixture dropwise at 0° C. and the mixture was stirred at 25° C. for 3 h. The mixture was poured into NH₄Cl (300 mL) at 0° C. The aqueous phase was extracted with EtOAc and the combined organic phase was washed with brine, dried over anhydrous Na₂SO₄, filtered, and concentrated under vacuum. The residue was purified by column chromatography to give 1-(5-bromo-2-chloropyridin-4-yl)-2-methylpropan-2-ols (18 g, 46.948 mmol, 40.389% yield). LCMS (ESI): m/z 263.9 [M+1]⁺.

5-Chloro-2,2-dimethyl-2,3-dihydrofuro[2,3-c]pyridine. Cuprous iodide (1.22 g, 6.43 mmol), 8-hydroxy-quinoline (1.4 g, 9.64 mmol), and cesium carbonate (41.87 g, 128.52 mmol) were added to a solution of 1-(5-bromo-2-chloro-4-pyridyl)-2-methyl-propan-2-ol (17. g, 64.26 mmol) in toluene (200 mL). The mixture was stirred at 120° C. for 16 h, filtered, and the filtrate was concentrated under vacuum. The residue was purified by column chromatography to give 5-chloro-2,2-dimethyl-2,3-dihydrofuro[2,3-c]pyridine (4 g, 20.367 mmol, 31.695% yield). ¹H NMR (400 MHz, DMSO-d₆) δ 7.83 (s, 1H), 7.35 (s, 1H), 3.07 (s, 2H), 1.43 (s, 6H). LCMS (ESI): m/z 184.1 [M+1]⁺.

5-(1-Ethoxyvinyl)-2,2-dimethyl-2,3-dihydrofuro[2,3-c]pyridine. [1,1′-bis(diphenylphosphino)ferrocene]dichloropalladium(II) (1.59 g, 2.18 mmol) was added to a solution of 5-chloro-2,2-dimethyl-2,3-dihydrofuro[2,3-c]pyridine (4 g, 21.78 mmol) and tributyl(1-ethoxyvinyl)stannane (9.44 g, 26.14 mmol) in 1,4-dioxane (40 mL) under nitrogen. The mixture was stirred at 100° C. for 16 h. Then tributyl(1-ethoxyvinyl)stannane (7.36 mL, 21.78 mmol) was added and the mixture was stirred at 100° C. for another 6 h. The mixture was filtered and the filtrate was concentrated under vacuum. The mixture was purified by column chromatography to give 5-(1-ethoxyvinyl)-2,2-dimethyl-3H-furo[2,3-c]pyridine (1.9 g, 6.8451 mmol, 31.424% yield). ¹H NMR (400 MHz, DMSO-d₆) δ 8.01 (d, J=0.7 Hz, 1H), 7.51 (s, 1H), 5.14 (s, 1H), 4.24 (d, J=1.3 Hz, 1H), 3.90 (q, J=7.0 Hz, 2H), 3.06 (s, 2H), 1.43 (s, 6H), 1.35 (t, J=7.0 Hz, 3H); LCMS (ESI): m/z 220.1 [M+1]⁺.

2-Bromo-1-(2,2-dimethyl-2,3-dihydrofuro[2,3-c]pyridin-5-yl)ethanone. 1-Bromopyrrolidine-2,5-dione (1.54 g, 8.66 mmol) was added to a solution of 5-(1-ethoxyvinyl)-2,2-dimethyl-3H-furo[2,3-c]pyridine (1.9 g, 8.66 mmol) in water (20 mL) and THE (60 mL) at 0° C. The mixture was stirred at 0° C. for 20 min. The mixture was poured into water. The aqueous phase was extracted with EtOAc and the combined organic phase was washed with brine, dried over anhydrous Na₂SO₄, filtered, and concentrated under vacuum. The residue was purified by column chromatography to give 2-bromo-1-(2,2-dimethyl-2,3-dihydrofuro[2,3-c]pyridin-5-yl)ethanone (1.6 g, 4.324 mmol, 49.904% yield). ¹H NMR (400 MHz, DMSO-d₆) δ 8.17 (d, J=0.6 Hz, 1H), 7.97 (d, J=0.6 Hz, 1H), 4.91 (s, 2H), 3.15 (s, 2H), 1.47 (s, 6H); LCMS (ESI): m/z 270.0 [M+1]⁺.

Tert-butyl (2-((4-(2,2-dimethyl-2,3-dihydrofuro[2,3-c]pyridin-5-yl)thiazol-2-yl)amino)-5-(trifluoromethyl)pyridin-3-yl)(methyl)carbamate. Tert-butylmethyl(2-thioureido-5-(trifluoromethyl)pyridin-3-yl)carbamate (389.12 mg, 1.11 mmol) was added to a solution of 2-bromo-1-(2,2-dimethyl-2,3-dihydrofuro[2,3-c]pyridin-5-yl)ethanone (300 mg, 1.11 mmol) in EtOH (5 mL). The mixture was stirred at 80° C. for 1 h. The solid precipitated and was filtered. The filtrate was concentrated under vacuum, then triturated with EtOAc (5 mL) and filtered. The filter cake was combined and dried under vacuum to give tert-butyl(2-((4-(2,2-dimethyl-2,3-dihydrofuro[2,3-c]pyridin-5-yl)thiazol-2-yl)amino)-5-(trifluoromethyl)pyridin-3-yl)(methyl)carbamate (500 mg, 0.7957 mmol, 71.644% yield). ¹H NMR (400 MHz, DMSO-d₆) δ 11.57-11.52 (m, 1H), 8.67 (s, 1H), 8.24 (s, 1H), 8.19 (s, 1H), 8.15 (d, J=2.0 Hz, 1H), 7.93 (s, 1H), 3.30 (s, 2H), 3.14 (s, 3H), 1.51 (s, 6H), 1.46-1.15 (m, 9H).

N2-(4-(2,2-Dimethyl-2,3-dihydrofuro[2,3-c]pyridin-5-yl)thiazol-2-yl)-N3-methyl-5-(trifluoromethyl)pyridine-2,3-diamine hydrochloride. To a solution of crude tert-butyl N-[2-[[4-(2,2-dimethyl-3H-furo[2,3-c]pyridin-5-yl)thiazol-2-yl]amino]-5-(trifluoromethyl)-3-pyridyl]-N-methyl-carbamate (500. mg, 0.9600 mmol) in EtOAc (5 mL) was added HCl in EtOAc (10. mL, 40 mmol, 4 M). The mixture was stirred at 20° C. for 1 h and concentrated under vacuum to give N2-[4-(2,2-dimethyl-3H-furo[2,3-c]pyridin-5-yl)thiazol-2-yl]-N3-methyl-5-(trifluoromethyl)pyridine-2,3-diamine hydrochloride (400 mg, 0.8736 mmol, 91.122% yield). LCMS (ESI): m/z 422.1 [M+1]⁺.

N-(2-((4-(2,2-dimethyl-2,3-dihydrofuro[2,3-c]pyridin-5-yl)thiazol-2-yl)amino)-5-(trifluoromethyl)pyridin-3-yl)-N-methylacetamide. Acetic anhydride (145.34 mg, 1.42 mmol) was added to a mixture of N2-[4-(2,2-dimethyl-3H-furo[2,3-c]pyridin-5-yl)thiazol-2-yl]-N3-methyl-5-(trifluoromethyl)pyridine-2,3-diamine hydrochloride (400 mg, 0.9500 mmol) and TEA (288.13 mg, 2.85 mmol) in DMF (3 mL). The mixture was stirred at 40° C. for 16 h. The mixture was concentrated under vacuum. The residue was purified by prep-HPLC followed by lyophilization to give N-[2-[[4-(2,2-dimethyl-3H-furo[2,3-c]pyridin-5-yl)thiazol-2-yl]amino]-5-(trifluoromethyl)-3-pyridyl]-N-methyl-acetamide (213.29 mg, 0.4501 mmol, 47.419% yield). LCMS (ESI): m/z 464.1 [M+1]⁺. ¹H NMR (400 MHz, DMSO-d₆) 11.77-11.40 (m, 1H), 8.78-8.63 (m, 1H), 8.29-8.01 (m, 3H), 7.68 (d, J=2.9 Hz, 1H), 3.28 (s, 1.4H), 3.18 (s, 2H), 3.09 (s, 1.5H), 2.23 (s, 1.3H), 1.74 (s, 1.6H), 1.48 (s, 6H).

### Example 58. N-(6-((4-(2,2-Dimethyl-2,3-dihydrofuro[2,3-c]pyridin-5-yl)thiazol-2-yl)amino)-5-(trifluoromethyl)pyridin-3-yl)-N-methylacetamide

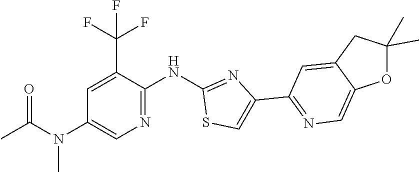 [SMILES: CC(=O)N(C)c1cnc(Nc2nc(-c3cc4c(cn3)OC(C)(C)C4)cs2)c(C(F)(F)F)c1]

Tert-butyl (6-((4-(2,2-dimethyl-2,3-dihydrofuro[2,3-c]pyridin-5-yl)thiazol-2-yl)amino)-5-(trifluoromethyl)pyridin-3-yl)(methyl)carbamate. Tert-butyl methyl(6-thioureido-5-(trifluoromethyl)pyridin-3-yl)carbamate (389.12 mg, 1.11 mmol) was added to a solution of 2-bromo-1-(2,2-dimethyl-2,3-dihydrofuro[2,3-c]pyridin-5-yl)ethanone (300. mg, 1.11 mmol) in EtOH (5 mL). The mixture was stirred at 80° C. for 1 h and was concentrated under vacuum to give crude tert-butyl (6-((4-(2,2-dimethyl-2,3-dihydrofuro[2,3-c]pyridin-5-yl)thiazol-2-yl)amino)-5-(trifluoromethyl)pyridin-3-yl)(methyl)carbamate (580 mg, crude). LCMS (ESI): m/z 521.9 [M+1]⁺.

N²-(4-(2,2-Dimethyl-2,3-dihydrofuro[2,3-c]pyridin-5-yl)thiazol-2-yl)-N⁵-methyl-3-(trifluoromethyl)pyridine-2,5-diamine. HCl in EtOAc (10. mL, 40 mmol, 4 M) was added to a solution of crude tert-butyl (6-((4-(2,2-dimethyl-2,3-dihydrofuro[2,3-c]pyridin-5-yl)thiazol-2-yl)amino)-5-(trifluoromethyl)pyridin-3-yl)(methyl)carbamate (580. mg, crude) in EtOAc (10 mL). The mixture was stirred at 20° C. for 1 h, concentrated under vacuum, and the residue was triturated with EtOAc (10 mL) and stirred for 1 h. The suspension was filtered and the filter cake was dried under vacuum to give N²-(4-(2,2-dimethyl-2,3-dihydrofuro[2,3-c]pyridin-5-yl)thiazol-2-yl)-N⁵-methyl-3-(trifluoromethyl)pyridine-2,5-diamine (600 mg, crude). LCMS (ESI): m/z 422.1 [M+1]⁺.

N-(6-((4-(2,2-Dimethyl-2,3-dihydrofuro[2,3-c]pyridin-5-yl)thiazol-2-yl)amino)-5-(trifluoromethyl)pyridin-3-yl)-N-methylacetamide Acetyl acetate (159.41 mg, 1.56 mmol) was added to a mixture of N2-[4-(2,2-dimethyl-3H-furo[2,3-c]pyridin-5-yl)thiazol-2-yl]-N5-methyl-3-(trifluoromethyl)pyridine-2,5-diamine hydrochloride (550. mg, 1.2 mmol) and TEA (364.63 mg, 3.6 mmol) in DMF (3 mL). The mixture was stirred at 40° C. for 16 h and concentrated under vacuum. The residue was purified by prep-HPLC followed by lyophilization to give N-[6-[[4-(2,2-dimethyl-3H-furo[2,3-c]pyridin-5-yl)thiazol-2-yl]amino]-5-(trifluoromethyl)-3-pyridyl]-N-methyl-acetamide (242.98 mg, 0.5138 mmol, 42.773% yield). LCMS (ESI): m/z 464.1 [M+1]⁺. ¹H NMR (400 MHz, DMSO-d₆) 8.64-8.50 (m, 1H), 8.18-8.05 (m, 2H), 7.96 (s, 1H), 7.36 (s, 1H), 3.38 (s, 0.6H), 3.16 (s, 2.3H), 3.12 (s, 2H), 2.21 (s, 0.5H), 1.81 (s, 2.3H), 1.47 (s, 6H).

### Example 59. 1-(2-((4-(4-Isopropoxypyridin-2-yl)thiazol-2-yl)amino)-5-(trifluoromethyl)pyridin-3-yl)pyrrolidin-2-one

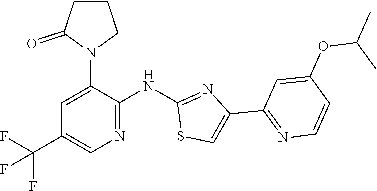 [SMILES: CC(C)Oc1ccnc(-c2csc(Nc3ncc(C(F)(F)F)cc3N3CCCC3=O)n2)c1]

1-(2-Azido-5-(trifluoromethyl)pyridin-3-yl)pyrrolidin-2-one. 1-(2-chloro-5-(trifluoromethyl)pyridin-3-yl)pyrrolidin-2-one (2.8 g, 10.58 mmol) was added to a solution of sodium azide (2.06 g, 31.74 mmol) in DMSO (30 mL). The mixture was stirred at 100° C. for 16 h and was then poured into saturated aqueous NaHCO₃. The aqueous phase was extracted with EtOAc and the combined organic phase was washed with brine, dried over anhydrous Na₂SO₄, filtered, and concentrated under vacuum to give 1-[2-azido-5-(trifluoromethyl)-3-pyridyl]pyrrolidin-2-one (2.8 g, crude).

1-(2-Amino-5-(trifluoromethyl)pyridin-3-yl)pyrrolidin-2-one. Pd/C (1098.73 mg, 10.32 mmol, 10% purity) and palladium hydroxide/carbon (1098.73 mg, 10.32 mmol, 20% purity) under nitrogen were added to a solution of 1-(2-azido-5-(trifluoromethyl)pyridin-3-yl)pyrrolidin-2-one (2.8 g, crude) in MeOH (100 mL). Then the mixture was stirred at 25° C. for 16 h under hydrogen (15 psi). The mixture was filtered with a pad of silica gel and celite, washed with MeOH and the filtrate was concentrated under vacuum. The residue was purified by column chromatography to give 1-(2-amino-5-(trifluoromethyl)pyridin-3-yl)pyrrolidin-2-one (1.5 g, 5.7504 mmol, 55.697% yield). ¹H NMR (DMSO-d₆, 400 MHz) δ 8.22 (s, 1H), 7.66 (d, J=2.3 Hz, 1H), 6.76 (s, 2H), 3.58 (t, J=7.0 Hz, 2H), 2.40-2.36 (m, 2H), 2.15-2.10 (m, 2H); LCMS (ESI): m/z 246.0[M+1]⁺.

1-(2-Isothiocyanato-5-(trifluoromethyl)pyridin-3-yl)pyrrolidin-2-one. To a solution of thiocarbonyl dichloride (0.94 mL, 12.23 mmol) in DCM (10 mL), a solution of 1-(2-amino-5-(trifluoromethyl)pyridin-3-yl)pyrrolidin-2-one (1.5 g, 6.12 mmol) in DCM (20 mL) was added at 0° C., the mixture was stirred at 20° C. for 16 h. The mixture was poured into aqueous NaHCO₃ at 0° C. The aqueous phase was extracted with DCM and the combined organic phase was concentrated under vacuum. The residue was purified by column chromatography to give 1-(2-isothiocyanato-5-(trifluoromethyl)pyridin-3-yl)pyrrolidin-2-one (900 mg, 2.8949 mmol, 47.323% yield). ¹H NMR (DMSO-d₆, 400 MHz) δ 8.80 (d, J=1.4 Hz, 1H), 8.45 (d, J=2.3 Hz, 1H), 3.87 (t, J=6.9 Hz, 2H), 2.49-2.47 (m, 2H), 2.21-2.16 (m, 2H); LCMS (ESI): m/z 287.9 [M+1]⁺.

1-(3-(2-Oxopyrrolidin-1-yl)-5-(trifluoromethyl)pyridin-2-yl)thiourea. Ammonium hydroxide (0.97 mL, 6.27 mmol) was added to a solution of 1-(2-isothiocyanato-5-(trifluoromethyl)pyridin-3-yl)pyrrolidin-2-one (900 mg, 3.13 mmol) in DCM (10 mL). The mixture was stirred at 20° C. for 2 h. The mixture was concentrated under vacuum and then dried by lyophilization to give [3-(2-oxopyrrolidin-1-yl)-5-(trifluoromethyl)-2-pyridyl]thiourea (900 mg, 2.9577 mmol, 94.403% yield). ¹H NMR (DMSO-d₆, 400 MHz) δ 10.17 (s, 1H), 9.45 (s, 1H), 9.27 (s, 1H), 8.62 (d, J=1.1 Hz, 1H), 8.23 (d, J=2.2 Hz, 1H), 3.80 (t, J=7.0 Hz, 2H), 2.49-2.46 (m, 2H), 2.20-2.17 (m, 2H); LCMS (ESI): m/z 304.9 [M+1]⁺.

1-(2-((4-(4-Isopropoxypyridin-2-yl)thiazol-2-yl)amino)-5-(trifluoromethyl)pyridin-3-yl)pyrrolidin-2-one. 2-Bromo-1-(4-isopropoxypyridin-2-yl)ethanone (475.01 mg, 1.84 mmol) was added to a solution of 1-(3-(2-oxopyrrolidin-1-yl)-5-(trifluoromethyl)pyridin-2-yl)thiourea (400. mg, 1.31 mmol) in EtOH (1 mL). The mixture was stirred at 80° C. for 1 h and then concentrated. The residue was purified by column chromatography to give the crude product. The crude product was purified by prep-HPLC then dried by lyophilization to give 1-(2-((4-(4-isopropoxypyridin-2-yl)thiazol-2-yl)amino)-5-(trifluoromethyl)pyridin-3-yl)pyrrolidin-2-one (207.34 mg, 0.4469 mmol, 33.997% yield). ¹H NMR (400 MHz, DMSO-d₆) δ 11.45-11.35 (m, 1H), 8.69 (d, J=1.0 Hz, 1H), 8.37 (d, J=5.8 Hz, 1H), 8.14 (d, J=2.0 Hz, 1H), 7.78 (s, 1H), 7.59 (d, J=2.5 Hz, 1H), 6.86 (dd, J₁=5.6, J₂=2.5 Hz, 1H), 4.79-4.73 (m, 1H), 3.78 (t, J=7.1 Hz, 2H), 2.48-2.46 (m, 2H), 2.24-2.20 (m, 2H), 1.33 (d, J=6.0 Hz, 6H); LCMS (ESI): m/z 464.2 [M+1]⁺.

### Example 60. 4-Methyl-N-[2-(3-methylpyridin-2-yl)-1,3-thiazol-4-yl]pyridin-2-amine

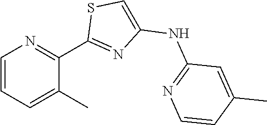 [SMILES: Cc1ccnc(Nc2csc(-c3ncccc3C)n2)c1]

2-(4-Bromo-1,3-thiazol-2-yl)-3-methylpyridine. CsF (312 mg, 2.06 mmol), Pd(PPh₃)₄ (60 mg, 0.05 mmol), and CuI (20 mg, 0.10 mmol) were added to a stirred, degassed solution of 2,4-dibromo-1,3-thiazole (250 mg, 1.03 mmol) and 3-methyl-2-(tributylstannyl)pyridine (432 mg, 1.13 mmol) in anhydrous DMF (3 mL) in a microwave vessel. The resulting mixture was irradiated under MW at 100° C. for 30 min. The reaction mixture was quenched with water and extracted with EtOAc. The combined organic layer was washed with brine, dried over anhydrous Na₂SO₄, filtered, and concentrated under reduced pressure. The crude mass was purified by column chromatography to get 2-(4-bromo-1,3-thiazol-2-yl)-3-methylpyridine (150 mg, 57%). MS (ESI): m/z 257.1 [M+2]+.

4-Methyl-N-[2-(3-methylpyridin-2-yl)-1,3-thiazol-4-yl]pyridin-2-amine. Cs₂CO₃ (383 mg, 1.17 mmol), Pd₂(dba)₃ (36 mg, 0.039 mmol), and xantphos (45 mg, 0.078 mmol) were added to a stirred, degassed solution of 2-(4-bromo-1,3-thiazol-2-yl)-3-methylpyridine (200 mg, 0.78 mmol) and 3-methylpyridin-2-amine (93 mg, 0.86 mmol) in dioxane (6 mL) in a sealed tube and the resulting mixture was heated at 100° C. for 16 h. The reaction mixture was filtered through a short pad of celite and washed with EtOAc. Organics were washed with water and brine, dried over anhydrous Na₂SO₄, filtered, and concentrated under reduced pressure. The crude mass was purified by column chromatography to get 4-methyl-N-[2-(3-methylpyridin-2-yl)-1,3-thiazol-4-yl]pyridin-2-amine (20 mg, 9%). MS (ESI): m/z 283.1 [M+1]⁺.

### Example 61. N-[2-(5-Methoxypyridin-2-yl)-1,3-thiazol-5-yl]-4-(propan-2-yloxy)pyridin-2-amine

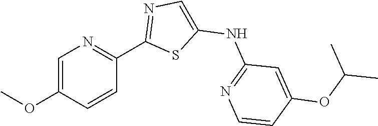 [SMILES: COc1ccc(-c2ncc(Nc3cc(OC(C)C)ccn3)s2)nc1]

Ethyl 2-[(5-methoxypyridin-2-yl)formamido]acetate. DIPEA (7.962 ml, 45.71 mmol) followed by EDCI·HCl (3.01 g, 15.672 mmol) and HOBT (2.1 g, 15.67 mmol) at 0° C. under an argon atmosphere were added to a stirred solution of 5-methoxypyridine-2-carboxylic acid (2 g, 13.06 mmol) in DCM (30 ml). The reaction mixture was stirred for 15 min and ethyl 2-aminoacetate hydrochloride (2.18 g, 15.67 mmol) was added and the mixture was stirred for another 16 h at 24° C. The reaction mixture was diluted with DCM, washed with water and brine, dried over anhydrous Na₂SO₄, filtered, and concentrated under reduced pressure. The crude mass was purified by column chromatography to afford ethyl 2-[(5-methoxypyridin-2-yl)formamido]acetate (2.3 g, 74%). MS (ESI): m/z 239.2 [M+1]⁺.

2-[(5-Methoxypyridin-2-yl)formamido]acetic acid. Lithium hydroxide mono hydrate (634 mg, 15.11 mmol) was added to a stirred solution of ethyl 2-[(5-methoxypyridin-2-yl)formamido]acetate (900 mg, 3.78 mmol) in THF:H₂O (1:1, 20 ml) and the reaction mixture was stirred at 24° C. for 16 h. The mixture was diluted with water and extracted with EtOAc. The aqueous layer was adjusted to pH 6 with citric acid and extracted with 15% IPA-DCM solution. The organics were washed with brine, dried over anhydrous Na₂SO₄, filtered, and concentrated under reduced pressure to get 2-[(5-methoxypyridin-2-yl)formamido]acetic acid (700 mg, 88%). MS (ESI): m/z 211.1 [M+1]⁺.

2-[(5-Methoxypyridin-2-yl)formamido]-N-[4-(propan-2-yloxy)pyridin-2-yl]acetamide. To a stirred solution of 2-[(5-methoxypyridin-2-yl)formamido]acetic acid (497 mg, 2.37 mmol) in DMF (8 ml) was added NMM (0.43 ml, 3.95 mmol) followed by 4-(propan-2-yloxy)pyridin-2-amine (200 mg, 1.32 mmol) and HATU (750 mg, 1.97 mmol) at 0° C. under an argon atmosphere. The resulting mixture was stirred at 24° C. for 16 h. The reaction mixture was diluted with EtOAc, washed with cold water and brine, dried over anhydrous Na₂SO₄, filtered, and concentrated under reduced pressure. The crude mass was purified by column chromatography to get 2-[(5-methoxypyridin-2-yl)formamido]-N-[4-(propan-2-yloxy)pyridin-2-yl]acetamide (200 mg, 44%). MS (ESI): m/z 345.3 [M+1]⁺.

N-[2-(5-Methoxypyridin-2-yl)-1,3-thiazol-5-yl]-4-(propan-2-yloxy)pyridin-2-amine. To a stirred solution of 2-[(5-methoxypyridin-2-yl)formamido]-N-[4-(propan-2-yloxy)pyridin-2-yl]acetamide (148 mg, 0.43 mmol) in toluene (5 ml), Lawesson's reagent (2,4-bis(4-methoxyphenyl)-1,3,2,4-dithiadiphosphetane-2,4-dithione) (348 mg, 0.86 mmol) and molecular sieves were added at 24° C. under a nitrogen atmosphere. The reaction mixture was heated at 110° C. for 7 h. It was then cooled to 24° C. and filtered through a short pad of celite. The filtrate was diluted with EtOAc, washed with water and brine, dried over anhydrous Na₂SO₄, filtered, and concentrated under reduced pressure. The crude mass was purified by prep HPLC to get N-[2-(5-methoxypyridin-2-yl)-1,3-thiazol-5-yl]-4-(propan-2-yloxy)pyridin-2-amine (42 mg, 28%). MS (ESI): m/z 343.0 [M+1]⁺.

### Example 62. 3-Methyl-N-{2-[5-(oxan-4-yloxy)pyridin-2-yl]-1,3-thiazol-5-yl}pyridin-2-amine

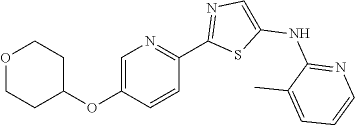 [SMILES: Cc1cccnc1Nc1cnc(-c2ccc(OC3CCOCC3)cn2)s1]

Methyl 5-(oxan-4-yloxy)pyridine-2-carboxylate. To a stirred solution of methyl 5-hydroxypyridine-2-carboxylate (2.5 g, 16.33 mmol) in DMF (30 ml) was added oxan-4-yl methanesulfonate (3.53 gm, 19.59 mmol) and K₂CO₃ (6.8 gm, 48.98 mmol). The resulting mixture was heated at 80° C. for 16 h under nitrogen atmosphere. The reaction mixture was cooled to 24° C., diluted with water, and extracted with EtOAc. The combined organic layer was washed with water and brine, dried over Na₂SO₄, filtered, and concentrated under reduced pressure. The crude mass was purified by column chromatography to afford methyl 5-(oxan-4-yloxy)pyridine-2-carboxylate (1.5 g, 39%). MS (ESI): m/z 238.0 [M+1]⁺.

5-(Oxan-4-yloxy)pyridine-2-carboxylic acid. Lithium hydroxide mono hydrate (1.06 g, 25.32 mmol) was added to a stirred solution of methyl 5-(oxan-4-yloxy)pyridine-2-carboxylate (1.5 g, 6.33 mmol) in a mixture of THF:H₂O (1:1, 20 ml) and the reaction mixture was stirred at 24° C. for 6 h. The mixture was diluted with water and extracted with EtOAc. The aqueous layer was adjusted to pH 6 with citric acid and extracted with 15% IPA-DCM solution. Organics were washed with brine, dried over anhydrous Na₂SO₄, filtered, and concentrated under reduced pressure to get 5-(oxan-4-yloxy)pyridine-2-carboxylic acid (1.2 g, 85%). MS (ESI): m/z 224.0 [M+1]⁺.

Ethyl 2-{[5-(oxan-4-yloxy)pyridin-2-yl]formamido}acetate. DIPEA (3.2 ml, 18.83 mmol) followed by EDCI·HCl (1.24 g, 6.46 mmol) and HOBT (873 mg, 6.457 mmol) at 0° C. under argon atmosphere were added to a stirred solution of 5-(oxan-4-yloxy)pyridine-2-carboxylic acid (1.2 g, 5.38 mmol) in DCM (15 ml). The resultant mixture was stirred for 15 min and then ethyl 2-aminoacetate hydrochloride (900 mg, 6.457 mmol) was added and reaction mixture was stirred at 24° C. for 16 h. The reaction mixture was diluted with DCM, washed with water and brine, dried over anhydrous Na₂SO₄, filtered, and concentrated under reduced pressure. The crude mass was purified by column chromatography to afford ethyl 2-{[5-(oxan-4-yloxy)pyridin-2-yl]formamido}acetate (900 mg, 54%). MS (ESI): m/z 309.3 [M+1]⁺.

2-{[5-(Oxan-4-yloxy)pyridin-2-yl]formamido}acetic acid. Lithium hydroxide mono hydrate (491 mg, 11.69 mmol) was added to a stirred solution of ethyl 2-{[5-(oxan-4-yloxy)pyridin-2-yl]formamido}acetate (900 mg, 2.92 mmol) in THF-H₂O (1:1, 20 ml). The resultant mixture was stirred at 24° C. for 5 h. The mixture was diluted with water and extracted with EtOAc. The aqueous layer was adjusted to pH 6 with citric acid and extracted with 15% IPA-DCM solution. The organic layer was washed with brine, dried over anhydrous Na₂SO₄, filtered, and concentrated under reduced pressure to get 2-{[5-(oxan-4-yloxy)pyridin-2-yl]formamido}acetic acid (780 mg, 95%). MS (ESI): m/z 281.2 [M+1]⁺.

N-(3-Methylpyridin-2-yl)-2-{[5-(oxan-4-yloxy)pyridin-2-yl]formamido}acetamide. NMM (0.8 ml, 6.94 mmol) followed by 3-methylpyridin-2-amine (250 mg, 2.31 mmol) and HATU (1.32 g, 3.47 mmol) at 0° C. under argon atmosphere were added to a stirred solution of 2-{[5-(oxan-4-yloxy)pyridin-2-yl]formamido}acetic acid (778 mg, 2.78 mmol) in DMF (10 ml). The resulting mixture was stirred at 24° C. for 16 h. The reaction mixture was diluted with EtOAc, washed with cold water and brine, dried over anhydrous Na₂SO₄, filtered, and concentrated under reduced pressure. The crude mass was purified by column chromatography to get N-(3-methylpyridin-2-yl)-2-{[5-(oxan-4-yloxy)pyridin-2-yl]formamido}acetamide (175 mg, 20%). MS (ESI): m/z 371.3 [M+1]⁺.

3-Methyl-N-{2-[5-(oxan-4-yloxy)pyridin-2-yl]-1,3-thiazol-5-yl}pyridin-2-amine. To a stirred solution of N-(3-methylpyridin-2-yl)-2-{[5-(oxan-4-yloxy)pyridin-2-yl]formamido}acetamide (175 mg, 0.47 mmol) in toluene (5 ml) was added Lawesson's reagent (383 mg, 0.94 mmol) and molecular sieves at 24° C. under nitrogen atmosphere. The reaction mixture was heated at 110° C. for 7 h. It was cooled to 24° C. and filtered through a short pad of celite. The filtrate was diluted with EtOAc, washed with water and brine, dried over anhydrous Na₂SO₄, filtered, and concentrated under reduced pressure. The crude mass was purified by prep HPLC to get 3-methyl-N-{2-[5-(oxan-4-yloxy)pyridin-2-yl]-1,3-thiazol-5-yl}pyridin-2-amine (20 mg, 11%). MS (ESI): m/z 369.2 [M+1]⁺.

### Example 63. 4-Phenyl-N-[3-(pyridin-2-yl)-1,2-thiazol-5-yl]pyridin-2-amine

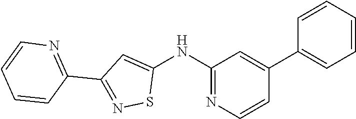 [SMILES: c1ccc(-c2ccnc(Nc3cc(-c4ccccn4)ns3)c2)cc1]

(2Z)-3-amino-3-(pyridin-2-yl)prop-2-enenitrile. Acetonitrile (5 mL, 96.05 mmol) followed by potassium-tertbutoxide (16.17 g, 144.07 mmol) in portions were added to a stirred solution of pyridine-2-carbonitrile (5 g, 48.02 mmol) in benzene (90 mL). The reaction mixture was stirred at 24° C. for 16 h. The reaction mixture was cooled to 0° C. and quenched with cold NaHCO₃ solution and extracted with MTBE. The organic layer was dried over Na₂SO₄, filtered and concentrated under reduced pressure. The crude mass was triturated with 10-20% MTBE-hexane to afford (2Z)-3-amino-3-(pyridin-2-yl)prop-2-enenitrile (5.2 g, 75%). MS (ESI): m/z 146.2 [M+1]⁺.

(2Z)-3-amino-3-(pyridin-2-yl)prop-2-enethioamide. Na₂S (4.03 g, 51.66 mmol) was added in portions to a stirred solution of P2S5 (11.48 g, 51.66 mmol) in THE (100 mL) and the resulting mixture was stirred for 20 min at 24° C. The reaction mixture was cooled to 0° C. and (2Z)-3-amino-3-(pyridin-2-yl)prop-2-enenitrile (1.5 g, 10.33 mmol) was added in portions. The resulting mixture was stirred at 24° C. for 15 min, was slowly quenched with NaHCO₃ solution, and was extracted with EtOAc. The combined organic layer was washed with water and brine, dried over anhydrous Na₂SO₄, filtered, and concentrated under reduced pressure to afford (2Z)-3-amino-3-(pyridin-2-yl)prop-2-enethioamide (1.6 g, 86%). MS (ESI): m/z 180.1 [M+1]⁺.

3-(Pyridin-2-yl)-1,2-thiazol-5-amine. H₂O₂ (30% aqueous solution, 1.2 mL, 11.72 mmol) was slowly added to a stirred solution of (2Z)-3-amino-3-(pyridin-2-yl)prop-2-enethioamide (1.4 g, 7.81 mmol) in MeOH (100 mL). The reaction mixture was stirred at 24° C. for 1 h. After completion, the reaction mixture was quenched with an aqueous solution of Na₂S203 and extracted with EtOAc. The combined organic layer was washed with water and brine, dried over anhydrous Na₂SO₄, filtered, and concentrated under reduced pressure. The crude product was purified by column chromatography to afford 3-(pyridin-2-yl)-1,2-thiazol-5-amine (425 mg, 31%). MS (ESI): m/z 177.7 [M+1]⁺.

4-Phenyl-N-[3-(pyridin-2-yl)-1,2-thiazol-5-yl]pyridin-2-amine. Cs₂CO₃ (519.30 mg, 1.60 mmol) was added to a stirred solution of 3-(pyridin-2-yl)-1,2-thiazol-5-amine (150 mg, 0.64 mmol) and 2-bromo-4-phenylpyridine (90.5 mg, 0.51 mmol) in toluene (5 mL), and the reaction mixture was degassed with argon for 10 min. Pd₂(dba)₃ (58.53 mg. 0.06 mmol) and BINAP (79.59 mg, 0.13 mmol) were added and the resulting mixture was heated at 100° C. for 16 h in a sealed tube. After completion, the reaction mixture was filtered through a short pad of celite. The filtrate was diluted with water and extracted with EtOAc. The combined organic layer was washed with water and brine, dried over anhydrous Na₂SO₄, filtered, and evaporated under reduced pressure. The crude product was purified to afford 4-phenyl-N-[3-(pyridin-2-yl)-1,2-thiazol-5-yl]pyridin-2-amine (30 mg, 14%). MS (ESI): m/z 331.0 [M+1]⁺.

### Example 64. 4-Methyl-N-[3-(3-methylpyridin-2-yl)-1,2-thiazol-5-yl]pyridin-2-amine

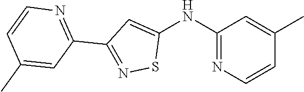

(2Z)-3-amino-3-(3-methylpyridin-2-yl)prop-2-enenitrile. Acetonitrile (3.34 mL, 63.50 mmol) followed by potassium-tertbutoxide (14.25 g, 127.01 mmol) were added portion wise to a stirred solution of 3-methylpyridine-2-carbonitrile (5 g, 42.33 mmol) in toluene (150 mL) at 0° C. The reaction mixture was warmed to 24° C. and stirred for 16 h. The reaction mixture was quenched with NaHCO₃ solution and extracted with EtOAc. The combined organic layer was washed with water and brine, dried over anhydrous Na₂SO₄, filtered, and concentrated under reduced pressure. The crude mass was purified by column chromatography to afford (2Z)-3-amino-3-(3-methylpyridin-2-yl)prop-2-enenitrile (3.2 g, 47%). MS (ESI): m/z 160.2 [M+1]⁺.

(2Z)-3-amino-3-(3-methylpyridin-2-yl)prop-2-enethioamide. Anhydrous Na₂S (4.9 g, 62.81 mmol) was added portion wise to a stirred solution of P2S5 (13.97 g, 62.81 mmol) in THE (120 mL) and the solution was stirred for 20 min at 24° C. The reaction mixture was cooled to 0° C. and (2Z)-3-amino-3-(3-methylpyridin-2-yl)prop-2-enenitrile (2 g, 12.56 mmol) was added in portions. The resulting mixture was stirred for 15 min. The reaction mixture was slowly quenched with NaHCO₃ solution and extracted with EtOAc. The combined organic layer was washed with water and brine, dried over anhydrous Na₂SO₄, filtered, and concentrated under reduced pressure to afford (2Z)-3-amino-3-(3-methylpyridin-2-yl)prop-2-enethioamide (1.0 g, 41%). MS (ESI): m/z 194.2 [M+1]⁺.

3-(3-Methylpyridin-2-yl)-1,2-thiazol-5-amine. H₂O₂ (30% aqueous solution, 1.89 mL, 18.62 mmol) was slowly added to a stirred solution of (2Z)-3-amino-3-(3-methylpyridin-2-yl)prop-2-enethioamide (2.4 g, 12.41 mmol) in MeOH (50 mL). The reaction mixture was stirred for 1 h at 24° C. After completion, the reaction mixture was quenched with aqueous Na₂S203 solution and extracted with EtOAc. The combined organic layer was washed with water and brine, dried over anhydrous Na₂SO₄, filtered, and concentrated under reduced pressure. The crude mass was purified by column chromatography to afford 3-(3-methylpyridin-2-yl)-1,2-thiazol-5-amine (800 mg, 34%). MS (ESI): m/z 192.0 [M+1]⁺.

4-Methyl-N-[3-(3-methylpyridin-2-yl)-1,2-thiazol-5-yl]pyridin-2-amine. To a stirred solution of 3-(3-methylpyridin-2-yl)-1,2-thiazol-5-amine (200 mg, 1.04 mmol) and 2-bromo-4-methylpyridine (215.8 mg, 1.25 mmol) in 1,4-dioxane (5 mL), Cs₂CO₃ (849.6 mg, 2.60 mmol) was added and the reaction mixture was degassed with argon for 10 min. Pd₂(dba)₃ (95.75 mg. 0.10 mmol) and Xantphos (120.88 mg, 0.20 mmol) were added and the resulting mixture was heated at 100° C. for 16 h in sealed tube. After completion, it was filtered through a short pad of celite. The filtrate was diluted with water and extracted with EtOAc. The combined organic layer was washed with water and brine, dried over anhydrous Na₂SO₄, filtered, and evaporated under reduced pressure. The crude mass was purified by column chromatography to afford 4-methyl-N-[3-(3-methylpyridin-2-yl)-1,2-thiazol-5-yl]pyridin-2-amine (80 mg, 27%). MS (ESI): m/z 283.3 [M+1]⁺.

### Example 65. 3-Methyl-N-{3-[5-(oxan-4-yloxy)pyridin-2-yl]-1,2-thiazol-5-yl}pyridin-2-amine

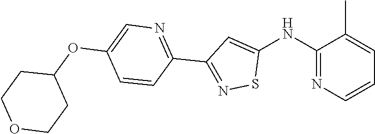 [SMILES: Cc1cccnc1Nc1cc(-c2ccc(OC3CCOCC3)cn2)ns1]

5-Hydroxypyridine-2-carbonitrile. To a stirred solution of 5-aminopyridine-2-carbonitrile (1 g, 8.39 mmol) in water (15 mL) was added concentrated H₂SO₄ (4.2 mL) at 0° C. followed by an aqueous solution of NaNO₂ (636 mg, 9.23 mmol, in 6 mL water) dropwise. The reaction mixture was stirred at 0° C. for 30 min. The resulting reaction mixture was poured into a solution of H₂SO₄ (1 mL) and water (11 mL) and heated at 80° C. for 1 h. The reaction was cooled to 24° C. and extracted with EtOAc. The combined organic part was washed with saturated brine solution, dried over anhydrous Na₂SO₄, filtered, and concentrated under reduced pressure to get crude 5-hydroxypyridine-2-carbonitrile (650 mg, 64%). MS (ESI): m/z 118.6 [M-1]⁺.

5-(Oxan-4-yloxy)pyridine-2-carbonitrile. K₂CO₃ (1.4 g, 9.91 mmol) was added to a stirred solution of 5-hydroxypyridine-2-carbonitrile (400 mg, 3.33 mmol) and oxan-4-yl methanesulfonate (660 mg, 3.66 mmol) in anhydrous DMF (4 mL) at 24° C. under an argon atmosphere. The resulting mixture was heated at 80° C. for 16 h. The reaction mixture was quenched with water and extracted with EtOAc. The combined organic part was washed with cold water and brine, dried over anhydrous Na₂SO₄, filtered, and concentrated under reduced pressure. The crude mass was purified by column chromatography to afford 5-(oxan-4-yloxy)pyridine-2-carbonitrile (450 mg, 66%). MS (ESI): m/z 205.2 [M+1]⁺.

(2Z)-3-amino-3-[5-(oxan-4-yloxy)pyridin-2-yl]prop-2-enenitrile. KOtBu (1.6 g, 14.68 mmol) was added to a stirred solution of 5-(oxan-4-yloxy)pyridine-2-carbonitrile (1 g, 4.89 mmol) in benzene (20 mL) and MeCN (1 mL) at 24° C. under an argon atmosphere and the resulting mixture was stirred for 16 h. Volatiles were removed under reduced pressure and residue was dissolved in EtOAc. The organic layer was washed with water and brine, dried over anhydrous Na₂SO₄, filtered, and concentrated under reduced pressure. The crude mass was purified by column chromatography to afford (2Z)-3-amino-3-[5-(oxan-4-yloxy)pyridin-2-yl]prop-2-enenitrile (800 mg, 59%). MS (ESI): m/z 246.1 [M+1]⁺.

(2Z)-3-amino-3-[5-(oxan-4-yloxy)pyridin-2-yl]prop-2-enethioamide. Na₂S (795 mg, 10.19 mmol) was added to a stirred suspension of P2S5 (4.5 g, 10.19 mmol) in THE (20 mL) and the mixture was stirred at 24° C. for 10 min. The reaction mixture was then cooled to 4° C. (2Z)-3-amino-3-[5-(oxan-4-yloxy)pyridin-2-yl]prop-2-enenitrile (500 mg, 2.03 mmol) was added and the mixture was stirred for 5 min at the same temperature. The reaction mixture was quenched with ice-cold saturated NaHCO₃ solution and extracted with EtOAc. The combined organic part was with brine, dried over Na₂SO₄, filtered, and concentrated under reduced pressure to afford (2Z)-3-amino-3-[5-(oxan-4-yloxy)pyridin-2-yl]prop-2-enethioamide (500 mg, crude). MS (ESI): m/z 280.3 [M+1]⁺.

3-[5-(Oxan-4-yloxy)pyridin-2-yl]-1,2-thiazol-5-amine. H₂O₂ (0.3 mL, 3.05 mmol, 30% aqueous solution) was added dropwise at 0° C. to a stirred solution of (2Z)-3-amino-3-[5-(oxan-4-yloxy)pyridin-2-yl]prop-2-enethioamide (569 mg, 2.04 mmol) in MeOH (10 mL). The resulting reaction mixture was stirred at 24° C. for 1 h. The reaction mixture was quenched with saturated aqueous solution of sodium thiosulphate at 0° C. and extracted with EtOAc. The combined organic layer was washed with brine, dried over anhydrous Na₂SO₄, filtered, and concentrated under reduced pressure. The crude mass was purified by column chromatography to get pure 3-[5-(oxan-4-yloxy)pyridin-2-yl]-1,2-thiazol-5-amine (130 mg, 23%). MS (ESI): m/z 278.3 [M+1]⁺.

3-Methyl-N-{3-[5-(oxan-4-yloxy)pyridin-2-yl]-1,2-thiazol-5-yl}pyridin-2-amine. Cs₂CO₃ (176 mg, 0.54 mmol), Pd₂(dba)₃ (16 mg, 0.18 mmol), and xantphos (20 mg, 0.036 mmol) were added to a stirred degassed solution of 3-[5-(oxan-4-yloxy)pyridin-2-yl]-1,2-thiazol-5-amine (150 mg, 0.36 mmol) and 2-bromo-3-methyl-pyridine (68 mg, 0.39 mmol) in dioxane (5 mL) in a sealed tube and the mixture was heated at 100° C. for 16 h. The reaction mixture was filtered through a short pad of celite and washed with EtOAc. The filtrate was washed with water and brine, dried over Na₂SO₄, filtered, and concentrated under reduced pressure. The crude mass was purified by column chromatography to afford 3-methyl-N-{3-[5-(oxan-4-yloxy)pyridin-2-yl]-1,2-thiazol-5-yl}pyridin-2-amine (40 mg, 56%). MS (ESI): m/z 369.4 [M+1]⁺.

### Example 66. 4-Methyl-N-[2-(3-methylpyridin-2-yl)-1,3-thiazol-5-yl]pyridin-2-amine

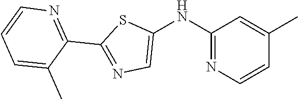 [SMILES: Cc1ccnc(Nc2cnc(-c3ncccc3C)s2)c1]

Ethyl 2-[(3-methylpyridin-2-yl)formamido]acetate. DIPEA (1.91 mL, 10.94 mmol), EDCI·HCl (1.68 g, 8.75 mmol), and HOBT (1.18 g, 8.75 mmol) were added to a stirred solution of 3-Methyl-pyridine-2-carboxylic acid (1.0 g, 7.29 mmol) in DCM (20 mL) and the mixture was stirred at 24° C. for 10 min. Amino-acetic acid ethyl ester hydrochloride (1.51 g, 10.94 mmol) was added and the resulting reaction mixture was stirred at 24° C. for 32 h. After completion, the reaction mixture was diluted with water and extracted with DCM. The combined organic layer was washed with brine, dried over Na₂SO₄, filtered, and concentrated under reduced pressure. The crude material was purified by column chromatography to afford ethyl 2-[(3-methylpyridin-2-yl)formamido]acetate (700 mg, 43%). MS (ESI): m/z 223 [M+1]⁺.

2-[(3-Methylpyridin-2-yl)formamido]acetic acid. Lithium hydroxide mono hydrate (0.38 g, 9.01 mmol) was added to a stirred solution of ethyl 2-[(3-methylpyridin-2-yl)formamido]acetate (1 g, 4.51 mmol) in a mixture of THE and water (1:1; 16 mL) and the resulting reaction mixture was stirred at 24° C. for 16 h. After completion, the reaction mixture was concentrated under reduced pressure, acidified with saturated citric acid solution and extracted with 10-15% IPA-DCM. The combined organic layer was washed with brine, dried over Na₂SO₄, filtered, and concentrated under reduced pressure to afford 2-[(3-methylpyridin-2-yl)formamido]acetic acid (550 mg, 63%). MS (ESI): m/z 192.7 [M-1]*.

N-(4-Methylpyridin-2-yl)-2-[(3-methylpyridin-2-yl)formamido]acetamide. NMM (1.7 mL, 15.46 mmol) followed by HATU (2.94 g, 7.73 mmol) were added to a stirred solution of 2-[(3-methylpyridin-2-yl)formamido]acetic acid (1.0 g, 5.16 mmol) in DMF (15 mL) and the mixture was stirred at 24° C. for 10 min. 4-Methyl-pyridin-2-ylamine (612 mg, 5.67 mmol) was added and the resulting reaction mixture was stirred for 32 h. After completion, the reaction mixture was diluted with cold water and extracted with 10% MeOH in DCM. The combined organic layer was washed with brine, dried over Na₂SO₄, filtered, and concentrated under reduced pressure. The crude material was purified by column chromatography to afford N-(4-methylpyridin-2-yl)-2-[(3-methylpyridin-2-yl)formamido]acetamide (500 mg, 34%). MS (ESI): m/z 285.2 [M+1]⁺.

4-Methyl-N-[2-(3-methylpyridin-2-yl)-1,3-thiazol-5-yl]pyridin-2-amine. To a stirred solution of N-(4-methylpyridin-2-yl)-2-[(3-methylpyridin-2-yl)formamido]acetamide (400 mg, 1.41 mmol) in toluene (15 mL) was added Lawesson's reagent (1.14 g, 2.82 mmol) followed by molecular sieves (2 g) at 24° C. The resulting reaction mixture was heated at 110° C. for 6 h. After completion, the reaction mixture was filtered through a small pad of celite. The filtrate was concentrated under reduced pressure, diluted with water and extracted with EtOAc. The combined organic layer was dried over Na₂SO₄, filtered, and concentrated under reduced pressure. The crude compound was purified by prep HPLC to get 4-methyl-N-[2-(3-methylpyridin-2-yl)-1,3-thiazol-5-yl]pyridin-2-amine (20 mg, yield: 5%). MS (ESI): m/z 283 [M+1]⁺.

### Example 67. 4-Phenyl-N-(3-(pyridin-2-yl)-1H-pyrazol-5-yl)pyridin-2-amine

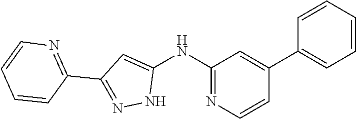 [SMILES: c1ccc(-c2ccnc(Nc3cc(-c4ccccn4)n[nH]3)c2)cc1]

3, 3-Bis (methylsulfanyl)-1-(pyridin-2-yl) prop-2-en-1-one. NaH (60% in oil, 6.9 g, 173.35 mmol) was added in portions to dry DMSO (30 mL) at 24° C. 1-(Pyridin-2-yl)ethan-1-one (5 g, 41.27 mmol) was added slowly over 15 min and the mixture was stirred for an additional 30 min at 24° C. CS₂ (3.14 g, 41.27 mmol) was slowly added to the reaction mixture over 30 min followed by addition of Mel (5.6 ml, 90.80 mmol) to the reaction mixture. The mixture was then stirred for 16 h. After completion, the reaction mixture was diluted with water and extracted with DCM. The combined organic layer was washed with water, brine, dried over anhydrous Na₂SO₄, filtered, and evaporated under reduced pressure. The crude product was purified by column chromatography to afford 3,3-bis(methylsulfanyl)-1-(pyridin-2-yl)prop-2-en-1-one (1.6 g, 17%). MS (ESI): m/z 226.0 [M+1]⁺.

(2E)-3-(methylsulfanyl)-3-[(4-phenylpyridin-2-yl)amino]-1-(pyridin-2-yl)prop-2-en-1-one. To a stirred solution of 4-phenylpyridin-2-amine (200 mg, 1.18 mmol) in THE (8 mL) was added nBuLi (2.4 M in hexane, 1 mL, 2.4 mmol) dropwise at 0° C. and the mixture was stirred for 30 min under cooling conditions. A solution of 3,3-bis(methylsulfanyl)-1-(pyridin-2-yl)prop-2-en-1-one (266 mg, 1.18 mmol) in THE (2 mL) was added at 0° C. and the resulting mixture was heated at 60° C. for 1 h. After completion, the reaction mixture was quenched with saturated NH₄Cl solution and extracted with EtOAc. The combined organic layer was washed with water and brine, dried over anhydrous Na₂SO₄, filtered, and concentrated under reduced pressure. The crude mass was purified by column chromatography to afford (2E)-3-(methylsulfanyl)-3-[(4-phenylpyridin-2-yl)amino]-1-(pyridin-2-yl)prop-2-en-1-one (150 mg, 36%). MS (ESI): m/z 348.0 [M+1]⁺.

4-Phenyl-N-[3-(pyridin-2-yl)-1H-pyrazol-5-yl]pyridin-2-amine. To a stirred solution of (2E)-3-(methylsulfanyl)-3-[(4-phenylpyridin-2-yl)amino]-1-(pyridin-2-yl)prop-2-en-1-one (200 mg, 0.57 mmol) in t-butanol (10 mL) was added hydrazine monohydrate (34 mg, 0.69 mmol) and AcOH (0.1 mL, 1.73 mmol) and the reaction mixture was heated to reflux for 1 h. After completion, the reaction mixture was cooled to 24° C., adjusted to a basic pH level with NaHCO₃, and extracted with EtOAc. The combined organic layer was washed with water and brine, dried over anhydrous Na₂SO₄, filtered, and concentrated under reduced pressure. The crude compound was purified by column chromatography followed by trituration with 30% ether-pentane to afford 4-phenyl-N-[3-(pyridin-2-yl)-1H-pyrazol-5-yl]pyridin-2-amine (20 mg, 11%). MS (ESI): m/z 313.9 [M+1]⁺.

### Example 68. 4-Methyl-N-[3-(3-methylpyridin-2-yl)-1H-pyrazol-5-yl]pyridin-2-amine

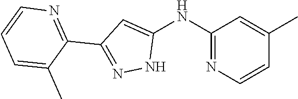 [SMILES: Cc1ccnc(Nc2cc(-c3ncccc3C)n[nH]2)c1]

1-(3-Methylpyridin-2-yl)ethan-1-one. MeMgBr (3 M in THF, 17 ml, 51 mmol) was added dropwise at 0° C. under argon atmosphere to a stirred solution of 3-methylpyridine-2-carbonitrile (3 g, 25.39 mmol) in anhydrous THE (30 mL) and the mixture was then stirred at 0° C. for 2 h. The reaction mixture was quenched with NH₄Cl and extracted with EtOAc. The combined organic layer was washed with water and brine, dried over anhydrous Na₂SO₄, filtered, and concentrated under reduced pressure. The crude product was purified by column chromatography to get pure 1-(3-methylpyridin-2-yl)ethan-1-one (2.7 g, 79%). MS (ESI): m/z 136.0 [M+1]⁺.

1-(3-Methylpyridin-2-yl)-3,3-bis(methylsulfanyl)prop-2-en-1-one. NaH (60% in oil, 1.48 g, 36.99 mmol) was added in portions in dry DMSO (10 mL) at 24° C. 1-(3-methylpyridin-2-yl)ethan-1-one (2 g, 14.79 mmol) was added slowly over 15 min and the mixture was stirred for an additional 30 min at 24° C. CS₂ (0.89 g, 14.80 mmol) was added slowly to the reaction mixture over 30 min followed by addition of Mel (2.03 ml, 32.55 mmol) and the mixture was stirred for 16 h. After completion, the reaction mixture was diluted with water and extracted with DCM. The combined organic layer was washed with water and brine, dried over anhydrous Na₂SO₄, filtered, and evaporated under reduced pressure. The crude product was purified by column chromatography to afford 1-(3-methylpyridin-2-yl)-3,3-bis(methylsulfanyl)prop-2-en-1-one (1.5 g, 42%). MS (ESI): m/z 240.2 [M+1]⁺.

(2E)-1-(3-methylpyridin-2-yl)-3-[(4-methylpyridin-2-yl)amino]-3-(methylsulfanyl)prop-2-en-1-one. nBuLi (2.5 M in hexane, 1.7 mL, 4.2 mmol) was added dropwise at 0° C. to a stirred solution of 4-methylpyridin-2-amine (271 mg, 2.50 mmol) in THE (5 mL) and the mixture was stirred for 30 min under cooling conditions. A solution of 1-(3-methylpyridin-2-yl)-3,3-bis(methylsulfanyl)prop-2-en-1-one (500 mg, 2.08 mmol) in THE (3 mL) was added at 0° C. The resulting mixture was heated at 60° C. for 1 h. After completion, the reaction mixture was quenched with saturated NH₄Cl solution and extracted with EtOAc. The combined organic layer was washed with water and brine, dried over anhydrous Na₂SO₄, filtered, and concentrated under reduced pressure to afford (2E)-1-(3-methylpyridin-2-yl)-3-[(4-methylpyridin-2-yl)amino]-3-(methylsulfanyl)prop-2-en-1-one (500 mg, crude). MS (ESI): m/z 300.3 [M+1]⁺.

4-Methyl-N-[3-(3-methylpyridin-2-yl)-1H-pyrazol-5-yl]pyridin-2-amine. To a stirred solution of (2E)-1-(3-methylpyridin-2-yl)-3-[(4-methylpyridin-2-yl)amino]-3-(methylsulfanyl)prop-2-en-1-one (150 mg, 0.50 mmol) in t-butanol (10 mL) was added hydrazine monohydrate (30 mg, 0.60 mmol) and AcOH (0.09 mL, 1.50 mmol) and the reaction mixture was heated to reflux for 1 h. After completion, the reaction mixture was cooled to 24° C., adjusted to a basic pH with saturated NaHCO₃ solution and extracted with EtOAc. The combined organic layer was washed with brine, dried over anhydrous Na₂SO₄, filtered, and concentrated under reduced pressure. The crude compound was purified by column chromatography followed by trituration to afford 4-methyl-N-[3-(3-methylpyridin-2-yl)-1H-pyrazol-5-yl]pyridin-2-amine (25 mg, 19%). MS (ESI): m/z 266.0 [M+1]⁺.

### Example 69. N-(1-Methyl-5-(pyridin-2-yl)-1H-pyrazol-3-yl)-4-phenylpyridin-2-amine and -(1-Methyl-3-(pyridin-2-yl)-1H-pyrazol-5-yl)-4-phenylpyridin-2-amine

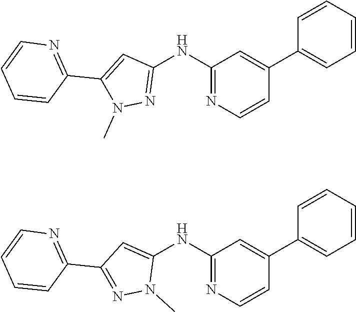 [SMILES: Cn1nc(Nc2cc(-c3ccccc3)ccn2)cc1-c1ccccn1 ; Cn1nc(-c2ccccn2)cc1Nc1cc(-c2ccccc2)ccn1]

N-[1-Methyl-3-(pyridin-2-yl)-1H-pyrazol-5-yl]-4-phenylpyridin-2-amine and N-[1-methyl-5-(pyridin-2-yl)-1H-pyrazol-3-yl]-4-phenylpyridin-2-amine. To a stirred solution of (2E)-3-(methylsulfanyl)-3-[(4-phenylpyridin-2-yl)amino]-1-(pyridin-2-yl)prop-2-en-1-one (200 mg, 0.57 mmol) in t-butanol (10 mL) was added methyl hydrazine (0.12 mL, 1.04 mmol) and AcOH (0.1 mL, 1.73 mmol) and the reaction mixture was heated to reflux for 1 h. After completion, the reaction mixture was cooled to 24° C., adjusted to a basic pH with saturated NaHCO₃ solution, and extracted with EtOAc. The combined organic layer was washed with water and brine, dried over anhydrous Na₂SO₄, filtered, and concentrated under reduced pressure. The crude compound was purified by column chromatography followed by trituration with 5% EtOAc/pentane to afford N-[1-methyl-3-(pyridin-2-yl)-1H-pyrazol-5-yl]-4-phenylpyridin-2-amine (40 mg, 14%) and N-[1-methyl-5-(pyridin-2-yl)-1H-pyrazol-3-yl]-4-phenylpyridin-2-amine (60 mg, 21%). MS (ESI): m/z 328.2 [M+1]⁺.

### Example 70. N-[5-(5-Methoxypyridin-2-yl)-1,3-oxazol-2-yl]-4-(propan-2-yloxy)pyridin-2-amine

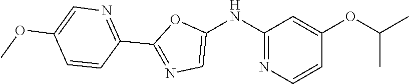 [SMILES: COc1ccc(-c2ncc(Nc3cc(OC(C)C)ccn3)o2)nc1]

1-(5-Methoxypyridin-2-yl)ethan-1-one. To a stirred solution of 5-methoxypyridine-2-carbonitrile (3 g, 22.36 mmol) in THE (75 mL) was added MeMgBr (3 M in ether, 16.9 mL, 50.79 mmol) dropwise at 0° C. under an argon atmosphere. The resulting mixture was stirred at 0° C. for 2 h. The reaction mixture was quenched with NH₄Cl and extracted with EtOAc. The combined organic layer was washed with water and brine, dried over anhydrous Na₂SO₄, filtered, and concentrated under reduced pressure. The crude product was purified by column chromatography to afford 1-(5-methoxypyridin-2-yl)ethan-1-one (1.5 g, 39%). MS (ESI): m/z 152.2 [M+1]⁺.

2-Bromo-1-(5-methoxypyridin-2-yl)ethan-1-one. HBr in glacial acetic acid (33%, 5 mL) was added drop wise at 0° C. under an argon atmosphere to a stirred solution of 1-(5-methoxypyridin-2-yl)ethan-1-one (800 mg, 5.29 mmol) in glacial acetic acid (10 mL). A solution of bromine (0.3 mL, 5.82 mmol) in AcOH (1 mL) was added to the reaction mixture dropwise at 0° C. and the resulting mixture was stirred at 24° C. for 2 h. The residue was filtered, washed with Et₂O and dried to afford 2-bromo-1-(5-methoxypyridin-2-yl)ethan-1-one hydrobromide (1.3 g, 78%). MS (ESI): m/z 230.1 [M+1]⁺.

2-Azido-1-(5-methoxypyridin-2-yl)ethan-1-one. NaHCO₃ (486 mg, 2.89 mmol) and NaN₃ (188 mg, 2.89 mmol) were added to a stirred solution of 2-bromo-1-(5-methoxypyridin-2-yl)ethan-1-one hydrobromide (400 mg, 1.45 mmol) in EtOH (15 mL) at 24° C. The resulting reaction mixture was stirred at the same temperature for 4 h. The reaction mixture was diluted with DCM and washed with water. The organic layer was washed with brine, dried over anhydrous Na₂SO₄, filtered, and concentrated under reduced pressure to get 2-azido-1-(5-methoxypyridin-2-yl)ethan-1-one (276 mg, 99%). MS (ESI): m/z 193.2 [M+1]⁺.

2-Isothiocyanato-4-(propan-2-yloxy)pyridine. 1-[(2-oxo-1,2-dihydropyridin-1-yl)carbothioyl]-1,2-dihydropyridin-2-one (397 g, 1.71 mmol) was added to a stirred solution of 4-(propan-2-yloxy)pyridin-2-amine (250 mg, 1.64 mmol) in DCM (10 mL) at 24° C. and the reaction mixture was stirred for 3 h. After completion, the reaction mixture was concentrated under reduced pressure and the crude product was purified by column chromatography to get 2-isothiocyanato-4-(propan-2-yloxy)pyridine (200 mg, 62%). MS (ESI): m/z 195.1 [M+1]⁺.

N-[5-(5-Methoxypyridin-2-yl)-1,3-oxazol-2-yl]-4-(propan-2-yloxy)pyridin-2-amine. PPh₃ (490 mg, 1.87 mmol) was added at 24° C. to a stirred solution of 2-azido-1-(5-methoxypyridin-2-yl)ethan-1-one (180 mg, 0.94 mmol) and 2-isothiocyanato-4-(propan-2-yloxy)pyridine (218 mg, 1.12 mmol) in DCM (10 mL). The reaction mixture was stirred at 24° C. for 16 h. After completion, the reaction mixture was diluted with water and extracted with DCM. The combined organic layer was washed with brine, dried over anhydrous Na₂SO₄, filtered, and concentrated under reduced pressure. The crude product was purified by column chromatography followed by trituration with ether and pentane to provide N-[5-(5-methoxypyridin-2-yl)-1,3-oxazol-2-yl]-4-(propan-2-yloxy)pyridin-2-amine (20 mg, 6%). MS (ESI): m/z 327.2 [M+1]⁺.

### Example 71. 4-Methyl-N-[5-(3-methylpyridin-2-yl)-1,3-oxazol-2-yl]pyridin-2-amine

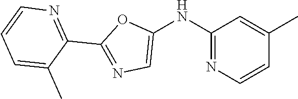 [SMILES: Cc1ccnc(Nc2cnc(-c3ncccc3C)o2)c1]

1-(3-Methylpyridin-2-yl)ethan-1-one. MeMgBr (3 M in THF, 17 ml, 51 mmol) was added dropwise at 0° C. under an argon atmosphere to a stirred solution of 3-methylpyridine-2-carbonitrile (3 g, 25.39 mmol) in anhydrous THE (30 mL). The resulting mixture was stirred at 0° C. for 2 h. The reaction mixture was quenched with NH₄Cl and extracted with EtOAc. The combined organic layer was washed with water and brine, dried over anhydrous Na₂SO₄, filtered, and concentrated under reduced pressure. The crude product was purified by column chromatography to get pure 1-(3-methylpyridin-2-yl)ethan-1-one (2.7 g, 79%). MS (ESI): m/z 136.0 [M+1]⁺.

2-Bromo-1-(3-methylpyridin-2-yl)ethan-1-one. To a stirred solution of 1-(3-methylpyridin-2-yl)ethan-1-one (2.3 g, 17.02 mmol) in glacial acetic acid (23 mL) was added 33% HBr in AcOH (11.5 mL) at 0° C. under an argon atmosphere. Then a solution of bromine (1 mL, 18.72 mmol) in AcOH (3 mL) was added dropwise. The resulting reaction mixture was stirred at 0° C. for 2 h. The solid was filtered, washed with ether, and dried to get 2-Bromo-1-(3-methylpyridin-2-yl)ethan-1-one (4.5 g, 90%, HBr salt). MS (ESI): m/z 214.2 [M+1]⁺.

2-Azido-1-(3-methylpyridin-2-yl)ethan-1-one. NaHCO₃ (1.14 g, 13.557 mmol) and NaN₃ (441 mg, 6.778 mmol) were added to a stirred solution of 2-bromo-1-(3-methylpyridin-2-yl)ethan-1-one (1 g, 3.389 mmol) in EtOH (10 mL) at 24° C. and stirred for 4 h. The reaction mixture was diluted with DCM and washed with water. The organic layer was washed with brine, dried over anhydrous Na₂SO₄, filtered, and concentrated under reduced pressure (bath temperature 25° C.) to get 2-azido-1-(3-methylpyridin-2-yl)ethan-1-one (450 mg, 75%). MS (ESI): m/z 177.2 [M+1]⁺.

4-Methyl-N-[5-(3-methylpyridin-2-yl)-1,3-oxazol-2-yl]pyridin-2-amine. PPh₃ (1.2 g, 4.54 mmol) was added to a stirred solution of 2-azido-1-(3-methylpyridin-2-yl)ethan-1-one (400 mg, 2.27 mmol) and 2-isothiocyanato-4-methylpyridine (409 mg, 2.73 mmol) in DCM (20 mL) at 24° C. and the reaction was stirred for 16 h. After completion, the reaction mixture was diluted with water and extracted with DCM. The combined organic layer was washed with brine, dried over anhydrous Na₂SO₄, filtered, and concentrated under reduced pressure. The crude mass was purified by column chromatography followed by trituration with ether and pentane to get 4-methyl-N-[5-(3-methylpyridin-2-yl)-1,3-oxazol-2-yl]pyridin-2-amine (100 mg, 16%). MS (ESI): m/z 267.2 [M+1]⁺.

### In Vitro Parasite Motility Assays

Parasite motility assays. Adult and microfilariae B. malayi and B. pahangi parasites, harvested from infected jirds, were procured from the NIAID/NIH Filariasis Research Reagent Resource Center (FR3). Adult and microfilariae of L. sigmodontis were procured from TRS labs Inc. (Athens, GA). Adult worms were plated in 24-well plates with 2 mL of Advanced RPMI 1640 medium (Invitrogen) supplemented with 25 mM HEPES, 2 mM L-Glutamine (Invitrogen), 100 U/mL Penicillin (Invitrogen), 100 g/mL Streptomycin (Invitro-gen), 2.5 g/mL Amphotericin B solution (Invitrogen), and 5% heat inactivated fetal bovine serum and placed in a 37° C. humidified incubator with 5% CO₂. After 24 h, adult worms were selected based upon motility as described below. After scoring for motility, 4-6 highly motile worms were selected for each treatment group and were transferred to new plates. Microfilariae were centrifuged at 5000×g for 5 min, and re-suspended in 2 ml of media. Microfilarial density was determined using a hemocytometer and were plated in a 96-well plate at 80 microfilariae/well with 200 μL of complete media. Treatment groups received compounds (0.1% DMSO) at 1 μM and 100 nM with 0.1% DMSO as a vehicle control. Cultures were incubated at 37° C. in a humidified incubator with 5% CO₂. Worms were transferred into a new plate containing fresh media and drug every 48 h. Parasite and microfilariae motility were given a score from 0 to 4 with 4, rapid movement and largely coiled; 3, moderated movement and uncoiled; 2, slow movement and uncoiled; 1, twitching movement and uncoiled; 0, no motility (dead). The motility of the worms and microfilariae were evaluated every 24 h and analyzed by a one sided unpaired Student's t-test using Microsoft Excel. Experiments were performed 2-3 times with similar results.

Onchocerciasis: In Vitro Screening Model Onchocerca gutturosa

Parasite and cell cultures. Onchocerca gutturosa adult male worms were obtained by dissection from the nuchal ligament connective tissues of naturally infected cattle, from Gambia, W Africa.

The worms were maintained for at least 24 h in culture before use in Eagles Minimum Essential Medium with Earl's Salts (Gibco, UK)+10% heat inactivated new born calf serum (Gibco, UK)+antibiotic cover of 200 units/ml penicillin, 200 μg/ml streptomycin and 0.5 μg/ml amphotericin B (Sigma, UK). Only normally active specimens were used in the test. All cultures and assays were conducted at 37° C. under an atmosphere of 5% CO₂ in air.

Drug sensitivity assays. Compound stock solutions were prepared in 100% DMSO unless otherwise indicated and diluted into the medium. Any unused compound stocks were stored at −20° C. Assays were performed in sterile 24-well (2 ml) plates (Falcon, UK).

Worms were then transferred individually to each well of the plate using fine forceps. Worm viability was assessed using 2 parameters:
The measurement of mean worm motility scores on a scale of 0 (immotile) to 10 (maximum) every 24 h, terminating at 120 h, using an Olympus inverted microscope.
The biochemical evaluation of worm viability using MTT/formazan colorimetry. The MTT assay was carried out after the last motility reading (120 h). Single intact worms were placed in each well of a 48-well plate (Falcon, UK) containing 0.5 ml of a solution consisting of 0.5 mg/ml MTT (Sigma UK) in phosphate buffered saline, and then incubated for 30 min at 37° C. The worms were removed, blotted carefully, and individually transferred to separate wells of a 96-well microtiter plate, each containing 200 μl of DMSO to solubilize the formazan. After 1 h the plate was gently agitated to disperse the color evenly and the absorbance value (optical density) of the resulting formazan solution was determined at 490 nm using a multi-well scanning spectrophotometer (Elisa-reader, Dynatech, UK). Inhibition of formazan formation was correlated with worm damage or death.

Primary screen. New compounds were usually tested at 1.25×10⁻⁵ M. Also expressed in μg/ml. Test drugs (2 worms/group) were compared to untreated controls (6 worms/group) and a positive control (standard drug, 6 worms/group). The standard used was Immiticide (Merial): this drug produces a reduction in motility of 100%, and mean inhibition of formazan formation of ˜85%. The approximate motility EC₅₀ for Immiticide was 3×10⁻⁷ M, and for ivermectin was 1×10⁻⁸ M. The readouts are: Motility score (mean % reduction at 120 h) MTT colorimetry (mean inhibition of formazan formation).

A test compound was considered active if there was a 50% or greater reduction in motility score and/or a 50% or greater inhibition of formazan formation compared to untreated controls.

Compounds were classified as moderately active if there was a 50-99% reduction in motility and/or inhibition of formazan, or highly active at 100%/lower concentrations.

Secondary screen. All active compounds were re-tested. Serial 1 in 4 drug dilutions was carried out to find activity endpoint and EC₅₀ values for motility reduction and inhibition of formazan formation were produced. EC₅₀ values were determined using Excel or Origin V7 scientific graphing and data analysis software.

Heartworm Screen Dirofilaria immitis (D. immitis)

Dirofilaria immitis, Microfilaria (DiMF) Assay. Compounds were dissolved and serially diluted in DMSO. Aliquots were spotted to the empty wells of assay plates. Media and microfilariae of Dirofilaria immitis were added to each well to dilute the test compounds to the desired concentrations. Assay plates were incubated for approximately 72 hours, and the larvae in each well were observed microscopically for drug effect. Microfilariae in each well were assessed subjectively for survival or paralysis, and results were reported as Minimum Effective Dose (MED).

Dirofilaria immitis, L4 stage (DiL4) Assay. Compounds were dissolved and serially diluted in DMSO. Aliquots were spotted to the empty wells of assay plates. Media and 4th stage larvae (L4) of Dirofilaria immitis were added to each well to dilute the test compounds to the desired concentrations. Assay plates were incubated for approximately 72 hours, and the larvae in each well were observed microscopically for drug effect. Larvae in each well were assessed subjectively for survival or paralysis, and results were reported as Minimum Effective Dose (MED).

The compounds described herein demonstrated nematocidal activity against either Dirofilaria immitis (Larva stage 4 (DiL4)) and/or Dirofilaria immitis (microfilaria (DiMF)) as determined by reductions in nematode motility either by paralysis or death. Active and selective (DiL4 vs. DiMF potency) example compounds were subsequently evaluated in heartworm positive dog studies to correlate the in vitro selectivity profile with in vivo effects on circulating microfilariae.

Activity of the Heterocyclic Compounds in the parasite motility assays is shown in Table 1, Table 2, Table 3, Table 4, and Table 5.

### In Vivo Filariasis Assays

L. sigmodontis in vivo assays. The infection of mice and jirds can be either initiated by the natural route, exposure of mites containing infective third stage larvae (L3) of L. sigmodontis, or via the injection (subcutaneous, intraperitoneal or intravenous) of a known number of L3 larvae (G. Karadjian et al., Migratory phase of Litomosoides sigmodontis filarial infective larvae is associated with pathology and transient increase of S100A9 expressing neutrophils in the lung, PLoS Negl Trop Dis 11, e0005596 (2017)). Upon infection L3 larvae migrate from the site of inoculation within 2-6 days via the lymphatics to the thoracic cavity, where they molt around 10 days post infection (dpi) into 4th stage larvae and around 30 dpi into adult worms. Approximately 56 dpi adult female worms start to release microfilariae that enter the peripheral blood. In BALB/c mice, adult worm burden starts to decline around 70 dpi and by 100 dpi at which most of the adult worms are cleared. Jirds harbor the adult worms for more than one year.

L. sigmodontis mouse model. The L. sigmodontis mouse model allows the analysis of the activity of compounds on the adult worm or the development into adult worms.

L. sigmodontis jird model. In order to assess the efficacy of drug candidates during chronic, patient infection the L. sigmodontis jird model was used. In general, treatment with drug candidates was initiated 12 weeks post infection and only microfilariae-positive jirds were included in the experiments. Necropsies were performed in general 8-16 weeks post treatment. This extended time between initiation of treatment and necropsy allowed to identify the macrofilaricidal (adult worm killing) efficacy of slow acting compounds. The jird model allowed the assessment of the in vivo impact of compounds on microfilariae over time. Compounds with strong microfilaricidal efficacy clear the microfilariae from peripheral blood within a short period of time. Compounds with an adult worm sterilizing or macrofilaricidal efficacy (lacking a microfilaricidal efficacy) lead to a delayed reduction of the microfilaremia that exceeds 4 weeks post treatment start. Additional analysis at the time of necropsy included the quantification of adult worms, ratios of female and male adult worms, and motility of adult worms at the time of necropsy. Remaining female adult worms were assessed for their embryogenesis and therefore sterilizing effects of compounds. Embryograms from female adult worms included the quantification of early developmental stages (egg/morulae) and later stages (pretzel stage & stretched microfilariae) according to (S. Ziewer et al., Immunization with L. sigmodontis Microfilariae Reduces Peripheral Microfilaraemia after Challenge Infection by Inhibition of Filarial Embryogenesis, PLoS Negl Trop Dis 6, e1558 (2012)). Lack of early and/or later developmental embryonic stages suggested a sterilizing effect of the compounds. Additional histological and TEM analysis was applied to analyze any tissue damages caused by the drug candidates that may be associated with permanent sterilization.

The L. sigmodontis jird model assessed the macrofilaricidal efficacy of compounds, their impact on microfilaremia, female worm embryogenesis and sterilization.

The Heterocyclic Compounds provided herein were tested and showed activity in both L. sigmodontis mouse and L. sigmodontis jird model assays performed as described herein, with some compounds showing macrofilaricidal activity and some compounds showing macrofilaricidal selectivity.

In some embodiments, the compounds disclosed herein surprisingly presented distinct activity between parasitic nematodes in adult and juvenile stage. In some such embodiments, the compounds disclosed herein were found to be selectively effective against adult filarial nematodes (i.e., were macroselective). Therefore, the compounds disclosed herein have the potential to be potent anti-filarial drugs.

In some embodiments, the compounds disclosed herein surprisingly presented distinct activity between parasitic nematodes in adult and juvenile stage. In some such embodiments, the compounds disclosed herein were found to be selectively effective against adult filarial nematodes (i.e., were macroselective). Therefore, the compounds disclosed herein have the potential to be potent anti-filarial drugs.

Heartworm Dog Studies. Dogs with pre-existing heartworm infections, via surgical transplantation were used for these studies. To confirm that the dogs had circulating microfilariae, blood samples were taken from each dog and examined for microfilariae by using the modified Knott's method. All dog cohorts included in the studies exhibited average microfilariae counts of at least 15,000 MF/mL of the blood (pre-dose). On approximately Day −7, dogs were randomly allocated to treatments (three animals per treatment group) based on Day −7 MF counts. Dogs were fasted overnight prior to dosing and fed immediately following dosing of the test articles. Compounds were administered by point dosing in oral liquid-filled capsules on Day 0. Blood samples were collected to measure MF counts on Days 0 (pre-dose and 2 hours post-dose), 1, 2, 7, 21 and 28. Clinical observations were conducted by a suitably experienced veterinarian on days −7, 0 (immediately prior to treatment, 1-2 hours post-treatment), 1 and 2 whereby any abnormal clinical signs were documented using standard veterinary medical terminology. Additionally, general health observations were conducted throughout the study including (but not limited to) general physical appearance and behavior, abnormalities of food and water consumption, vomiting/regurgitation, appearance of urine and feces and any sign of MF anaphylaxis.

The Heterocyclic Compounds provided herein were tested and showed activity, or will be shown to have activity, on circulating microfilariae in vivo.

### Activity Table

Each of the compounds in Table 1, Table 2, Table 3, Table 4, and Table 5 was tested in at least one of the in vitro filarial motility assays and was found to have activity therein, with all of the Heterocyclic Compounds of formula (I) formula (II), formula (III), formula (IIIa), formula (IIIb), formula (IIIc), formula (IIId), formula (IV), formula (IVa), formula (IVb), and formula (IVc), having an IC₅₀ below or at 5 μM in one or more of the assays, with some compounds having an IC₅₀ between 0.5 μM and 5 μM (activity level A), some having an IC₅₀ between 0.2 μM and 0.5 μM (activity level B), and some having an IC₅₀ below 0.2 μM (activity level C). Heterocyclic Compounds of formula (I) and formula (II), were tested in one or more of the assays and were shown to have activity therein, with some of the Heterocyclic Compounds of formula (I) formula (II), formula (III), formula (IIIa), formula (IIIb), formula (IIIc), formula (IIId), formula (IV), formula (IVa), formula (IVb), and formula (IVc) having activity against microfilaria at compound concentrations below 1 μM (activity level D) with some compounds having activity against adult filaria at compound concentrations below 1 μM (activity level E).

TABLE 1

| | | | | | | | L. | D. |
|---|---|---|---|---|---|---|---|---|
| | | | | | B. | B. | sigmo | immi |
| | | | | O. gutt | malayi | phangi | dontis | tis |
| Com. | | | | Act | Act | Act | Act | Act |
| No. | Structure | Name | M + 1 | class | class | class | class | class |
| 1 | 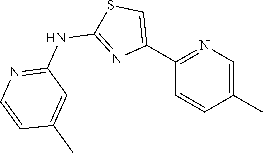 [SMILES: Cc1ccc(-c2csc(Nc3cc(C)ccn3)n2)nc1] | N-(4-methylpyridin-2- yl)-4-(5- methylpyridin-2- yl)thiazol-2-amine | 283.40 | C | D | | | C |
| 2 | 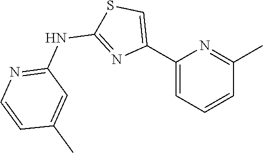 [SMILES: Cc1ccnc(Nc2nc(-c3cccc(C)n3)cs2)c1] | N-(4-methylpyridin-2- yl)-4-(6- methylpyridin-2- yl)thiazol-2-amine | 283.20 | B | D | | | |
| 3 | 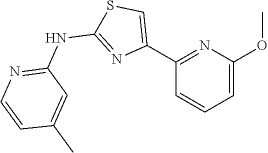 [SMILES: COc1cccc(-c2csc(Nc3cc(C)ccn3)n2)n1] | 4-(6-methoxypyridin- 2-yl)-N-(4- methylpyridin-2- yl)thiazol-2-amine | 299.30 | A | | | | |
| 4 | 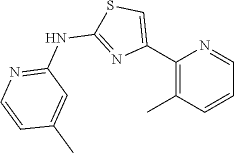 [SMILES: Cc1ccnc(Nc2nc(-c3ncccc3C)cs2)c1] | 4-(3-methylpyridin-2- yl)-N-(4- methylpyridin-2- yl)thiazol-2-amine | 283.20 | C | D | | | |
| 5 | 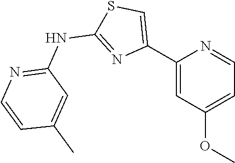 [SMILES: COc1ccnc(-c2csc(Nc3cc(C)ccn3)n2)c1] | 4-(4-methoxypyridin- 2-yl)-N-(4- methylpyridin-2- yl)thiazol-2-amine | 299.30 | C | D, E | | D, E | C |
| 6 | 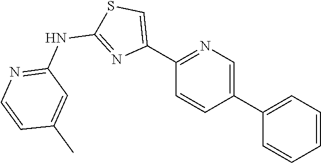 [SMILES: Cc1ccnc(Nc2nc(-c3ccc(-c4ccccc4)cn3)cs2)c1] | N-(4-methylpyridin-2- yl)-4-(5- phenylpyridin-2- yl)thiazol-2-amine | 345.30 | C | | | | C |
| 7 | 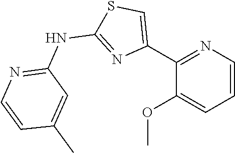 [SMILES: COc1cccnc1-c1csc(Nc2cc(C)ccn2)n1] | 4-(3-methoxypyridin- 2-yl)-N-(4- methylpyridin-2- yl)thiazol-2-amine | 299.30 | A | | | | B |
| 8 | 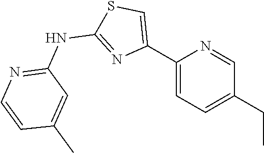 [SMILES: CCc1ccc(-c2csc(Nc3cc(C)ccn3)n2)nc1] | 4-(5-ethylpyridin-2- yl)-N-(4- methylpyridin-2- yl)thiazol-2-amine | 381.30 | B | D | | | C |
| 9 | 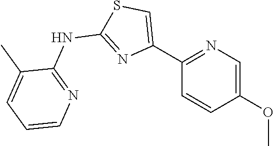 | 4-(5-methoxypyridin- 2-yl)-N-(3- methylpyridin-2- yl)thiazol-2-amine | 299.22 | C | D, E | | D | |
| 10 | 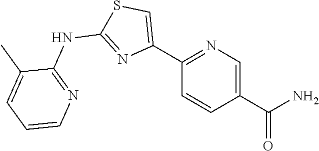 [SMILES: Cc1cccnc1Nc1nc(-c2ccc(C(N)=O)cn2)cs1] | 6-(2-(3- methylpyridin-2- ylamino)thiazol-4- yl)nicotinamide | 294.2 | A | | | | |
| 11 | 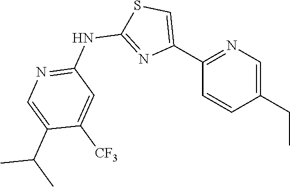 [SMILES: CCc1ccc(-c2csc(Nc3cc(C(F)(F)F)c(C(C)C)cn3)n2)nc1] | 4-(5-ethylpyridin-2- yl)-N-(5-isopropyl-4- (trifluoromethyl) pyridin-2-yl)thiazol-2- amine | 379.2 | C | D, E | | D | C |
| 12 | 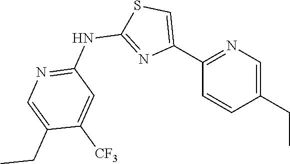 [SMILES: CCc1ccc(-c2csc(Nc3cc(C(F)(F)F)c(CC)cn3)n2)nc1] | N-(5-ethyl-4- (trifluoromethyl) pyridin-2-yl)-4-(5- ethylpyridin-2- yl)thiazol-2-amine | 379.2 | A | D, E | | D | B |
| 13 | 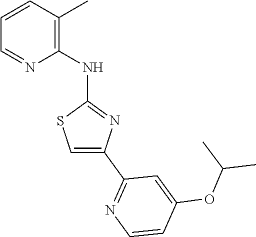 [SMILES: Cc1cccnc1Nc1nc(-c2cc(OC(C)C)ccn2)cs1] | 4-(4- isopropoxypyridin-2- yl)-N-(3- methylpyridin-2- yl)thiazol-2-amine | 327.10 | C | D, E | | | C |
| 14 | 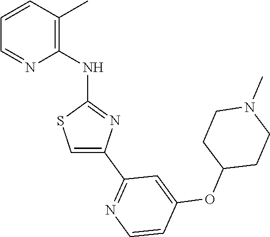 [SMILES: Cc1cccnc1Nc1nc(-c2cc(OC3CCN(C)CC3)ccn2)cs1] | 4-(4-(1- methylpiperidin-4- yloxy)pyridin-2-yl)- N-(3-methylpyridin-2- yl)thiazol-2-amine | 382.10 | C | D, E | | | A |
| 15 | 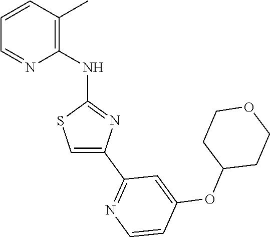 [SMILES: Cc1cccnc1Nc1nc(-c2cc(OC3CCOCC3)ccn2)cs1] | N-(3-methylpyridin-2- yl)-4-(4-(tetrahydro- 2H-pyran-4- yloxy)pyridin-2- yl)thiazol-2-amine | 369.10 | C | D, E | | | C |
| 16 | 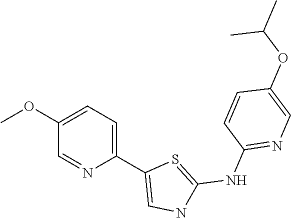 | N-(5- isopropoxypyridin-2- yl)-4-(5- methoxypyridin-2- yl)thiazol-2-amine | 343.10 | C | D, E | | | C |
| 17 | 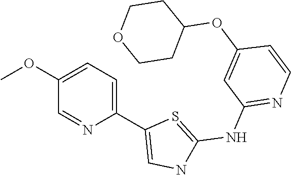 | 4-(5-methoxypyridin- 2-yl)-N-(4- (tetrahydro-2H-pyran- 4-yloxy)pyridin-2- yl)thiazol-2-amine | 385.10 | B | D, E | E | D, E | C |
| 18 | 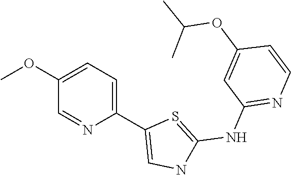 | N-(4- isopropoxypyridin-2- yl)-4-(5- methoxypyridin-2- yl)thiazol-2-amine | 343.2 | B | D, E | | D, E | C |
| 19 | 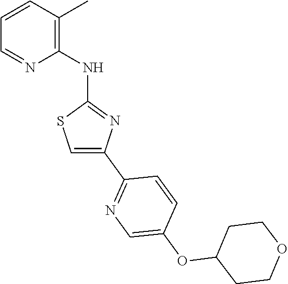 [SMILES: Cc1cccnc1Nc1nc(-c2ccc(OC3CCOCC3)cn2)cs1] | N-(3-methylpyridin-2- yl)-4-(5-(tetrahydro- 2H-pyran-4- yloxy)pyridin-2- yl)thiazol-2-amine | 369.1 | C | D | | | C |
| 20 | 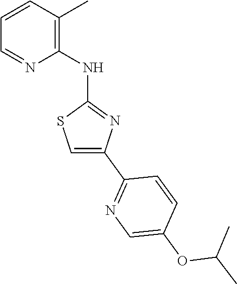 [SMILES: Cc1cccnc1Nc1nc(-c2ccc(OC(C)C)cn2)cs1] | 4-(5- isopropoxypyridin-2- yl)-N-(3- methylpyridin-2- yl)thiazol-2-amine | 327.29 | C | D, E | | D, E | C |
| 21 | 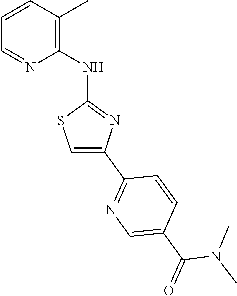 [SMILES: Cc1cccnc1Nc1nc(-c2ccc(C(=O)N(C)C)cn2)cs1] | N,N-dimethyl-6-(2-(3- methylpyridin-2- ylamino)thiazol-4- yl)nicotinamide | 340.2 | C | D | | D, E | A |
| 22 | 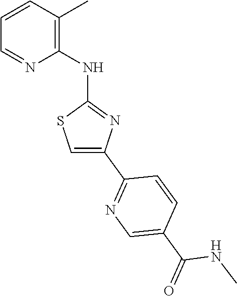 [SMILES: CNC(=O)c1ccc(-c2csc(Nc3ncccc3C)n2)nc1] | N-methyl-6-(2-(3- methylpyridin-2- ylamino)thiazol-4- yl)nicotinamide | 326.00 | C | | | | |
| 23 | 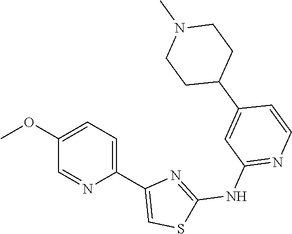 [SMILES: COc1ccc(-c2csc(Nc3cc(C4CCN(C)CC4)ccn3)n2)nc1] | 4-(5-methoxypyridin- 2-yl)-N-(4-(1- methylpiperidin-4- yl)pyridin-2- yl)thiazol-2-amine | 382.20 | C | | | | |
| 24 | 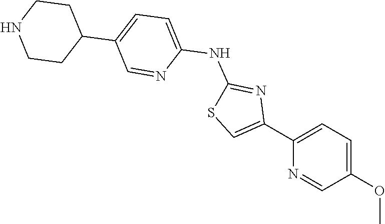 [SMILES: COc1ccc(-c2csc(Nc3ccc(C4CCNCC4)cn3)n2)nc1] | 4-(5-methoxypyridin- 2-yl)-N-(5-(piperidin- 4-yl)pyridin-2- yl)thiazol-2-amine | 386.10 | B | | | | |
| 25 | 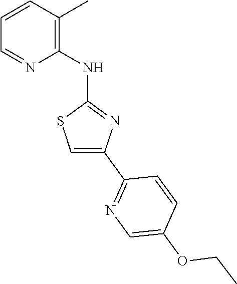 [SMILES: CCOc1ccc(-c2csc(Nc3ncccc3C)n2)nc1] | 4-(5-ethoxypyridin-2- yl)-N-(3- methylpyridin-2- yl)thiazol-2-amine | 313.1 | B | D, E | | | |
| 26 | 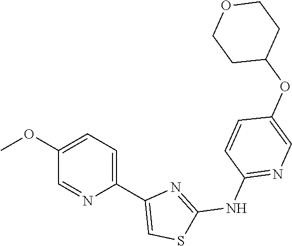 [SMILES: COc1ccc(-c2csc(Nc3ccc(OC4CCOCC4)cn3)n2)nc1] | 4-(5-methoxypyridin- 2-yl)-N-(5- (tetrahydro-2H-pyran- 4-yloxy)pyridin-2- yl)thiazol-2-amine | 385.1 | C | D, E | | D, E | C |
| 27 | 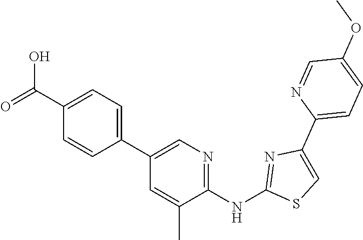 [SMILES: COc1ccc(-c2csc(Nc3ncc(-c4ccc(C(=O)O)cc4)cc3C)n2)nc1] | 4-(6-(4-(5- methoxypyridin-2- yl)thiazol-2-ylamino)- 5-methylpyridin-3- yl)benzoic acid | 419.00 | C | | | | |
| 28 | 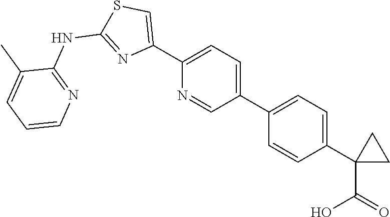 [SMILES: Cc1cccnc1Nc1nc(-c2ccc(-c3ccc(C4(C(=O)O)CC4)cc3)cn2)cs1] | 1-(4-(6-(2-(3- methylpyridin-2- ylamino)thiazol-4- yl)pyridin-3- yl)phenyl) cyclopropanecarboxylic acid | 429.10 | B | | | | |
| 29 | 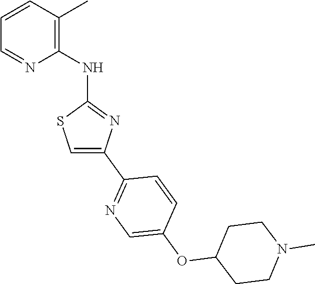 [SMILES: Cc1cccnc1Nc1nc(-c2ccc(OC3CCN(C)CC3)cn2)cs1] | 4-(5-(1- methylpiperidin-4- yloxy)pyridin-2-yl)- N-(3-methylpyridin-2- yl)thiazol-2-amine | 382.10 | C | D, E | E | D, E | A |
| 30 | 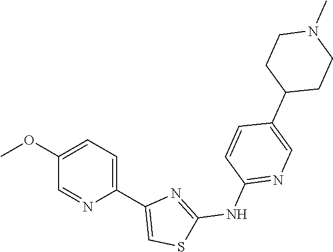 [SMILES: COc1ccc(-c2csc(Nc3ccc(C4CCN(C)CC4)cn3)n2)nc1] | 4-(5-methoxypyridin- 2-yl)-N-(5-(1- methylpiperidin-4- yl)pyridin-2- yl)thiazol-2-amine | 482.10 | C | E | | | |
| 31 | 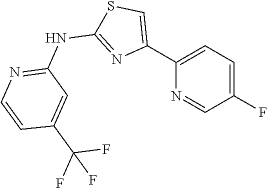 [SMILES: Fc1ccc(-c2csc(Nc3cc(C(F)(F)F)ccn3)n2)nc1] | 4-(5-fluoropyridin-2- yl)-N-(4- (trifluoromethyl) pyridin-2-yl)thiazol-2- amine | 341.10 | C | D, E | E | D, E | |
| 32 | 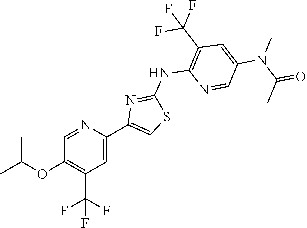 [SMILES: CC(=O)N(C)c1cnc(Nc2nc(-c3cc(C(F)(F)F)c(OC(C)C)cn3)cs2)c(C(F)(F)F)c1] | N-(6-(4-(5- isopropoxy-4- (trifluoromethyl) pyridin-2-yl)thiazol-2- ylamino)-5- (trifluoromethyl) pyridin-3-yl)-N- methylacetamide | 520.10 | | | | | |
| 33 | 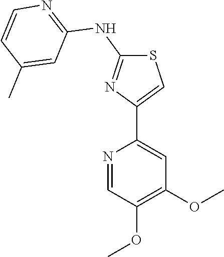 [SMILES: COc1cnc(-c2csc(Nc3cc(C)ccn3)n2)cc1OC] | 4-(4,5- dimethoxypyridin-2- yl)-N-(4- methylpyridin-2- yl)thiazol-2-amine | 329.40 | | | | | C |
| 34 | 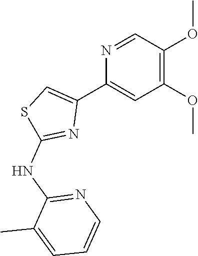 | 4-(4,5- dimethoxypyridin-2- yl)-N-(3- methylpyridin-2- yl)thiazol-2-amine | 329.40 | | | | | C |
| 35 | 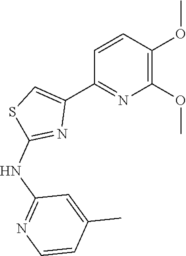 [SMILES: COc1ccc(-c2csc(Nc3cc(C)ccn3)n2)nc1OC] | 4-(5,6- dimethoxypyridin-2- yl)-N-(4- methylpyridin-2- yl)thiazol-2-amine | 329.50 | | | | | |
| 36 | 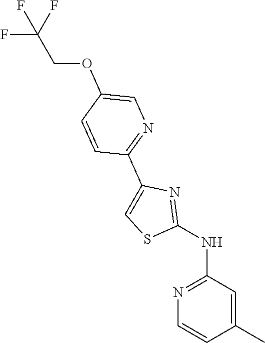 [SMILES: Cc1ccnc(Nc2nc(-c3ccc(OCC(F)(F)F)cn3)cs2)c1] | N-(4-methylpyridin-2- yl)-4-(5-(2,2,2- trifluoroethoxy) pyridin-2-yl)thiazol- 2-amine | 367.10 | | | | | C |
| 37 | 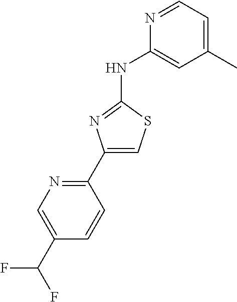 [SMILES: Cc1ccnc(Nc2nc(-c3ccc(C(F)F)cn3)cs2)c1] | 4-(5- (difluoromethyl) pyridin-2-yl)-N-(4- methylpyridin-2- yl)thiazol-2-amine | 319.30 | | | | | C |
| 38 | 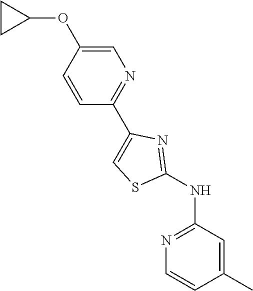 [SMILES: Cc1ccnc(Nc2nc(-c3ccc(OC4CC4)cn3)cs2)c1] | 4-(5- cyclopropoxypyridin- 2-yl)-N-(4- methylpyridin-2- yl)thiazol-2-amine | 325.40 | | | | | C |
| 39 | 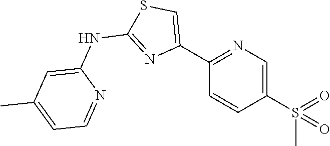 [SMILES: Cc1ccnc(Nc2nc(-c3ccc(S(C)(=O)=O)cn3)cs2)c1] | N-(4-methylpyridin-2- yl)-4-(5- (methylsulfonyl) pyridin-2-yl) thiazol-2-amine | 347.00 | | | | | A |
| 40 | 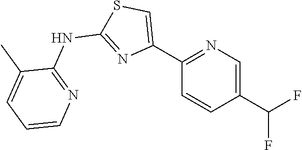 [SMILES: Cc1cccnc1Nc1nc(-c2ccc(C(F)F)cn2)cs1] | 4-(5- (difluoromethyl) pyridin-2-yl)-N-(3- methylpyridin-2- yl)thiazol-2-amine | 319.40 | | | | | |
| 41 | 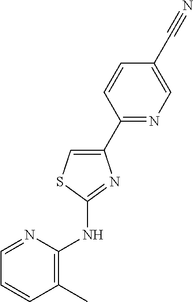 [SMILES: Cc1cccnc1Nc1nc(-c2ccc(C#N)cn2)cs1] | 6-(2-(3- methylpyridin-2- ylamino)thiazol-4- yl)nicotinonitrile | 294.10 | | | | | A |
| 42 | 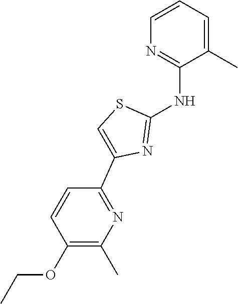 [SMILES: CCOc1ccc(-c2csc(Nc3ncccc3C)n2)nc1C] | 4-(5-ethoxy-6- methylpyridin-2-yl)- N-(3-methylpyridin-2- yl)thiazol-2-amine | 327.10 | | | | | A |
| 43 | 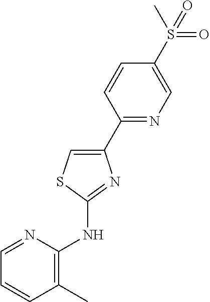 [SMILES: Cc1cccnc1Nc1nc(-c2ccc(S(C)(=O)=O)cn2)cs1] | N-(3-methylpyridin-2- yl)-4-(5- (methylsulfonyl) pyridin-2-yl)thiazol- 2-amine | 347.10 | | | | | A |
| 44 | 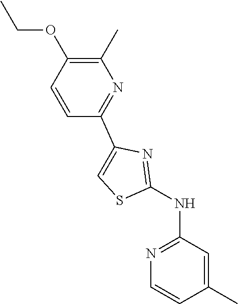 [SMILES: CCOc1ccc(-c2csc(Nc3cc(C)ccn3)n2)nc1C] | 4-(5-ethoxy-6- methylpyridin-2-yl)- N-(4-methylpyridin-2- yl)thiazol-2-amine | 327.20 | | | | | A |
| 45 | 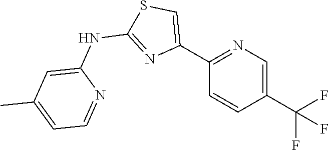 [SMILES: Cc1ccnc(Nc2nc(-c3ccc(C(F)(F)F)cn3)cs2)c1] | N-(4-methylpyridin-2- yl)-4-(5- (trifluoromethyl) pyridin-2-yl)thiazol-2- amine | 337.10 | | | | | A |
| 46 | 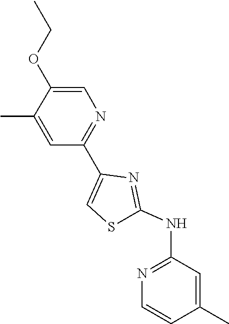 | 4-(5-ethoxy-4- methylpyridin-2-yl)- N-(4-methylpyridin-2- yl)thiazol-2-amine | 327.20 | | | | | C |
| 47 | 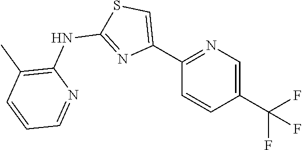 [SMILES: Cc1cccnc1Nc1nc(-c2ccc(C(F)(F)F)cn2)cs1] | N-(3-methylpyridin-2- yl)-4-(5- (trifluoromethyl) pyridin-2-yl)thiazol-2- amine | 337.10 | | | | | A |
| 48 | 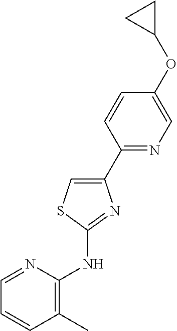 [SMILES: Cc1cccnc1Nc1nc(-c2ccc(OC3CC3)cn2)cs1] | 4-(5- cyclopropoxypyridin- 2-yl)-N-(3- methylpyridin-2- yl)thiazol-2-amine | 325.30 | | | | | B |
| 49 | 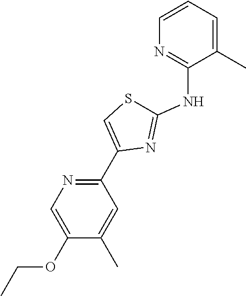 [SMILES: CCOc1cnc(-c2csc(Nc3ncccc3C)n2)cc1C] | 4-(5-ethoxy-4- methylpyridin-2-yl)- N-(3-methylpyridin-2- yl)thiazol-2-amine | 327.30 | | | | | C |
| 50 | 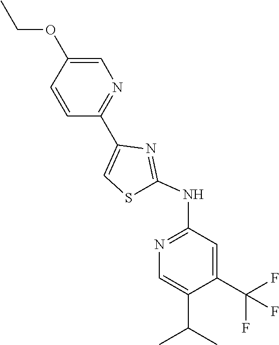 | 4-(5-ethoxypyridin-2- yl)-N-(5-isopropyl-4- (trifluoromethyl) pyridin-2-yl)thiazol-2- amine | 409.20 | | | | | C |
| 51 | 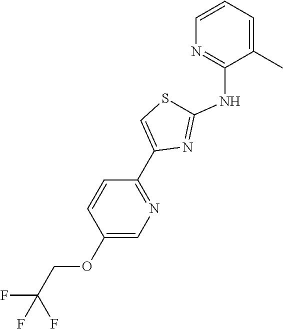 [SMILES: Cc1cccnc1Nc1nc(-c2ccc(OCC(F)(F)F)cn2)cs1] | N-(3-methylpyridin-2- yl)-4-(5-(2,2,2- trifluoroethoxy)pyridin- 2-yl)thiazol-2-amine | 367.10 | | | | | B |
| 52 | 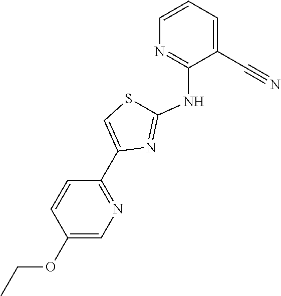 [SMILES: CCOc1ccc(-c2csc(Nc3ncccc3C#N)n2)nc1] | 2-(4-(5-ethoxypyridin- 2-yl)thiazol-2- ylamino) nicotinonitrile | 324.30 | | D | | | B |
| 53 | 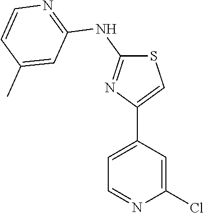 [SMILES: Cc1ccnc(Nc2nc(-c3ccnc(Cl)c3)cs2)c1] | 4-(2-chloropyridin-4- yl)-N-(4- methylpyridin-2- yl)thiazol-2-amine | 303.30 | | | | | A |
| 54 | 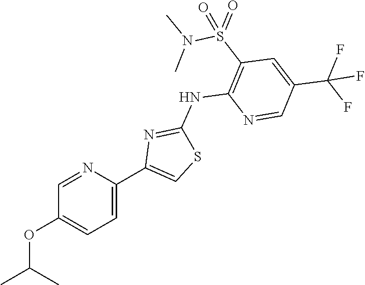 [SMILES: CC(C)Oc1ccc(-c2csc(Nc3ncc(C(F)(F)F)cc3S(=O)(=O)N(C)C)n2)nc1] | 2-(4-(5- isopropoxypyridin-2- yl)thiazol-2-ylamino)- N,N-dimethyl-5- (trifluoromethyl) pyridine-3-sulfonamide | 488.10 | | | | | |
| 55 | 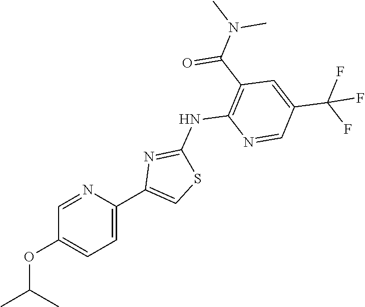 [SMILES: CC(C)Oc1ccc(-c2csc(Nc3ncc(C(F)(F)F)cc3C(=O)N(C)C)n2)nc1] | 2-(4-(5- isopropoxypyridin-2- yl)thiazol-2-ylamino)- N,N-dimethyl-5- (trifluoromethyl) nicotinamide | 452.20 | | | | | |
| 56 | 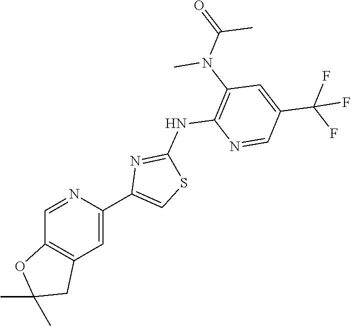 [SMILES: CC(=O)N(C)c1cc(C(F)(F)F)cnc1Nc1nc(-c2cc3c(cn2)OC(C)(C)C3)cs1] | N-(2-(4-(2,2- dimethyl-2,3- dihydrofuro[2,3- c]pyridin-5-yl)thiazol- 2-ylamino)-5- (trifluoromethyl) pyridin-3-yl)-N- methylacetamide | 464.10 | | | | | |
| 57 | 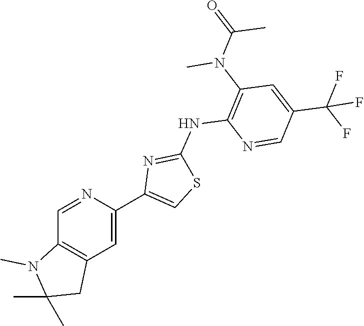 [SMILES: CC(=O)N(C)c1cc(C(F)(F)F)cnc1Nc1nc(-c2cc3c(cn2)N(C)C(C)(C)C3)cs1] | N-methyl-N-(5- (trifluoromethyl)-2-(4- (1,2,2-trimethyl-2,3- dihydro-1H- pyrrolo[2,3-c]pyridin- 5-yl)thiazol-2- ylamino)pyridin-3- yl)acetamide | 477.10 | | | | | |
| 58 | 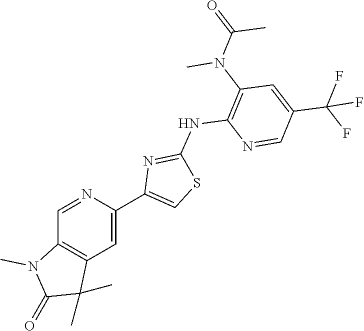 [SMILES: CC(=O)N(C)c1cc(C(F)(F)F)cnc1Nc1nc(-c2cc3c(cn2)N(C)C(=O)C3(C)C)cs1] | N-methyl-N-(5- (trifluoromethyl)-2-(4- (1,3,3-trimethyl-2- oxo-2,3-dihydro-1H- pyrrolo[2,3-c]pyridin- 5-yl)thiazol-2- ylamino)pyridin-3- yl)acetamide | 491.10 | | | | | |
| 59 | 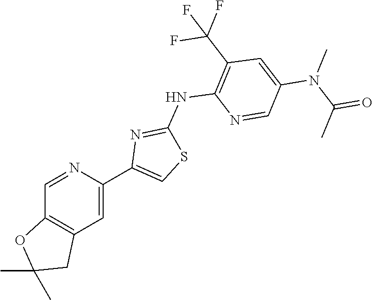 [SMILES: CC(=O)N(C)c1cnc(Nc2nc(-c3cc4c(cn3)OC(C)(C)C4)cs2)c(C(F)(F)F)c1] | N-(6-(4-(2,2- dimethyl-2,3- dihydrofuro[2,3- c]pyridin-5-yl)thiazol- 2-ylamino)-5- (trifluoromethyl) pyridin-3-yl)-N- methylacetamide | 464.10 | | | | | |
| 60 | 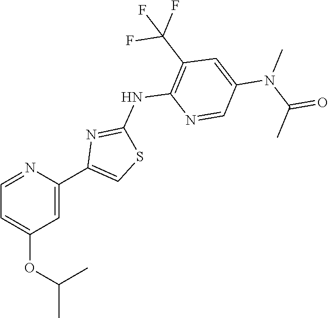 [SMILES: CC(=O)N(C)c1cnc(Nc2nc(-c3cc(OC(C)C)ccn3)cs2)c(C(F)(F)F)c1] | N-(6-(4-(4- isopropoxypyridin-2- yl)thiazol-2-ylamino)- 5-(trifluoromethyl) pyridin-3-yl)-N- methylacetamide | 452.30 | C | D, E | | | |
| 61 | 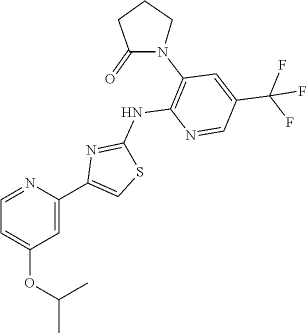 [SMILES: CC(C)Oc1ccnc(-c2csc(Nc3ncc(C(F)(F)F)cc3N3CCCC3=O)n2)c1] | 1-(2-(4-(4- isopropoxypyridin-2- yl)thiazol-2-ylamino)- 5-(trifluoromethyl) pyridin-3-yl) pyrrolidin-2-one | 464.20 | C | D, E | | | |
| 62 | 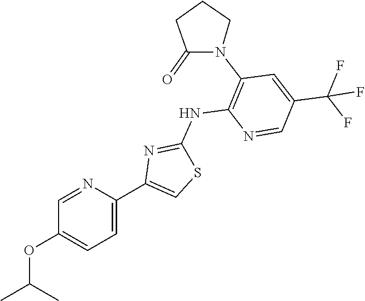 [SMILES: CC(C)Oc1ccc(-c2csc(Nc3ncc(C(F)(F)F)cc3N3CCCC3=O)n2)nc1] | 1-(2-(4-(5- isopropoxypyridin-2- yl)thiazol-2-ylamino)- 5-(trifluoromethyl) pyridin-3-yl) pyrrolidin-2- one | 464.30 | C | D, E | | | |
| 63 | 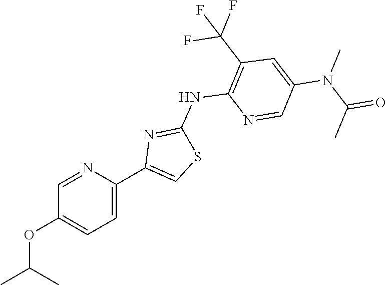 [SMILES: CC(=O)N(C)c1cnc(Nc2nc(-c3ccc(OC(C)C)cn3)cs2)c(C(F)(F)F)c1] | N-(6-(4-(5- isopropoxypyridin-2- yl)thiazol-2-ylamino)- 5-(trifluoromethyl) pyridin- 3-yl)-N- methylacetamide | 452.20 | C | D, E | | | |
| 64 | 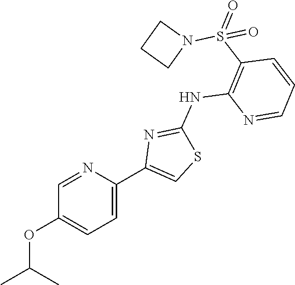 [SMILES: CC(C)Oc1ccc(-c2csc(Nc3ncccc3S(=O)(=O)N3CCC3)n2)nc1] | N-(3-(azetidin-1- ylsulfonyl)pyridin-2- yl)-4-(5- isopropoxypyridin-2- yl)thiazol-2-amine | 432.10 | A | E | | | |
| 65 | 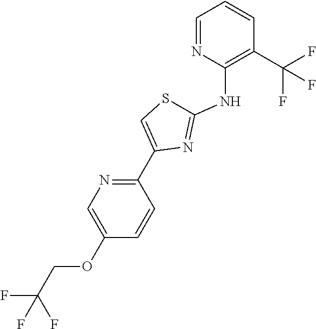 [SMILES: FC(F)(F)COc1ccc(-c2csc(Nc3ncccc3C(F)(F)F)n2)nc1] | 4-(5-(2,2,2- trifluoroethoxy) pyridin-2-yl)-N-(3- (trifluoromethyl) pyridin-2-yl)thiazol-2- amine | 421.10 | A | | | | A |
| 66 | 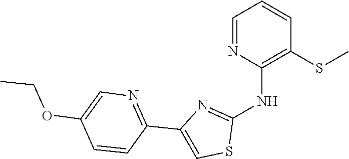 [SMILES: CCOc1ccc(-c2csc(Nc3ncccc3SC)n2)nc1] | 4-(5-ethoxypyridin-2- yl)-N-(3- (methylthio)pyridin-2- yl)thiazol-2-amine | 345.70 | A | | | | A |
| 67 | 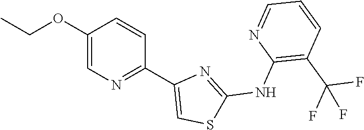 [SMILES: CCOc1ccc(-c2csc(Nc3ncccc3C(F)(F)F)n2)nc1] | 4-(5-ethoxypyridin-2- yl)-N-(3- (trifluoromethyl) pyridin-2-yl)thiazol- 2-amine | 367.10 | A | | | | A |
| 68 | 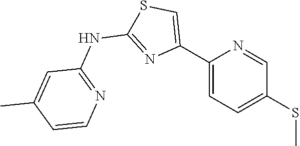 | N-(4-methylpyridin-2- yl)-4-(5- (methylthio)pyridin-2- yl)thiazol-2-amine | 315.10 | C | | | | C |
| 69 | 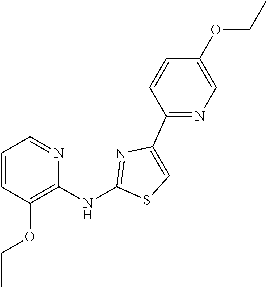 [SMILES: CCOc1ccc(-c2csc(Nc3ncccc3OCC)n2)nc1] | N-(3-ethoxypyridin-2- yl)-4-(5- ethoxypyridin-2- yl)thiazol-2-amine | 343.40 | A | | | | A |
| 70 | 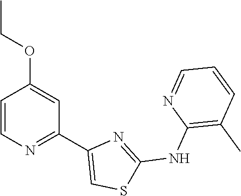 [SMILES: CCOc1ccnc(-c2csc(Nc3ncccc3C)n2)c1] | 4-(4-ethoxypyridin-2- yl)-N-(3- methylpyridin-2- yl)thiazol-2-amine | 313.40 | C | | | | C |
| 71 | 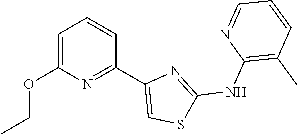 [SMILES: CCOc1cccc(-c2csc(Nc3ncccc3C)n2)n1] | 4-(6-ethoxypyridin-2- yl)-N-(3- methylpyridin-2- yl)thiazol-2-amine | 313.10 | A | | | | A |
| 72 | 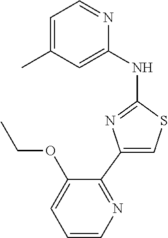 [SMILES: CCOc1cccnc1-c1csc(Nc2cc(C)ccn2)n1] | 4-(3-ethoxypyridin-2- yl)-N-(4- methylpyridin-2- yl)thiazol-2-amine | 313.10 | C | | | | C |
| 73 | 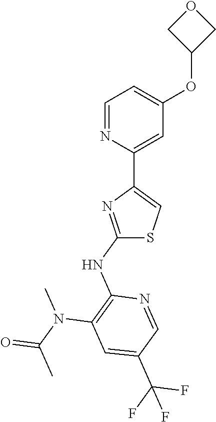 [SMILES: CC(=O)N(C)c1cc(C(F)(F)F)cnc1Nc1nc(-c2cc(OC3COC3)ccn2)cs1] | N-methyl-N-(2-(4-(4- (oxetan-3- yloxy)pyridin-2- yl)thiazol-2-ylamino)- 5-(trifluoromethyl) pyridin- 3-yl)acetamide | 466.10 | B | D, E | D, E | D | A |
| 74 | 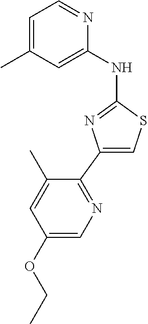 [SMILES: CCOc1cnc(-c2csc(Nc3cc(C)ccn3)n2)c(C)c1] | 4-(5-ethoxy-3- methylpyridin-2-yl)- N-(4-methylpyridin-2- yl)thiazol-2-amine | 327.40 | C | D | D, E | D | C |
| 75 | 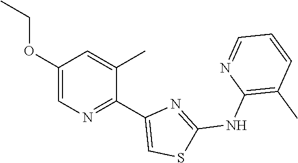 [SMILES: CCOc1cnc(-c2csc(Nc3ncccc3C)n2)c(C)c1] | 4-(5-ethoxy-3- methylpyridin-2-yl)- N-(3-methylpyridin-2- yl)thiazol-2-amine | 327.10 | C | D | D, E | D | C |
| 76 | 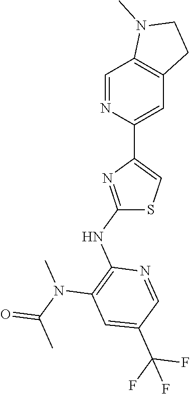 [SMILES: CC(=O)N(C)c1cc(C(F)(F)F)cnc1Nc1nc(-c2cc3c(cn2)N(C)CC3)cs1] | N-methyl-N-(2-(4-(1- methyl-2,3-dihydro- 1H-pyrrolo[2,3- c]pyridin-5-yl)thiazol- 2-ylamino)-5- (trifluoromethyl) pyridin-3-yl)acetamide | 449.40 | C | D, E | D, E | D | A |
| 77 | 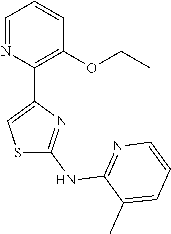 [SMILES: CCOc1cccnc1-c1csc(Nc2ncccc2C)n1] | 4-(3-ethoxypyridin-2- yl)-N-(3- methylpyridin-2- yl)thiazol-2-amine | 313.20 | C | | | | C |
| 78 | 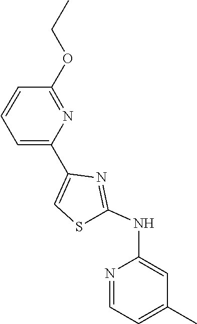 | 4-(6-ethoxypyridin-2- yl)-N-(4- methylpyridin-2- yl)thiazol-2-amine | 313.30 | A | | | | A |
| 79 | 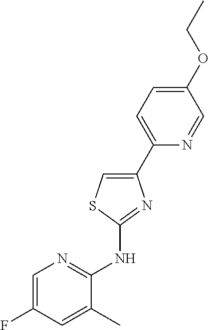 [SMILES: CCOc1ccc(-c2csc(Nc3ncc(F)cc3C)n2)nc1] | 4-(5-ethoxypyridin-2- yl)-N-(5-fluoro-3- methylpyridin-2- yl)thiazol-2-amine | 331.40 | C | | | | B |
| 80 | 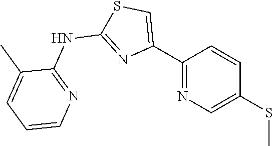 | N-(3-methylpyridin-2- yl)-4-(5- (methylthio)pyridin-2- yl)thiazol-2-amine | 315.10 | C | | | | C |
| 81 | 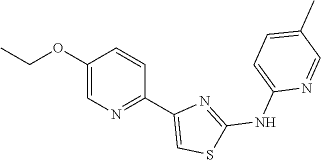 [SMILES: CCOc1ccc(-c2csc(Nc3ccc(C)cn3)n2)nc1] | 4-(5-ethoxypyridin-2- yl)-N-(5- methylpyridin-2- yl)thiazol-2-amine | 313.30 | C | | | | C |
| 82 | 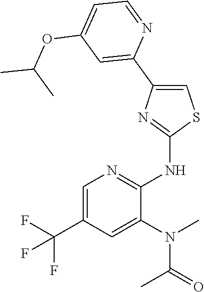 [SMILES: CC(=O)N(C)c1cc(C(F)(F)F)cnc1Nc1nc(-c2cc(OC(C)C)ccn2)cs1] | N-(2-(4-(4- isopropoxypyridin-2- yl)thiazol-2-ylamino)- 5-(trifluoromethyl) pyridin-3-yl)-N- methylacetamide | 452.40 | C | D, E | D, E | D | B |
| 83 | 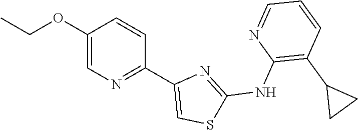 [SMILES: CCOc1ccc(-c2csc(Nc3ncccc3C3CC3)n2)nc1] | N-(3- cyclopropylpyridin-2- yl)-4-(5- ethoxypyridin-2- yl)thiazol-2-amine | 339.20 | B | D | D, E | D | A |
| 84 | 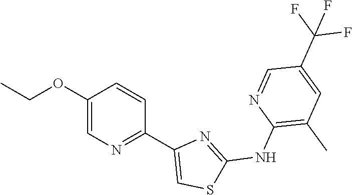 [SMILES: CCOc1ccc(-c2csc(Nc3ncc(C(F)(F)F)cc3C)n2)nc1] | 4-(5-ethoxypyridin-2- yl)-N-(3-methyl-5- (trifluoromethyl) pyridin-2-yl)thiazol-2- amine | 381.10 | B | D | D, E | D | B |
| 85 | 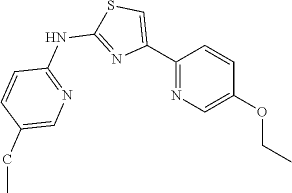 | N-(5-chloropyridin-2- yl)-4-(5- ethoxypyridin-2- yl)thiazol-2-amine | 333.30 | B | | | | A |
| 86 | 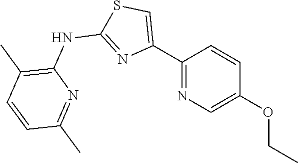 | N-(3,6- dimethylpyridin-2-yl)- 4-(5-ethoxypyridin-2- yl)thiazol-2-amine | 327.30 | C | | | | B |
| 87 | 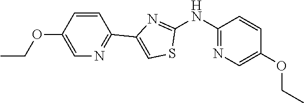 [SMILES: CCOc1ccc(Nc2nc(-c3ccc(OCC)cn3)cs2)nc1] | N,4-bis(5- ethoxypyridin-2- yl)thiazol-2-amine | 343.30 | C | | | | C |
| 88 | 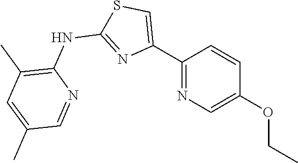 | N-(3,5- dimethylpyridin-2-yl)- 4-(5-ethoxypyridin-2- yl)thiazol-2-amine | 327.40 | C | | | | C |
| 89 | 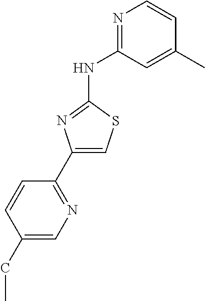 | 4-(5-chloropyridin-2- yl)-N-(4- methylpyridin-2- yl)thiazol-2-amine | 303.00 | B | | | | B |
| 90 | 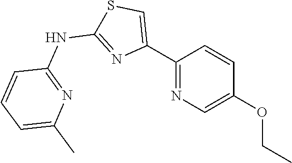 | 4-(5-ethoxypyridin-2- yl)-N-(6- methylpyridin-2- yl)thiazol-2-amine | 313.30 | C | | | | A |
| 91 | 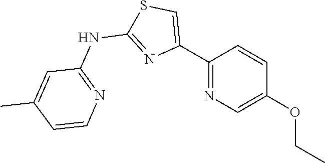 | 4-(5-ethoxypyridin-2- yl)-N-(4- methylpyridin-2- yl)thiazol-2-amine | 313.30 | C | | | | C |
| 92 | 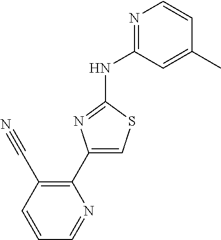 [SMILES: Cc1ccnc(Nc2nc(-c3ncccc3C#N)cs2)c1] | 2-(2-(4- methylpyridin-2- ylamino)thiazol-4- yl)nicotinonitrile | 294.10 | C | D | D, E | D | A |
| 93 | 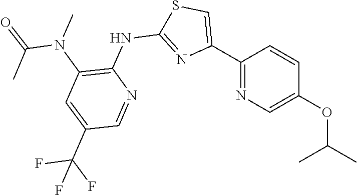 | N-(2-(4-(5- isopropoxypyridin-2- yl)thiazol-2-ylamino)- 5-(trifluoromethyl) pyridin-3-yl)-N- methylacetamide | 452.40 | C | D, E | D, E | D | A |
| 94 | 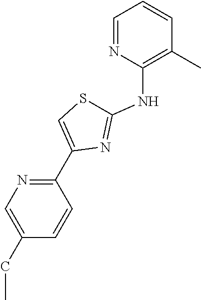 [SMILES: CCc1ccc(-c2csc(Nc3ncccc3C)n2)nc1] | 4-(5-chloropyridin-2- yl)-N-(3- methylpyridin-2- yl)thiazol-2-amine | 303.10 | C | D | D, E | D | B |
| 95 | 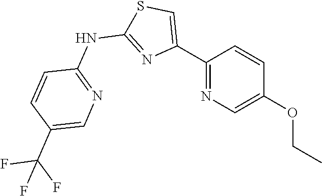 | 4-(5-ethoxypyridin-2- yl)-N-(5- (trifluoromethyl) pyridin-2-yl)thiazol-2- amine | 367.10 | C | D | D, E | D | C |
| 96 | 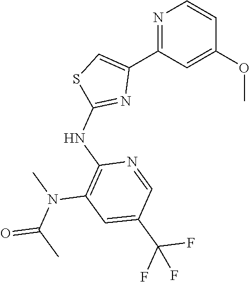 [SMILES: COc1ccnc(-c2csc(Nc3ncc(C(F)(F)F)cc3N(C)C(C)=O)n2)c1] | N-(2-(4-(4- methoxypyridin-2- yl)thiazol-2-ylamino)- 5-(trifluoromethyl) pyridin-3-yl)-N- methylacetamide | 424.40 | B | D | D, E | D | B |
| 97 | 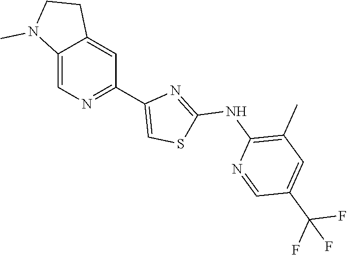 [SMILES: Cc1cc(C(F)(F)F)cnc1Nc1nc(-c2cc3c(cn2)N(C)CC3)cs1] | 4-(1-methyl-2,3- dihydro-1H- pyrrolo[2,3-c]pyridin- 5-yl)-N-(3-methyl-5- (trifluoromethyl) pyridin-2-yl)thiazol-2- amine | 392.30 | C | D | D, E | D | C |
| 98 | 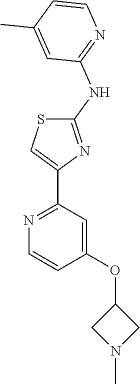 [SMILES: Cc1ccnc(Nc2nc(-c3cc(OC4CN(C)C4)ccn3)cs2)c1] | 4-(4-(1- methylazetidin-3- yloxy)pyridin-2-yl)- N-(4-methylpyridin-2- yl)thiazol-2-amine | 354.30 | C | | | | A |
| 99 | 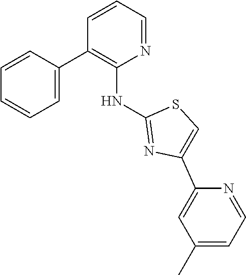 [SMILES: Cc1ccnc(-c2csc(Nc3ncccc3-c3ccccc3)n2)c1] | 4-(4-methylpyridin-2- yl)-N-(3- phenylpyridin-2- yl)thiazol-2-amine | 345.10 | A | | | | C |
| 100 | 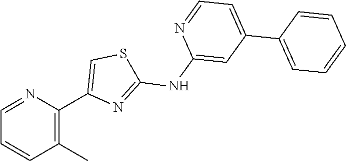 [SMILES: Cc1cccnc1-c1csc(Nc2cc(-c3ccccc3)ccn2)n1] | 4-(3-methylpyridin-2- yl)-N-(4- phenylpyridin-2- yl)thiazol-2-amine | 345.10 | C | | | | B |
| 101 | 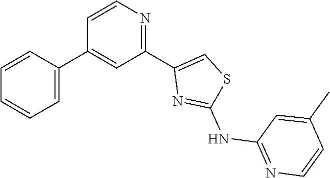 [SMILES: Cc1ccnc(Nc2nc(-c3cc(-c4ccccc4)ccn3)cs2)c1] | N-(4-methylpyridin-2- yl)-4-(4- phenylpyridin-2- yl)thiazol-2-amine | 345.10 | C | | | | C |
| 102 | 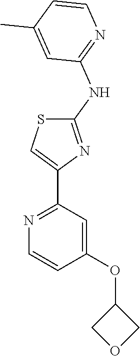 [SMILES: Cc1ccnc(Nc2nc(-c3cc(OC4COC4)ccn3)cs2)c1] | N-(4-methylpyridin-2- yl)-4-(4-(oxetan-3- yloxy)pyridin-2- yl)thiazol-2-amine | 341.00 | C | | | | A |
| 103 | 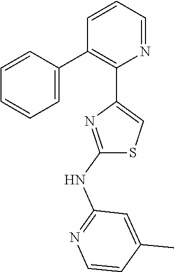 | N-(4-methylpyridin-2- yl)-4-(3- phenylpyridin-2- yl)thiazol-2-amine | 345.10 | C | | | | C |
| 104 | 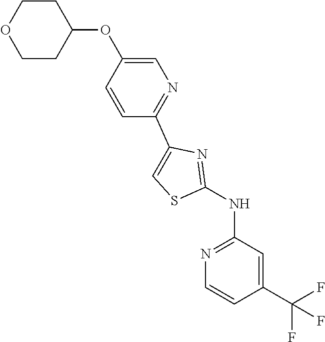 [SMILES: FC(F)(F)c1ccnc(Nc2nc(-c3ccc(OC4CCOCC4)cn3)cs2)c1] | 4-(5-(tetrahydro-2H- pyran-4- yloxy)pyridin-2-yl)- N-(4-(trifluoromethyl) pyridin-2-yl)thiazol-2- amine | 437.10 | C | D, E | | D | |
| 105 | 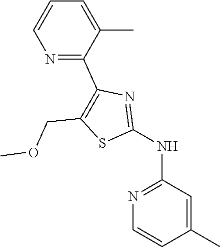 | 5-(methoxymethyl)-4- (3-methylpyridin-2- yl)-N-(4- methylpyridin-2- yl)thiazol-2-amine | 327.40 | | | | | |
| 106 | 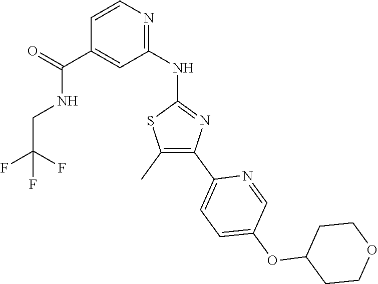 [SMILES: Cc1sc(Nc2cc(C(=O)NCC(F)(F)F)ccn2)nc1-c1ccc(OC2CCOCC2)cn1] | 2-(5-methyl-4-(5- (tetrahydro-2H-pyran- 4-yloxy)pyridin-2- yl)thiazol-2-ylamino)- N-(2,2,2- trifluoroethyl) isonicotinamide | 494.20 | B | | | | |
| 107 | 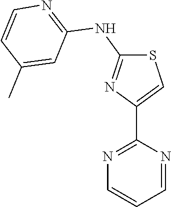 [SMILES: Cc1ccnc(Nc2nc(-c3ncccn3)cs2)c1] | N-(4-methylpyridin-2- yl)-4-(pyrimidin-2- yl)thiazol-2-amine | 270.40 | A | D | | | |
| 108 | 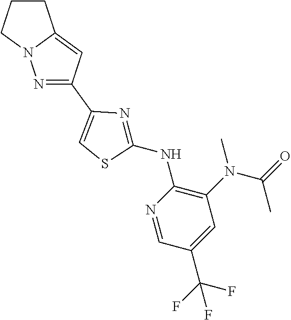 [SMILES: CC(=O)N(C)c1cc(C(F)(F)F)cnc1Nc1nc(-c2cc3n(n2)CCC3)cs1] | N-(2-(4-(5,6-dihydro- 4H-pyrrolo[1,2- b]pyrazol-2- yl)thiazol-2-ylamino)- 5-(trifluoromethyl) pyridin-3-yl)-N- methylacetamide | 423.20 | C | D | D, E | D | |
| 109 | 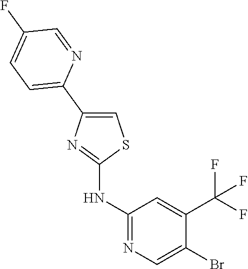 [SMILES: Fc1ccc(-c2csc(Nc3cc(C(F)(F)F)c(Br)cn3)n2)nc1] | N-(5-bromo-4- (trifluoromethyl) pyridin-2-yl)-4-(5- fluoropyridin-2- yl)thiazol-2-amine | 419.00 | B | | | | |
| 110 | 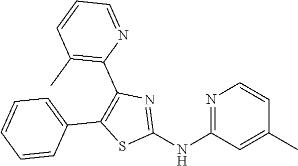 [SMILES: Cc1ccnc(Nc2nc(-c3ncccc3C)c(-c3ccccc3)s2)c1] | 4-(3-methylpyridin-2- yl)-N-(4- methylpyridin-2-yl)-5- phenylthiazol-2-amine | 359.3 | A | | | D | |
| 111 | 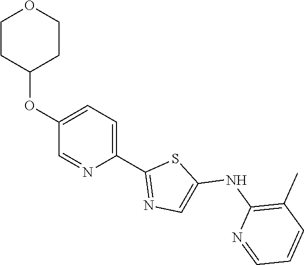 [SMILES: Cc1cccnc1Nc1cnc(-c2ccc(OC3CCOCC3)cn2)s1] | N-(3-methylpyridin-2- yl)-2-(5-(tetrahydro- 2H-pyran-4- yloxy)pyridin-2- yl)thiazol-5-amine | 369.2 | C | | | D | |
| 112 | 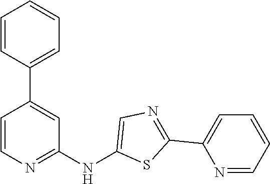 [SMILES: c1ccc(-c2ccnc(Nc3cnc(-c4ccccn4)s3)c2)cc1] | N-(4-phenylpyridin-2- yl)-2-(pyridin-2- yl)thiazol-5-amine | 331.4 | B | | | | |
| 113 | 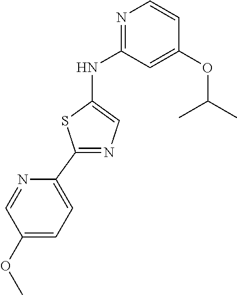 [SMILES: COc1ccc(-c2ncc(Nc3cc(OC(C)C)ccn3)s2)nc1] | N-(4- isopropoxypyridin-2- yl)-2-(5- methoxypyridin-2- yl)thiazol-5-amine | 343 | A | | | D | |
| 114 | 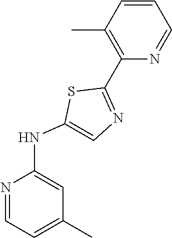 [SMILES: Cc1ccnc(Nc2cnc(-c3ncccc3C)s2)c1] | 2-(3-methylpyridin-2- yl)-N-(4- methylpyridin-2- yl)thiazol-5-amine | 283 | C | | | D | |
| 115 | 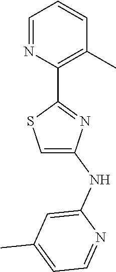 | 2-(3-methylpyridin-2- yl)-N-(4- methylpyridin-2- yl)thiazol-4-amine | 283.1 | A | | | | |
| 116 | 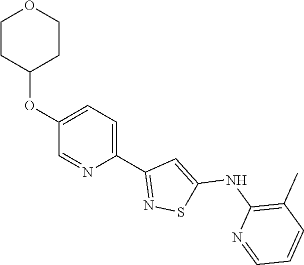 [SMILES: Cc1cccnc1Nc1cc(-c2ccc(OC3CCOCC3)cn2)ns1] | N-(3-methylpyridin-2- yl)-3-(5-(tetrahydro- 2H-pyran-4- yloxy)pyridin-2- yl)isothiazol-5-amine | 369.4 | A | | | D | |
| 117 | 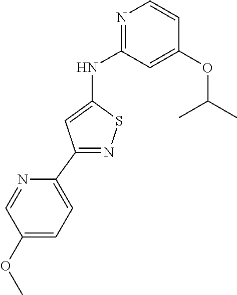 [SMILES: COc1ccc(-c2cc(Nc3cc(OC(C)C)ccn3)sn2)nc1] | N-(4- isopropoxypyridin-2- yl)-3-(5- methoxypyridin-2- yl)isothiazol-5-amine | 343.4 | A | | | D | |
| 118 | 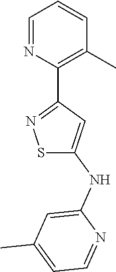 | 3-(3-methylpyridin-2- yl)-N-(4- methylpyridin-2- yl)isothiazol-5-amine | 283.3 | A | | | D | |
| 119 | 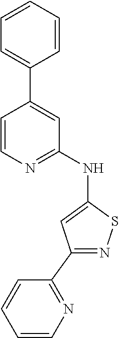 [SMILES: c1ccc(-c2ccnc(Nc3cc(-c4ccccn4)ns3)c2)cc1] | N-(4-phenylpyridin-2- yl)-3-(pyridin-2- yl)isothiazol-5-amine | 331 | A | | | D | |

TABLE 2

| | | | | O. | B. | B. | L. | |
|---|---|---|---|---|---|---|---|---|
| | | | | gutt | malayi | phangi | sigmodontis | D. |
| Com. | | | | Act | Act | Act | Act | immitis |
| No. | Structure | Name | M + 1 | class | class | class | class | Act class |
| 120 | 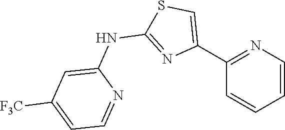 [SMILES: FC(F)(F)c1ccnc(Nc2nc(-c3ccccn3)cs2)c1] | 4-(pyridin- 2-yl)-N- (4- (trifluoromethyl) pyridin- 2-yl)thiazol- 2-amine | 322.6 | C | D | | | |
| 121 | 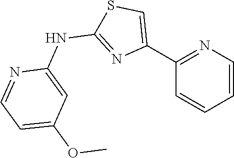 [SMILES: COc1ccnc(Nc2nc(-c3ccccn3)cs2)c1] | N-(4- methoxypyridin- 2-yl)-4- (pyridin-2- yl)thiazol- 2-amine | 285.2 | B | D | | | B |
| 122 | 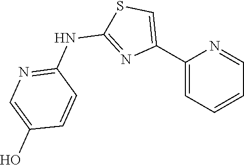 [SMILES: Oc1ccc(Nc2nc(-c3ccccn3)cs2)nc1] | 6-(4- (pyridin-2-yl) thiazol-2- ylamino) pyridin-3-ol | 271.4 | A | D | | | |
| 123 | 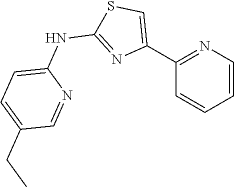 [SMILES: CCc1ccc(Nc2nc(-c3ccccn3)cs2)nc1] | N-(5- ethylpyridin-2- yl)-4- (pyridin-2- yl)thiazol- 2-amine | 283.3 | C | | | D | |
| 124 | 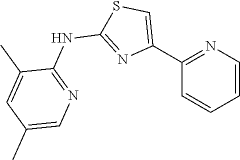 [SMILES: Cc1cnc(Nc2nc(-c3ccccn3)cs2)c(C)c1] | N-(3,5- dimethyl- pyridin-2- yl)-4- (pyridin-2- yl)thiazol- 2-amine | 282.36 | C | D | | | C |
| 125 | 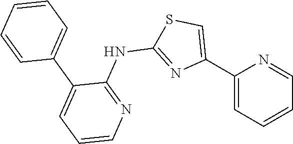 [SMILES: c1ccc(-c2cccnc2Nc2nc(-c3ccccn3)cs2)cc1] | N-(3- phenylpyridin- 2-yl)-4- (pyridin-2- yl)thiazol- 2-amine | 331.2 | A | D | | | A |
| 126 | 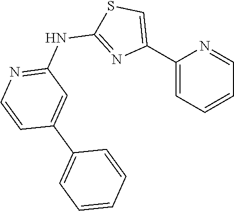 [SMILES: c1ccc(-c2ccnc(Nc3nc(-c4ccccn4)cs3)c2)cc1] | N-(4- phenylpyridin- 2-yl)-4- (pyridin-2- yl)thiazol- 2-amine | 331.2 | C | D, E | | | C |
| 127 | 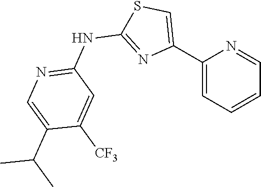 [SMILES: CC(C)c1cnc(Nc2nc(-c3ccccn3)cs2)cc1C(F)(F)F] | N-(5- isopropyl-4- (trifluoro- methyl) pyridin- 2-yl)- 4-(pyridin- 2-yl)thiazol- 2-amine | 365.23 | A | D, E | | D | C |
| 128 | 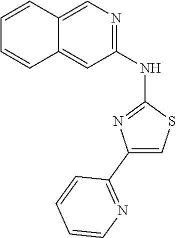 [SMILES: c1ccc(-c2csc(Nc3cc4ccccc4cn3)n2)nc1] | N- (isoquinolin- 3-yl)- 4-(pyridin-2- yl)thiazol- 2-amine | 305.2 | C | D, E | | D | B |
| 129 | 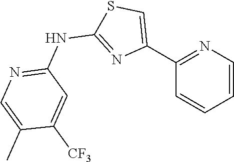 [SMILES: Cc1cnc(Nc2nc(-c3ccccn3)cs2)cc1C(F)(F)F] | N-(5- methyl-4- (trifluoro- methyl) pyridin-2-yl)- 4-(pyridin- 2-yl)thiazol- 2-amine | 337.2 | C | D, E | | D | C |
| 130 | 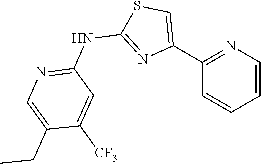 [SMILES: CCc1cnc(Nc2nc(-c3ccccn3)cs2)cc1C(F)(F)F] | N-(5-ethyl-4- (trifluoro- methyl) pyridin-2-yl)- 4-(pyridin- 2-yl)thiazol- 2-amine | 351.2 | C | D, E | | D | C |
| 131 | 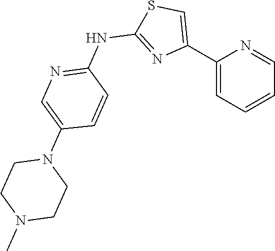 [SMILES: CN1CCN(c2ccc(Nc3nc(-c4ccccn4)cs3)nc2)CC1] | N-(5-(4- methyl- piperazin-1- yl)pyridin- 2-yl)-4- (pyridin-2- yl)thiazol- 2-amine | 353.2 | C | D, E | | | |
| 132 | 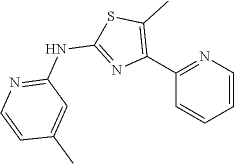 [SMILES: Cc1ccnc(Nc2nc(-c3ccccn3)c(C)s2)c1] | 5-methyl- N-(4- methyl- pyridin-2-yl)- 4-(pyridin-2- yl)thiazol- 2-amine | 283.2 | B | D | | D, E | B |
| 133 | 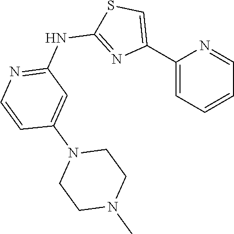 [SMILES: CN1CCN(c2ccnc(Nc3nc(-c4ccccn4)cs3)c2)CC1] | N-(4-(4- methyl- piperazin-1- yl)pyridin- 2-yl)-4- (pyridin-2- yl)thiazol- 2-amine | 353.2 | | D | | | |
| 134 | 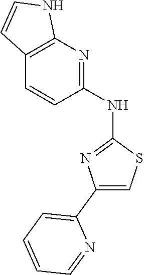 [SMILES: c1ccc(-c2csc(Nc3ccc4cc[nH]c4n3)n2)nc1] | 4-(pyridin- 2-yl)-N- (1H-pyrrolo [2,3- b]pyridin-6- yl)thiazol- 2-amine | 294.2 | C | | | | A |
| 135 | 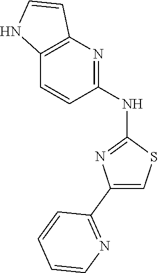 [SMILES: c1ccc(-c2csc(Nc3ccc4[nH]ccc4n3)n2)nc1] | 4-(pyridin- 2-yl)-N- (1H-pyrrolo [3,2- b]pyridin-5- yl)thiazol- 2-amine | 294.2 | A | | | | |
| 136 | 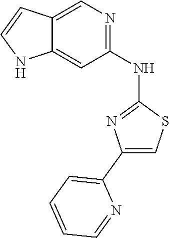 [SMILES: c1ccc(-c2csc(Nc3cc4[nH]ccc4cn3)n2)nc1] | 4-(pyridin- 2-yl)-N- (1H-pyrrolo [3,2- c]pyridin-6- yl)thiazol- 2-amine | 305.2 | C | | | | A |
| 137 | 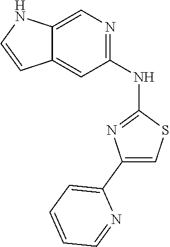 [SMILES: c1ccc(-c2csc(Nc3cc4cc[nH]c4cn3)n2)nc1] | 4-(pyridin- 2-yl)-N- (1H-pyrrolo [2,3-c] pyridin-5- yl)thiazol- 2-amine | 294.2 | A | D | | | |
| 138 | 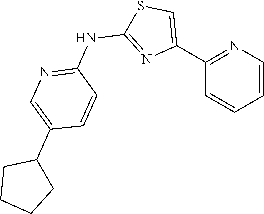 [SMILES: c1ccc(-c2csc(Nc3ccc(C4CCCC4)cn3)n2)nc1] | N-(5- cyclopentyl- pyridin- 2- yl)-4- (pyridin-2- yl)thiazol- 2-amine | 323.3 | C | D, E | | D, E | C |
| 139 | 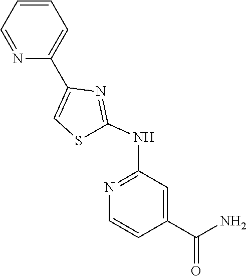 [SMILES: NC(=O)c1ccnc(Nc2nc(-c3ccccn3)cs2)c1] | 2-(4- (pyridin-2- yl)thiazol-2- ylamino) iso- nicotinamide | 298.1 | C | D | | D, E | |
| 140 | 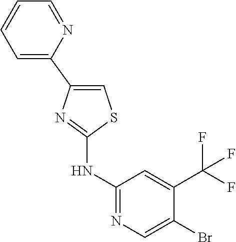 [SMILES: FC(F)(F)c1cc(Nc2nc(-c3ccccn3)cs2)ncc1Br] | N-(5- bromo-4- (trifluoro- methyl) pyridin-2-yl)- 4-(pyridin- 2-yl)thiazol- 2-amine | 402 | A | | | | |
| 141 | 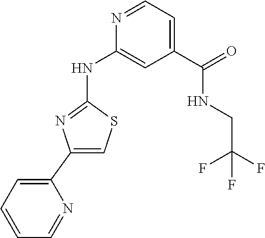 [SMILES: O=C(NCC(F)(F)F)c1ccnc(Nc2nc(-c3ccccn3)cs2)c1] | 2-(4- (pyridin-2- yl)thiazol-2- ylamino)- N-(2,2,2- trifluoroethyl) isonicotinamide | 380 | B | | | | |
| 142 | 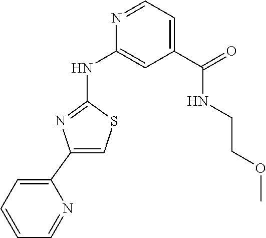 [SMILES: COCCNC(=O)c1ccnc(Nc2nc(-c3ccccn3)cs2)c1] | N-(2- methoxyethyl)- 2-(4-(pyridin-2- yl)thiazol-2- ylamino) isonicotinamide | 356.1 | B | D | | D, E | |
| 143 | 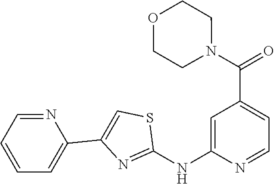 [SMILES: O=C(c1ccnc(Nc2nc(-c3ccccn3)cs2)c1)N1CCOCC1] | morpholino (2-(4- (pyridin-2- yl)thiazol- 2-ylamino) pyridin-4- yl)methanone | 368.1 | B | | | | |
| 144 | 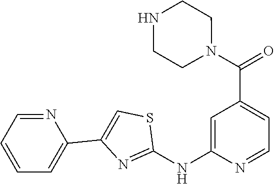 [SMILES: O=C(c1ccnc(Nc2nc(-c3ccccn3)cs2)c1)N1CCNCC1] | piperazin- 1-yl(2-(4- (pyridin-2- yl)thiazol- 2-ylamino) pyridin-4- yl)methanone | 367.1 | B | | | | |
| 145 | 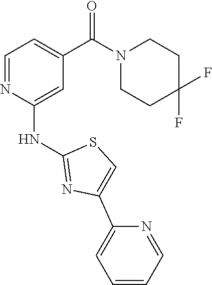 [SMILES: O=C(c1ccnc(Nc2nc(-c3ccccn3)cs2)c1)N1CCC(F)(F)CC1] | (4,4- difluoro- piperidin-1- yl)(2-(4- (pyridin-2- yl)thiazol-2- ylamino) pyridin-4- yl)methanone | 402.1 | C | D, E | | D, E | |
| 146 | 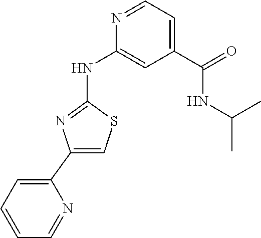 [SMILES: CC(C)NC(=O)c1ccnc(Nc2nc(-c3ccccn3)cs2)c1] | N-isopropyl- 2-(4- (pyridin-2- yl)thiazol- 2-ylamino) isonicotinamide | 340.2 | C | D | | | |
| 147 | 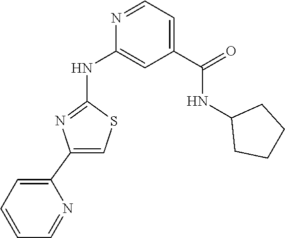 [SMILES: O=C(NC1CCCC1)c1ccnc(Nc2nc(-c3ccccn3)cs2)c1] | N-cyclopentyl- 2-(4- (pyridin-2- yl)thiazol- 2-ylamino) isonicotinamide | 366.2 | C | D | | D | |
| 148 | 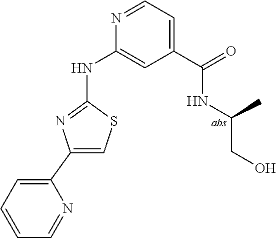 [SMILES: C[C@@H](CO)NC(=O)c1ccnc(Nc2nc(-c3ccccn3)cs2)c1] | (S)-N-(1- hydroxy- propan-2-yl)- 2-(4-(pyridin-2- yl)thiazol-2- ylamino) isonicotinamide | 356.1 | B | | | | |
| 149 | 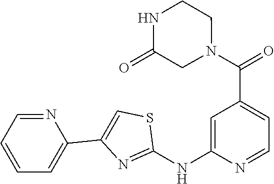 [SMILES: O=C1CN(C(=O)c2ccnc(Nc3nc(-c4ccccn4)cs3)c2)CCN1] | 4-(2-(4- (pyridin-2- yl)thiazol-2- ylamino) isonicotinoyl) piperazin- 2-one | 381.1 | A | | | | |
| 150 | 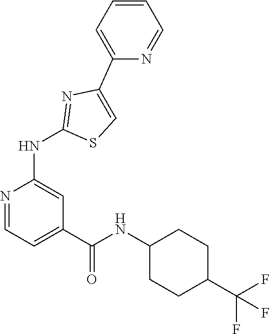 [SMILES: O=C(NC1CCC(C(F)(F)F)CC1)c1ccnc(Nc2nc(-c3ccccn3)cs2)c1] | 2-(4-(pyridin-2- yl)thiazol-2- ylamino)-N-(4- (trifluoromethyl) cyclohexyl) isonicotinamide | 448.1 | C | D | | E | |
| 151 | 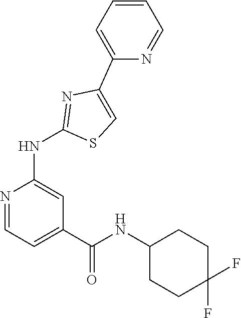 [SMILES: O=C(NC1CCC(F)(F)CC1)c1ccnc(Nc2nc(-c3ccccn3)cs2)c1] | N-(4,4- difluoro- cyclohexyl)- 2-(4-(pyridin-2- yl)thiazol-2- ylamino) isonicotinamide | 416.1 | C | D | | | |
| 152 | 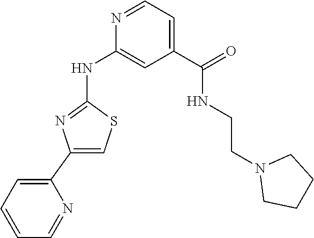 [SMILES: O=C(NCCN1CCCC1)c1ccnc(Nc2nc(-c3ccccn3)cs2)c1] | 2-(4-(pyridin-2- yl)thiazol-2- ylamino)-N-(2- (pyrrolidin-1- yl)ethyl) isonicotinamide | 395.2 | B | D, E | | | |
| 153 | 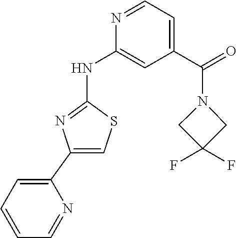 [SMILES: O=C(c1ccnc(Nc2nc(-c3ccccn3)cs2)c1)N1CC(F)(F)C1] | (3,3- difluoroazetidin- 1-yl)(2-(4- (pyridin-2- yl)thiazol-2- ylamino) pyridin-4- yl)methanone | 374.1 | C | D | | D | |
| 154 | 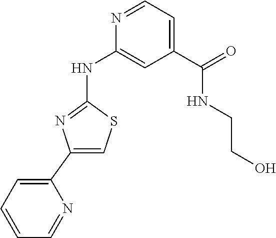 [SMILES: O=C(NCCO)c1ccnc(Nc2nc(-c3ccccn3)cs2)c1] | N-(2- hydroxyethyl)- 2-(4-(pyridin-2- yl)thiazol-2- ylamino) isonicotinamide | 342.1 | B | D | | D | |
| 155 | 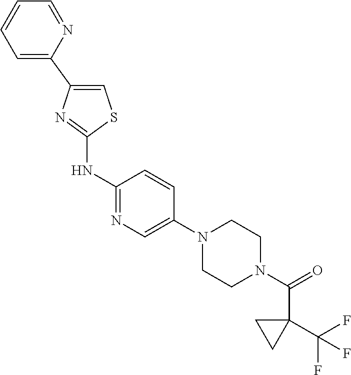 [SMILES: O=C(N1CCN(c2ccc(Nc3nc(-c4ccccn4)cs3)nc2)CC1)C1(C(F)(F)F)CC1] | (4-(6-(4- (pyridin-2- yl)thiazol-2- ylamino) pyridin-3- yl)piperazin- 1-yl)(1- (trifluoromethyl) cyclopropyl) methanone | 475.1 | C | D | | | |
| 156 | 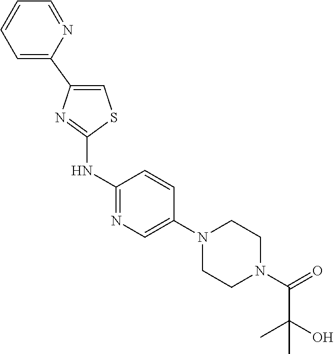 [SMILES: CC(C)(O)C(=O)N1CCN(c2ccc(Nc3nc(-c4ccccn4)cs3)nc2)CC1] | 2-hydroxy- 2-methyl- 1-(4-(6-(4- (pyridin-2- yl)thiazol-2- ylamino) pyridin-3- yl)piperazin-1- yl)propan-1-one | 425.1 | C | D | | | |
| 157 | 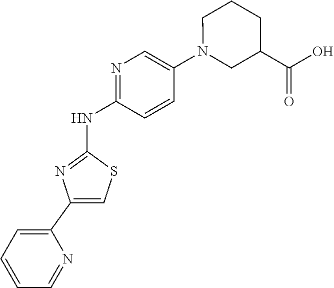 [SMILES: O=C(O)C1CCCN(c2ccc(Nc3nc(-c4ccccn4)cs3)nc2)C1] | 1-(6-(4- (pyridin-2- yl)thiazol-2- ylamino) pyridin-3- yl)piperidine-3- carboxylic acid | 382.1 | A | D, E | | D | |
| 158 | 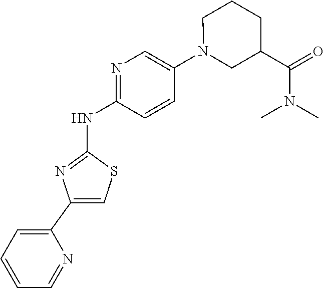 [SMILES: CN(C)C(=O)C1CCCN(c2ccc(Nc3nc(-c4ccccn4)cs3)nc2)C1] | N,N- dimethyl-1-(6- (4-(pyridin-2- yl)thiazol-2- ylamino)pyridin- 3- yl)piperidine-3- carboxamide | 409.2 | C | D | | | |
| 159 | 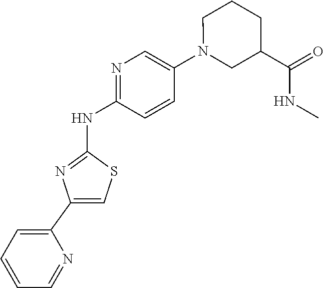 [SMILES: CNC(=O)C1CCCN(c2ccc(Nc3nc(-c4ccccn4)cs3)nc2)C1] | N-methyl- 1-(6-(4- (pyridin-2- yl)thiazol- 2-ylamino) pyridin-3- yl)piperidine-3- carboxamide | 395.2 | B | D | | D, E | |
| 160 | 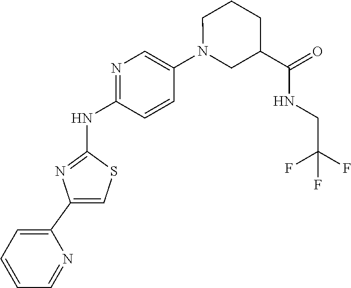 [SMILES: O=C(NCC(F)(F)F)C1CCCN(c2ccc(Nc3nc(-c4ccccn4)cs3)nc2)C1] | 1-(6-(4- (pyridin-2- yl)thiazol-2- ylamino) pyridin-3- yl)-N-(2,2,2- trifluoroethyl) piperidine-3- carboxamide | 463.2 | C | D | | | |
| 161 | 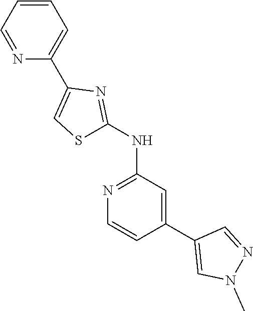 [SMILES: Cn1cc(-c2ccnc(Nc3nc(-c4ccccn4)cs3)c2)cn1] | N-(4-(1- methyl-1H- pyrazol-4- yl)pyridin- 2-yl)-4- (pyridin-2- yl)thiazol- 2-amine | 335 | C | | | | |
| 162 | 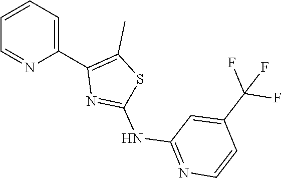 [SMILES: Cc1sc(Nc2cc(C(F)(F)F)ccn2)nc1-c1ccccn1] | 5-methyl- 4-(pyridin- 2-yl)-N-(4- (trifluoromethyl) pyridin-2-yl) thiazol-2- amine | 337.1 | C | D, E | | D, E | |
| 163 | 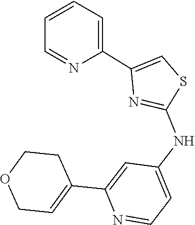 [SMILES: C1=C(c2cc(Nc3nc(-c4ccccn4)cs3)ccn2)CCOC1] | N-(4-(3,6- dihydro- 2H-pyran-4- yl)pyridin- 2-yl)-4- (pyridin-2-yl) thiazol- 2-amine | 337 | C | | | | |
| 164 | 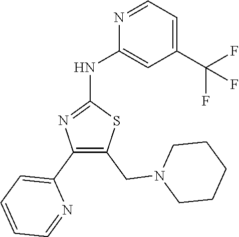 [SMILES: FC(F)(F)c1ccnc(Nc2nc(-c3ccccn3)c(CN3CCCCC3)s2)c1] | 5-(piperidin-1- ylmethyl)-4- (pyridin- 2-yl)-N-(4- (trifluoromethyl) pyridin-2- yl)thiazol-2- amine | 420.2 | C | D, E | | D | |
| 165 | 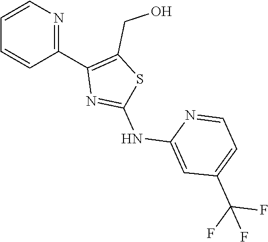 [SMILES: OCc1sc(Nc2cc(C(F)(F)F)ccn2)nc1-c1ccccn1] | (4-(pyridin- 2-yl)-2- (4- (trifluoromethyl) pyridin-2- ylamino) thiazol-5- yl)methanol | 353.1 | C | D, E | | D, E | |
| 166 | 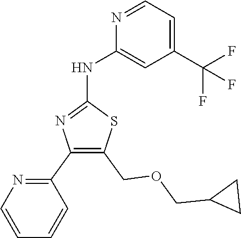 [SMILES: FC(F)(F)c1ccnc(Nc2nc(-c3ccccn3)c(COCC3CC3)s2)c1] | 5- ((cyclopropyl- methoxy)methyl)- 4-(pyridin- 2-yl)-N-(4- (trifluoromethyl) pyridin-2-yl) thiazol-2- amine | 407.1 | B | D | | D | |
| 167 | 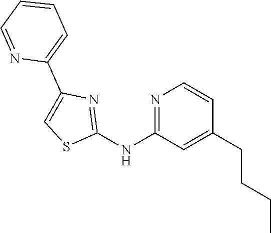 [SMILES: CCCCc1ccnc(Nc2nc(-c3ccccn3)cs2)c1] | N-(4- butylpyridin-2- yl)-4- (pyridin-2- yl)thiazol- 2-amine | 310.7 | C | | | D | C |
| 168 | 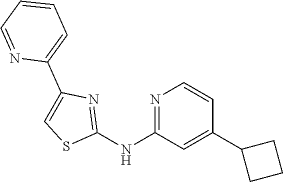 [SMILES: c1ccc(-c2csc(Nc3cc(C4CCC4)ccn3)n2)nc1] | N-(4- cyclobutyl- pyridin-2- yl)-4- (pyridin-2- yl)thiazol- 2-amine | 309.2 | C | | D | D | C |
| 169 | 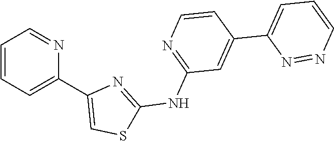 [SMILES: c1ccc(-c2csc(Nc3cc(-c4cccnn4)ccn3)n2)nc1] | N-(4- (pyridazin-3- yl)pyridin- 2-yl)-4- (pyridin-2- yl)thiazol- 2-amine | 333.1 | C | | D | D | A |
| 170 | 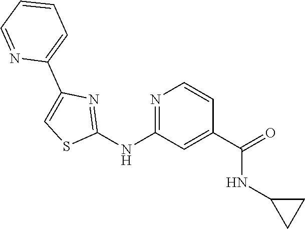 [SMILES: O=C(NC1CC1)c1ccnc(Nc2nc(-c3ccccn3)cs2)c1] | N-cyclopropyl- 2-(4- (pyridin-2- yl)thiazol- 2-ylamino) isonicotinamide | 338.1 | C | | D | D | A |
| 171 | 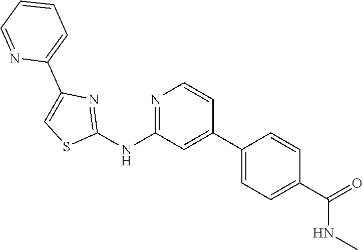 [SMILES: CNC(=O)c1ccc(-c2ccnc(Nc3nc(-c4ccccn4)cs3)c2)cc1] | N-methyl- 4-(2-(4- (pyridin-2- yl)thiazol- 2-ylamino) pyridin-4- yl)benzamide | 388 | C | | D | D | A |
| 172 | 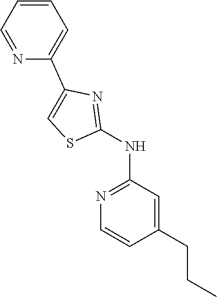 [SMILES: CCCc1ccnc(Nc2nc(-c3ccccn3)cs2)c1] | N-(4- propylpyridin- 2-yl)-4- (pyridin-2- yl)thiazol- 2-amine | 296.8 | B | | | | C |
| 173 | 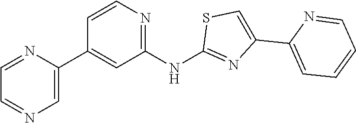 [SMILES: c1ccc(-c2csc(Nc3cc(-c4cnccn4)ccn3)n2)nc1] | N-(4- (pyrazin-2- yl)pyridin- 2-yl)-4- (pyridin-2- yl)thiazol- 2-amine | 333.1 | C | | | | B |
| 174 | 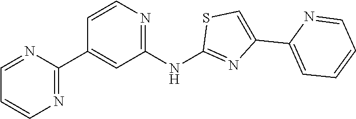 [SMILES: c1ccc(-c2csc(Nc3cc(-c4ncccn4)ccn3)n2)nc1] | 4-(pyridin- 2-yl)-N- (4- (pyrimidin-2- yl)pyridin-2- yl)thiazol- 2-amine | 333 | B | | | | B |
| 175 | 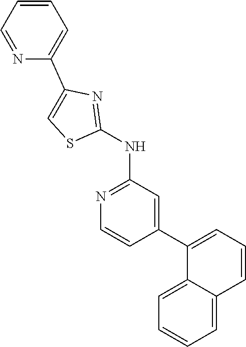 [SMILES: c1ccc(-c2csc(Nc3cc(-c4cccc5ccccc45)ccn3)n2)nc1] | N-(4- (naphthalen-1- yl)pyridin- 2-yl)-4- (pyridin-2- yl)thiazol- 2-amine | 381 | C | | | | C |
| 176 | 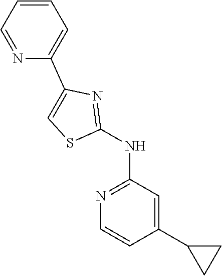 [SMILES: c1ccc(-c2csc(Nc3cc(C4CC4)ccn3)n2)nc1] | N-(4- cyclopropyl- pyridin- 2-yl)-4- (pyridin-2- yl)thiazol- 2-amine | 294.8 | C | | | | C |
| 177 | 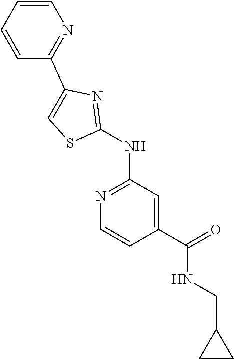 [SMILES: O=C(NCC1CC1)c1ccnc(Nc2nc(-c3ccccn3)cs2)c1] | N- (cyclopropyl- methyl)- 2-(4- (pyridin-2- yl)thiazol-2- ylamino) isonicotinamide | 352.1 | A | | | | |
| 178 | 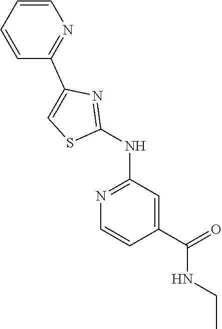 [SMILES: CCNC(=O)c1ccnc(Nc2nc(-c3ccccn3)cs2)c1] | N-ethyl-2-(4- (pyridin-2- yl)thiazol- 2-ylamino) isonicotinamide | 326.1 | C | | | | |
| 179 | 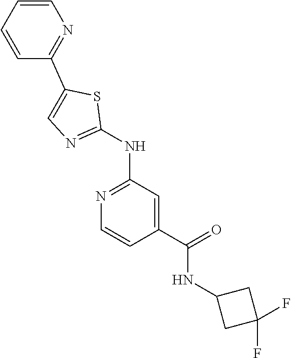 [SMILES: O=C(NC1CC(F)(F)C1)c1ccnc(Nc2ncc(-c3ccccn3)s2)c1] | N-(3,3- difluoro- cyclobutyl)- 2-(5-(pyridin-2- yl)thiazol-2- ylamino) isonicotinamide | 388.1 | C | D | | | |
| 180 | 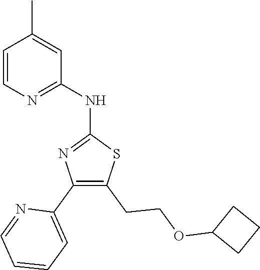 [SMILES: Cc1ccnc(Nc2nc(-c3ccccn3)c(CCOC3CCC3)s2)c1] | 5-(2- cyclobutoxy- ethyl)-N- (4- methylpyridin-2- yl)-4- (pyridin-2- yl)thiazol- 2-amine | 367.1 | C | | D | D, E | C |
| 181 | 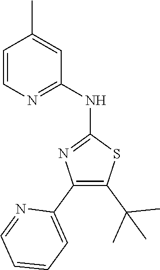 [SMILES: Cc1ccnc(Nc2nc(-c3ccccn3)c(C(C)(C)C)s2)c1] | 5-tert-butyl- N-(4- methylpyridin- 2-yl)- 4-(pyridin-2- yl)thiazol- 2-amine | 325.3 | B | | | | A |
| 182 | 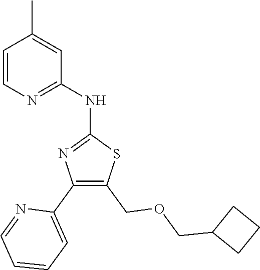 [SMILES: Cc1ccnc(Nc2nc(-c3ccccn3)c(COCC3CCC3)s2)c1] | 5- ((cyclobutyl- methoxy) methyl)-N-(4- methylpyridin- 2-yl)- 4-(pyridin-2- yl)thiazol- 2-amine | 367.2 | B | | D | D, E | C |
| 183 | 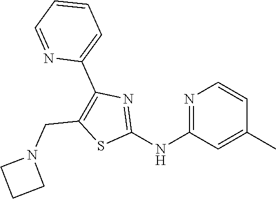 [SMILES: Cc1ccnc(Nc2nc(-c3ccccn3)c(CN3CCC3)s2)c1] | 5-(azetidin-1- ylmethyl)- N-(4- methylpyridin- 2-yl)- 4-(pyridin-2- yl)thiazol- 2-amine | 338 | C | | | | A |
| 184 | 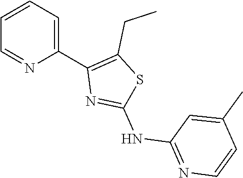 [SMILES: CCc1sc(Nc2cc(C)ccn2)nc1-c1ccccn1] | 5-ethyl- N-(4- methylpyridin- 2-yl)- 4-(pyridin-2- yl)thiazol- 2-amine | 297.1 | C | | | | B |
| 185 | 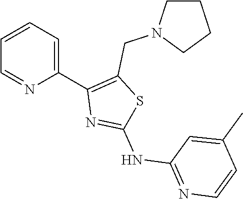 [SMILES: Cc1ccnc(Nc2nc(-c3ccccn3)c(CN3CCCC3)s2)c1] | N-(4- methylpyridin- 2-yl)-4- (pyridin-2- yl)-5- (pyrrolidin-1- ylmethyl) thiazol-2- amine | 352.1 | B | | | | |
| 186 | 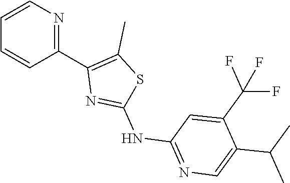 [SMILES: Cc1sc(Nc2cc(C(F)(F)F)c(C(C)C)cn2)nc1-c1ccccn1] | N-(5- isopropyl-4- (trifluoromethyl) pyridin-2-yl)-5- methyl-4- (pyridin-2- yl)thiazol- 2-amine | 379.1 | C | | | | |
| 187 | 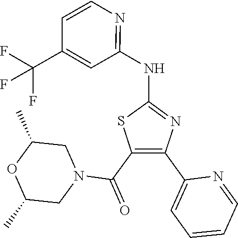 [SMILES: C[C@@H]1CN(C(=O)c2sc(Nc3cc(C(F)(F)F)ccn3)nc2-c2ccccn2)C[C@H](C)O1] | ((2S,6R)-2,6- dimethyl- morpholino) (4-(pyridin- 2-yl)-2- (4- (trifluoromethyl) pyridin-2- ylamino) thiazol-5- yl)methanone | 463.7 | C | | | | |
| 188 | 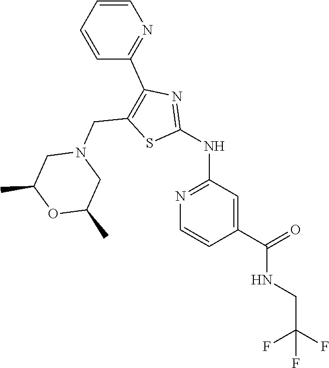 [SMILES: C[C@@H]1CN(Cc2sc(Nc3cc(C(=O)NCC(F)(F)F)ccn3)nc2-c2ccccn2)C[C@H](C)O1] | 2-(5- (((2S,6R)-2,6- dimethyl- morpholino) methyl)-4- (pyridin-2- yl)thiazol-2- ylamino)- N-(2,2,2- trifluoroethyl) isonicotinamide | 506.8 | C | | | | |
| 189 | 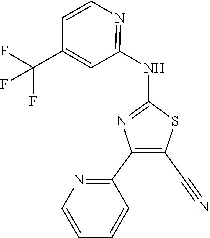 [SMILES: N#Cc1sc(Nc2cc(C(F)(F)F)ccn2)nc1-c1ccccn1] | 4-(pyridin- 2-yl)-2-(4- (trifluoromethyl) pyridin-2- ylamino) thiazole-5- carbonitrile | 347.8 | A | | | | |
| 190 | 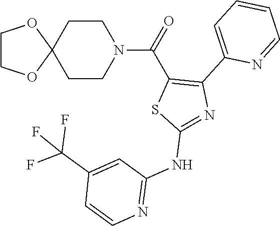 [SMILES: O=C(c1sc(Nc2cc(C(F)(F)F)ccn2)nc1-c1ccccn1)N1CCC2(CC1)OCCO2] | (4-(pyridin- 2-yl)-2- (4- (trifluoromethyl) pyridin-2- ylamino) thiazol-5- yl)(1,4-dioxa-8- azaspiro[4.5] decan- 8-yl)methanone | 491.9 | A | | | | |
| 191 | 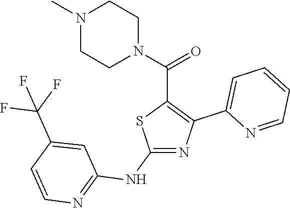 [SMILES: CN1CCN(C(=O)c2sc(Nc3cc(C(F)(F)F)ccn3)nc2-c2ccccn2)CC1] | (4- methylpiperazin- 1-yl)(4- (pyridin-2- yl)-2-(4- (trifluoromethyl) pyridin-2- ylamino) thiazol-5- yl)methanone | 448.9 | A | | | | |
| 192 | 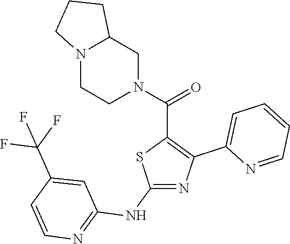 [SMILES: O=C(c1sc(Nc2cc(C(F)(F)F)ccn2)nc1-c1ccccn1)N1CCN2CCCC2C1] | (hexahydro- pyrrolo [1,2-a] pyrazin-2(1H)- yl)(4- (pyridin-2-yl)- 2-(4- (trifluoromethyl) pyridin-2- ylamino) thiazol-5- yl)methanone | 474.9 | B | | | | |
| 193 | 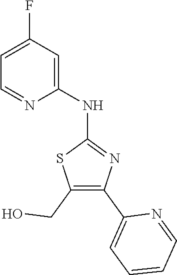 [SMILES: OCc1sc(Nc2cc(F)ccn2)nc1-c1ccccn1] | (2-(4- fluoropyridin- 2-ylamino)-4- (pyridin-2- yl)thiazol- 5-yl)methanol | 303 | C | | | | |
| 194 | 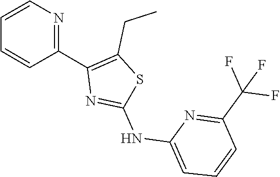 [SMILES: CCc1sc(Nc2cccc(C(F)(F)F)n2)nc1-c1ccccn1] | 5-ethyl-4- (pyridin-2- yl)-N-(6- (trifluoromethyl) pyridin-2- yl)thiazol- 2-amine | 350.9 | A | | | | |
| 195 | 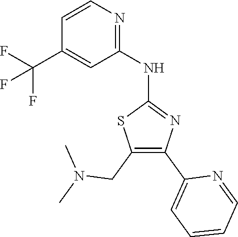 [SMILES: CN(C)Cc1sc(Nc2cc(C(F)(F)F)ccn2)nc1-c1ccccn1] | 5- ((dimethylamino) methyl)-4- (pyridin- 2-yl) N-(4- (trifluoromethyl) pyridin-2- yl)thiazol- 2-amine | 379.9 | B | | | | |
| 196 | 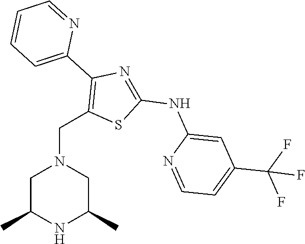 [SMILES: C[C@@H]1CN(Cc2sc(Nc3cc(C(F)(F)F)ccn3)nc2-c2ccccn2)C[C@H](C)N1] | 5- (((3R,5S)-3,5- dimethyl- piperazin-1- yl)methyl)-4- (pyridin-2- yl)-N-(4- (trifluoromethyl) pyridin-2- yl)thiazol- 2-amine | 449.1 | C | | | | |
| 197 | 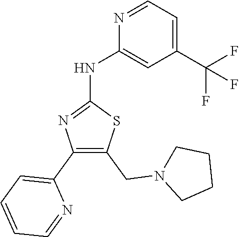 [SMILES: FC(F)(F)c1ccnc(Nc2nc(-c3ccccn3)c(CN3CCCC3)s2)c1] | 4-(pyridin- 2-yl)-5- (pyrrolidin-1- ylmethyl)- N-(4- (trifluoromethyl) pyridin-2- yl)thiazol- 2-amine | 406.1 | A | | | | |
| 198 | 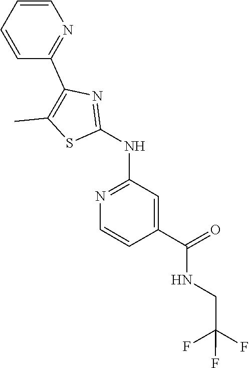 [SMILES: Cc1sc(Nc2cc(C(=O)NCC(F)(F)F)ccn2)nc1-c1ccccn1] | 2-(5-methyl-4- (pyridin-2- yl)thiazol- 2-ylamino)- N-(2,2,2- trifluoroethyl) isonicotinamide | 394 | C | | | | |
| 199 | 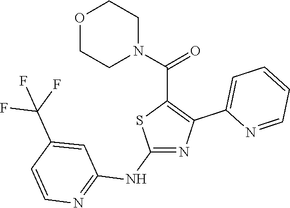 [SMILES: O=C(c1sc(Nc2cc(C(F)(F)F)ccn2)nc1-c1ccccn1)N1CCOCC1] | morpholino(4- (pyridin-2- yl)-2-(4- (trifluoromethyl) pyridin-2- ylamino) thiazol-5- yl)methanone | 435.6 | A | | | | |
| 200 | 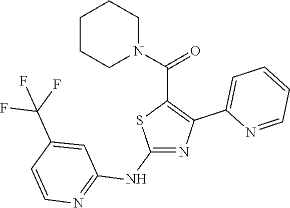 [SMILES: O=C(c1sc(Nc2cc(C(F)(F)F)ccn2)nc1-c1ccccn1)N1CCCCC1] | piperidin- 1-yl(4- (pyridin-2- yl)-2-(4- (trifluoromethyl) pyridin-2- ylamino) thiazol-5- yl)methanone | 433.6 | A | | | | |
| 201 | 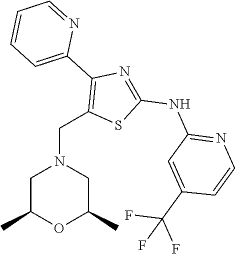 [SMILES: C[C@@H]1CN(Cc2sc(Nc3cc(C(F)(F)F)ccn3)nc2-c2ccccn2)C[C@H](C)O1] | 5- (((2R,6S)-2,6- dimethyl- morpholino) methyl)-4- (pyridin-2- yl)-N-(4- (trifluoromethyl) pyridin-2- yl)thiazol- 2-amine | 450.2 | C | | | | |
| 202 | 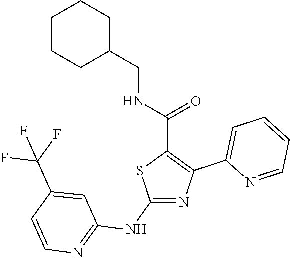 [SMILES: O=C(NCC1CCCCC1)c1sc(Nc2cc(C(F)(F)F)ccn2)nc1-c1ccccn1] | N- (cyclohexyl- methyl)- 4-(pyridin- 2-yl)-2-(4- (trifluoromethyl) pyridin-2- ylamino) thiazole-5- carboxamide | 450.2 | A | | | | |
| 203 | 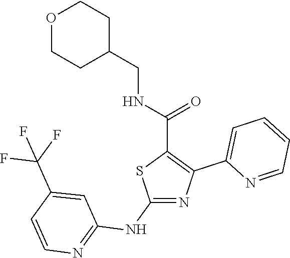 [SMILES: O=C(NCC1CCOCC1)c1sc(Nc2cc(C(F)(F)F)ccn2)nc1-c1ccccn1] | 4-(pyridin- 2-yl)-N- ((tetrahydro- 2H- pyran-4- yl)methyl)- 2-(4- (trifluoromethyl) pyridin-2- ylamino) thiazole-5- carboxamide | 463.6 | A | | | | |
| 204 | 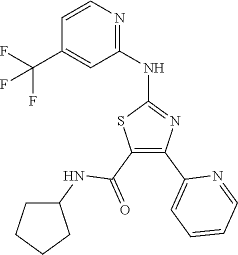 [SMILES: O=C(NC1CCCC1)c1sc(Nc2cc(C(F)(F)F)ccn2)nc1-c1ccccn1] | N- cyclopentyl-4- (pyridin-2- yl)-2-(4- (trifluoromethyl) pyridin-2- ylamino) thiazole- 5-carboxamide | 433.7 | A | | | | |
| 205 | 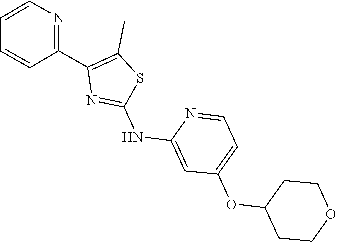 [SMILES: Cc1sc(Nc2cc(OC3CCOCC3)ccn2)nc1-c1ccccn1] | 5-methyl-4- (pyridin- 2-yl)-N-(4- (tetrahydro-2H- pyran-4- yloxy) pyridin-2- yl)thiazol- 2-amine | 369.2 | C | D, E | | D | |
| 206 | 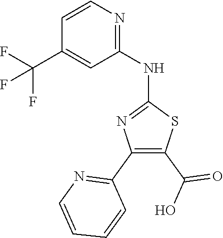 [SMILES: O=C(O)c1sc(Nc2cc(C(F)(F)F)ccn2)nc1-c1ccccn1] | 4-(pyridin- 2-yl)- 2-(4- (trifluoromethyl) pyridin- 2- ylamino) thiazole-5- carboxylic acid | 366.7 | C | | | | |
| 207 | 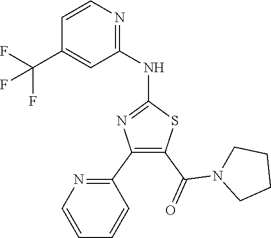 [SMILES: O=C(c1sc(Nc2cc(C(F)(F)F)ccn2)nc1-c1ccccn1)N1CCCC1] | (4-(pyridin- 2-yl)-2- (4- (trifluoromethyl) pyridin-2- ylamino) thiazol-5- yl)(pyrrolidin-1- yl)methanone | 419.7 | C | | | | |
| 208 | 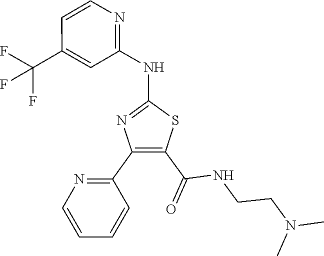 [SMILES: CN(C)CCNC(=O)c1sc(Nc2cc(C(F)(F)F)ccn2)nc1-c1ccccn1] | N-(2- (dimethylamino) ethyl)- 4-(pyridin- 2-yl)-2- (4- (trifluoro- methyl) pyridin-2- ylamino) thiazole-5- carboxamide | 436.7 | C | | | | |
| 209 | 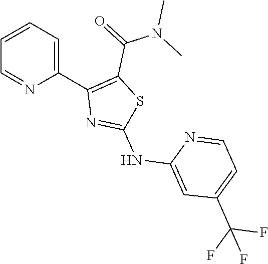 [SMILES: CN(C)C(=O)c1sc(Nc2cc(C(F)(F)F)ccn2)nc1-c1ccccn1] | N,N- dimethyl-4- (pyridin- 2-yl)-2-(4- (trifluoromethyl) pyridin-2- ylamino) thiazole-5- carboxamide | 393.7 | C | | | | |
| 210 | 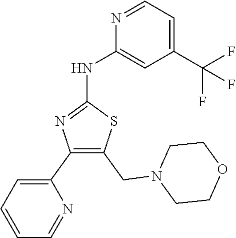 [SMILES: FC(F)(F)c1ccnc(Nc2nc(-c3ccccn3)c(CN3CCOCC3)s2)c1] | 5- (morpholino- methyl)- 4-(pyridin- 2-yl)-N- (4- (trifluoromethyl) pyridin-2-yl) thiazol-2-amine | 422.2 | C | | | | |
| 211 | 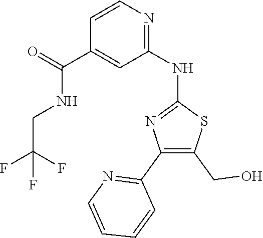 [SMILES: O=C(NCC(F)(F)F)c1ccnc(Nc2nc(-c3ccccn3)c(CO)s2)c1] | 2-(5- (hydroxy- methyl)-4- (pyridin-2- yl)thiazol- 2-ylamino)- N-(2,2,2- trifluoroethyl) isonicotinamide | 410.1 | B | | | | |
| 212 | 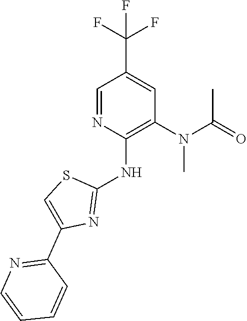 [SMILES: CC(=O)N(C)c1cc(C(F)(F)F)cnc1Nc1nc(-c2ccccn2)cs1] | N-methyl- N-(2-(4- (pyridin-2- yl)thiazol-2- ylamino)-5- (trifluoro- methyl) pyridin-3-yl) acetamide | 394.30 | C | D, E | D, E | D | A |
| 213 | 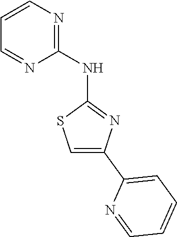 [SMILES: c1ccc(-c2csc(Nc3ncccn3)n2)nc1] | 4-(pyridin- 2-yl)-N- (pyrimidin-2- yl)thiazol- 2-amine | 256.20 | A | D | | | |

TABLE 3

| | | | | | | | L. | |
|---|---|---|---|---|---|---|---|---|
| | | | | O. gutt | B. | B. | sigmodontis | D. |
| Com. | | | | Act | malayi | phangi | Act | immitis |
| No. | Structure | Name | M + 1 | class | Act class | Act class | class | Act class |
| 214 | 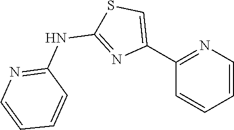 [SMILES: c1ccc(Nc2nc(-c3ccccn3)cs2)nc1] | N,4-di(pyridin-2- yl)thiazol-2-amine | 255.3 | B | D, E | | D | |
| 215 | 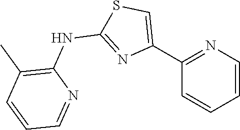 [SMILES: Cc1cccnc1Nc1nc(-c2ccccn2)cs1] | N-(3-methylpyridin-2- yl)-4-(pyridin-2- yl)thiazol-2-amine | 269.4 | C | D | | D | |
| 216 | 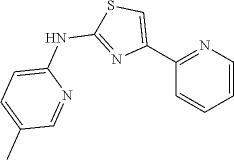 [SMILES: Cc1ccc(Nc2nc(-c3ccccn3)cs2)nc1] | N-(5-methylpyridin-3- yl)-4-(pyridin-2- yl)thiazol-2-amine | 269.4 | C | D | | D | B |
| 217 | 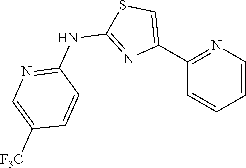 [SMILES: FC(F)(F)c1ccc(Nc2nc(-c3ccccn3)cs2)nc1] | 4-(pyridin-2-yl)-N-(5- (trifluoromethyl)pyridin- 2-yl)thiazol-2-amine | 323.17 | A | D, E | | D, E | C |
| 218 | 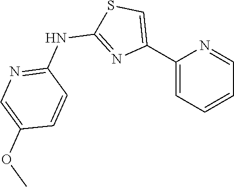 [SMILES: COc1ccc(Nc2nc(-c3ccccn3)cs2)nc1] | N-(5-methoxypyridin- 2-yl)-4-(pyridin-2- yl)thiazol-2-amine | 285.4 | B | D, E | | D, E | A |
| 219 | 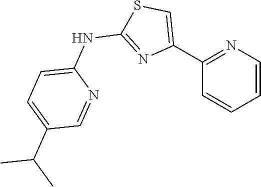 [SMILES: CC(C)c1ccc(Nc2nc(-c3ccccn3)cs2)nc1] | N-(5-isopropylpyridin- 2-yl)-4-(pyridin-2- yl)thiazol-2-amine | 297.4 | C | D, E | | | C |
| 220 | 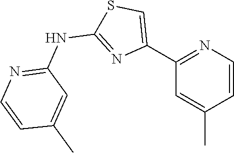 [SMILES: Cc1ccnc(Nc2nc(-c3cc(C)ccn3)cs2)c1] | N,4-bis(4- methylpyridin- 2-yl)thiazol-2-amine | 285.30 | C | D | | | C |
| 221 | 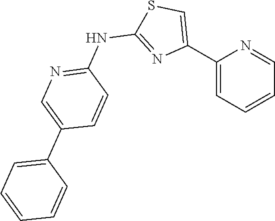 [SMILES: c1ccc(-c2ccc(Nc3nc(-c4ccccn4)cs3)nc2)cc1] | N-(5-phenylpyridin- 2-yl)-4-(pyridin-2- yl)thiazol-2-amine | 331.2 | C | D, E | | | C |
| 222 | 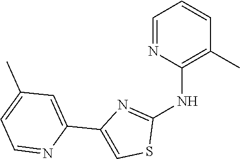 [SMILES: Cc1ccnc(-c2csc(Nc3ncccc3C)n2)c1] | N-(3-methylpyridin-2- yl)-4-(4-methylpyridin- 2-yl)thiazol-2-amine | 283.3 | A | | D | D, E | C |
| 223 | 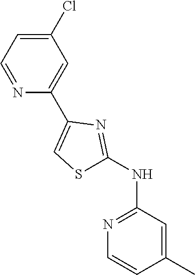 [SMILES: Cc1ccnc(Nc2nc(-c3cc(Cl)ccn3)cs2)c1] | 4-(4-chloropyridin- 2-yl)- N-(4-methylpyridin-2- yl)thiazol-2-amine | 302.9 | C | | | | C |
| 224 | 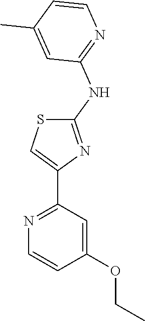 [SMILES: CCOc1ccnc(-c2csc(Nc3cc(C)ccn3)n2)c1] | 4-(4-ethoxypyridin-2- yl)-N-(4-methylpyridin- 2-yl)thiazol-2-amine | 313.1 | B | | | | B |
| 225 | 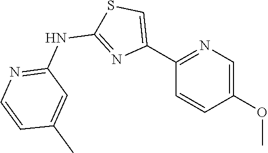 [SMILES: COc1ccc(-c2csc(Nc3cc(C)ccn3)n2)nc1] | 4-(5-methoxypyridin-2- yl)-N-(4-methylpyridin- 2-yl)thiazol-2-amine | 299.3 | C | D | | D, E | B |
| 226 | 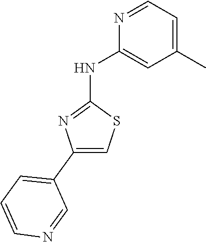 [SMILES: Cc1ccnc(Nc2nc(-c3cccnc3)cs2)c1] | N-(4-methylpyridin-2- yl)-4-(pyridin-3- yl)thiazol-2-amine | 269.1 | | | | | B |
| 227 | 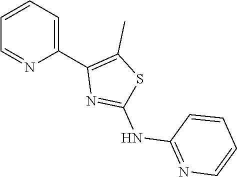 [SMILES: Cc1sc(Nc2ccccn2)nc1-c1ccccn1] | 5-methyl-N,4-di(pyridin- 2-yl)thiazol-2-amine | 269.1 | C | | | | C |

TABLE 4

| | | | | O | | | L. | |
|---|---|---|---|---|---|---|---|---|
| | | | | gutt | B. | B. | sigmodontis | D. |
| Com. | | | | Act | malayi | phangi | Act | immitis |
| No. | Structure | Name | M + 1 | class | Act class | Act class | class | Act class |
| 228 | 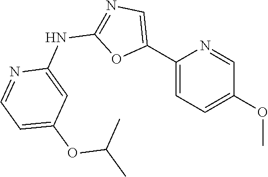 [SMILES: COc1ccc(-c2cnc(Nc3cc(OC(C)C)ccn3)o2)nc1] | N-(4- isopropoxy- pyridin- 2-yl)-5-(5- methoxypyridin- 2- yl)oxazol- 2-amine | 327.2 | A | | | | |
| 229 | 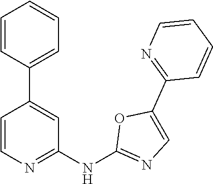 [SMILES: c1ccc(-c2ccnc(Nc3ncc(-c4ccccn4)o3)c2)cc1] | N-(4- phenylpyridin- 2- yl)-5-(pyridin- 2- yl)oxazol-2- amine | 315.3 | A | | | | |
| 230 | 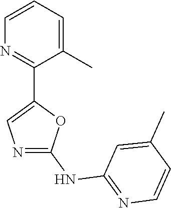 [SMILES: Cc1ccnc(Nc2ncc(-c3ncccc3C)o2)c1] | 5-(3- methylpyridin- 2- yl)-N-(4- methylpyridin- 2- yl)oxazol- 2-amine | 267.2 | A | | | | |
| 231 | 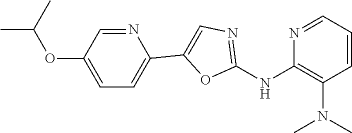 [SMILES: CC(C)Oc1ccc(-c2cnc(Nc3ncccc3N(C)C)o2)nc1] | N2-(5-(5- isopropoxy- pyridin- 2-yl)oxazol- 2-yl)- N3,N3- dimethyl- pyridine- 2,3-diamine | 340.2 | A | | | D | |
| 232 | 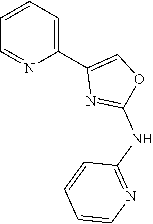 [SMILES: c1ccc(Nc2nc(-c3ccccn3)co2)nc1] | N,4-di (pyridin-2- yl)oxazol- 2-amine | 239.1 | C | D | | D | |

TABLE 5

| | | | | O | | | L. | |
|---|---|---|---|---|---|---|---|---|
| | | | | gutt | B. | B. | sigmodontis | D. |
| Com. | | | | Act | malayi | phangi | Act | immitis |
| No. | Structure | Name | M + 1 | class | Act class | Act class | class | Act class |
| 233 | 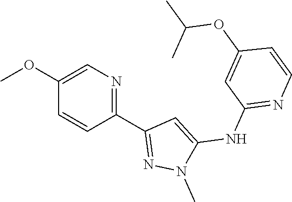 [SMILES: COc1ccc(-c2cc(Nc3cc(OC(C)C)ccn3)n(C)n2)nc1] | 4-isopropoxy-N-(3-(5- methoxypyridin-2- yl)-1-methyl- 1H-pyrazol-5-yl)pyridin-2- amine | 340.2 | A | | | | |
| 234 | 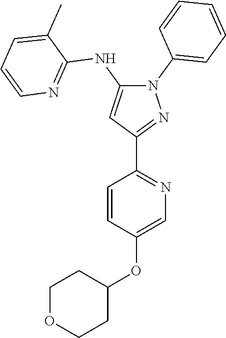 [SMILES: Cc1cccnc1Nc1cc(-c2ccc(OC3CCOCC3)cn2)nn1-c1ccccc1] | 3-methyl-N-(1-phenyl-3-(5- (tetrahydro-2H-pyran-4- yloxy)pyridin-2-yl)- 1H-pyrazol- 5-yl)pyridin-2-amine | 428.3 | A | | | | |
| 235 | 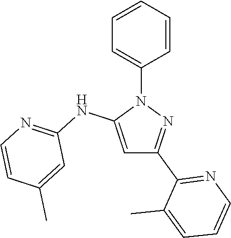 [SMILES: Cc1ccnc(Nc2cc(-c3ncccc3C)nn2-c2ccccc2)c1] | 4-methyl-N-(3-(3- methylpyridin-2-yl)- 1-phenyl-1H-pyrazol- 5-yl)pyridin-2- amine | 342 | A | | | | |
| 236 | 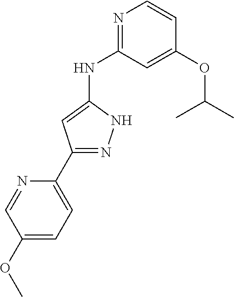 [SMILES: COc1ccc(-c2cc(Nc3cc(OC(C)C)ccn3)[nH]n2)nc1] | 4-isopropoxy-N-(3-(5- methoxypyridin- 2-yl)-1H-pyrazol- 5-yl)pyridin-2-amine | 326.2 | A | | D, E | D | |
| 237 | 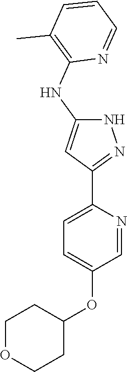 [SMILES: Cc1cccnc1Nc1cc(-c2ccc(OC3CCOCC3)cn2)n[nH]1] | 3-methyl-N-(3-(5- (tetrahydro- 2H-pyran-4-yloxy) pyridin-2-yl)-1H- pyrazol-5-yl)pyridin-2- amine | 352.2 | A | | | | |
| 238 | 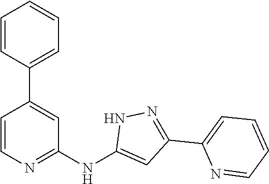 [SMILES: c1ccc(-c2ccnc(Nc3cc(-c4ccccn4)n[nH]3)c2)cc1] | 4-phenyl-N-(3- (pyridin-2-yl)-1H- pyrazol-5-yl)pyridin-2- amine | 313.9 | C | | | | |

A number of references have been cited, the disclosures of which are incorporated herein by reference in their entirety for all intent and purposes.

What is claimed is:

1. A compound of formula (I) 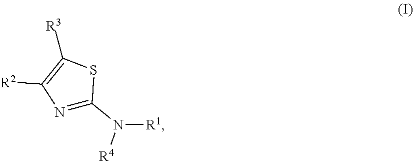
or a pharmaceutically acceptable salt, tautomer, isotopologue, or stereoisomer thereof,
wherein:
R¹ is isoquinolyl; pyrrolopyridyl; 2-pyrimidyl, or 2-pyridyl, wherein the 2-pyridyl is substituted with one or more substituents independently selected from halogen, —CN, substituted or unsubstituted C₁₋₄ alkyl, substituted or unsubstituted C₃₋₇ cycloalkyl, substituted or unsubstituted C₆₋₁₀ aryl, substituted or unsubstituted monocyclic heteroaryl, substituted or unsubstituted 3-6 membered heterocyclyl, —OR⁵, —SR, —CONR⁶₂, —CON(C₁₋₃ alkyl)(substituted or unsubstituted C₃₋₇ cycloalkyl), —NRCO(C₁₋₃ alkyl), —CO(substituted or unsubstituted 3-6 membered heterocyclyl), —SO₂NR₂, and SO₂R⁵;
R² is 5,6-dihydro-4H-pyrrolo[1,2-b]pyrazolyl, 2-pyrimidyl, or 2-pyridyl, substituted with one or more substituents independently selected from halogen, —CN, substituted or unsubstituted C₁₋₄ alkyl, substituted or unsubstituted C₆₋₁₀ aryl, —OR⁵, —SR, —CONR₂, and —SO₂R⁵, or two atoms together with the carbons to which they are attached form a substituted or unsubstituted 5-6 membered heterocyclyl;
R³ is H, —CN, substituted or unsubstituted C₁₋₄ alkyl, (C₁₋₃ alkyl)O(C₁₋₃ alkyl)(substituted or unsubstituted C₃₋₇ cycloalkyl), (C₁₋₃ alkyl)OR, (C₁₋₃ alkyl)(substituted or unsubstituted 3-6 membered heterocyclyl), C(O)(substituted or unsubstituted 3-10 membered heterocyclic), —C(O)OR, substituted or unsubstituted C₆₋₁₀ aryl, (C₁₋₃ alkyl)NR⁶₂, (C₁₋₃ alkyl)N(C₁₋₃ alkyl)(substituted or unsubstituted C₃₋₇ cycloalkyl), CONR⁶₂, or —C(O)N(C₁₋₃ alkyl)NR₂;
R⁴ is H, substituted or unsubstituted C₁₋₃ alkyl, or substituted or unsubstituted (C₁₋₃ alkyl) C₆₋₁₀ aryl;
R⁵ is H, substituted or unsubstituted C₁₋₅ alkyl, substituted or unsubstituted C₃₋₇ cycloalkyl, or substituted or unsubstituted 3-6 membered heterocyclyl;
each R⁶ is independently selected from H, substituted or unsubstituted C₁₋₅ alkyl; substituted or unsubstituted C₃₋₆ cycloalkyl, substituted or unsubstituted 3-6 membered heterocyclyl, and (C₁₋₃ alkyl)(substituted or unsubstituted 3-6 membered heterocyclyl); and
each R is independently selected from H and substituted or unsubstituted C₁₋₄ alkyl;
provided the compound is not 4-methyl-N-[4-(4-methyl-2-pyridinyl)-2-thiazolyl]-2-pyridinamine or N-(5-chloropyridin-2-yl)-4-(pyrimidin-2-yl)thiazol-2-amine.

2. The compound of claim 1, wherein R¹ is 2-pyridyl, substituted with one or more substituents independently selected from F, Br, Cl, —CN, —CH₃, —CH₂CH₃, —CH₂CH₂CH₃, —CH₂CH₂CH₂CH₃, —CH(CH₃)₂, CF₃, cyclopropyl, cylobutyl, cyclopentyl, —OR⁵, —SR, substituted or unsubstituted phenyl, substituted or unsubstituted pyrazolyl, substituted or unsubstituted dihydropyranyl, substituted or unsubstituted piperidyl, substituted or unsubstituted piperazinyl, substituted or unsubstituted pyrrolidinonyl, —CONR⁶₂, —CON(C₁₋₃ alkyl)(substituted or unsubstituted C₃₋₇ cycloalkyl), —NRCO(C₁₋₃ alkyl), —CO(substituted or unsubstituted 3-6 membered heterocyclyl), —SO₂NR₂ and —SO₂R⁵.

3. The compound of claim 2, wherein R⁵ is H, —CH₃, —CH₂CH₃, —CH₂CH₂CH₃, —CH(CH₃)₂, tetrahydrofuranyl, tetrahydropyranyl, or 1-methylpiperidyl.

4. The compound of claim 2, wherein R⁵ is H, —CH₃, —CH(CH₃)₂, tetrahydropyranyl, or 1-methylpiperidyl.

5. The compound of claim 2, wherein each R⁶ is independently selected from H, substituted or unsubstituted C₁₋₅ alkyl; substituted or unsubstituted C₃₋₆ cycloalkyl; wherein the alkyl and cycloalkyl are optionally substituted with one or more substituents independently selected from OH, OCH₃ and F.

6. The compound of claim 5, wherein the C₁₋₅ alkyl is selected from —CH₃, —CH₂CH₃, —CH₂CH₂CH₃, and —CH(CH₃)₂.

7. The compound of claim 5, wherein the C₁₋₅ alkyl is —CH(CH₃)₂.

8. The compound of claim 5, where the C₃₋₆ cycloalkyl is selected from cyclopropyl, cyclobutyl, cyclopentyl, and cyclohexyl.

9. The compound of claim 5, where the C₃₋₆ cycloalkyl is selected from cyclopentyl, and cyclohexyl.

10. The compound of claim 1, wherein R¹ is 2-pyridyl, substituted with one or more substituents independently selected from F, Br, Cl, —CN, —CH₃, —CH₂CH₃, —CH₂CH₂CH₃, —CH₂CH₂CH₂CH₃, —CH(CH₃)₂, CF₃, cyclopropyl, cylobutyl, cyclopentyl, —OH, —OCH₃, —OCH₂CH₃, —OCH₂CH₂CH₃, —OCH(CH₃)₂, —O-tetrahydrofuranyl, —O-tetrahydropyranyl, —SCH₃, substituted or unsubstituted phenyl, substituted or unsubstituted pyrazolyl, substituted or unsubstituted dihydropyranyl, substituted or unsubstituted piperidyl, substituted or unsubstituted piperazinyl, substituted or unsubstituted pyrrolidinonyl, —CONH₂, —CONH₂, —CONHCH₃, —CON(CH₃)₂, —CONHCH₂CH₃, —CON(CH₂CH₃)₂, —CONHCH₂CH₂CH₃—CONHCH(CH₃)₂, —CONHCH₂CH₂OH, —CONHCH₂CH₂OCH₃, —CONHCH(CH₃)CH₂OH, —CONHCH₂CF₃, —CONHCH₂CH₂-pyrrolidyl, —CONH(cyclopropyl), —CONH(cyclobutyl), —CONH(cyclopentyl), —CONH(cyclohexyl), —NCH₃COCH₃, —SO₂N(CH₃)₂, —SO₂(aziridinyl), —CO(azetidyl), CO(piperidyl), —CO(piperazinyl), and —CO(morpholinyl); wherein the cyclopropyl, cyclobutyl, cyclopentyl, cyclohexyl, azetidyl, piperidyl and piperazinyl are optionally fluorinated.

11. The compound of claim 1, wherein R¹ is 2-pyridyl, substituted with one or more substituents independently selected from F, Br, Cl, —CN, —CH₃, —CH₂CH₃, —CH₂CH₂CH₃, —CH₂CH₂CH₂CH₃, —CH(CH₃)₂, —CF₃, cyclopropyl, cyclopentyl, —OH, —OCH₃, —OCH₂CH₃, —OCH(CH₃)₂, —O-tetrahydropyranyl, —SCH₃, substituted or unsubstituted phenyl, substituted or unsubstituted pyrazolyl, substituted or unsubstituted dihydropyranyl, substituted or unsubstituted piperidyl, substituted or unsubstituted piperazinyl, substituted or unsubstituted pyrrolidinonyl, —CONH₂, —CONHCH(CH₃)₂, —CONHCH₂CH₂OH, —CONHCH₂CH₂OCH₃, —CONHCH(CH₃)CH₂OH, —CONHCH₂CF₃, —CONHCH₂CH₂-pyrrolidyl, —CONH(cyclopentyl), —CONH(cyclohexyl), —NCH₃COCH₃, —SO₂N(CH₃)₂, —SO₂(aziridinyl), —CO(azetidyl), —CO(piperidyl), —CO(piperazinyl), and —CO(morpholinyl); wherein the cyclopropyl, cyclobutyl, cyclopentyl, cyclohexyl, azetidyl, piperidyl and piperazinyl are optionally fluorinated.

12. The compound of claim 1, wherein R¹ is 2-pyridyl, substituted with one or more substituents independently selected from F, Br, Cl, —CN, —CH₃, —CH₂CH₃, —CH₂CH₂CH₃, —CH₂CH₂CH₂CH₃, —CH(CH₃)₂, CF₃, cyclopropyl, cyclopentyl, —OH, —OCH₃, OCH₂CH₃, —OCH(CH₃)₂, —O-tetrahydropyranyl, —SCH₃, phenyl; phenyl(COOH); pyrrolidinonyl, 1-methylpyrazolyl; dihydropyranyl; 1-methyl-piperidyl; piperidyl substituted with —COOH; —CONHCH₃, —CON(CH₃)₂, or —CONHCH₂CF₃; 1-methyl-piperazinyl; piperazinyl substituted with —COC(CH₃)₂OH or —CO-cyclopropyl-CF₃; —CONH₂, —CON(CH₃)₂, —CONHCH(CH₃)₂, —CONHCH₂CH₂OH, —CONHCH₂CH₂OCH₃, —CONHCH(CH₃)CH₂OH, —CONHCH₂CF₃, —CONHCH₂CH₂-pyrrolidyl, —CONH(cyclopentyl), —CONH(difluorocyclohexyl), —NCH₃COCH₃, —SO₂N(CH₃)₂, —SO₂(aziridinyl), —CO(difluoroazetidyl), CO(difluoropiperidyl), —CO(piperazinyl), and —CO(morpholinyl).

13. The compound of claim 1, wherein R² is 2-pyridyl, substituted with F, Cl, —CN, —CH₃, —CH₂CH₃, —CF₃, —CHF₂, substituted or unsubstituted phenyl, —OR⁵, —SR, —CONR₂, or —SO₂R⁵.

14. The compound of claim 13, wherein R⁵ is H, —CH₃, —CH₂CH₃, —CH₂CH₂CH₃, —CH(CH₃)₂, —CH₂CF₃, cyclopropyl, oxetanyl, 1-methyl-azetidinyl, piperidyl, 1-methyl-piperidyl, tetrahydrofuranyl, or tetrahydropyranyl.

15. The compound of claim 13, wherein R⁵ is CH₃, —CH₂CH₃, —CH(CH₃)₂, —CHF₂, —CH₂CF₃, cyclopropyl, oxetanyl, 1-methyl-azetidinyl, 1-methyl-piperidyl, or tetrahydropyranyl.

16. The compound of claim 13, wherein R⁵ is H or —CH₃.

17. The compound of claim 1, wherein R² is 2-pyridyl, substituted with one or more substituents independently selected from F, Cl, —CN, —CH₃, —CH₂CH₃, —CF₃, —CHF₂, —OCH₃, —OCH₂CH₃, —OCH₂CH₂CH₃, —OCH(CH₃)₂, —OCH₂CF₃, —O-cyclopropyl, —O-oxetanyl, —O-(1-methyl-azetidinyl), —O-(1-methyl-piperidyl), —O-tetrahydrofuranyl, —O-tetrahydropyranyl, —SCH₃, —CONH₂, —CONHCH₃, —CON(CH₃)₂, —CONHCH₂CH₃, —CON(CH₂CH₃)₂, —SO₂CH₃, and substituted or unsubstituted phenyl.

18. The compound of claim 1, wherein R² is 2-pyridyl, substituted with one or more substituents independently selected from F, Cl, —CN, —CH₃, —CH₂CH₃, —CF₃, —CHF₂, —OCH₃, —OCH₂CH₃, —OCH(CH₃)₂, —OCH₂CF₃, —O-cyclopropyl, —O-oxetanyl, —O-(1-methyl-azetidinyl), —O-(1-methyl-piperidyl), —O-tetrahydropyranyl, —SCH₃, —CONH₂, —CON(CH₃)₂, —SO₂CH₃, and substituted or unsubstituted phenyl.

19. The compound of claim 1, wherein R² is 2-pyridyl, substituted with one or more substituents independently selected from F, Cl, —CN, —CH₃, —CH₂CH₃, —CF₃, —CHF₂, —OCH₃, —OCH₂CH₃, —OCH(CH₃)₂, —OCH₂CF₃, —O-cyclopropyl, —O-oxetanyl, —O-(1-methyl-azetidinyl), —O-(1-methyl-piperidyl), —O-tetrahydropyranyl, —SCH₃, —CONH₂, —CON(CH₃)₂, —SO₂CH₃, phenyl; and phenyl, substituted with cyclopropyl(COOH).

20. The compound of claim 1, wherein R² is 2-pyridyl, wherein two atoms together with the carbons to which they are attached form a substituted or unsubstituted 5-6 membered heterocyclyl.

21. The compound of claim 20, wherein R² is a substituted or unsubstituted 2,3-dihydrofuro[2,3-c]pyridyl, 2,3-dihydro-1H-pyrrolo[2,3-c]pyridyl, or 1,3-dihydro-2H-pyrrolo[2,3-c]pyridyl-2-one.

22. The compound of claim 1, wherein R² is 5,6-dihydro-4H-pyrrolo[1,2-b]pyrazolyl, or 2-pyrimidyl.

23. The compound of claim 1, wherein R³ is H, —CN, —CH₃, —CH₂CH₃, —CH(CH₃)₃, —CH₂OH, —CH₂CH₂OH, —CH₂OCH₂-cyclopropyl, —CH₂OCH₂-cyclobutyl, —CH₂CH₂O-cyclobutyl, —CH₂CH₂OCH₂-cyclopropyl, —CH₂OCH₂CH₂-cyclopropyl, —CH₂N(CH₃)₂, —CH₂-azetidyl, —CH₂-piperidyl, —CH₂(dimethyl morpholinyl), —CH₂(dimethyl piperazyl), —CH₂-pirrolidyl, —CH₂(morpholinyl), —COOH, —CO(dimethyl morpholinyl), —CO(morpholinyl), —CO(1,3-dioxolane-piperidyl), —CO(piperidyl), —CO(pirrolidyl), —CO(1-methyl-piperazyl), —CO(octahydropyrrolo[1,2-a]pyrazyl), —CONHCH₂-cyclohexyl, —CONHCH₂-tetrahydropiranyl, —CONHCH₂-cyclopentyl, —CONH(CH₂)₂N(CH₃)₂, —CON(CH₃)₂, or phenyl.

24. The compound of claim 1, wherein R⁴ is H or —CH₃, or CH₂-phenyl.

25. The compound of claim 1, wherein the compound is selected from Table 1.

26. A compound of Table 2.

27. A compound of formula (III): 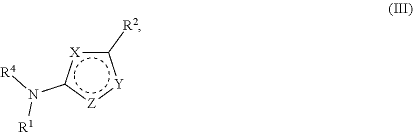
or a pharmaceutically acceptable salt, tautomer, isotopologue, or stereoisomer thereof,
wherein:
X is CR³, N, or S;
Y is N, or S;
Z is CR³, or S;
R¹ is 2-pyridyl, unsubstituted or substituted with one or more substituents independently selected from halogen, substituted or unsubstituted C₁₋₄ alkyl, substituted or unsubstituted C₃₋₇ cycloalkyl, substituted or unsubstituted C₆₋₁₀ aryl, substituted or unsubstituted monocyclic heteroaryl, substituted or unsubstituted 3-6 membered heterocyclyl, —OR⁵, —CONR⁶₂, and —CO(substituted or unsubstituted 3-6 membered heterocyclyl);
R² is 2-pyridyl, unsubstituted or substituted with one or more substituents independently selected from halogen, substituted or unsubstituted C₁₋₄ alkyl, substituted or unsubstituted C₆₋₁₀ aryl, —OR⁵, and —CONR₂;
R³ is H, substituted or unsubstituted C₁₋₄ alkyl, or (C₁₋₃ alkyl)O(C₁₋₃ alkyl)(substituted or unsubstituted C₃₋₇ cycloalkyl), (C₁₋₃ alkyl)OR;
R⁴ is H, substituted or unsubstituted C₁₋₃ alkyl;
R⁵ is H, substituted or unsubstituted C₁₋₅ alkyl, or unsubstituted 3-6 membered heterocyclyl;
each R⁶ is independently selected from H, substituted or unsubstituted C₁₋₅ alkyl; substituted or unsubstituted C₃₋₆ cycloalkyl, substituted or unsubstituted 3-6 membered heterocyclyl, and (C₁₋₃ alkyl)(substituted or unsubstituted 3-6 membered heterocyclyl); and
each R is independently selected from H and substituted or unsubstituted C₁₋₄ alkyl.

28. The compound of claim 27, wherein the compound is a compound of formula (IIIa): 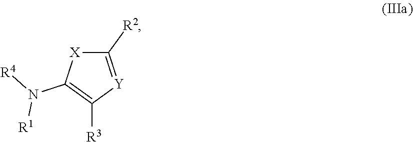
or a pharmaceutically acceptable salt, tautomer, isotopologue, or stereoisomer thereof.

29. The compound of claim 28, wherein X is N or S, and Y is N or S.

30. The compound of claim 28 or 29, wherein the compound is a compound of formula (IIIb): 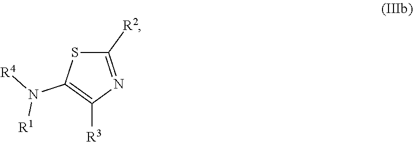
or a pharmaceutically acceptable salt, tautomer, isotopologue, or stereoisomer thereof.

31. The compound of claim 28 or 29, wherein the compound is a compound of formula (IIIc): 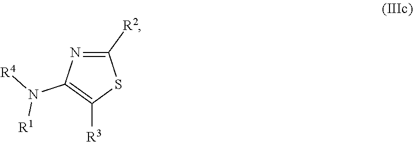
or a pharmaceutically acceptable salt, tautomer, isotopologue, or stereoisomer thereof.

32. The compound of claim 27, wherein the compound is a compound of formula (IIId): 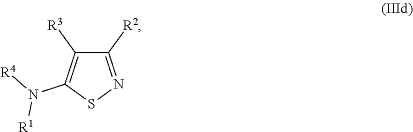
or a pharmaceutically acceptable salt, tautomer, isotopologue, or stereoisomer thereof.

33. The compound of any one of claims 27-32, wherein R¹ is substituted or unsubstituted with one or more substituents independently selected from halogen, substituted or unsubstituted C₁₋₄ alkyl, substituted or unsubstituted C₃₋₇ cycloalkyl, substituted or unsubstituted C₆₋₁₀ aryl, substituted or unsubstituted monocyclic heteroaryl, substituted or unsubstituted 3-6 membered heterocyclyl, —OR⁵, —CONR₆₂, and —CO(substituted or unsubstituted 3-6 membered heterocyclyl).

34. The compound of any one of claims 27-33, wherein R¹ is unsubstituted or substituted with one or more substituents independently selected from —CH₃, —CH₂CH₃, —CH₂CH₂CH₃, —CH(CH₃)₂, cyclopropyl, cylobutyl, cyclopentyl, —OR⁵, substituted or unsubstituted phenyl, substituted or unsubstituted pyrazolyl, substituted or unsubstituted dihydropyranyl, substituted or unsubstituted piperidyl, substituted or unsubstituted piperazinyl, —CONR₆₂, and —CO(substituted or unsubstituted 3-6 membered heterocyclyl).

35. The compound of any one of claims 27-34, wherein R¹ is unsubstituted or substituted with one or more substituents independently selected from —CH₃, —CH₂CH₃, —CH₂CH₂CH₃, —CH(CH₃)₂, cyclopropyl, cylobutyl, cyclopentyl, —OR⁵, substituted or unsubstituted phenyl, and CONR⁶₂.

36. The compound of any one of claims 27-35, wherein R² is 2-pyridyl, unsubstituted or substituted with one or more substituents independently selected from —CH₃, —CH₂CH₃, —CH₂CH₂CH₃, —CH(CH₃)₂, cyclopropyl, cyclobutyl, cyclopentyl, substituted or unsubstituted phenyl, —OR⁵, and —CONR₂.

37. The compound of any one of claims 27-36, wherein R⁵ is H, —CH₃, —CH₂CH₃, —CH₂CH₂CH₃, —CH(CH₃)₂, tetrahydrofuranyl, tetrahydropyranyl, or 1-methylpiperidine.

38. The compound of any one of claims 27-37, wherein R⁵ is H, —CH₃, —CH₂CH₃, —CH(CH₃)₂, or tetrahydropyranyl.

39. The compound of any one of claims 27-38, wherein R⁶ is independently selected from H, substituted or unsubstituted C₁₋₅ alkyl; substituted or unsubstituted C₃₋₆ cycloalkyl, substituted or unsubstituted 3-6 membered heterocyclyl, and (C₁₋₃ alkyl)(substituted or unsubstituted 3-6 membered heterocyclyl).

40. The compound of any one of claims 27-39, wherein R¹ is 2-pyridyl, unsubstituted or substituted with one or more substituents independently selected from —CH₃, —OCH(CH₃)₂, and substituted or unsubstituted phenyl.

41. The compound of any one of claims 27-40, wherein R² is 2-pyridyl, unsubstituted or substituted with one or more substituents independently selected from —CH₃, —OCH₃, —O— tetrahydrofuranyl, and substituted or unsubstituted phenyl.

42. The compound of any one of claims 27-41, wherein R³ is H, or —CH₃.

43. The compound of any one of claims 27-42, wherein R⁴ is H, or —CH₃.

44. The compound of any one of claims 27-43, wherein the compound is selected from Table 1.

45. A compound of formula (IV): 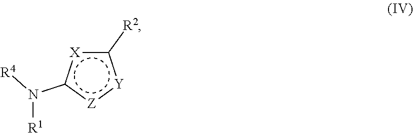
or a pharmaceutically acceptable salt, tautomer, isotopologue, or stereoisomer thereof,
wherein:
X is O, or CR³;
Y is NRⁿ, or CR³;
Z is N, or NRⁿ;
R¹ is 2-pyridyl, unsubstituted or substituted with one or more substituents independently selected from halogen, substituted or unsubstituted C₁₋₄ alkyl, substituted or unsubstituted C₃₋₇ cycloalkyl, substituted or unsubstituted C₆₋₁₀ aryl, substituted or unsubstituted monocyclic heteroaryl, substituted or unsubstituted 3-6 membered heterocyclyl, —OR⁵, —CONR⁶₂, —CO(substituted or unsubstituted 3-6 membered heterocyclyl), and —NR₂;
R² is 2-pyridyl, unsubstituted or substituted with one or more substituents independently selected from halogen, substituted or unsubstituted C₁₋₄ alkyl, substituted or unsubstituted C₆₋₁₀ aryl, —OR⁵, and —CONR₂;
R³ is H, substituted or unsubstituted C₁₋₄ alkyl, or (C₁₋₃ alkyl)O(C₁₋₃ alkyl)(substituted or unsubstituted C₃₋₇ cycloalkyl), (C₁₋₃ alkyl)OR;
R⁴ is H, substituted or unsubstituted C₁₋₃ alkyl;
R⁵ is H, substituted or unsubstituted C₁₋₅ alkyl, or unsubstituted 3-6 membered heterocyclyl;
each R⁶ is independently selected from H, substituted or unsubstituted C₁₋₅ alkyl; substituted or unsubstituted C₃₋₆ cycloalkyl, substituted or unsubstituted 3-6 membered heterocyclyl, and (C₁₋₃ alkyl)(substituted or unsubstituted 3-6 membered heterocyclyl); and
each R is independently selected from H and substituted or unsubstituted C₁₋₄ alkyl;
each Rⁿ is independently H, substituted or unsubstituted C₁₋₄ alkyl, or substituted or unsubstituted C₆₋₁₀ aryl.

46. The compound of claim 45, wherein the compound has formula (IVa): 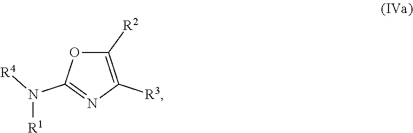
or a pharmaceutically acceptable salt, tautomer, isotopologue, or stereoisomer thereof.

47. The compound of claim 45, wherein the compound has formula (IVb): 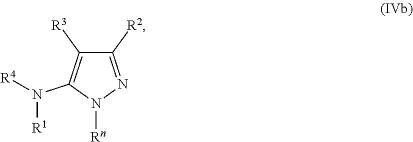
or a pharmaceutically acceptable salt, tautomer, isotopologue, or stereoisomer thereof.

48. The compound of claim 45, wherein the compound has formula (IVc): 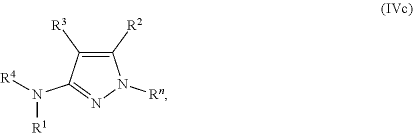
or a pharmaceutically acceptable salt, tautomer, isotopologue, or stereoisomer thereof.

49. The compound of any one of claims 45-48, wherein R¹ is 2-pyridyl, substituted or unsubstituted with one or more substituents independently selected from halogen, substituted or unsubstituted C₁₋₄ alkyl, substituted or unsubstituted C₃₋₇ cycloalkyl, substituted or unsubstituted C₆₋₁₀ aryl, substituted or unsubstituted monocyclic heteroaryl, substituted or unsubstituted 3-6 membered heterocyclyl, —OR⁵, —CONR⁶₂, —CO(substituted or unsubstituted 3-6 membered heterocyclyl) and —NR₂.

50. The compound of any one of claims 45-49, wherein R¹ is 2-pyridyl, unsubstituted or substituted with one or more substituents independently selected from —CH₃, —CH₂CH₃, —CH₂CH₂CH₃, —CH(CH₃)₂, cyclopropyl, cylobutyl, cyclopentyl, —OR⁵, substituted or unsubstituted phenyl, substituted or unsubstituted pyrazolyl, substituted or unsubstituted dihydropyranyl, substituted or unsubstituted piperidyl, substituted or unsubstituted piperazinyl, —CONR⁶₂, —CO(substituted or unsubstituted 3-6 membered heterocyclyl) and —N(CH₃)₂.

51. The compound of any one of claims 45-50, wherein R¹ is 2-pyridyl, unsubstituted or substituted with one or more substituents independently selected from —CH₃, —CH₂CH₃, —CH₂CH₂CH₃, —CH(CH₃)₂, cyclopropyl, cyclobutyl, cyclopentyl, —OR⁵, —CONR⁶₂, substituted or unsubstituted phenyl, and —N(CH₃)₂.

52. The compound of any one of claims 45-51, wherein R² is 2-pyridyl, unsubstituted or substituted with one or more substituents independently selected from —CH₃, —CH₂CH₃, —CH₂CH₂CH₃, —CH(CH₃)₂, cyclopropyl, cyclobutyl, cyclopentyl, substituted or unsubstituted phenyl, —OR⁵, and —CONR₂.

53. The compound of any one of claims 45-52, wherein R⁵ is H, —CH₃, —CH₂CH₃, —CH₂CH₂CH₃, —CH(CH₃)₂, tetrahydrofuranyl, tetrahydropyranyl, or 1-methylpiperidine.

54. The compound of any one of claims 45-53, wherein R⁵ is H, —CH₃, —CH₂CH₃, —CH(CH₃)₂, or tetrahydropyranyl.

55. The compound of any one of claims 45-54, wherein R⁶ is independently selected from H, substituted or unsubstituted C₁₋₅ alkyl; substituted or unsubstituted C₃₋₆ cycloalkyl, substituted or unsubstituted 3-6 membered heterocyclyl, and (C₁₋₃ alkyl)(substituted or unsubstituted 3-6 membered heterocyclyl).

56. The compound of any one of claims 45-55, wherein R¹ is 2-pyridyl, unsubstituted or substituted with one or more substituents independently selected from —CH₃, —OCH(CH₃)₂, substituted or unsubstituted phenyl, and —N(CH₃)₂.

57. The compound of any one of claims 45-56, wherein R² is 2-pyridyl, unsubstituted or substituted with one or more substituents independently selected from —CH₃, —OCH₃, —OCH(CH₃)₂, —O-tetrahydrofuranyl, and substituted or unsubstituted phenyl.

58. The compound of any one of claims 45-57, wherein R³ is H, or —CH₃.

59. The compound of any one of claims 45-58, wherein R⁴ is H, or —CH₃.

60. The compound of any one of claims 45-59, wherein Rⁿ is H, —CH₃, or unsubstituted or substituted phenyl.

61. The compound of any one of claims 45-60, wherein the compound is selected from Table 4.

62. The compound of any one of claims 45-61, wherein the compound is selected from Table 5.

63. A pharmaceutical composition comprising an effective amount of a compound of any one of claims 1, 25-62, or a pharmaceutically acceptable salt, tautomer, isotopologue, or stereoisomer thereof, and a pharmaceutically acceptable carrier, excipient or vehicle.

64. A method of killing a filarial worm, comprising contacting the filarial worm with a compound of any one of claim 1, or 25-62, or a pharmaceutically acceptable salt, tautomer, isotopologue, or stereoisomer thereof, in an amount effective to kill the filarial worm.

65. A method of inhibiting growth or molt of a filarial worm, comprising contacting the filarial worm with a compound of any one of claim 1, or 25-62, or a pharmaceutically acceptable salt, tautomer, isotopologue, or stereoisomer thereof, in an amount effective to inhibit growth or molt of the filarial worm.

66. A method of killing a filarial worm, comprising contacting the filarial worm with a compound of Table 3, or a pharmaceutically acceptable salt, tautomer, isotopologue, or stereoisomer thereof, in an amount effective to kill the filarial worm.

67. A method of inhibiting growth or molt of a filarial worm, comprising contacting the filarial worm with a compound of Table 3, or a pharmaceutically acceptable salt, tautomer, isotopologue, or stereoisomer thereof, in an amount effective to inhibit growth or molt of the filarial worm.

68. A method of inhibiting motility of a filarial worm, comprising contacting the filarial worm with a compound of any one of claim 1, or 25-62, or a pharmaceutically acceptable salt, tautomer, isotopologue, or stereoisomer thereof, in an amount effective to inhibit motility of the filarial worm.

69. A method of inhibiting motility of a filarial worm, comprising contacting the filarial worm with a compound of Table 3, or a pharmaceutically acceptable salt, tautomer, isotopologue, or stereoisomer thereof, in an amount effective to inhibit motility of the filarial worm.

70. A method for the treatment or prevention of helminthic infections and diseases, the methods comprising administering to a subject an effective amount of a compound of any one of claims 1, 25-62, or a pharmaceutically acceptable salt, tautomer, isotopologue, or stereoisomer thereof.

71. The method of claim 70, wherein the helminthic infection is a filarial worm infection.

72. A method for the treatment or prevention of helminthic infections and diseases, the methods comprising administering to a subject an effective amount of a compound of Table 3, or a pharmaceutically acceptable salt, tautomer, isotopologue, or stereoisomer thereof.

73. The method of claim 72, wherein the helminthic infection is a filarial worm infection.

74. A method for the treatment or prevention of helminthic infections and diseases, the methods comprising administering to a subject an effective amount of a compound of any one of claim 1, or 25-62, or a pharmaceutically acceptable salt, tautomer, isotopologue, or stereoisomer thereof in combination with one or more antihelminthic agent.

75. The method of claim 74, wherein the helminthic infection is a filarial worm infection.

76. A method for the treatment or prevention of helminthic infections and diseases, the methods comprising administering to a subject an effective amount of a compound of Table 3, or a pharmaceutically acceptable salt, tautomer, isotopologue, or stereoisomer thereof in combination with one or more antihelminthic agent.

77. The method of claim 76, wherein the helminthic infection is a filarial worm infection.

78. The method of claim 74 or 76, wherein the antihelminthic agent is selected from flubendazole, albendazole, mebendazole, thiabendazole, fenbendazole, triclabendazole, ivermectin, abamectin, diethylcarbamazine (DEC), suramin, pyrantel pamoate, levamisole, niclosamide, nitazoxanide, oxyclozanide, praziquantel, emodepside, monepantel, derquantel, or pelletierine sulphate.

79. The method of claim 74 or 76, wherein the antihelminthic agent is a Wolbachia targeting agent.

80. The method of claim 79, wherein the Wolbachia targeting agent is doxycycline.
